(19) United States
(12) Patent Application Publication
(10) Pub. No.: US 2025/0110134 A1
(43) Pub. Date: Apr. 3, 2025

(54) Stabilized Single Immunoglobulin Variable Domains

(71) Applicant: Tentarix Biotherapeutics, Inc., San Diego, CA (US)

(72) Inventors: Stephen John Demarest, San Diego, CA (US); Michael Lajos Gallo, North Vancouver, BC (CA); David Forrest Thieker, Durham, NC (US); Brian Arthur Kuhlman, Chapel Hill, NC (US)

(21) Appl. No.: 18/725,332

(22) PCT Filed: Jan. 10, 2023
(86) PCT No.: PCT/US2023/060405

§ 371 Date: Jun. 28, 2024

Related U.S. Application Data
(60) Provisional application No. 63/298,051, filed on Jan. 10, 2022.

(51) Int. Cl. G01N 33/68; C12N 15/10

(52) U.S. Cl. CPC G01N 33/6845; C12N 15/1055

(57) ABSTRACT

This disclosure relates to single immunoglobulin variable domains with amino substitutions that result in improved thermal stability, cellular expression, and other biophysical properties.

### CROSS-REFERENCE TO RELATED APPLICATIONS

This application claims priority to U.S. Provisional Application 63/298,051, filed Jan. 10, 2022, which is incorporated by reference in its entirety.

### REFERENCE TO AN ELECTRONIC SEQUENCE LISTING

The contents of the electronic sequence listing (21-1467-WO_ST26_Sequence_Listing.xml; Size: 960,098 bytes; and Date of Creation: Jan. 9, 2023) is herein incorporated by reference in its entirety.

### FIELD OF THE DISCLOSURE

This disclosure generally relates to single immunoglobulin variable domains with amino acid substitutions resulting in improved biophysical properties.

### BACKGROUND

Immunoglobulin therapeutics have become a large and growing sector of the pharmaceutical sector. Given their high specificity directed to single targets, minimal off-target cross-reactivity and generally good biophysical behavior, Immunoglobulin G (IgG) antibodies in particular, represent powerful tools to intercede in a highly specific manner in various disease processes. IgGs typically consist of two heavy chains (HCs) and two light chains (LCs) amino acid sequences of either kappa or lambda isotype that assemble into a heterotetramer. Once assembled, IgGs consist of two major subunits, the crystallizable fragment (Fc) and the antigen binding fragment (Fab), that perform different functions.

The Fab region of natural IgGs are highly diverse and comprise two variable domains, variable heavy (VH) and variable light (VL) from the HC and LC, that get further diversified by recombinant V-D-J (VH) or V-J (VL) joining as well as hypersomatic mutation to achieve nearly unlimited diversity that gets harnessed to optimize interactions towards target antigens. Fabs also contain a CH1/CL domain from the HC and LC, respectively, that are disulfide linked and exist to stabilize the VH/VL pairing. The VH domain, and particularly the HC complimentary determining region (HCDR) 3, is the most diverse region of an antibody based on the complexity of V-D-J joining and thus typically drives the specificity of antibody/antigen interactions.

IgG thermodynamics are relatively complex. The Fab and Fc subunits are thermodynamically distinct from one another. Demarest S J & Glaser S M, Curr Opin Drug Discov (2007) 11:675-87. Typically, IgG-Fcs unfold with two independent unfolding transitions with the CH2 domain demonstrating a midpoint of thermal unfolding (Tₘ) at ˜70° C. and the CH3 domain unfolding between 7° and 85° C. depending on the IgG subclass. Demarest S J, et al., J Biol Chem (2006) 281:30755-67; Garber E & Demarest S J, Biochem Biophys Res Commun (2007) 355:751-7). The domains within IgG Fabs that comprise kappa LCs (VH, Vkappa, CH1, Ckappa) are thermodynamically coupled and unfold in a cooperative fashion (Garber & Demarest 2007 (above); Toughiri R, et al., MAbs (2016) 8:1276-85), while Fabs with lambda LCs typically unfold using with two independent transitions, VH/Vlambda and CH1/Clambda, with each subunit highly stabilized by the heterodimeric interaction of the partnered domains.

The ability to isolate the VH domain to use as therapeutic results in both advantages and disadvantage over traditional IgG antibody therapeutics. Given the relatively small size of a VH domain (about 14 kDa) compared to a full-length antibody (about 150 kDa), and the fact VH domains drive both antigen specificity and much of the antibody binding strength, VH domains have the theoretical utility of being used as single domain binder to various antigens (Holt L J, Herring C, et al., Trends Biotechnol (2003) 21:484-90). This allows the use of small and modular binding units that do not require multi-chain heterodimerization to achieve a binding event. On the other hand, of the removal of VH domains from their Fab subunits, particularly for kappa-containing Fabs, leads to an approximate 20-25° C. decrease in Tm that can lead to significant challenges related to their thermal stability and folding (Michaelson J S, et al., MAbs (2009) 1:128-41; Demarest & Garber 2007 (above); Kim et al., Biochem Biophys Acta (2014) 1844:1983-2001.2014) making the VH domains challenging to use as a therapeutic due to poor expression and reduced pharmacokinetic profiles as compared to a complete Fab or antibody. Thus, optimization is typically required for VH domains to be used as therapeutic moieties independent of a full IgG.

Thus, there remains a need in the art to find substitutions to the VH germline families, VH1, VH2, VH3, VH4, VH5, VH6, and VH7, that can be used to improve their biophysical properties, including thermal stability and/or expression.

### SUMMARY

In various aspects, the disclosure is directed to a single immunoglobulin variable domain having an amino acid sequence of a human heavy chain V-gene portion (IGHV) of an antibody, wherein the IGHV amino acid sequence includes one or more amino acid substitutions that result in one or more of increased cellular expression, increased thermal stability, decreased dimerization, and decreased light chain pairing, as compared to a wild-type IGHV sequence lacking the one or more amino acid substitutions. The single chain immunoglobulin variable domain may also include a D gene sequence and/or a J gene sequence.

In another aspect, the disclosure is directed to single immunoglobulin variable domain, including an amino acid sequence of a framework region of a human heavy chain V-gene portion (IGHV) of an antibody, wherein the IGHV amino acid sequence comprises one or more amino acid substitutions or combinations thereof as described herein. The framework sequence may include a J gene sequence.

In another aspect, the disclosure is directed to at least one framework sequence selected from FR1, FR2, FR3, and FR4 of a single immunoglobulin heavy chain variable domain wherein the framework sequence comprises at least one of the substitutions or combinations thereof as described herein.

In the various aspects of the disclosure, the one or more substitutions may include at least one of the following amino acids, according to the Kabat numbering system: 1E, 2A, 5Q, 10Q, 10T, 14E, 15G, 16D, 16Q, 19I, 23K, 23Q, 23Y, 25F, 25Y, 28D, 28E, 28K, 28N, 28R, 30K, 30S, 31K, 33P, 35A, 35G, 35S, 37F, 37Y, 37H, 39R, 40P, 44D, 45E, 48I, 49A, 52E, 52D, 55E, 56E, 60A, 60D, 65D, 68E, 73D, 73P, 74E, 76K, 76N, 77Q, 82bD, 82bN, 83D, 83K, 83L, 83Q, 83T, 84E, 84P, 84Y, 85K, 85R, 85S, 85T, 89I, 105D, 107I, 107Y, 110I, and 110V. The substitutions may also include a non-natural disulfide bond including at least one cysteine residue at a non-naturally occurring amino acid position, for example, the non-natural disulfide bond may be present between two cysteine residues at positions 2 and 102; 17 and 82a; 19 and 81; 23 and 77; 34 and 78; 35 and 50, according to the Kabat numbering system.

Also, in the various aspects of the disclosure, the substitutions may include one of the the following combinations of amino acids, according to the Kabat numbering system:

| | | |
|---|---|---|
| 5Q/23Q | 28D/39R/48I/83D | 37Y (or 39R)/ |
| | | 10T/82bD |
| 10Q/48I/84E | 28D/39R/48I/84E | 37Y (or 39R) / |
| | | 82bD/84P |
| 10T/82bD | 28D/39R/76N/83D | 37Y/39R/83T |
| 10T/82bD | 28D/39R/76N/84E | 37Y/39R/45E/83T |
| 10T/82bN | 28D/48I/83D | 37Y/44D |
| 10T/84P | 28D/48I/84E | 37Y/48I |
| 15G/37Y | 28D/49A | 37Y/49A/74E |
| 15G/44D | 28D/49A/77Q | 37Y/85S |
| 15G/85S | 28D/55E | 37Y/83T |
| 15G/83T | 28D/55E/74E | 39R/28D |
| 16D/37F | 28D/76N/83D | 39R/45E |
| 16D/37Y | 28D/76N/84E | 39R/48I |
| 16D/39R/48I | 28K/49A | 39R/60A |
| 16D/48I | 28K/49A/77Q | 39R/60D |
| 16D/110I | 28K/49A/55E/84E | 39R/68E |
| 23Q/77Q | 28K/49A/55E/ | 39R/76N |
| 28D/37Y/48I/83D | 84E/10T/82bN | 39R/83D |
| 28D/37Y/48I/84E | 28K/55E | 39R/84E |
| 28D/37Y/76N/83D | 28K/55E/74E | 39R/83T |
| 28D/37Y/76N/84E | 37F/48I | 39R/45E/48I |
| 28D/39R/45E/76N/84E | 37Y (or 39R)/10T/84P | 39R/45E/49A/74E |
| 39R/45E/82bD/84P | 49A/55E/84E | 45E/82bD/84P |
| 44D/85S | 49A/74E | 49A/84E |
| 44D/83T | 49A/74E/77Q | 82bD/84P |
| 45E/82bD/84P | 49A/77Q | 82bN/84P |
| 49A/55E | 49A/77Q/55E | 83T/44D |
| 49A/55E/77Q | 49A/77Q/84E | |

In each of the foregoing combinations, the combinations may include at least one of 39R, 45E, and 37Y if not already present.

In another aspect of the disclosure, the single immunoglobulin variable domain (or framework region(s) thereof) may have an origin of a human germline gene selected from germline family 1, germline family 2, germline family 3, germline family 4, germline family 5, or germline family 7.

As an example of a human germline sequence, the germline gene family 1 may include germline gene family members 1-2 (SEQ ID NO: 1), 1-3 (SEQ ID NO: 2), 1-8 (SEQ ID NO: 3), 1-18 (SEQ ID NO: 4), 1-24 (SEQ ID NO: 5), 1-45 (SEQ ID NO: 6), 1-46 (SEQ ID NO: 7), 1-58 (SEQ ID NO: 8), 1-69 (SEQ ID NO: 9), and 1-69.2 (SEQ ID NO: 10), and alleles thereof, and the single immunoglobulin variable domain (or framework region(s) thereof) may include one or more of the following substitutions: 10Q, 16D, 16Q, 25Y, 25F, 37F, 37Y, 39R, 45E, 48I, 84E, 84P, 110V, and 110I. In addition, the single immunoglobulin variable domains (or framework region(s) thereof) may include one the following combinations of substitutions:

| | | |
|---|---|---|
| 10Q/48I/84E | 16D/48I | 39R/45E/48I |
| 16D/37F | 16D/110I | 39R/48I |
| 16D/37Y | 37F/48I | |
| 16D/39R/48I | 37Y/48I | |

In additional embodiments of the disclosure having an origin of human germline gene family 1, the single immunoglobulin variable domain (or framework region(s) thereof) may include one of the following combinations of substitutions:

| | | |
|---|---|---|
| 17C/82aC/10Q/48I/84E | 17C/82aC/16D/48I | 17C/82aC/84E |
| 17C/82aC/16D | 17C/82aC/37F | 34C/78C/16D |
| 17C/82aC/16D/37F | 17C/82aC/37Y | 34C/78C/37F |
| 17C/82aC/16D/37Y | 17C/82aC/37Y/48I | 34C/78C/84E |
| 17C/82aC/16D/37Y/39R | 17C/82aC/39R | 34C/78C/16D/37F |
| 17C/82aC/16D/39R | 17C/82aC/39R/45E/48I | 34C/78C/16D/48I |
| 17C/82aC/16D/39R/48I | 17C/82aC/39R/48I | 34C/78C/10Q/ |
| | | 48I/84E |

In each of the foregoing combinations, the combinations may include at least one of 39R, 45E, and 37Y if not already present.

As another example of a human germline sequence, the germline gene family 2 may include germline gene family members 2-5 (SEQ ID NO: 11), 2-26 (SEQ ID NO: 12), and 2-70 (SEQ ID NO: 13), and alleles thereof, and the single immunoglobulin variable domain (or framework region(s) thereof) may include one or more of the following substitutions: one or more of the following substitutions: 15G, 16D, 37Y, 37H, 39R, 44D, 45E, 65D, 73D, 73P, 83L, 83Q, 83K, 83T, 84Y, 85R, 85S, 85K, 85T, 89I, 105D, and 107I.

In additional embodiments of the disclosure having an origin of human germline gene family 2 the single immunoglobulin variable domain (or framework region(s) thereof) may include one of the following combinations of substitutions:

| | | |
|---|---|---|
| 15G/37Y | 37Y/39R/45E/83T | 37Y/83T |
| 15G/44D | 37Y/39R/83T | 39R/83T |
| 15G/85S | 37Y/44D | 44D/85S |
| 15G/83T | 37Y/85S | 44D/83 |

Still further, in additional embodiments of the disclosure having an origin of human germline gene family 2, the single immunoglobulin variable domain (and framework regions thereof) may include one of the following combinations of substitutions:

| | | |
|---|---|---|
| 19C/81C/15G | 19C/81C/37Y/39R/83T | 19C/81C/44D |
| 19C/81C/15G/37Y | 19C/81C/37Y/39R/45E/83T | 19C/81C/44D/85S |
| 19C/81C/15G/44D | 19C/81C/37Y/44D | 19C/81C/85S |
| 19C/81C/15G/85S | 19C/81C/37Y/83T | 19C/81C/83T |
| 19C/81C/15G/83T | 19C/81C/37Y/85S | 19C/81C/83T/44D |
| 19C/81C/37Y | 19C/81C/39R/83T | |

In each of the foregoing combinations, the combinations may include at least one of 39R, 45E, and 37Y if not already present.

As another example of a human germline sequence, the germline gene family 3 may include germline gene family members 3-7 (SEQ ID NO: 14), 3-9 (SEQ ID NO: 15), 3-11 (SEQ ID NO: 16), 3-13 (SEQ ID NO: 17), 3-15 (SEQ ID NO: 18), 3-20 (SEQ ID NO: 19), 3-21 (SEQ ID NO: 20), 3-23 (SEQ ID NO: 21), 3-30 (SEQ ID NO: 22), 3-33 (SEQ ID NO: 23), 3-43 (SEQ ID NO: 24), 3-48 (SEQ ID NO: 25), 3-49 (SEQ ID NO: 26), 3-53 (SEQ ID NO: 27), 3-64 (SEQ ID NO: 28), 3-66 (SEQ ID NO: 29), 3-72 (SEQ ID NO: 30), 3-73 (SEQ ID NO: 31), 3-74 (SEQ ID NO: 32), 3-d (SEQ ID NO: 33), and 3-NL1 (SEQ ID NO: 34), and alleles thereof, and the single immunoglobulin variable domain (or framework region(s) thereof) may include one or more of the following substitutions: one or more of the following substitutions: 2A, 5Q, 14E, 23K, 23Q, 23Y, 28D, 28E, 28N, 28K, 28R, 30K, 30S, 31K, 33P, 35G, 35A, 35S, 37Y, 39R, 40P, 45E, 49A, 52E, 52D, 55E, 56E, 74E, 76K, 77Q, 82bD, 84E, 84P, 110V, and 110I.

In additional embodiments of the disclosure having an origin of human germline gene family 3 the single immunoglobulin variable domain (and framework region(s) thereof) may include one of the following combinations of substitutions:

| | | |
|---|---|---|
| 5Q/23Q | 28D/55E/74E | 28K/55E/74E |
| 23Q/77Q | 28K/49A | 37Y/49A/74E |
| 28D/49A | 28K/49A/55E/84E | 39R/45E/49A/74E |
| 28D/49A/77Q | 28K/49A/77Q | 39R/49A/84E |
| 28D/55E | 28K/55E | 39R/84E |
| 49A/55E | 49A/74E/77Q | 49A/77Q/84E |
| 49A/55E/77Q | 49A/77Q | 49A/84E |
| 49A/55E/84E | 49A/77Q/55E | |

Still further, in additional embodiments of the disclosure having an origin of human germline gene family 3, the single immunoglobulin variable domain (and framework regions thereof) may include one of the following combinations of substitutions:

| | | |
|---|---|---|
| 23C/77C/28K/49A | 23C/77C/39R/45E/49A/74E | 34C/78C/28K |
| 23C/77C/28D/49A | 23C/77C/39R/49A/74E | 34C/78C/49A |
| 23C/77C/28K/55E | 23C/77C/39R/49A/84E | 34C/78C/55E |
| 23C/77C/28K/55E/74E | 23C/77C/39R/49A/84E | 34C/78C/74E |
| 23C/77C/28K/49A/55E/84E | 23C/77C/49A/55E/84E | 34C/78C/77Q |
| 23C/77C/37Y/49A/74E | 34C/78C/28D | 34C/78C/84E |

In each of the foregoing combinations, the combinations may include at least one of 39R, 45E, and 37Y if not already present.

As another example of a human germline sequence, the germline gene family 4 may include germline gene family members 4-4 (SEQ ID NO: 35), 4-28 (SEQ ID NO: 36), 4-30-1 (SEQ ID NO: 37), 4-30-2 (SEQ ID NO: 38), 4-30-4 (SEQ ID NO: 39), 4-31 (SEQ ID NO: 40), 4-34 (SEQ ID NO: 41), 4-38-2 (SEQ ID NO: 42), 4-39 (SEQ ID NO: 43), 4-59 (SEQ ID NO: 44) and 4-61 (SEQ ID NO: 45), 4-b (SEQ ID NO: 46), and alleles thereof, and the single immunoglobulin variable domain (or framework region(s) thereof) may include one or more of the following substitutions: one or more of the following substitutions: 1E, 10Q, 10T, 15G, 19I, 37Y, 39R, 45E, 82bD, 82bN, 84P, 107I, and 107Y.

In additional embodiments of the disclosure having an origin of human germline gene family 4 the single immunoglobulin variable domain (and framework region(s) thereof) may include one of the following combinations of substitutions:

| | | |
|---|---|---|
| 10T/82bN | 10T/82bD | 37Y (and/or 39R)/10T/84P |
| 10T/84P | 37Y (and/or 39R)/82bN/84P | |
| 37Y (and/or | 37Y (and/or 39R)/10T/82bN | 45E/82bD/84P |
| 39R)/10T/82bD | 39R/45E/82bD/84P | |

Still further, in additional embodiments of the disclosure having an origin of human germline gene family 4, the single immunoglobulin variable domain (and framework regions thereof) may include one of the following combinations of substitutions:

| | |
|---|---|
| 17C/82aC/10T | 23C/77C/45E/82bD/84P |
| 17C/82aC/10T/82bN | 23C/77C/82bD/84P |
| 17C/82aC/10T/82bD | 23C/77C/82bN/84P |
| 17C/82aC/82bN/84P | 23C/77C/37Y (and/or 39R)/10T/82bD |
| 17C/82aC/37Y (and/or 39R)/10T/82bD | 23C/77C/37Y (and/or 39R)/10T/82bN |
| 17C/82aC/37Y (and/or 39R)/10T/84P | 23C/77C/37Y (and/or 39R)/10T/84P |
| 17C/82aC/37Y (and/or 39R)/82bD/84P | 23C/77C/37Y (and/or 39R)/82bD/84P |
| 23C/77C/10T/84P | 23C/77C/37Y (and/or 39R)/82bD/84P |
| 23C/77C/39R/45E/82bD/84P | |

In each of the foregoing combinations, the combinations may include at least one of 39R, 45E, and 37Y if not already present.

As another example of a human germline sequence, the germline gene family 5 may include germline gene family members 5-51 (SEQ ID NO: 47) and 5-a (SEQ ID NO: 48), and alleles thereof; and the single immunoglobulin variable domain (or framework region(s) thereof) may include one or more of the following substitutions: one or more of the following substitutions: 28D, 37Y, 39R, 45E, 48I, 60D, 60A, 68E, 76N, 83D, and 84E.

In additional embodiments of the disclosure having an origin of human germline gene family 5 the single immunoglobulin variable domain (and framework region(s) thereof) may include one of the following combinations of substitutions:

| | | |
|---|---|---|
| 39R/28D | 39R/60A | 39R/68E |
| 39R/48I | 39R/60D | 39R/76N |
| 39R/83D | 28D/48I/83D | 28D/37Y/48I/84E |
| 39R/84E | 28D/39R/48I/84E | 28D/37Y/76N/83D |
| 28D/48I/84E | 28D/39R/76N/83D | 28D/37Y/76N/84E |
| 28D/76N/83D | 28D/39R/76N/84E | 28D/37Y/48I/83D |
| 28D/76N/84E | 28D/39R/48I/83D | 28D/39R/45E/76N/84E |

In each of the foregoing combinations, the combinations may include at least one of 39R, 45E, and 37Y if not already present.

As another example of a human germline sequence, the germline gene family 6 may include germline gene family member 6-1 (SEQ ID NO: 49) and alleles thereof.

As another example of a human germline sequence, the germline gene family 7 may include germline gene family member 7-4-1 (SEQ ID NO: 50) and alleles thereof.

In embodiments of the disclosure having an origin of human germline gene family 7 the single immunoglobulin variable domain (and framework region(s) thereof) may include one of the following combinations of substitutions:
17C/82aC/39R
17C/82aC/39R/45E
17C/82aC/37Y
35C/50C/39R
35C/50C/39R/45E
35C/50C/37Y
   In each of the foregoing combinations, the combinations may include at least one of 39R, 45E, and 37Y if not already present.

In another aspect, the disclosure is directed to a polynucleotide encoding the single immunoglobulin variable domain any framework region(s) thereof of the disclosure.

In another aspect, the disclosure is directed to pharmaceutical acceptable composition including the single immunoglobulin variable domain any framework region(s) thereof.

In another aspect, the disclosure is directed to a VH domain library including a plurality of the single immunoglobulin variable domains as disclosed herein.

In another aspect, the disclosure is directed to a polynucleotide library including a plurality of polynucleotides encoding for a plurality of the single immunoglobulin variable domains as disclosed herein.

In another aspect, the disclosure is directed a method for identifying an antigen binding molecule. The method includes contacting a single immunoglobulin variable domain library of the disclosure with a target, and (ii) identifying single immunoglobulin variable domains of the library binding to the target.

### BRIEF DESCRIPTION OF THE FIGURES

The following detailed description of the embodiments of the present invention can be best understood when read in conjunction with the following drawings, where like structure is indicated with like reference numerals and in which:

FIGS. 1A-1H show V-gene amino acids sequences for the most commonly observed allele of functional human IGHV genes from a number of human antibody germlines.

FIGS. 2A-2O show amino acid sequences of disulfide stabilized full length VH domains according to the disclosure for germline gene family members VH1-8, VH1-18, VH1-69.2, VH3-9, VH3-11, 3-15, VH3-20, VH3-21, VH-30, VH3-53, VH4-34, VH4-39, and VH7-4-1.

FIGS. 3A-3N show the amino acid sequences for a number of the modified full length VH domains according to the disclosure for germline gene family members VH3-20, VH3-21, VH3-15, VH1-69.2, and VH4-39.

FIGS. 4A-4F show the amino acid sequences of modified full length VH domains of the disclosure having the selected combinations of the amino acid substitutions in full length VH's from members of germline families 1, 3, and 4 as shown in FIGS. 2A-M and 3A-E, which are designated Opt1 and Opt2 designs according to the disclosure.

FIGS. 5A-5I show V-gene amino acid sequences of modified VH domains according to the disclosure.

FIG. 6 shows a summary of amino acid substitutions and the fold improvement in expression titers for selected sequences in germlines families VH Family 1 and VH Family 3 according to the disclosure.

FIGS. 7A-7L show the amino acid sequences for each of the wild type and modified amino acid sequences for the germline families in FIG. 6.

FIG. 8 shows a summary of amino acid substitutions, the expression titers, and the thermal melting point (Tₘ) for selected sequences in germline family VH4 members, 4-34 and 4-39, according to the disclosure.

FIG. 9 shows a summary of amino acid substitutions for selected wild type and modified sequences in germline family VH4 members 4-4, 4-28, 4-30-1, 4-30-2, 4-30-4, 4-31, 4-34, 4-38, 4-59 and 4-61 according to the disclosure.

FIGS. 10A-10N show the amino acid sequences for each of the wild type and modified amino acid sequences for the germline VH4 sequences in FIGS. 8 and 9.

FIGS. 11 and 12 show a summary of amino acid substitutions for selected wild type and modified sequences in germline family VH2 members 2-5 and 2-26, some including 137Y, according to the disclosure.

FIGS. 13A-13H show the amino acid sequences for each of the wild type and modified amino acid sequences for the germline VH2 members 2-5 and 2-26 sequences in FIGS. 11 and 12.

FIGS. 14 and 15 show a summary of amino acid substitutions for selected wild type and modified sequences in germline family VH5 member 5-51.

FIGS. 16A-16F shows the amino acid sequences for each of the wild type and modified amino acid sequences for the VH5-51 sequences in FIGS. 14 and 15.

FIG. 17 shows a summary of substitutions in germline family members 1-8, 3-30, and 4-34 and reflecting the impact of a 37Y variant according to the disclosure.

FIGS. 18A-18C show the amino acid sequences for each of the amino acid sequences in FIG. 17.

FIG. 19, Panels A and B show size exclusion chromatography (SEC) of the Gr6 human VH domains with and without various substitutions designed to reduce dimerization.

FIGS. 20A-20U shows the amino acid sequences of members of several germline families that have been modified to with substitutions according to the disclosure.

FIGS. 21A-21H show a summary of amino acid substitutions and combinations thereof in VH families 1-69.2, 3-15, 3-21, 4-39, and 3-20 along with expression and Tm data.

### DESCRIPTION

The disclosure is directed to design and characterization of single immunoglobulin variable domains with substitutions in the variable regions resulting in one or more of improved thermal stability, improved cellular expression, decreased dimerization, and decreased light chain pairing.

All publications, patents and patent applications cited herein are hereby expressly incorporated by reference for all purposes.

Before describing the various aspects of the disclosure, a number of terms will be defined. Unless otherwise required by context, singular terms shall include pluralities and plural terms shall include the singular. For example, the singular forms “a,” “an,” and “the” include plural referents unless the context clearly dictates otherwise.

As utilized in accordance with the present disclosure, unless otherwise indicated, all technical and scientific terms shall be understood to have the same meaning as commonly understood by one of ordinary skill in the art.

The term “amino acid” or “residue” as used within this application denotes the group of naturally occurring carboxy α-amino acids including alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

The term “immunoglobulin” refers to a protein having the structure of a naturally occurring antibody, as described herein.

An “antibody” refers to a glycoprotein including at least two heavy (H) chains and two light (L) chains inter-connected by disulfide bonds and having a structure substantially similar to a native antibody structure. For example, native IgG-class antibodies are heterotetrameric glycoproteins of about 150 kilodaltons (kD), composed of two light chains and two heavy chains that are disulfide-bonded. From N- to C-terminus, each heavy chain has a variable region (VH), followed by three constant domains (CH1, CH2, and CH3) (also called a heavy chain constant region). Similarly, from N- to C-terminus, each light chain has a variable region (VL) followed by a light chain constant domain (CL) (also called a light chain constant region). The heavy chain of an antibody may be assigned to one of five types, called α (IgA), δ (IgD), ε (IgE), γ (IgG), or μ, (IgM), some of which may be further divided into subtypes, e.g., γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

“Germline” as used herein refers to the DNA encoded amino acid sequences that are transmitted from generation to generation. Human antibody germline gene and polypeptide sequences, including the wild-type functional V-D-J gene segments, can be found at the ImMunnoGeneTics (IMGT®), website (http://www.imgt.org/). IMGT® is the global reference in immunogenetics and immunoinformatics for integrated knowledge resources specialized in. among other things, the immunoglobulins (IG) or antibodies. IMGT® provides a common access to sequence, genome and structure immunogenetics data. IMGT® works in close collaboration with EBI (Europe), DDBJ (Japan) and NCBI (USA). See also, Barker, et al., The IPD-IMGT/HLA database, Nucleic Acids Research, gkac1011, November 2022, https://doi.org/10.1093/nar/gkac1011.

Many gene family members have one or several known polymorphs (referenced by IMGT® as—*01,-*02, etc., e.g., “3-64—*01”). Unless otherwise indicated, for each of the V gene sequences identified in the disclosure, the *01 allele is shown as representative for the family member.

The term “variable region” or “variable domain” refers to the domain of an antibody heavy or light chain that is involved in binding the antigen binding molecule to antigen. The variable domains of the heavy chain and light chain (VH and VL, respectively) of a native antibody generally have similar structures, with each full length domain including four conserved framework regions (FRs) and three hypervariable regions (HVRs). A single full length VH or VL domain may be sufficient to confer antigen-binding specificity, although the disclosure herein is focused on VH domains and, in several embodiments, the V-gene portions thereof.

The term “complementarity determining region(s)” or “CDR(s)” as used herein refers to each of the regions of an antibody variable domain which are hypervariable in sequence and/or form structurally defined loops (“hypervariable loops”) and/or contain the antigen-contacting residues (“antigen contacts”). Generally, antibodies include six CDRs: three in the full length VH (HCDR1, HCDR2, HCDR3), and three in the full length VL (LCDR1, LCDR2, LCDR3).

“Framework” or “FR” refers to variable domain residues other than CDR residues. The FR of a full length variable domain generally consists of four FR regions: FR1, FR2, FR3, and FR4. Accordingly, the CDR and FR sequences generally appear in the following sequence either a VH or VL: FR1-CDR1-FR2-CDR2-FR3-CDR3-FR4. For simplicity in the context of the VH domains described herein, references to FR1, FR2, FR3 and FR4 are intended to refer the FR regions of the VH domains (with the understanding that VL domains also have FRs).

“IGHV” as used herein refers to the amino acid sequence of the V-gene portion of a full length VH and includes FR1, CDR1, FR2, CDR2, and FR3. In some instances, the V-gene encodes a few amino acids of CDR3. The V-gene portion gets recombinantly fused to one of approximately 23 functional D chains and one of six J chains to form a mature, full-length VH domain. The HCDR3 region is the most diverse region of a full length VH domain consisting of sequences from the V-gene, D chains, and J chains and includes significant diversity generated by insertions, deletions, and mutations that occur at the junction sites during recombination. The J chains comprise the latter portions of HCDR3 and the entirety of FR4. The FR4 regions of the six J chains are fairly well conserved (i.e., little diversity), and shown here with the amino acids of FR4 underlined:

(SEQ ID NO: 51)
JH1 AEYFQHWGQGTLVTVSS
(SEQ ID NO: 52)
JH2 YWYFDLWGRGTLVTVSS
(SEQ ID NO: 53)
JH3 DAFDVWGQGTMVTVSS
(SEQ ID NO: 54)
JH4 YFDYWGQGTLVTVSS
(SEQ ID NO: 55)
JH5 NWFDSWGOGTLVTVSS
(SEQ ID NO: 56)
JH6 YYYYYGMDVWGQGTTVTVSS

As used herein, “Kabat numbering” refers to the numbering system set forth by Kabat et al., U.S. Dept. of Health and Human Services, “Sequence of Proteins of Immunological Interest” (1983). Unless otherwise indicated, CDR residues and other residues in the variable domain (e.g., FR residues) are numbered herein with the “the Kabat numbering system” to assign a position to any variable region sequence, without reliance on any experimental data beyond the sequence itself. According to the Kabat numbering system, CDR1 includes amino acids 23-35 (including amino acids 31a and 31b when present), CDR2 includes amino acids 50-58 (including amino acids 52a, 52b, and 52c when present), and CDR3 includes amino acids 93-102 (including amino acids 100a, 100b, 100c, 100d, 100e, 100f, 100g, 100h, 100i, 100j, 100k, and 100l) when present (see e.g., North et al 2013, J Mol Biol. 2011 406 (2): 228-256). Positions with lower case letters (a, b, c, etc) are used in accordance with the Kabat numbering system because many of the VH sequences of disclosure encompass different lengths as the result of variability in the length of the CDRs. For example, many of the sequences of the disclosure do not have an amino acid at one or more of positions 31a, 31b, 52a, 52b, and 52c, 100a, 100b, 100c, 100d, 100e, 100f, 100g, 100h, 100i, 100j, 100k, and 100l. Accordingly, several of the Tables and Figures of the disclosure herein reflect positions within the Kabat numbering system that do not have an amino acid at that position (shown herein as “·” or blank at that position).

The polypeptide sequences of the Sequence Listing are not numbered according to the Kabat numbering system. However, it is well within the ordinary skill of one in the art to convert the numbering of the sequences of the Sequence Listing to the Kabat numbering system, and vice versa.

As used herein, term “polypeptide” refers to a molecule composed of monomers (amino acids) linearly linked by amide bonds (also known as peptide bonds). The term “polypeptide” refers to any chain of two or more amino acids and does not refer to a specific length of the product. Thus, peptides, dipeptides, tripeptides, oligopeptides, “protein,” “amino acid chain,” or any other term used to refer to a chain of two or more amino acids, are included within the definition of “polypeptide,” and the term “polypeptide” may be used instead of, or interchangeably with any of these terms.

The term “nucleic acid molecule” or “polynucleotide” includes any compound and/or substance that includes a polymer of nucleotides. Each nucleotide is composed of a base, specifically a purine or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular, messenger RNA (mRNA), synthetic forms of DNA or RNA, and mixed polymers including two or more of these molecules. The nucleic acid molecule may be linear or circular. In addition, the term nucleic acid molecule includes both sense and antisense strands, as well as single stranded and double stranded forms. Moreover, the herein described nucleic acid molecule can contain naturally occurring or non-naturally occurring nucleotides.

An “isolated” nucleic acid molecule or polynucleotide refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The terms “pharmaceutical composition” or “therapeutic composition” as used herein refer to a compound or composition capable of inducing a desired therapeutic effect when properly administered to a patient. In some embodiments, the disclosure provides a pharmaceutical composition including a pharmaceutically acceptable carrier and a therapeutically effective amount of immunotoxin fusion proteins of the disclosure.

The terms “pharmaceutically acceptable carrier” or “physiologically acceptable carrier” as used herein refer to one or more formulation materials suitable for accomplishing or enhancing the delivery of one or more heavy chain variable domains of the disclosure.

Turning now to the various aspects of the disclosure, the inventors have identified approaches to modify the biophysical properties of single chain VH domains from a number of human immunoglobulin germline sequences. Substitution of the VH domains can lead to improvement of the biophysical properties and enhance the therapeutic utility of VH domains, either alone or in combination, for human and non-human medicine.

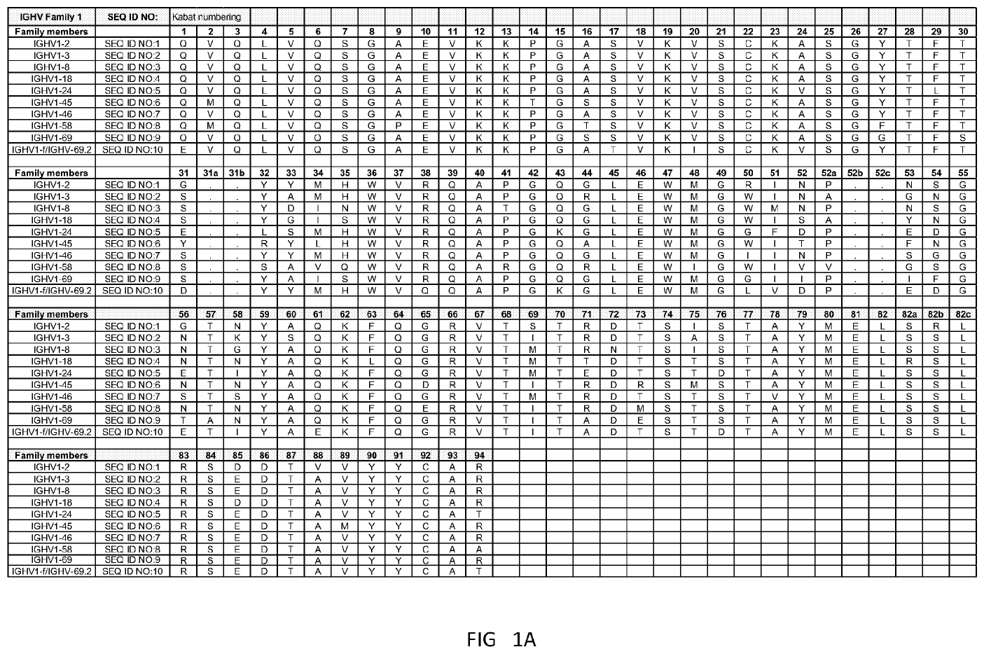

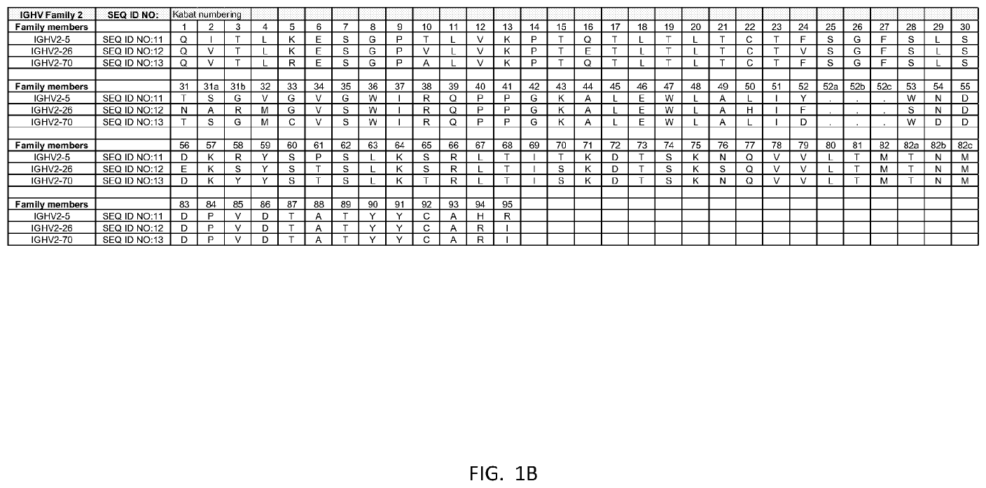

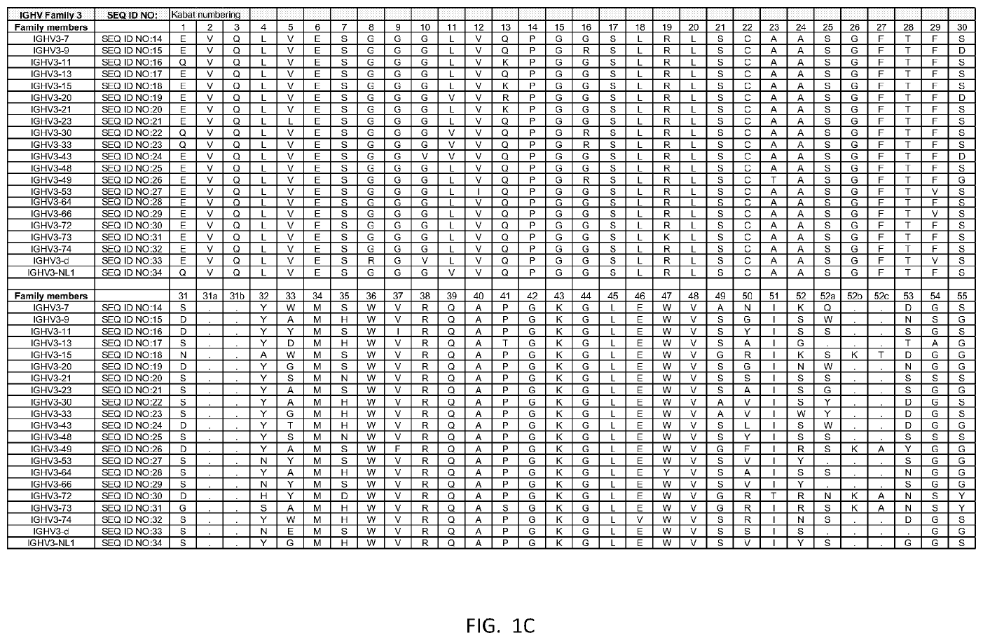

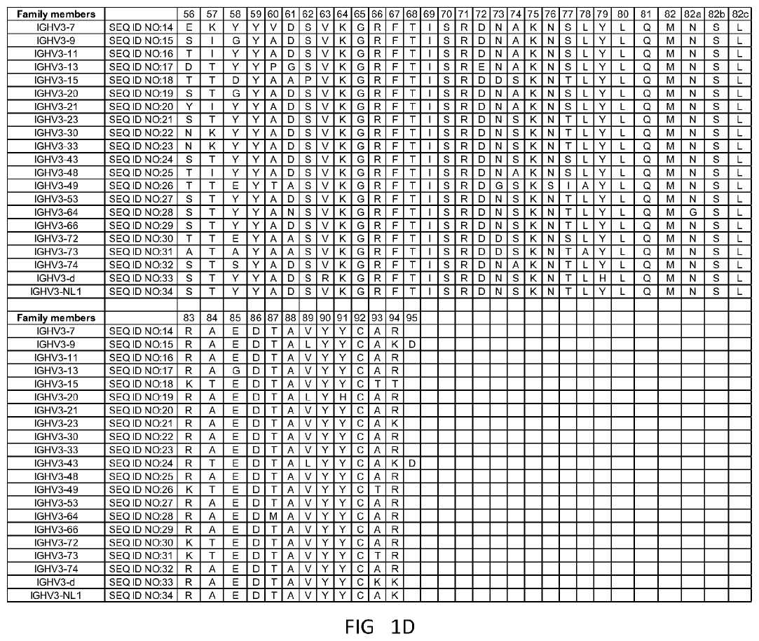

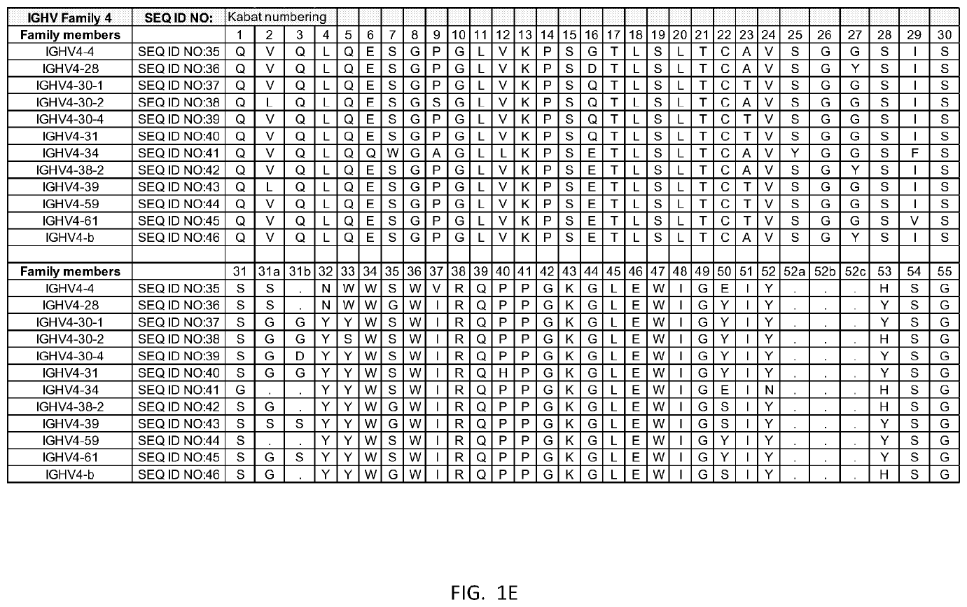

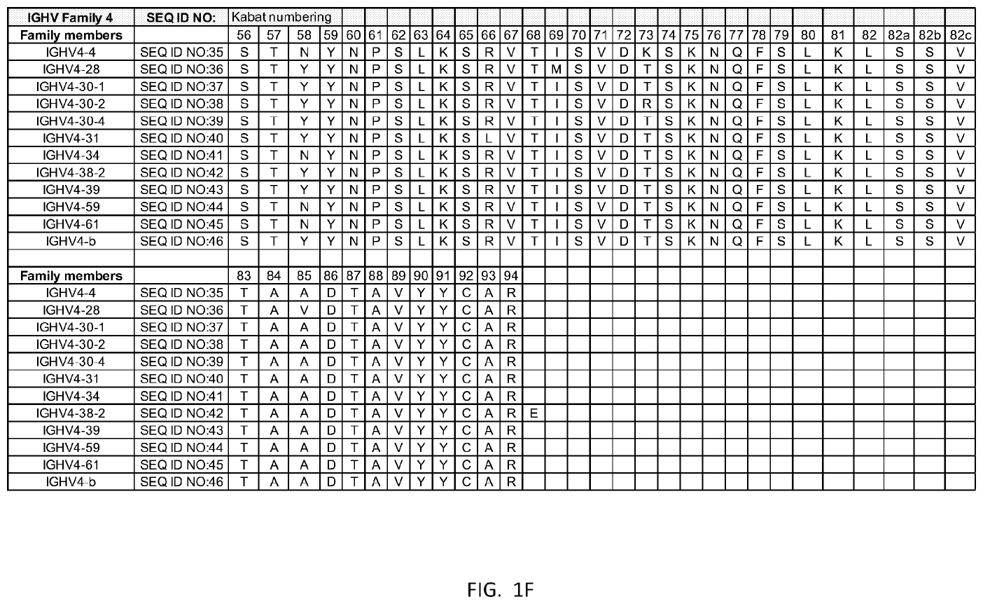

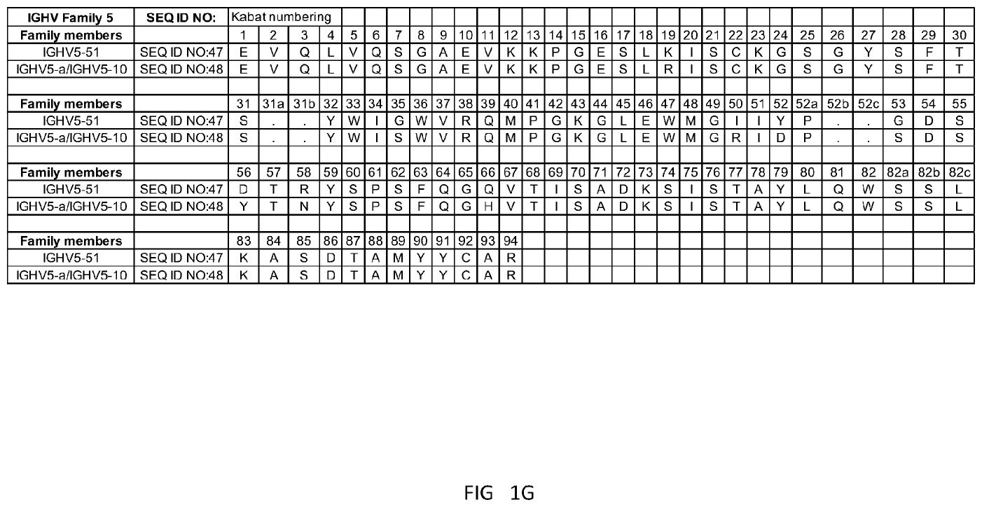

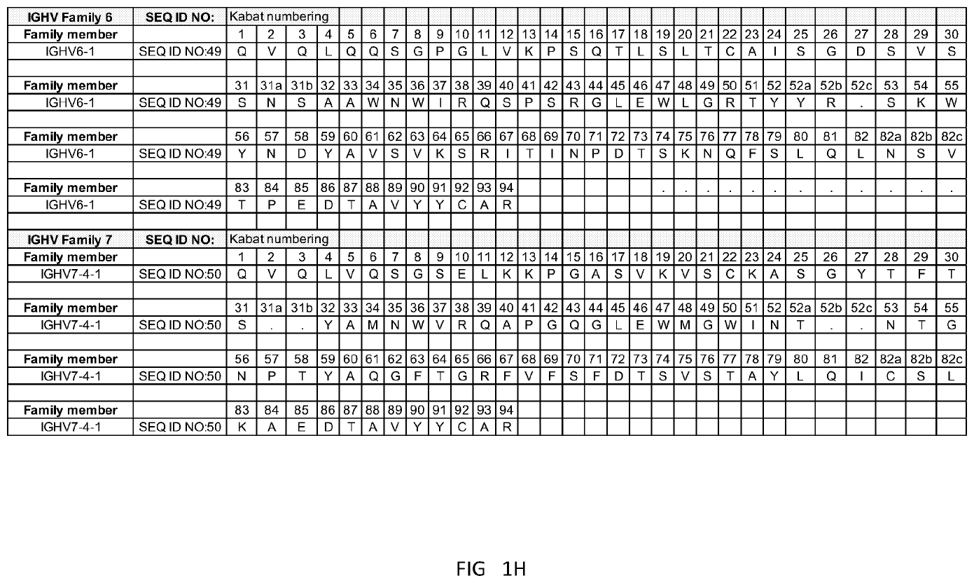

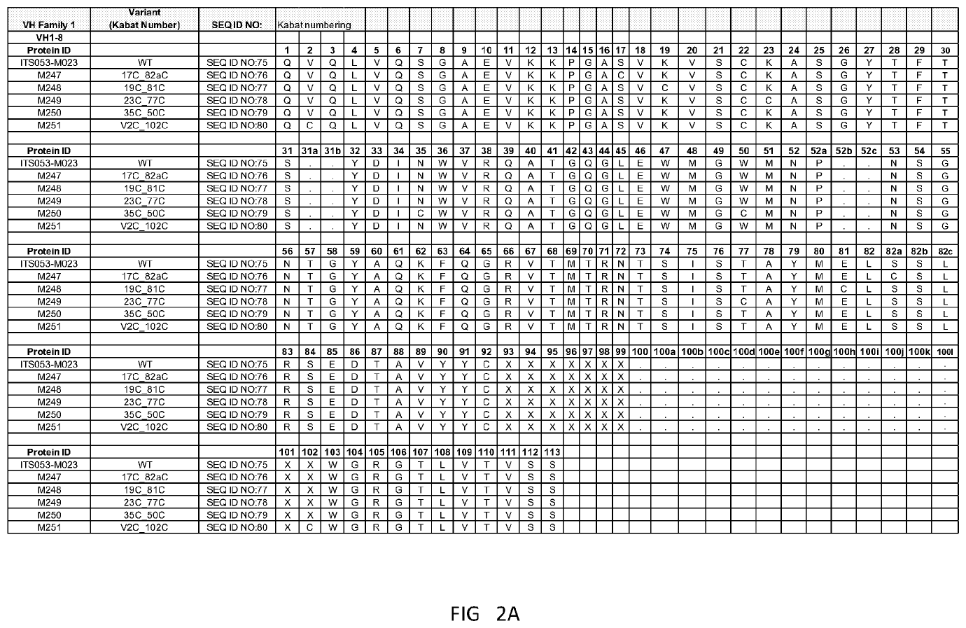

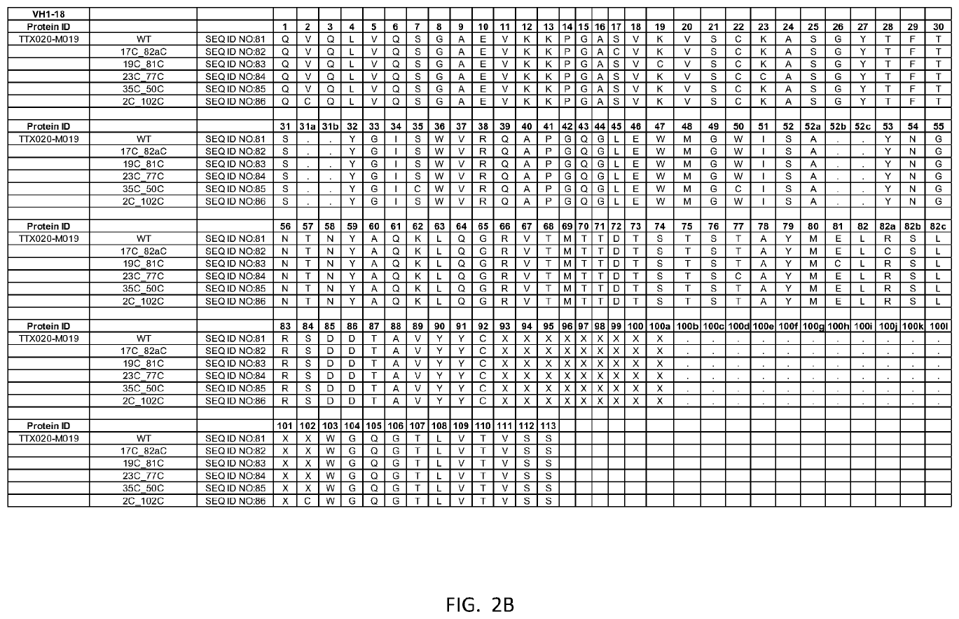

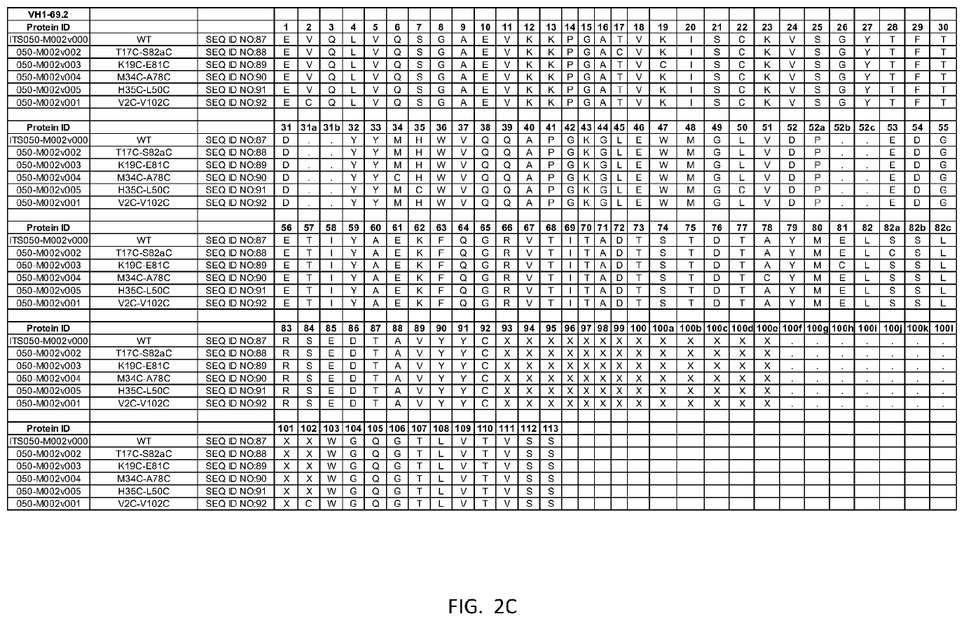

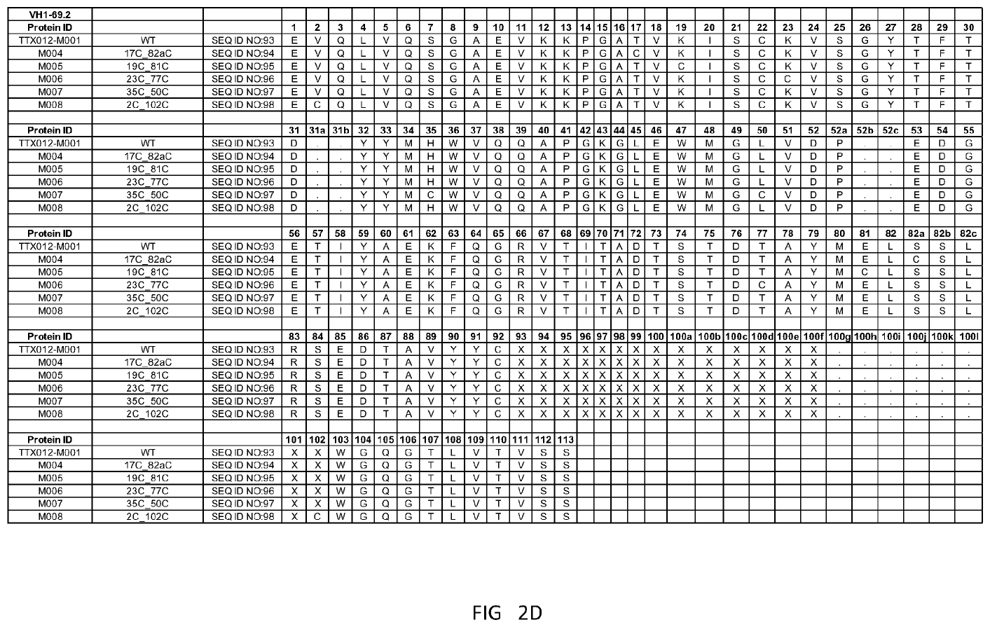

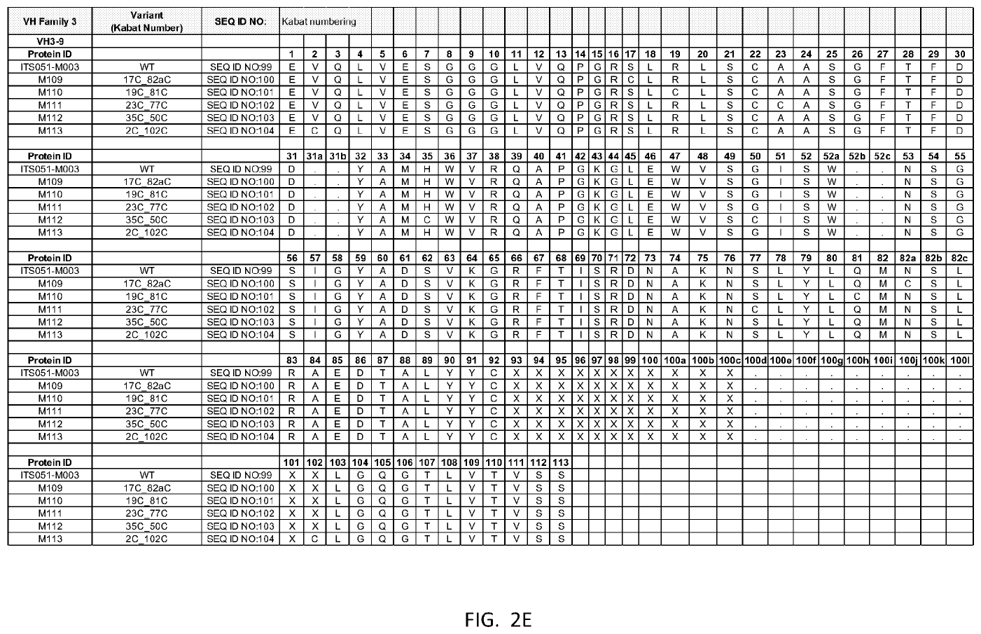

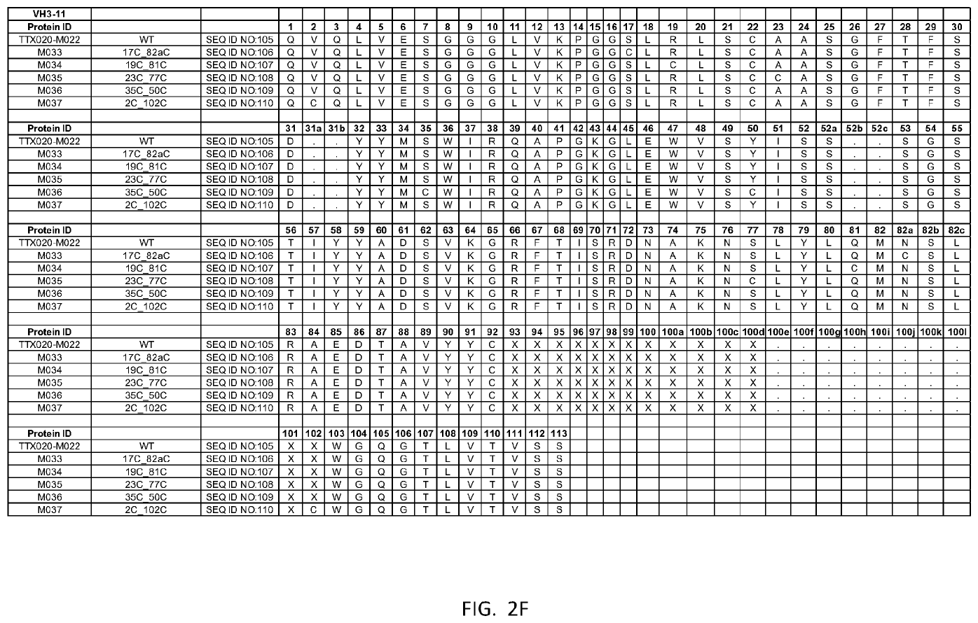

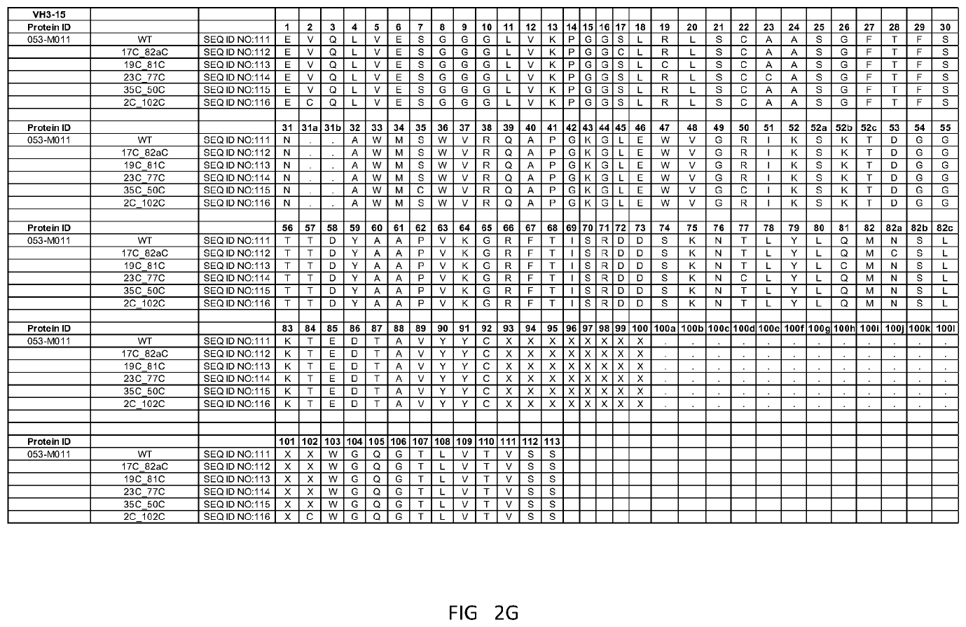

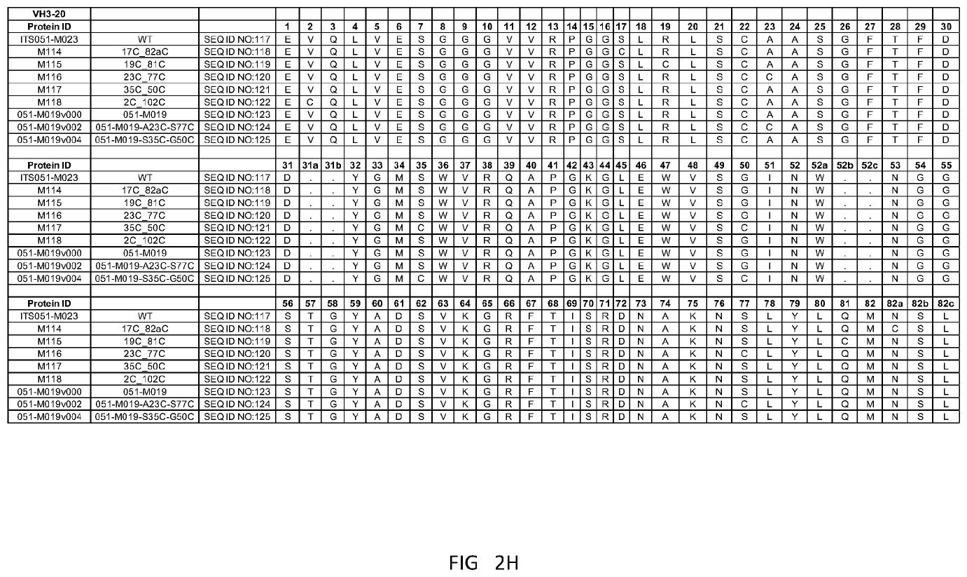

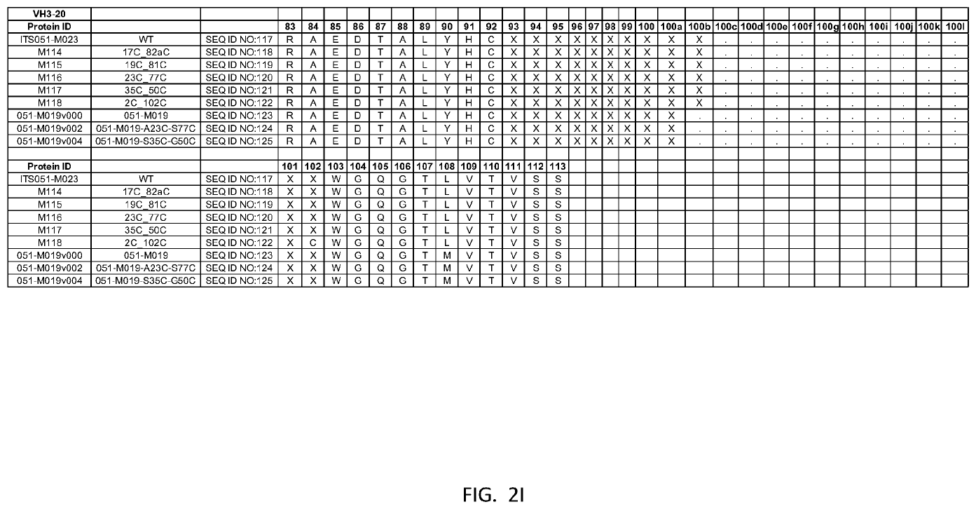

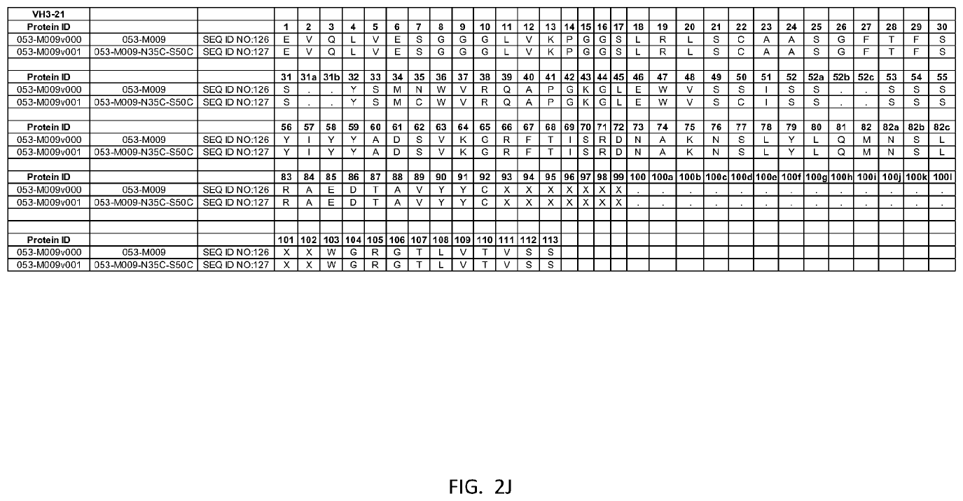

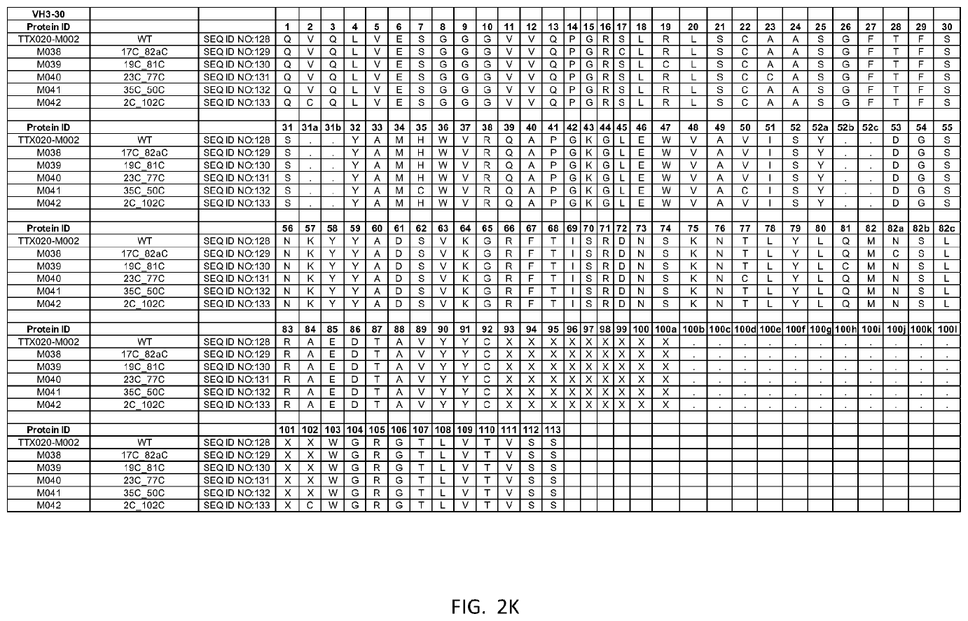

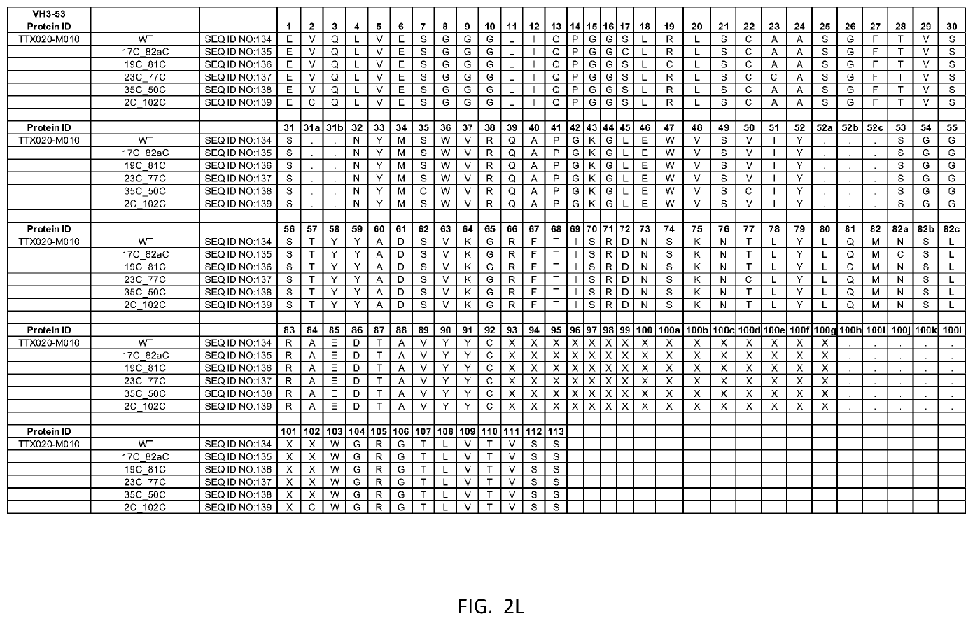

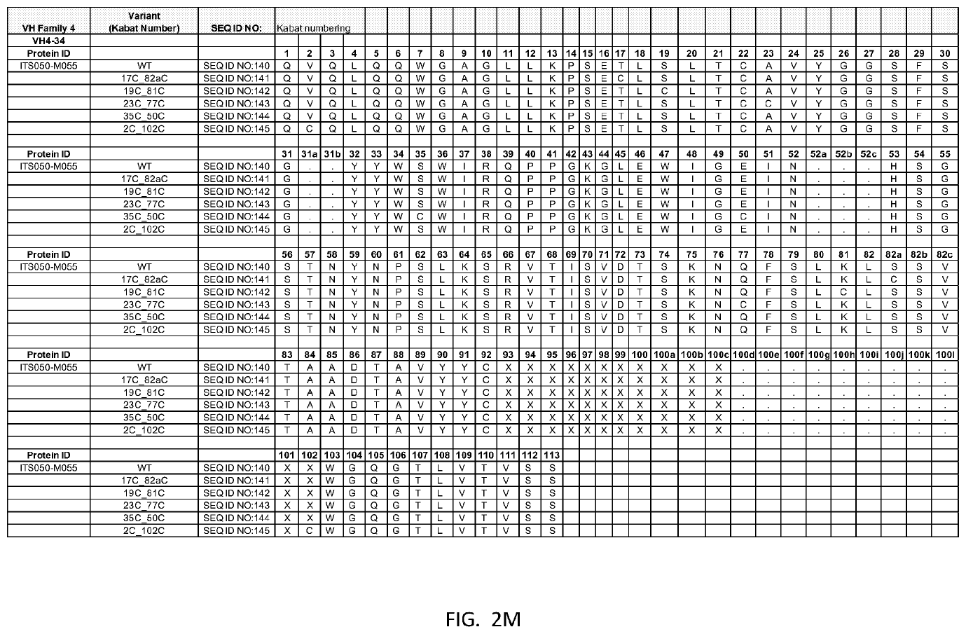

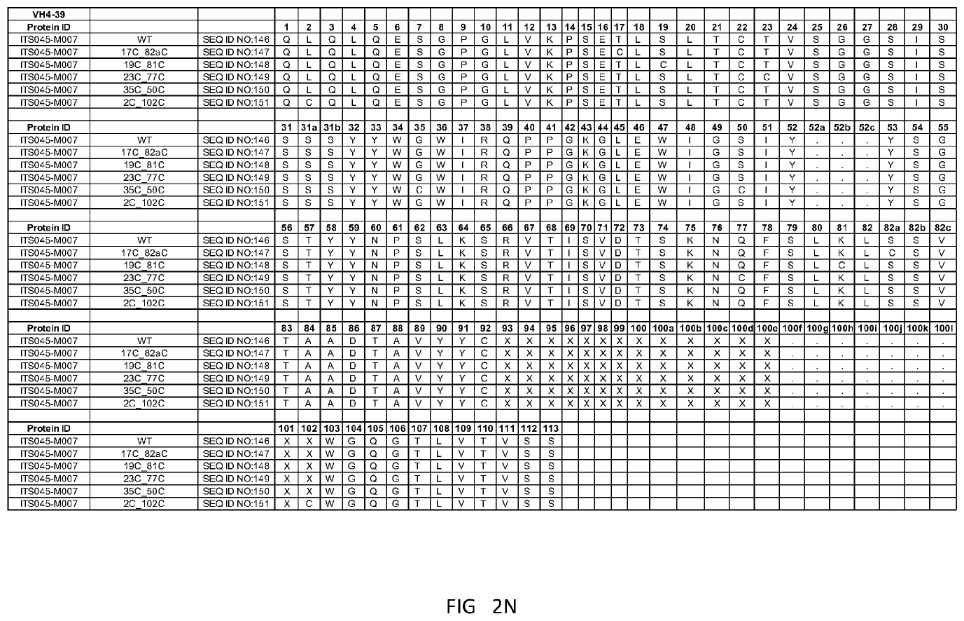

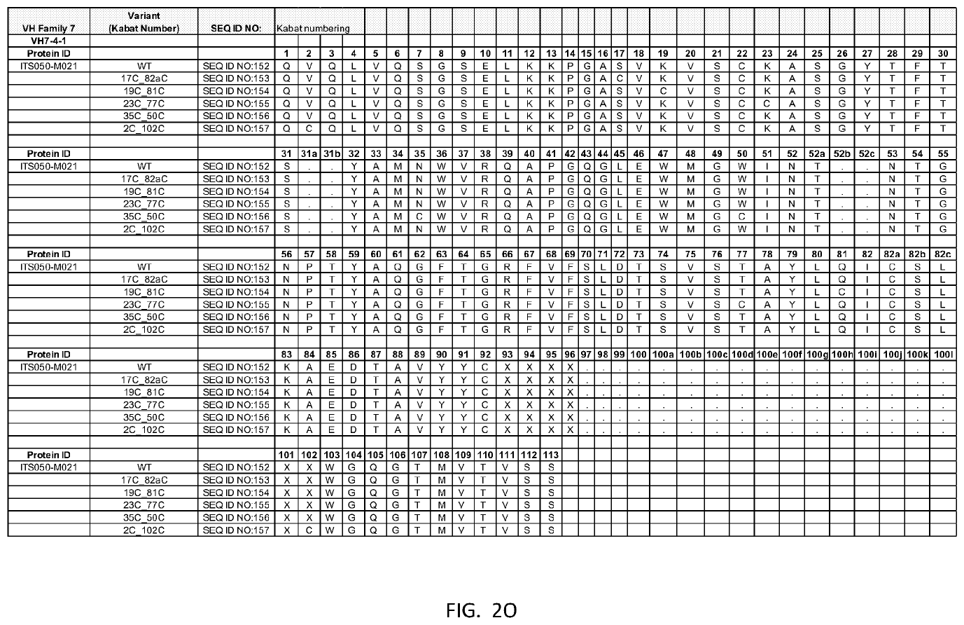

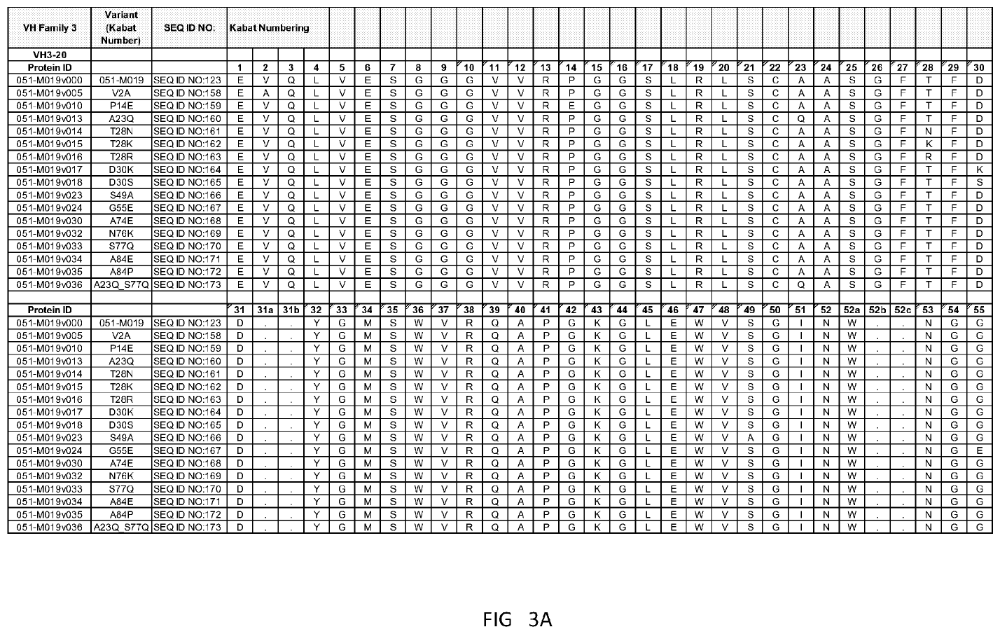

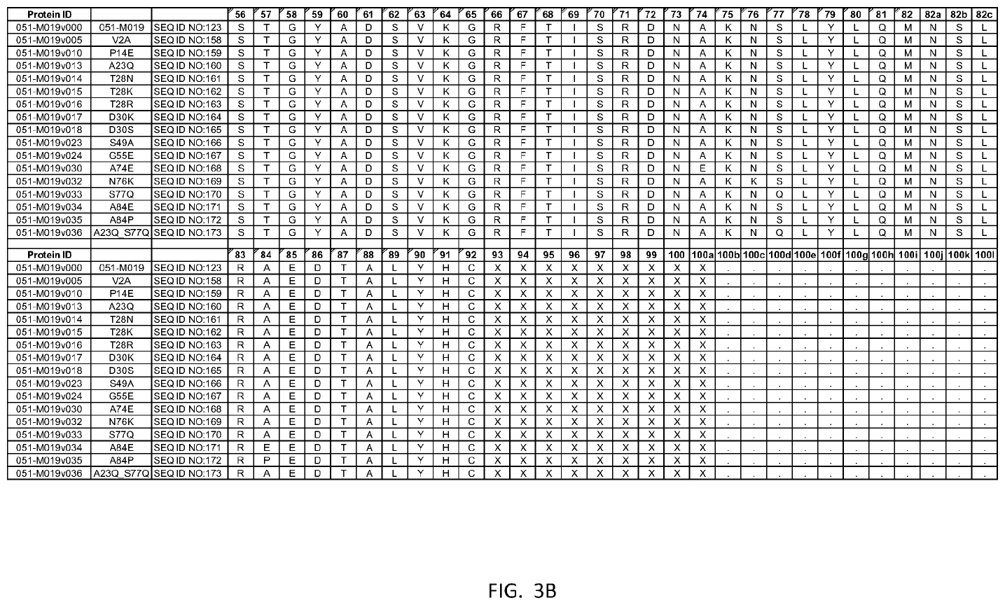

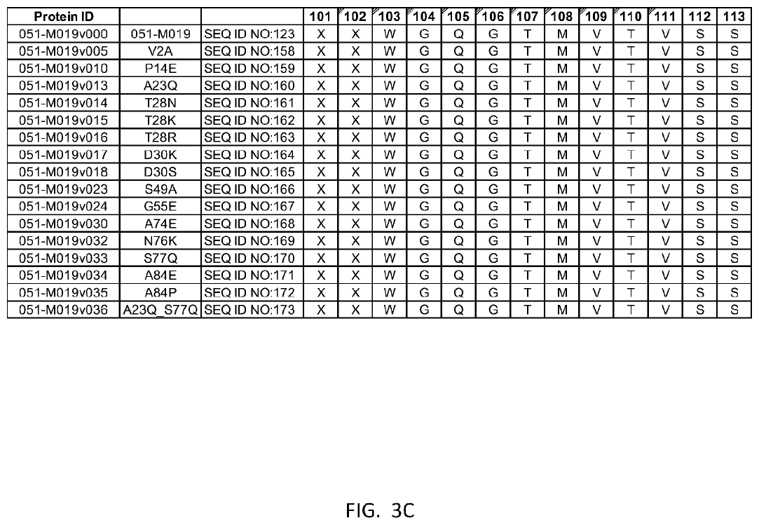

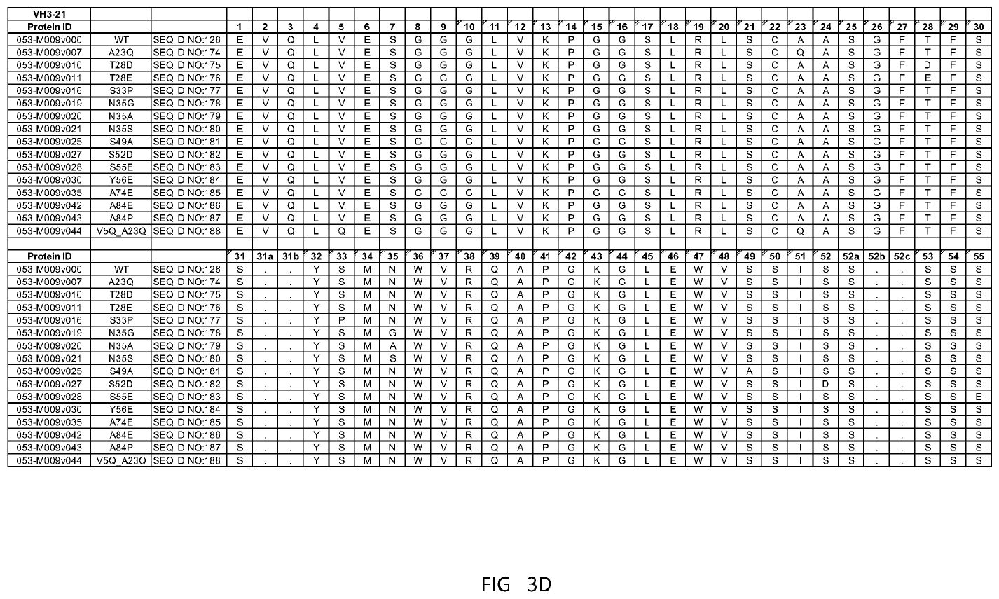

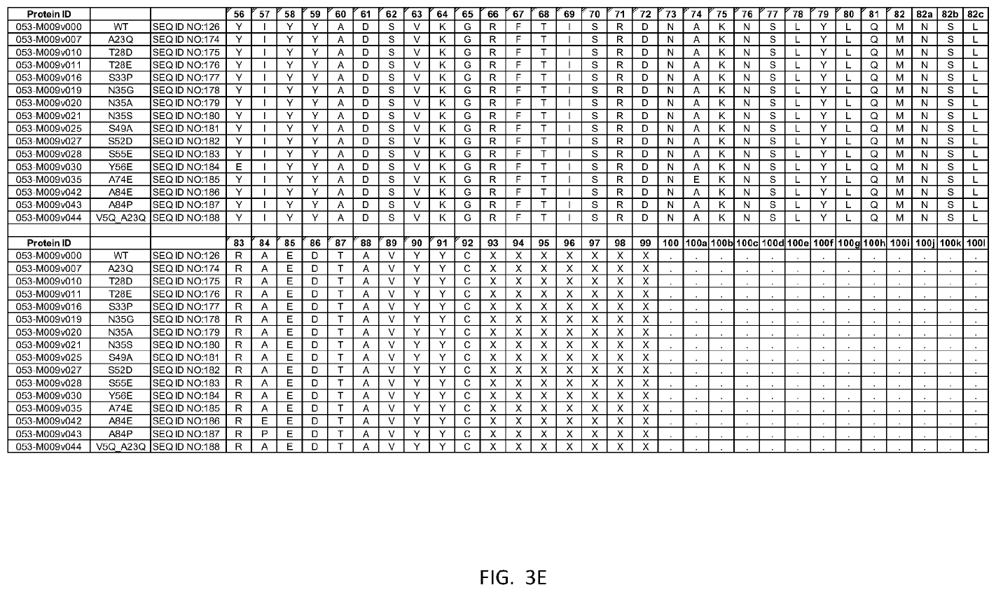

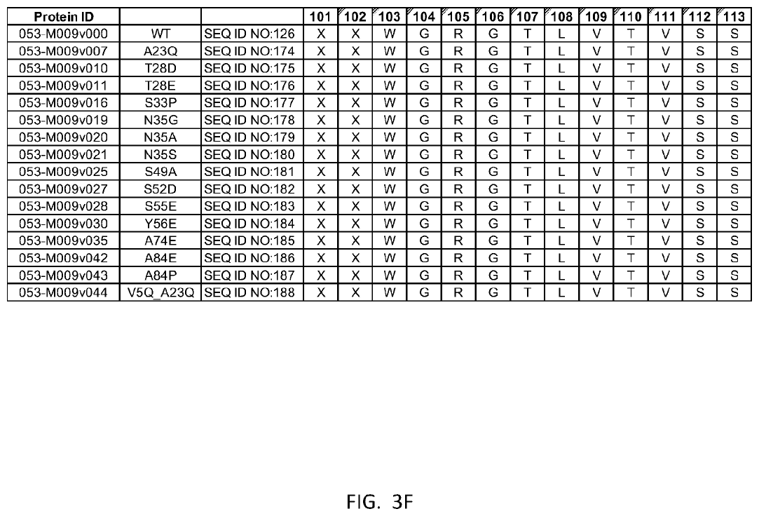

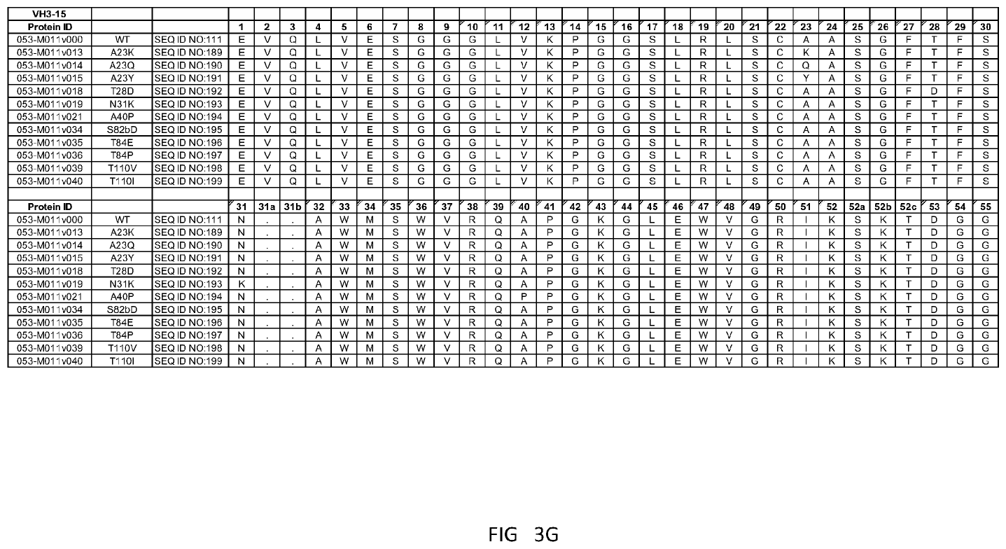

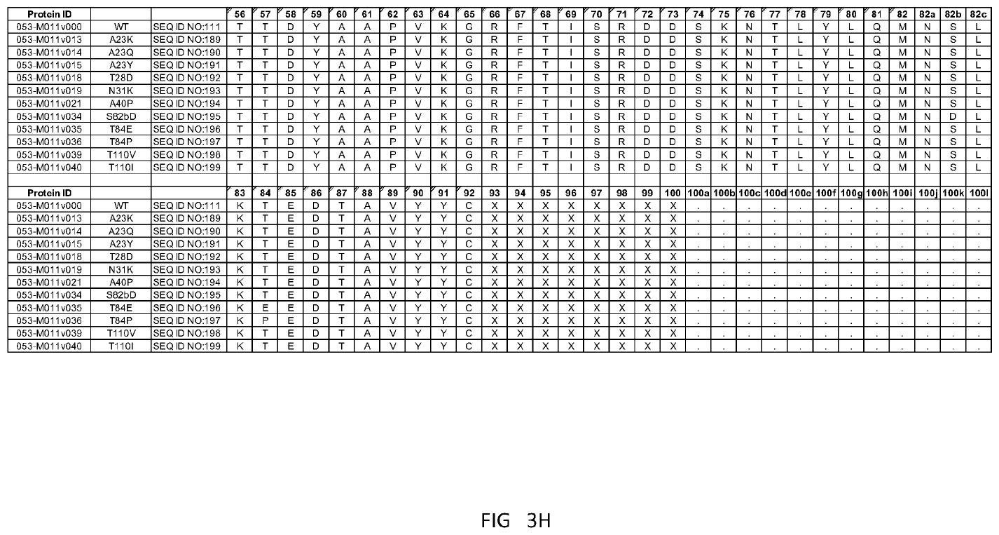

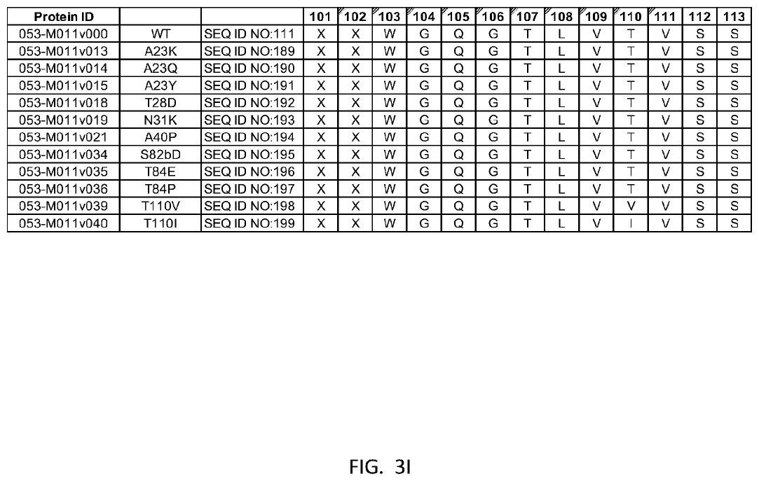

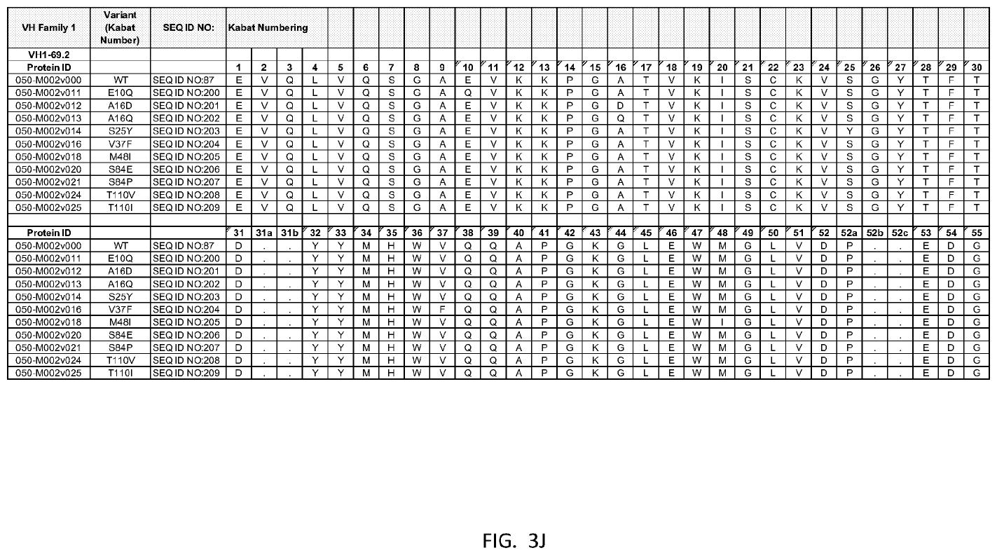

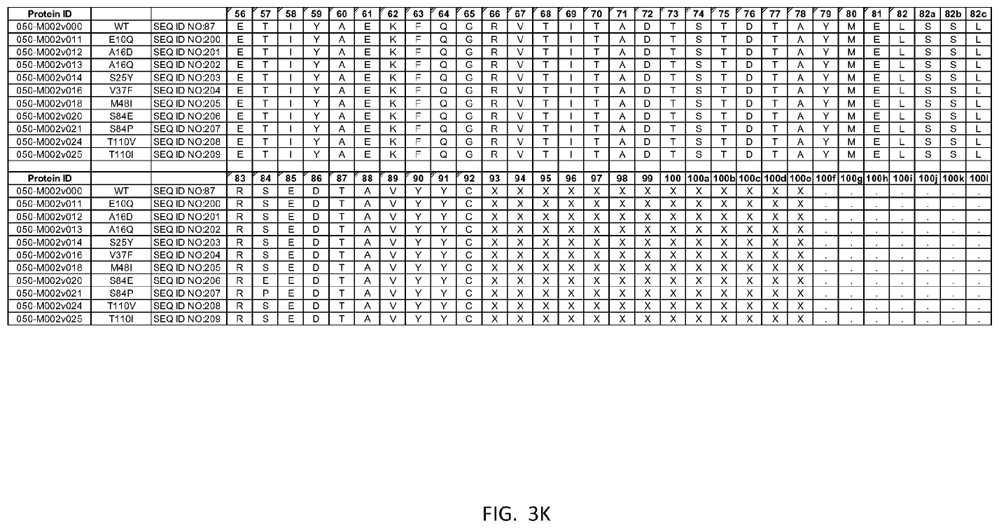

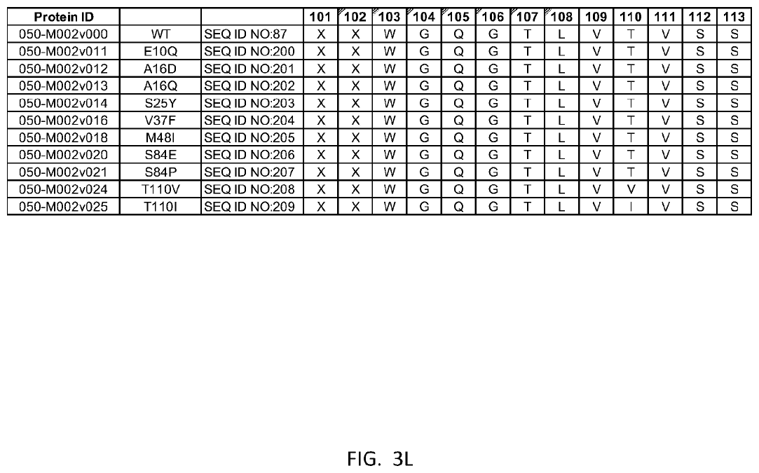

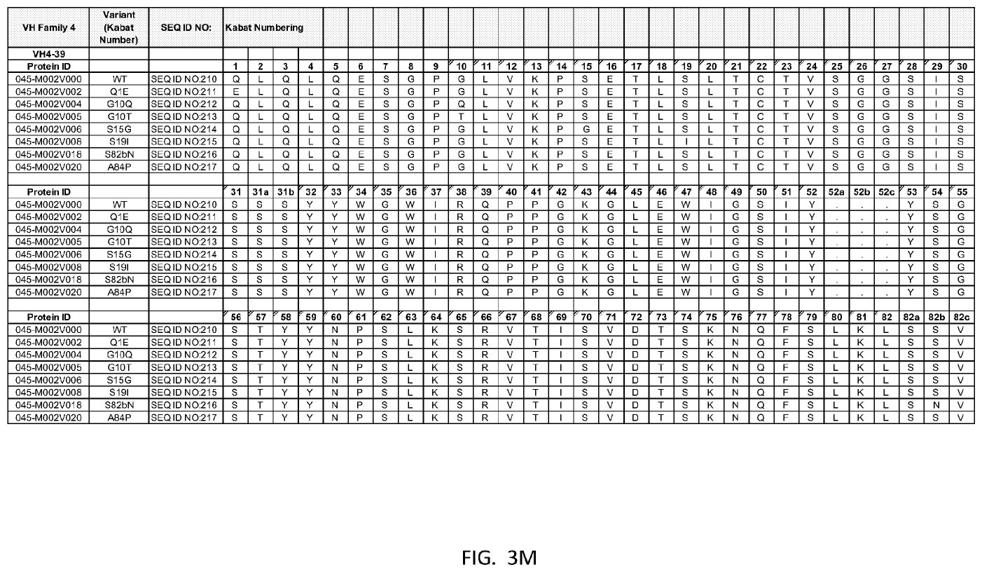

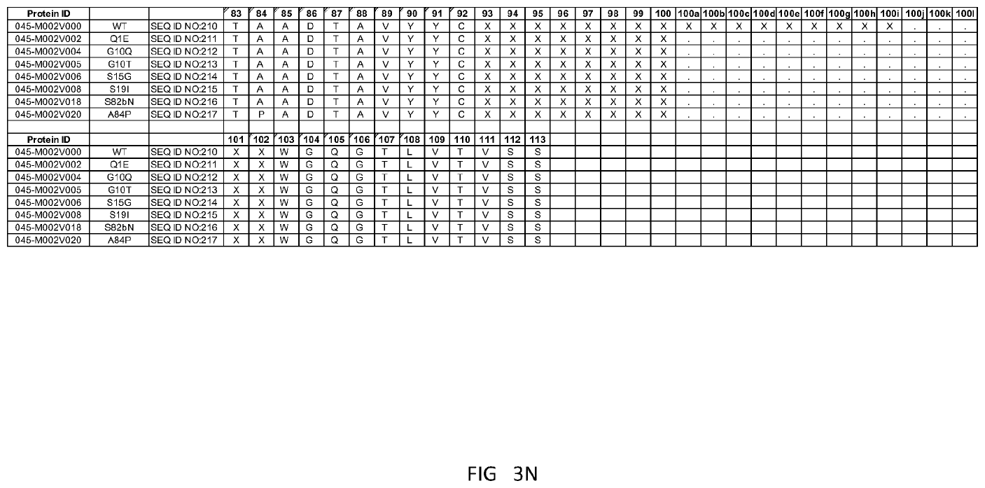

FIGS. 1A-1H shows the wild-type IGHV amino acid sequences of the functional germlines from a number of VH genes. These WT IGHV sequences have been modified to provide example modified IGHV sequences according to the disclosure herein. The variable domain germline sequences shown in FIGS. 1A-1H include the regions from FR1, CDR1, FR2, CDR2 and FR3, amino acids 1 to 94 or 95 (according to the Kabat numbering system), and encode the region encoding the optimized sequence variants. The sequences do not include CDR3 or FR4 because these segments come from the D and J chains as the result of homologous recombination to generate diversity and thus, are highly variable across antibodies. Accordingly, while CDR3 and FR4 may be present in full length VH embodiments of the disclosure and may themselves affect stability of the full length VH domains, the several of the embodied improvements of the disclosure are independent of CDR3 and FR4, with the exception, for example, of those variants that have a substitution at amino acid 102, 105, 107, or 110. Example of these full length sequences are shown in, for example, in FIGS. 2A-2O and 3A-3N. Where FR4 amino acids are shown, these are intended to be representative of the six J segments that are available in the human genome. But some embodiments of the disclosure include only V-gene portion of a full-length VH, even if D and J chain sequences are shown as part of the sequences disclosed herein.

In a first approach to modify the IGHV domains according to the disclosure, IGHV sequences from several human germlines were modified to introduce cysteine residues and create novel cysteine bonds between the residues. In a second approach, IGHV sequences were modified to substitute amino acids at various positions. In a third approach, a combination of both novel cysteine bonds and other modified amino acids were introduced. Each of the approaches can be used for IGHV sequences and full length VH domains across one or more germline families to modify at least one of the following properties of the domains: thermal stability, cellular expression, VH dimerization and light chain pairing.

Following one or more of the approaches identified herein, one or more substitutions introduce cysteine residues that create one or more novel disulfide bonds in the IGHV sequences or full length VH. In particular embodiments, the IGHV sequences or the full length VH of the disclosure include cysteine residues in combinations at the following positions (according to the Kabat numbering system): positions 2 and 102; 17 and 82a; 19 and 81; 23 and 77; 34 and 78; 35 and 50, which result in the following amino acid combinations: 2C/102C; 17C/82aC; 19C/81C; 23C/77C; 34C/78C; and 35C/50C. Cysteine bonds between these positions can conformationally lock down and stabilize the modified VH domains. FIGS. 2A-2O, for example, shows a number VH domains of the disclosure with cysteine residues that form novel disulfide bonds, which may be referred to herein as “cys clamp(s).” In addition, several other sets of Figures herein include one of these sets of cysteine substitutions as further described herein.

In another approach for modifying and/or improving the biophysical properties of the VH domains of the disclosure, VH domains from a number of human germline families were modified to provide the following amino acids (according to the Kabat numbering system): 1E, 2A, 5Q, 10Q, 10T, 14E, 15G, 16D, 16Q, 19I, 23K, 23Q, 23Y, 25F, 25Y, 28D, 28E, 28K, 28N, 28R, 30K, 30S, 31K, 33P, 35A, 35G, 35S, 37F, 37Y, 37H, 39R, 40P, 44D, 45E, 48I, 49A, 52E, 52D, 55E, 56E, 60A, 60D, 65D, 68E, 73D, 73P, 74E, 76K, 76N, 77Q, 82bD, 82bN, 83D, 83K, 83L, 83Q, 83T, 84E, 84P, 84Y, 85K, 85R, 85S, 85T, 89I, 105D, 107I, 107Y, 110I, and 110V

In addition, combinations of two or more of these (or other) amino acids can be used to modify and/or improve the biophysical properties of the VH domains. In various aspects of the disclosure, the combinations may include, for example the following:

| | | |
|---|---|---|
| 5Q/23Q | 16D/37F | 28D/39R/45E/76N/84E |
| 10Q/48I/84E | 16D/37Y | 28D/39R/48I/83D |
| 10T/82bD | 16D/39R/48I | 28D/39R/48I/84E |
| 10T/82bD | 16D/48I | 28D/39R/76N/83D |
| 10T/82bN | 16D/110I | 28D/39R/76N/84E |
| 10T/84P | 23Q/77Q | 28D/48I/83D |
| 15G/37Y | 28D/37Y/48I/83D | 28D/48I/84E |
| 15G/44D | 28D/37Y/48I/84E | 28D/49A |
| 15G/85S | 28D/37Y/76N/83D | 28D/49A/77Q |
| 15G/83T | 28D/37Y/76N/84E | 28D/55E |
| 28D/55E/74E | 37Y/48I | 44D/85S |
| 28D/76N/83D | 37Y/49A/74E | 44D/83T |
| 28D/76N/84E | 37Y/85S | 45E/82bD/84P |
| 28K/49A | 37Y/83T | 49A/55E |
| 28K/49A/77Q | 39R/28D | 49A/55E/77Q |
| 28K/49A/55E/84E | 39R/45E | 49A/55E/84E |
| 28K/49A/55E/84E/10T/ | 39R/48I | 49A/74E |
| 82bN | 39R/60A | 49A/74E/77Q |
| 28K/55E | 39R/60D | 49A/77Q |
| 28K/55E/74E | 39R/68E | 49A/77Q/55E |
| 37F/48I | 39R/76N | 49A/77Q/84E |
| 37Y (or 39R)/10T/84P | 39R/83D | 45E/82bD/84P |
| 37Y (or 39R)/10T/82bD | 39R/84E | 49A/84E |
| 37Y (or 39R)/82bD/84P | 39R/83T | 82bD/84P |
| 37Y/39R/83T | 39R/45E/48I | 82bN/84P |
| 37Y/39R/45E/83T | 39R/45E/49A/74E | 83T/44D |
| 37Y/44D | 39R/45E/82bD/84P | |

In a number of embodiments of the modified IGHV sequences and the VH domains of the disclosure, position 39 is modified to arginine (39R), which can result in increased solubility and decreased propensity to pair with VL domains. FIGS. 3A-3N shows a number VH domains of the disclosure with selected amino acid substitutions according to the disclosure. In some aspects, modified IGHV sequences and VH domains of the disclosure include substitution of position 37 to tyrosine (37Y), which reduces light chain pairing as well as dimerization with other VH domains. In some aspects of the disclosure, the modified IGHV sequences and VH domains include one or both 39R and 37Y. In addition, adding 37Y to the VH domains can have a neutral or positive affect on expression and stability across VH domains from multiple VH families. Accordingly, each of the IGHV sequences and VH domains according to the disclosure may include 37Y and/or 39R if not already present.

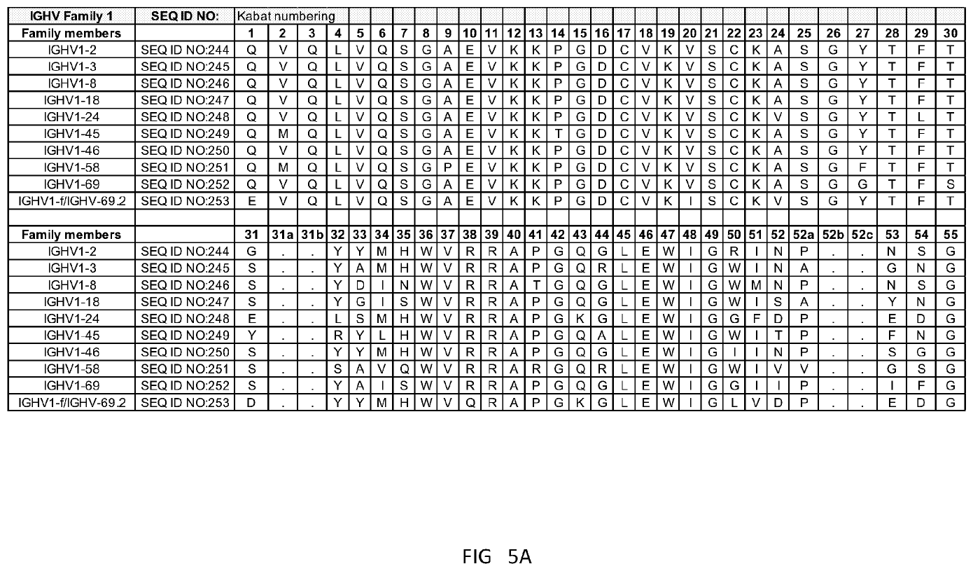

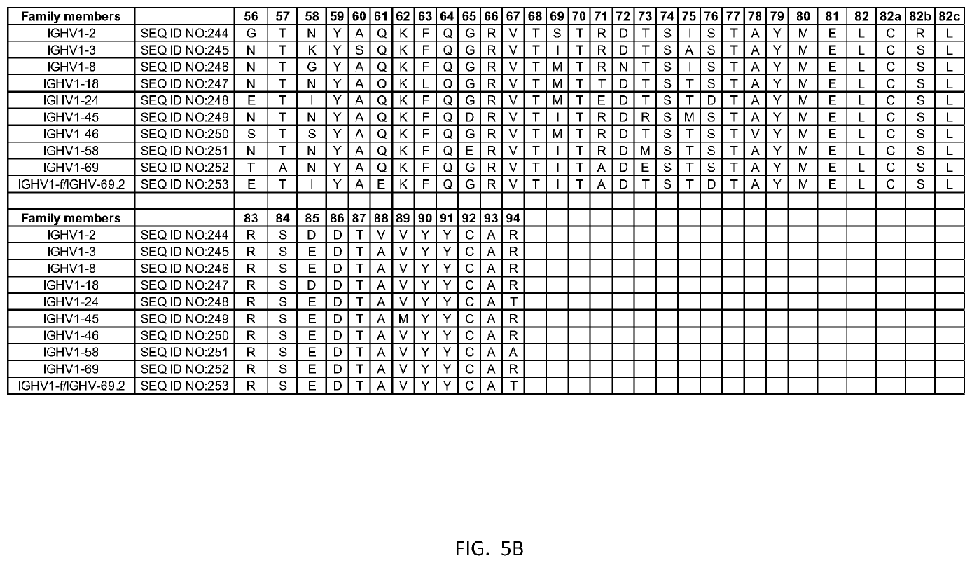

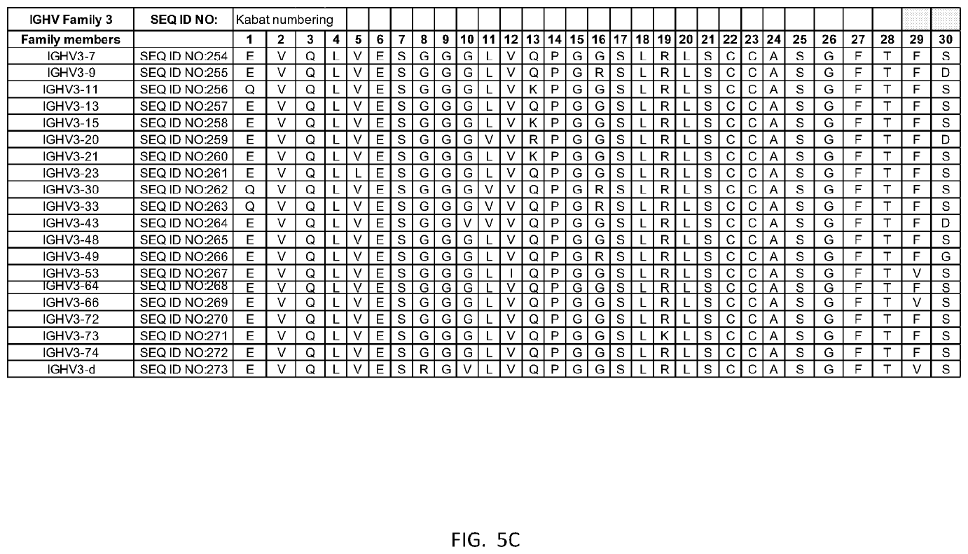

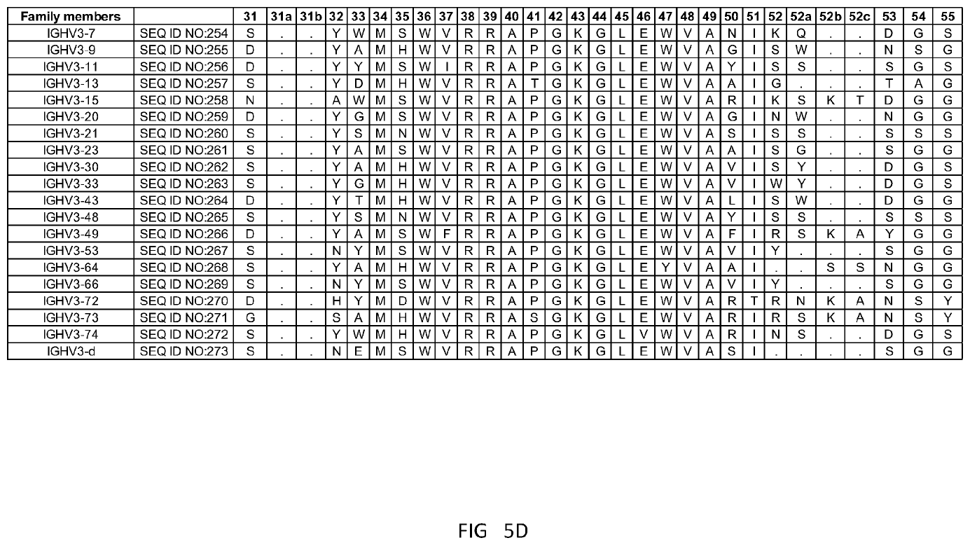

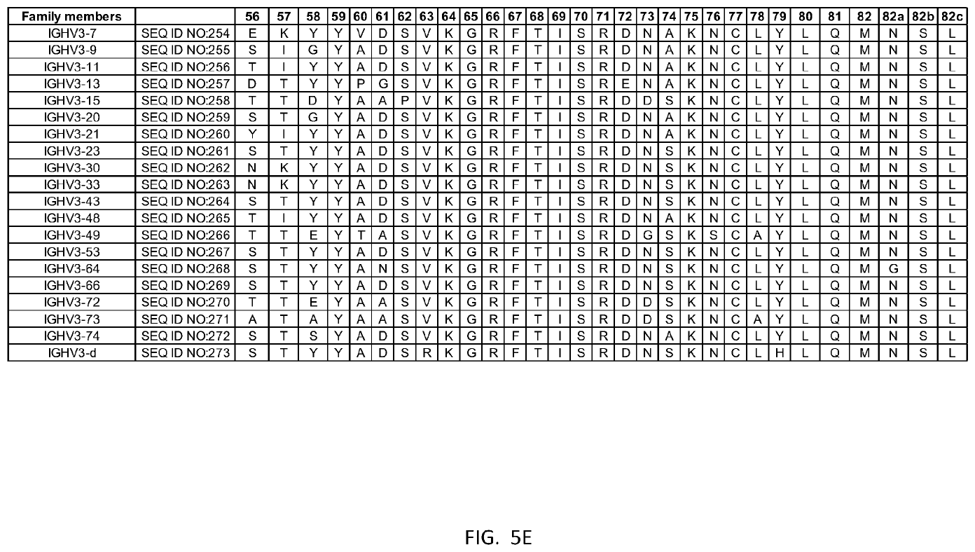

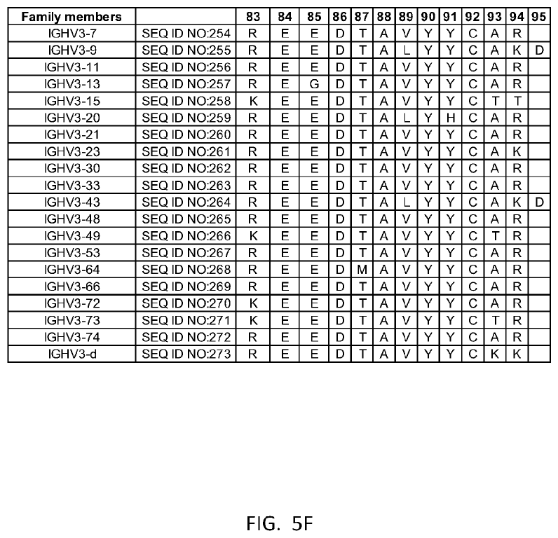

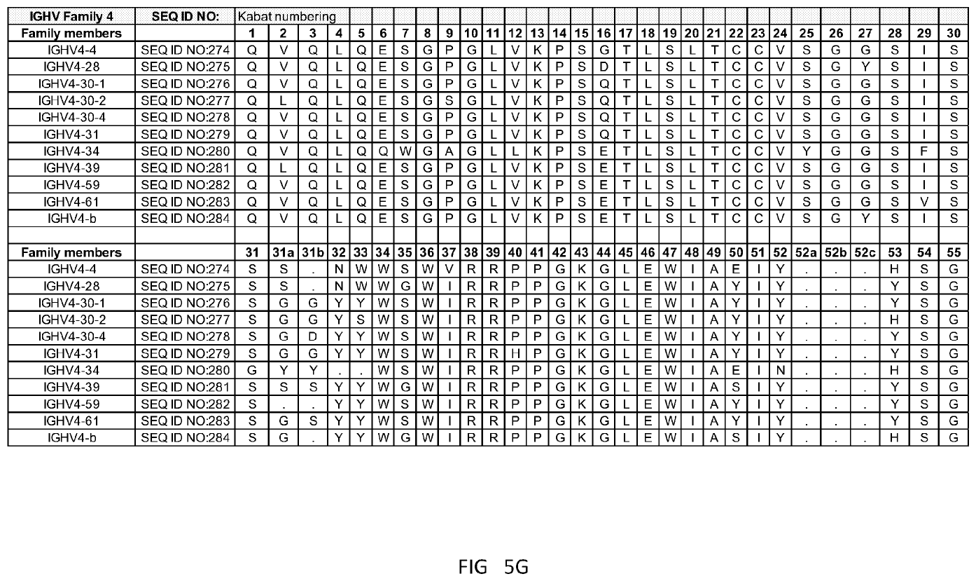

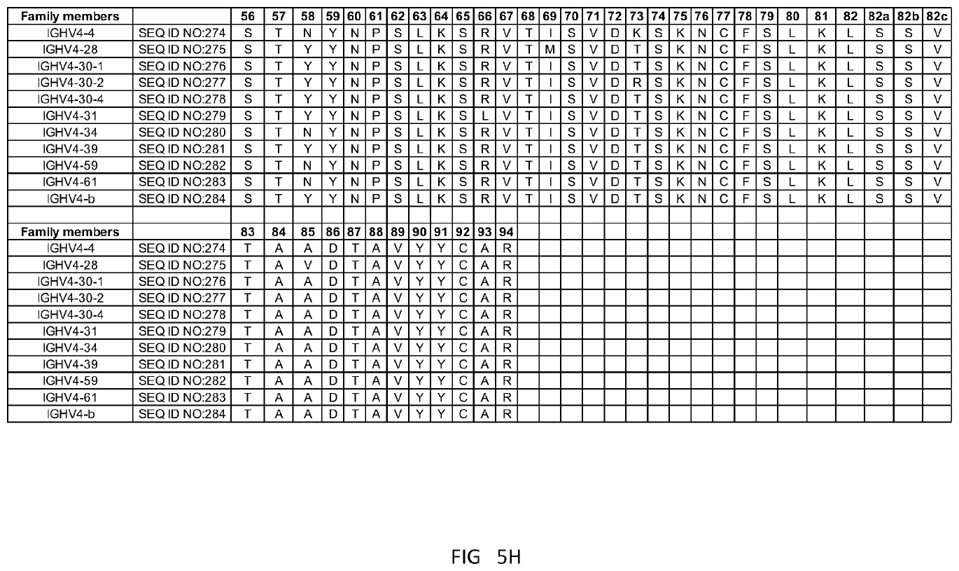

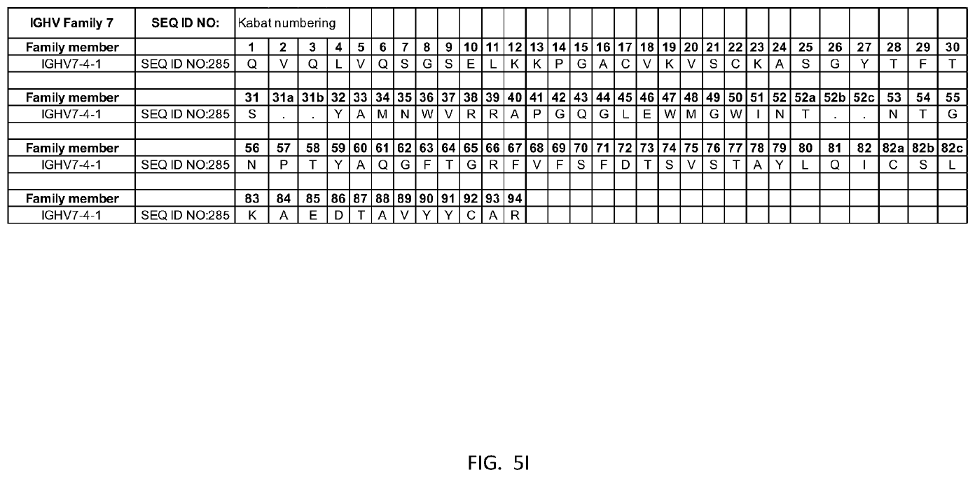

In some embodiments of the germline sequences described herein, amino acids that may be modified in one IGHV sequence or VH domain are natural in another IGHV sequence or VH domain. For example, amino acid 49 in the germline VH IGHV3-7 sequence in FIG. 1C is alanine, but the germline amino acid sequence in position 49 of IGHV 3-9 is a serine, which was modified to alanine in the stabilized variant, as shown in FIG. 5D. Additionally, the sequences shown in all of the figures use the—*01 allele as representative for the all the family polymorphs, which are readily available from the IMGT® database.

A combination of the above approaches can lead to further improved properties for the VH domains. Accordingly, any one or more of the non-cysteine substitutions or combination thereof described above can be combined with any one of the cysteine combinations (cys clamps). In particular examples, any one of the foregoing cysteine residue combinations can be further combined with one or more of the of the amino acid substitutions of the disclosure and combinations thereof, which may include any of the combinations described above.

In addition, if not already included in a combination, 39R and 37Y may also be included. The outcome of the combinations, result in IGHV sequences or VH domains having one of the following cys clamps: 2C/102C; 17C/82aC; 19C/81C; 23C/77C; 34C/78C; and 35C/50C, combined with one or more of the single amino acid substitutions or combinations thereof as disclosed herein.

IGHV sequences and VH domains from a number of human antibody germlines are suitable for substitution to provide improved properties according to the various aspects of the disclosure, including, for example, VH family 1, VH family 2, VH family 3, VH family 4, VH family 5, and VH family 7. Additionally, a number of examples of substitutions in particular human antibody germlines are provided below.

### Example Substitutions to Germline Family 1

Examples of the IGHV sequences include members of germline V-gene family 1, for example germline family gene members 1-2 (SEQ ID NO: 1), 1-3 (SEQ ID NO: 2), 1-8 (SEQ ID NO: 3), 1-18 (SEQ ID NO: 4), 1-24 (SEQ ID NO: 5), 1-45 (SEQ ID NO: 6), 1-46 (SEQ ID NO: 7), 1-58 (SEQ ID NO:8), 1-69 (SEQ ID NO: 9), and 1-69.2 (also known as 1-f) (SEQ ID NO: 10), and alleles thereof.

In various aspects of the disclosure, of the members of germline family 1 can be modified to include cysteine residue combinations at the following positions (according to the Kabat numbering system): positions 2 and 102; 17 and 82a; 19 and 81; 23 and 77; 34 and 78; 35 and 50, which result in the following amino acid combinations: 2C/102C; 17C/82aC; 19C/81C; 23C/77C; 34C/78C; and 35C/50C.

In addition, example family 1 substitutions may include one or more of the following: 10Q, 16D, 16Q, 25Y, 25F, 37F, 37Y, 39R, 45E, 48I, 84E, 84P, 110V, and 110I.

Example family 1 substitution combinations include, but are not limited to, the following:

| | | |
|---|---|---|
| 10Q/48I/84E | 16D/48I | 39R/45E/48I |
| 16D/37F | 16D/110I | 39R/48I |
| 16D/37Y | 37F/48I | |
| 16D/39R/48I | 37Y/48I | |

In addition, family 1 substitutions include either 17C/82aC or 34C/78C along with other single or multiple substitutions to provide the following example combinations of substitutions:

| | | |
|---|---|---|
| 17C/82aC/10Q/48I/84E | 17C/82aC/16D/48I | 17C/82aC/84E |
| 17C/82aC/16D | 17C/82aC/37F | 34C/78C/16D |
| 17C/82aC/16D/37F | 17C/82aC/37Y | 34C/78C/37F |
| 17C/82aC/16D/37Y | 17C/82aC/37Y/48I | 34C/78C/84E |
| 17C/82aC/16D/37Y/39R | 17C/82aC/39R | 34C/78C/16D/37F |
| 17C/82aC/16D/39R | 17C/82aC/39R/45E/48I | 34C/78C/16D/48I |
| 17C/82aC/16D/39R/48I | 17C/82aC/39R/48I | 34C/78C/10Q/48I/84E |

As described herein, each of the combinations may include one or more of 37Y, 39R, and 45E, or if not already included.

### Example Substitutions to Germline Family 2

Examples of the IGHV sequences include members of germline V-gene family 2, for example germline family gene members 2-5 (SEQ ID NO: 11) 2-26 (SEQ ID NO: 12) and 2-70 (SEQ ID NO: 13), and alleles thereof.

In various aspects of the disclosure, of the members of germline family 2 can be modified to include cysteine residue combinations at the following positions (according to the Kabat numbering system): positions 2 and 102; 17 and 82a; 19 and 81; 23 and 77; 34 and 78; 35 and 50, which result in the following amino acid combinations: 2C/102C; 17C/82aC; 19C/81C; 23C/77C; 34C/78C; and 35C/50C.

In addition, example family 2 substitutions may include one or more of the following: 15G, 16D, 37Y, 37H, 39R, 44D, 45E, 65D, 73D, 73P, 83L, 83Q, 83K, 83T, 84Y, 85R, 85S, 85K, 85T, 89I, 105D, 107I.

Example family 2 substitution combinations include, but are not limited to, the following:

| | | |
|---|---|---|
| 15G/37Y | 37Y/39R/45E/83T | 37Y/83T |
| 15G/44D | 37Y/39R/83T | 39R/83T |
| 15G/85S | 37Y/44D | 44D/85S |
| 15G/83T | 37Y/85S | 44D/83T |

In addition, family 2 substitutions include 19C/82C along with other single or multiple substitutions to provide the following example combinations of substitutions:

| | | |
|---|---|---|
| 19C/81C/15G | 19C/81C/37Y/39R/83T | 19C/81C/44D |
| 19C/81C/15G/37Y | 19C/81C/37Y/39R/45E/83T | 19C/81C/44D/85S |
| 19C/81C/15G/44D | 19C/81C/37Y/44D | 19C/81C/85S |
| 19C/81C/15G/85S | 19C/81C/37Y/83T | 19C/81C/83T |
| 19C/81C/15G/83T | 19C/81C/37Y/85S | 19C/81C/83T/44D |
| 19C/81C/37Y | 19C/81C/39R/83T | |

As described herein, each of the combinations may include one or more of 37Y, 39R, and 45E if not already included.

### Example Substitutions to Germline Family 3

Examples of the VH domains of the disclosure include members of germline V-gene family 3, for example germline family gene members 3-7 (SEQ ID NO: 14), 3-9 (SEQ ID NO: 15), 3-11 (SEQ ID NO: 16), 3-13 (SEQ ID NO: 17), 3-15 (SEQ ID NO: 18), 3-20 (SEQ ID NO: 19), 3-21 (SEQ ID NO: 20), 3-23 (SEQ ID NO: 21), 3-30 (SEQ ID NO: 22), 3-33 (SEQ ID NO: 23), 3-43 (SEQ ID NO: 24), 3-48 (SEQ ID NO: 25), 3-49 (SEQ ID NO: 26), 3-53 (SEQ ID NO: 27), 3-64 (SEQ ID NO: 28), 3-66 (SEQ ID NO: 29), 3-72 (SEQ ID NO: 30), 3-73 (SEQ ID NO: 31), 3-74 (SEQ ID NO: 32), 3-d (SEQ ID NO: 33), and 3-NL1 (SEQ ID NO: 34), and alleles thereof.

In various aspects of the disclosure, of the members of germline family 3 can be modified to include cysteine residue combinations at the following positions (according to the Kabat numbering system): positions 2 and 102; 17 and 82a; 19 and 81; 23 and 77; 34 and 78; 35 and 50, which result in the following amino acid combinations: 2C/102C; 17C/82aC; 19C/81C; 23C/77C; 34C/78C; and 35C/50C.

In addition, example family 3 substitutions may include one or more of the following: 2A, 5Q, 14E, 23K, 23Q, 23Y, 28D, 28E, 28N, 28K, 28R, 30K, 30S, 31K, 33P, 35G, 35A, 35S, 37Y, 39R, 40P, 45E, 49A, 52E, 52D, 55E, 56E, 74E, 76K, 77Q, 82bD, 84E, 84P, 110V, 110I

Example family 3 substitution combinations include, but are not limited to the following:

| | | |
|---|---|---|
| 5Q/23Q | 28K/49A | 39R/45E/49A/74E |
| 23Q/77Q | 28K/49A/55E/84E | 39R/49A/84E |
| 28D/49A | 28K/49A/77Q | 39R/84E |
| 28D/49A/77Q | 28K/55E | 49A/55E |
| 28D/55E | 28K/55E/74E | 49A/55E/77Q |
| 28D/55E/74E | 37Y/49A/74E | 49A/55E/84E |
| 49A/74E/77Q | 49A/77Q/55E | 49A/84E |
| 49A/77Q | 49A/77Q/84E | |

In addition, family 3 substitutions include either 23C/77C along with other single or multiple substitutions to provide the following example combinations of substitutions:

| | | |
|---|---|---|
| 23C/77C/28K/49A | 23C/77C/39R/45E/49A/74E | 34C/78C/28K |
| 23C/77C/28D/49A | 23C/77C/39R/49A/74E | 34C/78C/49A |
| 23C/77C/28K/55E | 23C/77C/39R/49A/84E | 34C/78C/55E |
| 23C/77C/28K/55E/74E | 23C/77C/39R/49A/84E | 34C/78C/74E |
| 23C/77C/28K/49A/55E/84E | 23C/77C/49A/55E/84E | 34C/78C/77Q |
| 23C/77C/37Y/49A/74E | 34C/78C/28D | 34C/78C/84E |

As described herein, each of the combinations may include one or more of 37Y, 39R or 45E, if not already included.

### Example Substitutions to Germline Family 4

Examples of the VH domains of the disclosure include members of germline V-gene family 4, for example germline family gene members include 4-4 (SEQ ID NO: 35), 4-28 (SEQ ID NO: 36), 4-30-1 (SEQ ID NO: 37), 4-30-2 (SEQ ID NO: 38), 4-30-4 (SEQ ID NO: 39), 4-31 (SEQ ID NO: 40), 4-34 (SEQ ID NO: 41), 4-38-2 (SEQ ID NO: 42), 4-39 (SEQ ID NO: 43), 4-59 (SEQ ID NO: 44) and 4-61 (SEQ ID NO: 45), 4-b (SEQ ID NO: 46), and alleles thereof.

In various aspects of the disclosure, of the members of germline family 4 can be modified to include cysteine residue combinations at the following positions (according to the Kabat numbering system): positions 2 and 102; 17 and 82a; 19 and 81; 23 and 77; 34 and 78; 35 and 50, which result in the following amino acid combinations: 2C/102C; 17C/82aC; 19C/81C; 23C/77C; 34C/78C; and 35C/50C.

In addition, example family 4 substitutions may include one or more of the following: 1E, 10Q, 10T, 15G, 19I, 82bD, 82bN, 84P, 107I, 107Y, and combinations thereof.

Example family 4 substitution combinations include the following:

| | | |
|---|---|---|
| 10T/82bN | 10T/84P | 10T/82bD |
| 37Y (and/or 39R)/82bN/84P | 37Y (and/or 39R)/10T/82bN | 82bN/84P |
| 37Y (and/or 39R)/10T/84P | 39R/45E/82bD/84P | 82bD/84P |
| 37Y (and/or 39R)/10T/82bD | 45E/82bD/84P | |

In addition, family 4 substitutions include either 17C/82aC or 23C/77C along with other single or multiple substitutions to provide the following example combinations of substitutions:

| | |
|---|---|
| 17C/82aC/10T | 23C/77C/45E/82bD/84P |
| 17C/82aC/10T/82bN | 23C/77C/82bD/84P |
| 17C/82aC/10T/82bD | 23C/77C/82bN/84P |
| 17C/82aC/82bN/84P | 23C/77C/37Y (and/or 39R)/10T/82bD |
| 17C/82aC/37Y (and/or 39R)/10T/82bD | 23C/77C/37Y (and/or 39R)/10T/82bN |
| 17C/82aC/37Y (and/or 39R)/10T/84P | 23C/77C/37Y (and/or 39R)/10T/84P |
| 17C/82aC/37Y (and/or 39R)/82bD/84P | 23C/77C/37Y (and/or 39R)/82bD/84P |
| 23C/77C/10T/84P | 23C/77C/37Y (and/or 39R)/82bD/84P |
| 23C/77C/39R/45E/82bD/84P | |

In each of the example family 4 combinations, the combinations may also include one more of 37Y, 39R and 45E if not already present.

### Example Substitutions to Germline Family 5

Examples of the VH domains of the disclosure include members of germline V-gene family 5, for example germline family gene members 5-51 (SEQ ID NO: 47) and 5-a (also known as 5-10) (SEQ ID NO: 48), and alleles thereof.

In various aspects of the disclosure, the members of germline family 5 can be modified to include cysteine residue combinations at the following positions (according to the Kabat numbering system): positions 2 and 102; 17 and 82a; 19 and 81; 23 and 77; 34 and 78; 35 and 50, which result in the following amino acid combinations: 2C/102C; 17C/82aC; 19C/81C; 23C/77C; 34C/78C; and 35C/50C.

In addition, example family 5 substitutions may include one or more of the following: 28D, 37Y, 39R, 45E, 48I, 60A, 60D, 68E, 76N, 83D, and 84E, either alone, in combination, or in combination with a one of the cys clamps described herein.

Example family 5 substitution combinations include, but are not limited to the following:

| | | |
|---|---|---|
| 39R/28D | 39R/84E | 28D/39R/76N/84E |
| 39R/48I | 28D/48I/84E | 28D/39R/48I/83D |
| 39R/60A | 28D/76N/83D | 28D/37Y/48I/84E |
| 39R/60D | 28D/76N/84E | 28D/37Y/76N/83D |
| 39R/68E | 28D/48I/83D | 28D/37Y/76N/84E |
| 39R/76N | 28D/39R/48I/84E | 28D/37Y/48I/83D |
| 39R/83D | 28D/39R/76N/83D | 28D/39R/45E/76N/84E |

In each of the example family 5 combinations, the combinations may also include one or more of 37Y, 39R and 45 E, if not already present, and one of cys clamps as described herein.

### Example Substitutions to Germline Family 7

An example of the VH domains of the disclosure include a member of germline V-gene family 7, for example germline family gene member 7-4-1 (SEQ ID NO: 50).

In various aspects of the disclosure, the members of germline family 7 can be modified to include cysteine residue combinations at the following positions (according to the Kabat numbering system): positions 2 and 102; 17 and 82a; 19 and 81; 23 and 77; 34 and 78; 35 and 50, which result in the following amino acid combinations: 2C/102C; 17C/82aC; 19C/81C; 23C/77C; 34C/78C; and 35C/50C. These may be combined with one or more of 37Y, 39R and 45E.

Example family 7 substitution combinations include, but are not limited to the following:

| | | |
|---|---|---|
| 17C/82aC/39R | 17C/82aC/37Y | 35C/50C/39R/45E |
| 17C/82aC/39R/45E | 35C/50C/39R | 35C/50C/37Y |

In other embodiments of the disclose, members of human antibody germline family 6 may be modified with any of the foregoing amino acid substitutions or substitutions thereof.

Table 1 provides a summary of single amino acid substitutions in particular gene families that provided improved expression and or stability to several VH domains of the disclosure.

TABLE 1

| AA | VH1 | VH2 | VH3 | VH4 | VH5 | VH7 | VH 1-5 All |
|---|---|---|---|---|---|---|---|
| 1 | | | | 1E | | | 1E |
| 2 | | | 2A | | | | 2A |
| 5 | | | 5Q | | | | 5Q |
| 10 | 10Q | | | 10Q, 10T | | | 10Q, 10T |
| 14 | | | 14E | | | | 14E |
| 15 | | 15G | | 15G | | | 15G |
| 16 | 16D, 16Q | 16D | | | | | 16D, 16Q |
| 19 | | | | 19I | | | 19I |
| 23 | | | 23K, 23Q, 23Y | 23Q | | | 23K, 23Q, 23Y |
| 25 | 25Y, 25F | | | | | | 25F, 25Y |
| 28 | | | 28D, 28E, 28N, | | 28D | | 28D, 28E, 28N, |
| | | | 28K 28R | | | | 28K, 28R |
| 30 | | | 30K, 30S | | | | 30K, 30S |
| 31 | | | 31K | | | | 31K |
| 33 | | | 33P | | | | 33P |
| 35 | | | 35A, 35G, 35S | | | | 35A, 35G, 35S |
| 37 | 37F, 37Y | 37Y, 37H | 37Y | 37Y | 37Y | 37Y | 37F, 37Y, 37H |
| 39 | 39R | 39R | 39R | 39R | 39R | 39R | 39R |
| 40 | | | 40P | | | | 40P |
| 44 | | 44D | | | | | 44D |
| 45 | 45E | 45E | 45E | 45E | 45E | 45E | 45E |
| 48 | 48I | | | | 48I | | 48I |
| 49 | | | 49A | | | | 49A |
| 52 | | | 52E, 52D | | | | 52E, 52D |
| 55 | | | 55E | | | | 55E |
| 56 | | | 56E | | | | 56E |
| 60 | | | | | 60A, 60D | | 60A, 60D |
| 65 | | 65D | | | | | 65D |
| 68 | | | | | 68E | | 68E |
| 73 | | 73D, 73P | | | | | 73D, 73P |
| 74 | | | 74E | | | | 74E |
| 76 | | | 76K | | 76N | | 76K, 76N |
| 77 | | | 77Q | | | | 77Q |
| 82b | | | 82bD | 82bD, 82bN | | | 82bD, 82bN |
| 83 | | 83L, 83Q, 83K, | | | 83D | | 83D, 83K, 83L, 83Q, 83T |
| 84 | 84E, 84P | 83T | 84E, 84P | 84P | 84E | | 84E, 84P, 84Y |
| 85 | | 84Y | | | | | 85K, 85R, 85S, 85T |
| 89 | | 85R, 85S, 85K, 85T | | | | | 89I |
| 105 | | 89I | | | | | 105D |
| 107 | | 105D | | 107I, 107Y | | | 107I, 107Y |
| 110 | 110V, 110I | 107I | 110V, 110I | | | | 110I, 110V |

Table 2 provides a summary of example combinations of amino acids that in particular germline families that provide improved stability and/or expression of several of the VH domains of the disclosure.

TABLE 2

| Cys | Family | | | | | |
|---|---|---|---|---|---|---|
| Clamp | VH1 | VH2 | VH3 | VH4 | VH5 | VH7 |
| NA | 37F/48I | | 23Q/77Q | 10T/84P | 39R/28D | |
| | 16D/110I | | 5Q/23Q | | 39R/48I | |
| | 16D/37F | | 28D/49A | | 39R/60A | |
| | 16D/48I | | 49A/74E/77Q | | 39R/60D | |
| | 10Q/48I/84E | | 49A/77Q/84E | | 39R/68E | |
| | | | 49A/77Q | | 39R/76N | |
| | | | 28K/49A/77Q | | 39R/83D | |
| | | | 28D/49A/77Q | | 39R/84E | |
| | | | 49A/55E/77Q | | 28D/48I/84E | |
| | | | 28K/49A | | 28D/76N/83D | |
| | | | 28D/55E | | 28D/76N/84E | |
| | | | 28D/55E/74E | | 28D/48I/83D | |
| | | | 28K/55E | | 28D/39R/48I/84E | |
| | | | 49A/55E | | 28D/39R/76N/83D | |
| | | | 49A/77Q/55E | | 28D/39R/76N/84E | |
| | | | | | 28D/39R/48I/83D | |
| | | | | | 28D/37Y/48I/84E | |
| | | | | | 28D/37Y/76N/83D | |
| | | | | | 28D/37Y/76N/84E | |
| | | | | | 28D/37Y/48I/83D | |
| | | | | | 28D/39R/45E/76N/ | |
| | | | | | 84E | |
| 17C/ | 17C/82aC/39R/48I | | | 17C/82aC/10T | | 17C/82aC/39R |
| 82aC | 17C/82aC/16D/39R/ | | | 17C/82aC/10T/82bN | | 17C/82aC/39R/45E |
| | 48I | | | 17C/82aC/82bN/84P | | 17C/82aC/37Y |
| | 17C/82aC/37Y/48I | | | 17C/82aC/10T/82bD | | |
| | 17C/82aC/16D | | | 17C/82aC/37Y (and/ | | |
| | 17C/83aC/16D/37Y | | | or 39R)/10T/82bD | | |
| | 17C/82aC/84E | | | 17C/82aC/37Y(and/ | | |
| | 17C/82aC/37F | | | or 39R)10T/84P | | |
| | 17C/82aC/37Y | | | 17C/82aC/37Y (and/ | | |
| | 17C/82aC/16D/37F | | | or 39R)/82bD/84P | | |
| | 17C/82aC/16D/48I | | | | | |
| | 17C/82aC/10Q/48I/ | | | | | |
| | 84E | | | | | |
| | 17C/82aC/39R/48I/ | | | | | |
| | 17C/82aC/39R/45E/ | | | | | |
| | 48I | | | | | |
| 19C/ | | 19C/81C/85S | | | | |
| 81C | | 19C/81C/15G/85S; | | | | |
| | | 19C/81C/15G; | | | | |
| | | 19C/81C/37Y; | | | | |
| | | 19C/81C/37Y/85S; | | | | |
| | | 19C/81C/15G/37Y; | | | | |
| | | 19C/81C/44D; | | | | |
| | | 19C/81C/37Y/44D; | | | | |
| | | 19C/81C/15G/44D; | | | | |
| | | 19C/81C/44D/85S; | | | | |
| | | 19C/81C/83T; | | | | |
| | | 19C/81C/83T/44D; | | | | |
| | | 19C/81C/37Y/83T; | | | | |
| | | 19C/81C/15G/83T; | | | | |
| | | 19C/81C/39R/83T | | | | |
| | | 19C/81C/37Y/39R/ | | | | |
| | | 83T | | | | |
| | | 19C/81C/37Y/39R/ | | | | |
| | | 45E/83T | | | | |
| 23C/ | | | 23C/77C/39R/49A/ | 23C/77C/10T/82bN | | |
| 77C | | | 74E | 23C/77C/10T/82bD | | |
| | | | 23C/77C/39R/49A/ | 23C/77C/10T/84P | | |
| | | | 84E | 23C/77C/82bN/84P | | |
| | | | 23C/77C/39R/45E/ | 23C/77C/82bD/84P | | |
| | | | 49A/74E | 23C/77C/37Y (and/ | | |
| | | | 23C/77C/37Y/49A/ | or 39R)/82bN/84P | | |
| | | | 74E | 23C/77C/37Y (and/ | | |
| | | | 23C/77C/28K/49A | or 39R)/10T/84P | | |
| | | | 23C/77C/28D/49A | 23C/77C/37Y (and/ | | |
| | | | 23C/77C/28K/55E | or 39R)/10T/82bN | | |
| | | | 23C/77C/28K/55E/ | 23C/77C/37Y (and/ | | |
| | | | 74E | or 39R)/10T/82bD | | |
| | | | 23C/77C/49A/55E/ | 23C/77C/37Y (and/ | | |
| | | | 84E | or 39R)/82bD/84P | | |
| | | | 23C/77C/28K/49A/ | 23C/77C/39R/45E/ | | |
| | | | 55E/84E | 82bD/84P | | |
| 34C/ | 34C/78C/16D | | 34C/78C/28K | | | |
| 78C | 34C/78C/37F | | 34C/78C/28D | | | |
| | 34C/78C/84E | | 34C/78C/49A | | | |
| | 34C/78C/16D/37F | | 34C/78C/55E | | | |
| | 34C/78C/16D/48I | | 34C/78C/74E | | | |
| | 34C/78C/10Q/48I/ | | 34C/78C/77Q | | | |
| | 84E | | 34C/78C/84E | | | |
| 35C/ | | | | | | 35C/50C/39R |
| 50C | | | | | | 35C/50C/39R/45E |
| | | | | | | 35C/50C/37Y |

Additional embodiments of the disclosure include only a framework section (FR1, FR2. FR3 or FR4) or sections of the IGHV sequences or the VH domains. For example, a framework section or sections are of a germline family member modified according to the disclosure. In addition, the disclosure includes an IGHV or full length VH such that the CDRs may be the same or different than those for the IGHV sequences or VH domains identified herein. Accordingly, aspects of the disclosure are directed to polypeptides comprising one framework region or two, three or four framework regions of a human heavy chain V-gene portion (IGHV) of an antibody or full length VH, wherein the IGHV amino acid sequence or full length VH comprises one or more amino acid substitutions that result in an improved biophysical property such as increased thermal stability, increased cellular expression, and decreased VH dimerization and light chain pairing, as compared to a wild-type IGHV sequence lacking the one or more amino acid substitutions. The IGHV sequences may also include the framework portion of the J chain. The polypeptides may include any one of the above-described amino acid substitutions or combinations thereof. To the extent that one of the modified amino acids falls within one of the CDRs of the IGHV or VH domain, the remainder of the CDR may be the same or different than those identified in the sequences disclosed herein.

In several of the Figures, CDR3 for several of the amino sequences (amino acid positions 93-102, including amino acids 100a, 100b, 100c, 100d, 100e, 100f, 100g, 100h, 100i, 100j, 100k, and 100l according to the Kabat numbering system) are identified with “X” amino acids. Consideration of the CDR3s across the several germlines reflects that the CDR3 sequences have only a limited amount of homology. As an example, with regard to the VH domains in FIGS. 2A-2O:
(a) there is a minimum of 6% and maximum of 50% identity between HCDR3s with an average identity near 25% across all the sequences, and
(b) there was a minimum length of 12 and a maximum length of 21 residues with an average length of 14.6 residues.

Similarly, with regard to the VH domains in FIGS. 3A-3N:
(a) there is a minimum of 6% and maximum of 50% identity between HCDR3s with an average identity near 25% across all the sequences, and
(b) there is a minimum length of 12 and a maximum length of 21 residues with an average length of 14.6 residues

These data indicate that the observed stabilization effects as a result of the various VH domain substitutions that were tested (see e.g., Example 1) were not HCDR3-dependent. Instead, the data indicate that the amino acid substitutions disclosed herein, regardless of CDR3, were surprisingly and unexpectedly stabilizing for each of their germline families. Several of the variable domain portions of germline origin modified VH domains of the disclosure that are shown in FIGS. 1A-1H and 5A-5I include amino acids 1 to 94 or 95 (according to the Kabat numbering system) and represent the V-genes.

The VH substitutions of the disclosure are shown to improve at least the stability and/or expression of the VH domains having origins over multiple germline origins. Accordingly, such substitutions are not limited to particular VH domain amino acid sequences and instead may be useful over a wide range germlines and sequences. In addition, the VH substitutions described herein can result increased stability and/or expression regardless of the CDRs and their corresponding antigen or epitope. Therefore, the VH substitutions described herein are suitable for use with any VH domain, regardless of germline and regardless of CDRs.

In other aspects of the disclosure, the substitutions may be used in sequences that are similar, but not identical, to the IGHV sequences or full length VH domains described herein. For instance, the substitutions described herein may be used in sequences that are at least 50%, 60, 70%, 80%, 85%, 80%, 85%, 90%, 91%, 92%, 93%, 94%, 95%, 96%, 97%, 95% or 99% identical to the IGVH sequences or full length VH domains described herein, wherein the CDRs the are excluded from the determination of the percent identity. For example, the substitutions of the disclosure may be used in IGVH sequences or the VH domains having at least 50%, 60, 70%, 80%, 85%, 90%, 91%, 92%, 93%, 94%, 95%, 96%, 97%, 95% or 99% identity to any one of the framework portions of SEQ ID NOS: 1-50, and 76-627 and their alleles, along with other IGHV and VH domains of human antibody germline sequences.

The IGVH sequences and VH domains of the disclosure may be synthesized or expressed by methods known in the art. For example, the IGVH sequences and VH domains of the disclosure may be synthesized or expressed in genetically engineered animals, for example, mice, rats, rabbits, cows either being substituted into the VH locus or a separate, transgene, with the endogenous heavy and light chain, lambda and kappa, loci being inactivated or unable to express endogenous heavy and light chain genes (Bruggeman et al., Human Antibody Production in Transgenic Animals Arch. Immunol. Ther. Exp. 63, 101-108 (2015). https://doi.org/10.1007/s00005-014-0322-x). In addition, the VH domains of the disclosure can be incorporated into polypeptide library display systems to enable the selection and engineering of sequences having the biophysical properties described herein and therapeutic relevance. Display systems include, for example, phage display, HuTARG™ mammalian display system (Kielczewska, A. et al. Development of a potent high-affinity human therapeutic antibody via novel application of recombination signal sequence-based affinity maturation. J Biol Chem 298, 101533, doi: 10.1016/j.jbc.2021.101533 (2022)); ribozyme display, yeast surface, display, bacterial display, and mammalian display.

In some embodiments, the IGVH sequences and the VH domains of the disclosure herein may be combined with other VH domains, in sequence (5′-3′ or 3′-5′) in order to provide a stabilized molecules that bind to one or more molecular targets that may be relevant to the control or regulation of biological processes such as the processes relevant to the treatment of human and non-human disease. Accordingly, the IGVH sequences and VH domains of the disclosure may be formulated with a pharmaceutically acceptable carrier, excipient, or stabilizer, as pharmaceutical compositions. In certain embodiments, such pharmaceutical compositions are suitable for administration to a human or non-human animal via any one or more routes of administration using methods known in the art. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also contain compatible solid or liquid fillers, diluents or encapsulating substances, which are suitable for administration into a human. Other contemplated carriers, excipients, and/or additives, which may be utilized in the formulations described herein include, for example, flavoring agents, antimicrobial agents, sweeteners, antioxidants, antistatic agents, lipids, protein excipients such as serum albumin, gelatin, casein, salt-forming counterions such as sodium, and the like. These and additional known pharmaceutical carriers, excipients, and/or additives suitable for use in the formulations described herein are known in the art, for example, as listed in “Remington: The Science & Practice of Pharmacy,” 21st ed., Lippincott Williams & Wilkins, (2005), and in the “Physician's Desk Reference,” 60th ed., Medical Economics, Montvale, N.J. (2005). Pharmaceutically acceptable carriers can be selected that are suitable for the mode of administration, solubility, and/or stability desired or required.

### EXAMPLES

The Examples that follow are illustrative of specific embodiments of the disclosure, and various uses thereof. They are set forth for explanatory purposes only, and should not be construed as limiting the scope of the invention in any way.

### Example 1-Stabilizing Disulfides

The first approach is to identify potential novel disulfides that could be used to stabilize VH domains of the different germline families. Homology models were created for eight diverse VH sequences that represent VH families 1 through 5, by identifying the most suitable crystal structures (considering resolution and sequence similarity) and modifying any non-germline residues to germline using RosettaScripts. The VH coordinates were all originally complexed within the multidomain context of an antibody Fab.

The starting VH structures were diversified by building two homology models from either a single structure or two separate structures for in silico mutagenesis. Computational prediction of possible stabilizing disulfide bonds was performed by modifying, in silico, two residues to cys at a time and evaluating all combinations within the structure based on geometric constraints, then evaluating them based on an energy function (Gaurav et al., Nature 538:7625 (2016): 329-335). The results were sorted based on the disulfide score (dslf_fa13), models scoring less than −0.3 were considered for experimental testing. Table 3 shows the starting structures for the eight frameworks that were built based on crystal structures deposited within the Protein Data Bank (PDB).

TABLE 3

| Framework | PDB 1 | PDB 2 |
|---|---|---|
| 1-69.2 | 1RZ7 | 6P9J |
| VH3-11 | 6J6Y | 6XKP |
| VH3-15 | 5JR1 | 7JX3 |
| VH3-20 | 1W72 | 7JOO |
| VH3-21 | 6APC | |
| VH4-39 | 5W6C | 6PZE |
| VH2-5 | 3QYC | |
| VH5-51 | 3NAC | |

Numerous disulfide pairs were evaluated experimentally. The VH domains that were tested all had unique HCDR3s and bound to a variety of antigens. The goal of using VH domains with a diverse set of HCDR3s was to test the generalizability of the results obtained for each novel disulfide, independently of the HCDR3 sequences.

For the testing, nucleotide sequences encoding the VH domain sequences were first cloned into mammalian expression plasmids. The plasmids contained a CMV promotor driven open reading frame and BGH polyA tail. Secretion was driven using a mouse IgG signal peptide. VH domains were recombinantly fused to a human IgG1-Fc at the hinge region.

Cloning and plasmid production were performed using standard molecular biology methods. Secreted protein was produced by transfecting plasmids into HEK293 cells for transient expression using the Thermofisher Expi293 system. Supernatants for protein characterization were collected via centrifugation and then filtered. VH-Fc protein titer determinations were performed on a GatorBio biointerferometry instrument using Protein A tips supplied by the manufacturer and a purified VH-Fc as a standard. Alternately, for VH-His tag proteins, titer determinations were performed (1) in a similar manner on a GatorBio instrument using Anti-His tag tips and a purified human PD1-His tag protein as a standard, or (2) using by performing SDS-PAGE analysis on HEK293 supernatants and using densitometry to quantify protein levels and using a purified VH-His tag protein as a standard. For stability measurements, mammalian supernatants were analyzed using differential scanning fluorimetry (DSF) using a QuantStudio3 according to the manufacturer's protocols (Applied Biosystems) and the fluorescence vs temperature curves were analyzed using Applied Biosystem's Protein Thermal Shift™ software version 1.4.

Five novel disulfides were tested (FIGS. 2A-2O), and consistently demonstrate an ability to either improve the expression yields of poorly expressing VH domains and/or improve the stability of VH domains, with some preference for individual disulfide pairs for each of the germline families (Table 4).

For the VH1 family germlines, several disulfide pairs improve the expression and stability. One particular disulfide, 17C-82aC, appears to be superior in improving both the expression and the stability of three different VH1 family member germlines (Table 4). The VH1-69.2 germline was tested for two VH domains that bind different antigens and contain significantly different HCDR3 residues and the 17C-82aC disulfide was superior in both VHs. The 35C-50C disulfide also increased the stability of all of the tested VH1 germlines. The 23C-77C and 19C-81C disulfides improved the stability of the majority of VH1 germlines (Table 4).

For the VH3 family germlines, several disulfides improve the expression and stability. One particular disulfide, 23C-77C, is superior in improving both the expression and the stability of all seven tested VH3 family member germlines (Table 4). The VH3-20 germline was tested for two VH domains that bind different antigens and contain significantly different HCDR3 residues and the 23C-77C disulfide improves expression and stability for both VHs. The 17C-82aC, 19C-81C, and 35C-50C disulfides improved the stability of the majority of VH3 germlines (Table 4).

For the disulfide-modified set of VH domains that were tested:
(1) There was a minimum of 0% and maximum of 50% identity between HCDR3s with one outlying pair having an 81% identity (for this outlier pair, one is a VH1-8 and the other is a VH3-20). There's an average identity near 25% across all the HCDR3 sequences, which sets the sequences very far apart from one another
(2) There was a minimum length of 6 and a maximum length of 17 residues with an average length of 12.2 residues.

The obtained data on CDR3 composition of the various VH domains that were tested indicates that the designed substitutions (see e.g., Examples 1 and 2; also discussed above) were stabilizing for each of their germline families and that the design of the constructs and the observed stabilization effects were not HCDR3-dependent.

Two VH4 family member germlines were tested, and the results were different for each VH4 member. The 23C-77C, 35C-50C, and 2C-102C disulfides significantly improve the expression of the VH4-34 germline. Whereas, the 17C-82aC and 19C-81C disulfides significantly improves the expression of the VH4-39 germline (Table 4).

Lastly, the VH7 family consists of one germline member, VH7-4-1. Both 17C-82aC and 35C-50C disulfides resulted in substantial increases in expression, as well as Tₘs above® C.

Table 4 shows the expression titers, the change in expression titers vs. wild type, and results of stability experiments for several of the disulfide stabilized VH domains of the disclosure. Amino acid sequences for the VH domains that are summarized in Table 4 are provided in FIGS. 2A-2O.

TABLE 4

| VH | | | Fold | | | | | | | | | | | | | | |
|---|---|---|---|---|---|---|---|---|---|---|---|---|---|---|---|---|---|
| Family | | | Expression | | | Amino Acid | | | | | | | | | | | |
| 1 | Variant | | Change | DSF | | Substitutions | | | | | | | | | | | |
| Protein | (Kabat | Titer | versus | Tₘ | Found in | (Kabat Number) | | | | | | | | | | | |
| ID | Number) | (∞g/mL) | Wild-Type | (° C.) | SEQ ID NO: | 2 | 17 | 19 | 23 | 34 | 35 | 50 | 77 | 78 | 81 | 82a | 102 |
| VH1-8 | | | | | | | | | | | | | | | | | |
| ITS053- | WT | 372 | 1 | 57 | SEQ ID NO: 75 | V | S | K | K | I | N | W | T | A | E | S | X |
| M023 | | | | | | | | | | | | | | | | | |
| M247 | 17C_82aC | 942 | 2.53 | 64 | SEQ ID NO: 76 | V | C | K | K | I | N | W | T | A | E | C | X |
| M248 | 19C_81C | 877 | 2.36 | 62.5 | SEQ ID NO: 77 | V | S | C | K | I | N | W | T | A | C | S | X |
| M249 | 23C_77C | 843 | 2.27 | 61.5 | SEQ ID NO: 78 | V | S | K | C | I | N | W | C | A | E | S | X |
| M250 | 35C_50C | 799 | 2.15 | 61 | SEQ ID NO: 79 | V | S | K | K | I | C | C | T | A | E | S | X |
| M251 | V2C_102C | 102 | 0.27 | n.d. | SEQ ID NO: 80 | C | S | K | K | I | N | W | T | A | E | S | C |
| VH1-18 | | | | | | | | | | | | | | | | | |
| TTX020- | WT | 536 | 1 | 65.5 | SEQ ID NO: 81 | V | S | K | K | I | S | W | T | A | E | R | X |
| M019 | | | | | | | | | | | | | | | | | |
| | 17C_82aC | 566 | 1.06 | 71.5 | SEQ ID NO: 82 | V | C | K | K | I | S | W | T | A | E | C | X |
| | 19C_81C | 568 | 1.06 | 69 | SEQ ID NO: 83 | V | S | C | K | I | S | W | T | A | C | R | X |
| | 23C_77C | 101 | 0.19 | n.d. | SEQ ID NO: 84 | V | S | K | C | I | S | W | C | A | E | R | X |
| | 35C_50C | 476 | 0.89 | 70 | SEQ ID NO: 85 | V | S | K | K | I | C | C | T | A | E | R | X |
| | 2C_102C | 116 | 0.22 | n.d. | SEQ ID NO: 86 | C | S | K | K | I | S | W | T | A | E | R | C |
| VH1-69.2 | | | | | | | | | | | | | | | | | |
| ITS050- | WT | 187 | 1 | 62.5 | SEQ ID NO: 87 | V | T | K | K | M | H | L | T | A | E | S | X |
| M002v000 | | | | | | | | | | | | | | | | | |
| 050- | T17C_S82aC | 242 | 1.29 | 70 | SEQ ID NO: 88 | V | C | K | K | M | H | L | T | A | E | C | X |
| M002v002 | | | | | | | | | | | | | | | | | |
| 050- | K19C_E81C | 6 | 0.03 | n.d. | SEQ ID NO: 89 | V | T | C | K | M | H | L | T | A | C | S | X |
| M002v003 | | | | | | | | | | | | | | | | | |
| 050- | M34C_A78C | 162 | 0.87 | 69.5 | SEQ ID NO: 90 | V | T | K | K | C | H | L | T | C | E | S | X |
| M002v004 | | | | | | | | | | | | | | | | | |
| 050- | H35C_L50C | 180 | 0.96 | 67 | SEQ ID NO: 91 | V | T | K | K | M | C | C | T | A | E | S | X |
| M002v005 | | | | | | | | | | | | | | | | | |
| 050- | V2C_V102C | 1 | 0.01 | n.d. | SEQ ID NO: 92 | C | T | K | K | M | H | L | T | A | E | S | C |
| M002v001 | | | | | | | | | | | | | | | | | |
| TTX012- | WT | 187 | 1.00 | 64 | SEQ ID NO: 93 | V | T | K | K | M | H | L | T | A | E | S | X |
| M001 | | | | | | | | | | | | | | | | | |
| M004 | 17C_82aC | 444 | 2.37 | 71 | SEQ ID NO: 94 | V | C | K | K | M | H | L | T | A | E | C | X |
| M005 | 19C_81C | 530 | 2.83 | 70.5 | SEQ ID NO: 95 | V | T | C | K | M | H | L | T | A | C | S | X |
| M006 | 23C_77C | 402 | 2.15 | 69 | SEQ ID NO: 96 | V | T | K | C | M | H | L | C | A | E | S | X |
| M007 | 35C_50C | 294 | 1.57 | 70 | SEQ ID NO: 97 | V | T | K | K | M | C | C | T | A | E | S | X |
| M008 | 2C_102C | 585 | 3.13 | 71.5 | SEQ ID NO: 98 | C | T | K | K | M | H | L | T | A | E | S | C |

| VH | | | Fold | | | | | | | | | | | | | | |
|---|---|---|---|---|---|---|---|---|---|---|---|---|---|---|---|---|---|
| Family | | | Expression | | | Amino Acid | | | | | | | | | | | |
| 3 | Variant | | Change | DSF | | Substitutions | | | | | | | | | | | |
| Protein | (Kabat | Titer | versus | Tₘ | Found in | (Kabat Number) | | | | | | | | | | | |
| ID | Number) | (∞g/mL) | Wild-Type | (° C.) | SEQ ID NO: | 2 | 17 | 19 | 23 | 34 | 35 | 50 | 77 | 78 | 81 | 82a | 102 |
| VH3-9 | | | | | | | | | | | | | | | | | |
| ITS051- | WT | 375 | 1 | 56 | SEQ ID NO: 99 | V | S | R | A | M | H | G | S | L | Q | N | X |
| M003 | | | | | | | | | | | | | | | | | |
| M109 | 17C_82aC | 516 | 1.38 | 62 | SEQ ID NO: 100 | V | C | R | A | M | H | G | S | L | Q | C | X |
| M110 | 19C_81C | 109 | 0.29 | n.d. | SEQ ID NO: 101 | V | S | C | A | M | H | G | S | L | C | N | X |
| M111 | 23C_77C | 399 | 1.06 | 64.5 | SEQ ID NO: 102 | V | S | R | C | M | H | G | C | L | Q | N | X |
| M112 | 35C_50C | 329 | 0.88 | 49 | SEQ ID NO: 103 | V | S | R | A | M | C | C | S | L | Q | N | X |
| M113 | 2C_102C | 718 | 1.91 | 62.5 | SEQ ID NO: 104 | C | S | R | A | M | H | G | S | L | Q | N | C |
| VH3-11 | | | | | | | | | | | | | | | | | |
| TTX020- | WT | 890 | 1 | 73 | SEQ ID NO: 105 | V | S | R | A | M | S | Y | S | L | Q | N | X |
| M022 | | | | | | | | | | | | | | | | | |
| M033 | 17C_82aC | 1050 | 1.18 | 74 | SEQ ID NO: 106 | V | C | R | A | M | S | Y | S | L | Q | C | X |
| M034 | 19C_81C | 1270 | 1.43 | 72 | SEQ ID NO: 107 | V | S | C | A | M | S | Y | S | L | C | N | X |
| M035 | 23C_77C | 1010 | 1.13 | 72 | SEQ ID NO: 108 | V | S | R | C | M | S | Y | C | L | Q | N | X |
| M036 | 35C_50C | 975 | 1.10 | 73 | SEQ ID NO: 109 | V | S | R | A | M | C | C | S | L | Q | N | X |
| M037 | 2C_102C | 531 | 0.60 | 73 | SEQ ID NO: 110 | C | S | R | A | M | S | Y | S | L | Q | N | C |
| VH3-15 | | | | | | | | | | | | | | | | | |
| 053- | WT | 964 | 1 | 87.5 | SEQ ID NO: 111 | V | S | R | A | M | S | R | T | L | Q | N | X |
| M011 | | | | | | | | | | | | | | | | | |
| | 17C_82aC | 1140 | 1.18 | 71 | SEQ ID NO: 112 | V | C | R | A | M | S | R | T | L | Q | C | X |
| | 19C_81C | 881 | 0.89 | 71 | SEQ ID NO: 113 | V | S | C | A | M | S | R | T | L | C | N | X |
| | 23C_77C | 1020 | 1.06 | 72 | SEQ ID NO: 114 | V | S | R | C | M | S | R | C | L | Q | N | X |
| | 35C_50C | 612 | 0.63 | 64 | SEQ ID NO: 115 | V | S | R | A | M | C | C | T | L | Q | N | X |
| | 2C_102C | 412 | 0.43 | 72.5 | SEQ ID NO: 116 | C | S | R | A | M | S | R | T | L | Q | N | C |
| VH3-20 | | | | | | | | | | | | | | | | | |
| ITS051- | WT | 1050 | 1 | 53 | SEQ ID NO: 117 | V | S | R | A | M | S | G | S | L | Q | N | X |
| M023 | | | | | | | | | | | | | | | | | |
| M114 | 17C_82aC | 990 | 0.94 | 66.5 | SEQ ID NO: 118 | V | C | R | A | M | S | G | S | L | Q | C | X |
| M115 | 19C_81C | 216 | 0.21 | n.d. | SEQ ID NO: 119 | V | S | C | A | M | S | G | S | L | C | N | X |
| M116 | 23C_77C | 1170 | 1.11 | 70 | SEQ ID NO: 120 | V | S | R | C | M | S | G | C | L | Q | N | X |
| M117 | 35C_50C | 955 | 0.92 | 60 | SEQ ID NO: 121 | V | S | R | A | M | C | C | S | L | Q | N | X |
| M118 | 2C_102C | 483 | 0.48 | 57 | SEQ ID NO: 122 | C | S | R | A | M | S | G | S | L | Q | N | C |
| 051- | 051-M019 | 1* | 1.00 | | SEQ ID NO: 123 | V | S | R | A | M | S | G | S | L | Q | N | X |
| M019v000 | | | | | | | | | | | | | | | | | |
| 051- | 051-M019- | 15 | 15.00 | n.d. | SEQ ID NO: 124 | V | S | R | C | M | S | G | C | L | Q | N | X |
| M019v002 | A23C-S77C | | | | | | | | | | | | | | | | |
| 051- | 051-M019- | 12 | 12.00 | n.d. | SEQ ID NO: 125 | V | S | R | A | M | C | C | S | L | Q | N | X |
| M019v004 | S35C-G50C | | | | | | | | | | | | | | | | |
| VH3-21 | | | | | | | | | | | | | | | | | |
| 053- | 053-M009 | 45 | | n.d. | SEQ ID NO: 126 | V | S | R | A | M | N | S | S | L | Q | N | X |
| M009v000 | | | | | | | | | | | | | | | | | |
| 053- | 053-M009- | 124 | 2.76 | n.d. | SEQ ID NO: 127 | V | S | R | A | M | C | C | S | L | Q | N | X |
| M009v001 | N35C-S50C | | | | | | | | | | | | | | | | |
| VH3-30 | | | | | | | | | | | | | | | | | |
| TTX020- | WT | 773 | 1 | 57.5 | SEQ ID NO: 128 | V | S | R | A | M | H | V | T | L | Q | N | X |
| M002 | | | | | | | | | | | | | | | | | |
| M038 | 17C_82aC | 544 | 0.70 | >72 | SEQ ID NO: 129 | V | C | R | A | M | H | V | T | L | Q | C | X |
| M039 | 19C_81C | 671 | 0.87 | >72 | SEQ ID NO: 130 | V | S | C | A | M | H | V | T | L | C | N | X |
| M040 | 23C_77C | 1090 | 1.41 | 69 | SEQ ID NO: 131 | V | S | R | C | M | H | V | C | L | Q | N | X |
| M041 | 35C_50C | 1140 | 1.47 | >72 | SEQ ID NO: 132 | V | S | R | A | M | C | C | T | L | Q | N | X |
| M042 | 2C_102C | 230 | 0.30 | 57 | SEQ ID NO: 133 | C | S | R | A | M | H | V | T | L | Q | N | C |
| VH3-53 | | | | | | | | | | | | | | | | | |
| TTX020- | WT | 560 | 1 | 72 | SEQ ID NO: 134 | V | S | R | A | M | S | V | T | L | Q | N | X |
| 0010 | | | | | | | | | | | | | | | | | |
| | 17C_82aC | 589 | 1.05 | 72 | SEQ ID NO: 135 | V | C | R | A | M | S | V | T | L | Q | C | X |
| | 19C_81C | 491 | 0.88 | 73 | SEQ ID NO: 136 | V | S | C | A | M | S | V | T | L | C | N | X |
| | 23C_77C | 532 | 1.06 | 72 | SEQ ID NO: 137 | V | S | R | C | M | S | V | C | L | Q | N | X |
| | 35C_50C | 1080 | 1.93 | 74 | SEQ ID NO: 138 | V | S | R | A | M | C | C | T | L | Q | N | X |
| | 2C_102C | 687 | 1.23 | 71 | SEQ ID NO: 139 | C | S | R | A | M | S | V | T | L | Q | N | C |

| | | | Fold | | | | | | | | | | | | | | |
|---|---|---|---|---|---|---|---|---|---|---|---|---|---|---|---|---|---|
| | | | Expression | | | Amino Acid | | | | | | | | | | | |
| | Variant | | Change | DSF | | Substitutions | | | | | | | | | | | |
| VH Family 4 | (Kabat | Titer | versus | Tₘ | Found in | (Kabat Number) | | | | | | | | | | | |
| Protein ID | Number) | (∞g/mL) | Wild-Type | (° C.) | SEQ ID NO: | 2 | 17 | 19 | 23 | 34 | 35 | 50 | 77 | 78 | 81 | 82a | 102 |
| VH4-34 | | | | | | | | | | | | | | | | | |
| ITS050-M055 | WT | 30 | 1 | n.d. | SEQ ID NO: 140 | V | T | S | A | W | S | E | Q | F | K | S | X |
| | 17C_82aC | 4 | 0.13 | n.d. | SEQ ID NO: 141 | V | C | S | A | W | S | E | Q | F | K | C | X |
| | 19C_81C | 4 | 0.13 | n.d. | SEQ ID NO: 142 | V | T | C | A | W | S | E | Q | F | C | S | X |
| | 23C_77C | 104 | 3.47 | n.d. | SEQ ID NO: 143 | V | T | S | C | W | S | E | C | F | K | S | X |
| | 35C_50C | 101 | 3.37 | n.d. | SEQ ID NO: 144 | V | T | S | A | W | C | C | Q | F | K | S | X |
| | 2C_102C | 153 | 5.10 | n.d. | SEQ ID NO: 145 | C | T | S | A | W | S | E | Q | F | K | S | C |
| VH4-39 | | | | | | | | | | | | | | | | | |
| ITS045-M007 | WT | 1 | 1 | n.d. | SEQ ID NO: 146 | L | T | S | T | W | G | S | Q | F | K | S | X |
| ITS045-M007 | 17C_82aC | 25 | 25.00 | n.d. | SEQ ID NO: 147 | L | C | S | T | W | G | S | Q | F | K | C | X |
| ITS045-M007 | 19C_81C | 16 | 16.00 | n.d. | SEQ ID NO: 148 | L | T | C | T | W | G | S | Q | F | C | S | X |
| ITS045-M007 | 23C_77C | 4 | 4.00 | n.d. | SEQ ID NO: 149 | L | T | S | C | W | G | S | C | F | K | S | X |
| ITS045-M007 | 35C_50C | 1 | 1.00 | n.d. | SEQ ID NO: 150 | L | T | S | T | W | C | C | Q | F | K | S | X |
| ITS045-M007 | 2C_102C | 4 | 4.00 | n.d. | SEQ ID NO: 151 | C | T | S | T | W | G | S | Q | F | K | S | C |
| | | | Fold | | | | | | | | | | | | | | |
| | | | Expression | | | Amino Acid | | | | | | | | | | | |
| | Variant | | Change | DSF | | Substitutions | | | | | | | | | | | |
| VH Family 7 | (Kabat | Titer | versus | Tₘ | Found in | (Kabat Number) | | | | | | | | | | | |
| Protein ID | Number) | (∞g/mL) | Wild-Type | (° C.) | SEQ ID NO: | 2 | 17 | 19 | 23 | 34 | 35 | 50 | 77 | 78 | 81 | 82a | 102 |
| VH7-4 | | | | | | | | | | | | | | | | | |
| ITS050-M021 | WT | 50 | 1 | n.d. | SEQ ID NO: 152 | V | S | K | K | M | N | W | T | A | Q | C | X |
| | 17C_82aC | 442 | 8.84 | 62.5 | SEQ ID NO: 153 | V | C | K | K | M | N | W | T | A | Q | C | X |
| | 19C_81C | 22 | 0.44 | n.d. | SEQ ID NO: 154 | V | S | C | K | M | N | W | T | A | C | C | X |
| | 23C_77C | 77 | 1.54 | n.d. | SEQ ID NO: 155 | V | S | K | C | M | N | W | C | A | Q | C | X |
| | 35C_50C | 521 | 10.42 | 67.5 | SEQ ID NO: 156 | V | S | K | K | M | C | C | T | A | Q | C | X |
| | 2C_102C | 23 | 0.46 | n.d. | SEQ ID NO: 157 | C | S | K | K | M | N | W | T | A | Q | C | C |

*Lower limit of quantitation
n.d. = not determined

The CH2 domain of the Fc unfolds with a Tm of about 71° C., thus interfering with the ability to quantify VH Tms with improved stabilities above 71° C. While the disulfides likely improve stability, the impact of the disulfides was difficult to characterize in unmodified molecules that have a Tm above 71° C.

### Example 2-Stabilizing Variant Discovery

Computational design was also utilized to identify additional residues where substitution of the amino acid may result in a stability increase. The same homology models used in Example 1 were utilized to create libraries of predominately single amino acid variants and a small number of combinatorial variants. The energy of these homology models were then minimized within the Rosetta software using existing protocols within RosettaScripts (Froning, K., et al. Computational stabilization of T cell receptors allows pairing with antibodies to form bispecifics. Nat Commun 11, 2330 (2020)). In silico site saturation mutagenesis was performed in which each position within the protein was replaced with all possible amino acids (excluding cys). Each point mutation was compared to the score of the WT sequence to calculate the difference in energy (ΔE). The average score for the target sequence were then sorted by value to rank the mutations for experimental testing.

VH domain-IgG1Fc variants were produced using the same methodology described in Example 1. Roughly, 200 variants were generated and screened across 3 VH families, including five (5) different germlines (VH3-15, VH3-20, VH3-21, VH1-69.2, and VH4-39) (FIGS. 3A-3N). A subset of these variants was found to improve the expression for each domain and are shown in Table 5 along with thermal stability data (DSF Tₘ) for some molecules.

TABLE 5

| | | | Fold | | | | | | | | | | |
|---|---|---|---|---|---|---|---|---|---|---|---|---|---|
| | | | Expression | | | | | | | | | | |
| | Variant | | Change | DSF | | Amino Acid Substitutions | | | | | | | |
| | (Kabat | Titer | versus | Tₘ | Found in | (Kabat Number) | | | | | | | |
| Protein ID | Number) | (∞g/mL) | Wild-Type | (° C.) | SEQ ID NO: | 1 | 2 | 5 | 10 | 14 | 15 | 16 | 19 |
| VH3-20 | | | | | | | | | | | | | |
| 051-M019v000 | 051-M019 | 1* | 1 | n.d. | SEQ ID NO: 123 | E | V | V | G | P | G | G | R |
| 051-M019v005 | V2A | 6 | 6 | n.d. | SEQ ID NO: 158 | E | A | V | G | P | G | G | R |
| 051-M019v010 | P14E | 3 | 3 | n.d. | SEQ ID NO: 159 | E | V | V | G | E | G | G | R |
| 051-M019v013 | A23Q | 3 | 3 | n.d. | SEQ ID NO: 160 | E | V | V | G | P | G | G | R |
| 051-M019v014 | T28N | 7 | 7 | n.d. | SEQ ID NO: 161 | E | V | V | G | P | G | G | R |
| 051-M019v015 | T28K | 11 | 11 | n.d. | SEQ ID NO: 162 | E | V | V | G | P | G | G | R |
| 051-M019v016 | T28R | 3 | 3 | n.d. | SEQ ID NO: 163 | E | V | V | G | P | G | G | R |
| 051-M019v017 | D30K | 7 | 7 | n.d. | SEQ ID NO: 164 | E | V | V | G | P | G | G | R |
| 051-M019v018 | D30S | 4 | 4 | n.d. | SEQ ID NO: 165 | E | V | V | G | P | G | G | R |
| 051-M019v023 | S49A | 145 | 145 | n.d. | SEQ ID NO: 166 | E | V | V | G | P | G | G | R |
| 051-M019v024 | G55E | 11 | 11 | n.d. | SEQ ID NO: 167 | E | V | V | G | P | G | G | R |
| 051-M019v030 | A74E | 7 | 7 | n.d. | SEQ ID NO: 168 | E | V | V | G | P | G | G | R |
| 051-M019v032 | N76K | 7 | 7 | n.d. | SEQ ID NO: 169 | E | V | V | G | P | G | G | R |
| 051-M019v033 | S77Q | 85 | 85 | n.d. | SEQ ID NO: 170 | E | V | V | G | P | G | G | R |
| 051-M019v034 | A84E | 3 | 3 | n.d. | SEQ ID NO: 171 | E | V | V | G | P | G | G | R |
| 051-M019v035 | A84P | 2 | 2 | n.d. | SEQ ID NO: 172 | E | V | V | G | P | G | G | R |
| 051-M019v036 | A23Q_S77Q | 201 | 201 | n.d. | SEQ ID NO: 173 | E | V | V | G | P | G | G | R |
| VH3-21 | | | | | | | | | | | | | |
| 053-M009v000 | WT | 45 | 1 | n.d. | SEQ ID NO: 126 | E | V | V | G | P | G | G | R |
| 053-M009v007 | A23Q | 58 | 1.3 | n.d. | SEQ ID NO: 174 | E | V | V | G | P | G | G | R |
| 053-M009v010 | T28D | 145 | 3.2 | n.d. | SEQ ID NO: 175 | E | V | V | G | P | G | G | R |
| 053-M009v011 | T28E | 94 | 2.1 | n.d. | SEQ ID NO: 176 | E | V | V | G | P | G | G | R |
| 053-M009v016 | S33P | 90 | 2 | n.d. | SEQ ID NO: 177 | E | V | V | G | P | G | G | R |
| 053-M009v019 | N35G | 63 | 1.4 | n.d. | SEQ ID NO: 178 | E | V | V | G | P | G | G | R |
| 053-M009v020 | N35A | 61 | 1.4 | n.d. | SEQ ID NO: 179 | E | V | V | G | P | G | G | R |
| 053-M009v021 | N35S | 61 | 1.4 | n.d. | SEQ ID NO: 180 | E | V | V | G | P | G | G | R |
| 053-M009v025 | S49A | 133 | 3.0 | n.d. | SEQ ID NO: 181 | E | V | V | G | P | G | G | R |
| 053-M009v027 | S52D | 81 | 1.8 | n.d. | SEQ ID NO: 182 | E | V | V | G | P | G | G | R |
| 053-M009v028 | S55E | 67 | 1.5 | n.d. | SEQ ID NO: 183 | E | V | V | G | P | G | G | R |
| 053-M009v030 | Y56E | 105 | 2.3 | n.d. | SEQ ID NO: 184 | E | V | V | G | P | G | G | R |
| 053-M009v035 | A74E | 67 | 1.5 | n.d. | SEQ ID NO: 185 | E | V | V | G | P | G | G | R |
| 053-M009v042 | A84E | 69 | 1.5 | n.d. | SEQ ID NO: 186 | E | V | V | G | P | G | G | R |
| 053-M009v043 | A84P | 55 | 1.2 | n.d. | SEQ ID NO: 187 | E | V | V | G | P | G | G | R |
| 053-M009v044 | V5Q_A23Q | 78 | 1.7 | n.d. | SEQ ID NO: 188 | E | V | Q | G | P | G | G | R |
| VH3-15 | | | | | | | | | | | | | |
| 053-M011v000 | WT | 566 | 1 | 67.5 | SEQ ID NO: 111 | E | V | V | G | P | G | G | R |
| 053-M011v013 | A23K | 539 | 1.0 | 69.5 | SEQ ID NO: 189 | E | V | V | G | P | G | G | R |
| 053-M011v014 | A23Q | 378 | 0.7 | 68.5 | SEQ ID NO: 190 | E | V | V | G | P | G | G | R |
| 053-M011v015 | A23Y | 417 | 0.7 | 68 | SEQ ID NO: 191 | E | V | V | G | P | G | G | R |
| 053-M011v018 | T28D | 465 | 0.8 | 71 | SEQ ID NO: 192 | E | V | V | G | P | G | G | R |
| 053-M011v019 | N31K | 498 | 0.9 | 68.5 | SEQ ID NO: 193 | E | V | V | G | P | G | G | R |
| 053-M011v021 | A40P | 408 | 0.7 | 68.5 | SEQ ID NO: 194 | E | V | V | G | P | G | G | R |
| 053-M011v034 | S82bD | 419 | 0.7 | 68.5 | SEQ ID NO: 195 | E | V | V | G | P | G | G | R |
| 053-M011v035 | T84E | 480 | 0.8 | 68.5 | SEQ ID NO: 196 | E | V | V | G | P | G | G | R |
| 053-M011v036 | T84P | 500 | 0.9 | 68 | SEQ ID NO: 197 | E | V | V | G | P | G | G | R |
| 053-M011v039 | T110V | 342 | 0.6 | 71 | SEQ ID NO: 198 | E | V | V | G | P | G | G | R |
| 053-M011v040 | T110I | 352 | 0.6 | 71 | SEQ ID NO: 199 | E | V | V | G | P | G | G | R |
| VH1-69.2 | | | | | | | | | | | | | |
| 050-M002v000 | WT | 187 | 1 | 62.5 | SEQ ID NO: 87 | E | V | V | E | P | G | A | K |
| 050-M002v011 | E10Q | 232 | 1.2 | 63 | SEQ ID NO: 200 | E | V | V | Q | P | G | A | K |
| 050-M002v012 | A16D | 181 | 1.0 | 66 | SEQ ID NO: 201 | E | V | V | E | P | G | D | K |
| 050-M002v013 | A16Q | 200 | 1.1 | 63.5 | SEQ ID NO: 202 | E | V | V | E | P | G | Q | K |
| 050-M002v014 | S25Y | 221 | 1.2 | 63 | SEQ ID NO: 203 | E | V | V | E | P | G | A | K |
| 050-M002v016 | V37F | 229 | 1.2 | 64 | SEQ ID NO: 204 | E | V | V | E | P | G | A | K |
| 050-M002v018 | M48I | 192 | 1.0 | 63.5 | SEQ ID NO: 205 | E | V | V | E | P | G | A | K |
| 050-M002v020 | S84E | 153 | 0.8 | 64 | SEQ ID NO: 206 | E | V | V | E | P | G | A | K |
| 050-M002v021 | S84P | 136 | 0.7 | 64 | SEQ ID NO: 207 | E | V | V | E | P | G | A | K |
| 050-M002v024 | T110V | 146 | 0.8 | 67 | SEQ ID NO: 208 | E | V | V | E | P | G | A | K |
| 050-M002v025 | T110I | 133 | 0.7 | 68 | SEQ ID NO: 209 | E | V | V | E | P | G | A | K |
| VH4-39 | | | | | | | | | | | | | |
| 045-M002V000 | WT | 162 | 1 | n.d. | SEQ ID NO: 210 | Q | L | Q | G | P | S | E | S |
| 045-M002V002 | Q1E | 205 | 1.3 | n.d. | SEQ ID NO: 211 | E | L | Q | G | P | S | E | S |
| 045-M002V004 | G10Q | 218 | 1.3 | n.d. | SEQ ID NO: 212 | Q | L | Q | Q | P | S | E | S |
| 045-M002V005 | G10T | 251 | 1.5 | n.d. | SEQ ID NO: 213 | Q | L | Q | T | P | S | E | S |
| 045-M002V006 | S15G | 195 | 1.2 | n.d. | SEQ ID NO: 214 | Q | L | Q | G | P | G | E | S |
| 045-M002V008 | S19I | 197 | 1.2 | n.d. | SEQ ID NO: 215 | Q | L | Q | G | P | S | E | I |
| 045-M002V018 | S82bN | 180 | 1.1 | n.d. | SEQ ID NO: 216 | Q | L | Q | G | P | S | E | S |
| 045-M002V020 | A84P | 184 | 1.1 | n.d. | SEQ ID NO: 217 | Q | L | Q | G | P | S | E | S |

| | Amino Acid Substitutions | | | | | | | | | | | | | | | | | | | |
|---|---|---|---|---|---|---|---|---|---|---|---|---|---|---|---|---|---|---|---|---|
| | (Kabat Number) | | | | | | | | | | | | | | | | | | | |
| Protein ID | 23 | 25 | 28 | 30 | 31 | 33 | 35 | 37 | 40 | 48 | 49 | 52 | 55 | 56 | 74 | 76 | 77 | 82b | 84 | 110 |
| VH3-20 | | | | | | | | | | | | | | | | | | | | |
| 051-M019v000 | A | S | T | D | D | G | S | V | A | V | S | N | G | S | A | N | S | S | A | T |
| 051-M019v005 | A | S | T | D | D | G | S | V | A | V | S | N | G | S | A | N | S | S | A | T |
| 051-M019v010 | A | S | T | D | D | G | S | V | A | V | S | N | G | S | A | N | S | S | A | T |
| 051-M019v013 | Q | S | T | D | D | G | S | V | A | V | S | N | G | S | A | N | S | S | A | T |
| 051-M019v014 | A | S | N | D | D | G | S | V | A | V | S | N | G | S | A | N | S | S | A | T |
| 051-M019v015 | A | S | K | D | D | G | S | V | A | V | S | N | G | S | A | N | S | S | A | T |
| 051-M019v016 | A | S | R | D | D | G | S | V | A | V | S | N | G | S | A | N | S | S | A | T |
| 051-M019v017 | A | S | T | K | D | G | S | V | A | V | S | N | G | S | A | N | S | S | A | T |
| 051-M019v018 | A | S | T | S | D | G | S | V | A | V | S | N | G | S | A | N | S | S | A | T |
| 051-M019v023 | A | S | T | D | D | G | S | V | A | V | A | N | G | S | A | N | S | S | A | T |
| 051-M019v024 | A | S | T | D | D | G | S | V | A | V | S | N | E | S | A | N | S | S | A | T |
| 051-M019v030 | A | S | T | D | D | G | S | V | A | V | S | N | G | S | E | N | S | S | A | T |
| 051-M019v032 | A | S | T | D | D | G | S | V | A | V | S | N | G | S | A | K | S | S | A | T |
| 051-M019v033 | A | S | T | D | D | G | S | V | A | V | S | N | G | S | A | N | Q | S | A | T |
| 051-M019v034 | A | S | T | D | D | G | S | V | A | V | S | N | G | S | A | N | S | S | E | T |
| 051-M019v035 | A | S | T | D | D | G | S | V | A | V | S | N | G | S | A | N | S | S | P | T |
| 051-M019v036 | Q | S | T | D | D | G | S | V | A | V | S | N | G | S | A | N | Q | S | A | T |
| VH3-21 | | | | | | | | | | | | | | | | | | | | |
| 053-M009v000 | A | S | T | S | S | S | N | V | A | V | S | S | S | Y | A | N | S | S | A | T |
| 053-M009v007 | Q | S | T | S | S | S | N | V | A | V | S | S | S | Y | A | N | S | S | A | T |
| 053-M009v010 | A | S | D | S | S | S | N | V | A | V | S | S | S | Y | A | N | S | S | A | T |
| 053-M009v011 | A | S | E | S | S | S | N | V | A | V | S | S | S | Y | A | N | S | S | A | T |
| 053-M009v016 | A | S | T | S | S | P | N | V | A | V | S | S | S | Y | A | N | S | S | A | T |
| 053-M009v019 | A | S | T | S | S | S | G | V | A | V | S | S | S | Y | A | N | S | S | A | T |
| 053-M009v020 | A | S | T | S | S | S | A | V | A | V | S | S | S | Y | A | N | S | S | A | T |
| 053-M009v021 | A | S | T | S | S | S | S | V | A | V | S | S | S | Y | A | N | S | S | A | T |
| 053-M009v025 | A | S | T | S | S | S | N | V | A | V | A | S | S | Y | A | N | S | S | A | T |
| 053-M009v027 | A | S | T | S | S | S | N | V | A | V | S | D | S | Y | A | N | S | S | A | T |
| 053-M009v028 | A | S | T | S | S | S | N | V | A | V | S | S | E | Y | A | N | S | S | A | T |
| 053-M009v030 | A | S | T | S | S | S | N | V | A | V | S | S | S | E | A | N | S | S | A | T |
| 053-M009v035 | A | S | T | S | S | S | N | V | A | V | S | S | S | Y | E | N | S | S | A | T |
| 053-M009v042 | A | S | T | S | S | S | N | V | A | V | S | S | S | Y | A | N | S | S | E | T |
| 053-M009v043 | A | S | T | S | S | S | N | V | A | V | S | S | S | Y | A | N | S | S | P | T |
| 053-M009v044 | Q | S | T | S | S | S | N | V | A | V | S | S | S | Y | A | N | S | S | A | T |
| VH3-15 | | | | | | | | | | | | | | | | | | | | |
| 053-M011v000 | A | S | T | S | N | W | S | V | A | V | G | K | G | T | S | N | T | S | T | T |
| 053-M011v013 | K | S | T | S | N | W | S | V | A | V | G | K | G | T | S | N | T | S | T | T |
| 053-M011v014 | Q | S | T | S | N | W | S | V | A | V | G | K | G | T | S | N | T | S | T | T |
| 053-M011v015 | Y | S | T | S | N | W | S | V | A | V | G | K | G | T | S | N | T | S | T | T |
| 053-M011v018 | A | S | D | S | N | W | S | V | A | V | G | K | G | T | S | N | T | S | T | T |
| 053-M011v019 | A | S | T | S | K | W | S | V | A | V | G | K | G | T | S | N | T | S | T | T |
| 053-M011v021 | A | S | T | S | N | W | S | V | P | V | G | K | G | T | S | N | T | S | T | T |
| 053-M011v034 | A | S | T | S | N | W | S | V | A | V | G | K | G | T | S | N | T | D | T | T |
| 053-M011v035 | A | S | T | S | N | W | S | V | A | V | G | K | G | T | S | N | T | S | E | T |
| 053-M011v036 | A | S | T | S | N | W | S | V | A | V | G | K | G | T | S | N | T | S | P | T |
| 053-M011v039 | A | S | T | S | N | W | S | V | A | V | G | K | G | T | S | N | T | S | T | V |
| 053-M011v040 | A | S | T | S | N | W | S | V | A | V | G | K | G | T | S | N | T | S | T | I |
| VH1-69.2 | | | | | | | | | | | | | | | | | | | | |
| 050-M002v000 | K | S | T | T | D | Y | H | V | A | M | G | D | G | E | S | D | T | S | S | T |
| 050-M002v011 | K | S | T | T | D | Y | H | V | A | M | G | D | G | E | S | D | T | S | S | T |
| 050-M002v012 | K | S | T | T | D | Y | H | V | A | M | G | D | G | E | S | D | T | S | S | T |
| 050-M002v013 | K | S | T | T | D | Y | H | V | A | M | G | D | G | E | S | D | T | S | S | T |
| 050-M002v014 | K | Y | T | T | D | Y | H | V | A | M | G | D | G | E | S | D | T | S | S | T |
| 050-M002v016 | K | S | T | T | D | Y | H | F | A | M | G | D | G | E | S | D | T | S | S | T |
| 050-M002v018 | K | S | T | T | D | Y | H | V | A | 1 | G | D | G | E | S | D | T | S | S | T |
| 050-M002v020 | K | S | T | T | D | Y | H | V | A | M | G | D | G | E | S | D | T | S | E | T |
| 050-M002v021 | K | S | T | T | D | Y | H | V | A | M | G | D | G | E | S | D | T | S | P | T |
| 050-M002v024 | K | S | T | T | D | Y | H | V | A | M | G | D | G | E | S | D | T | S | S | V |
| 050-M002v025 | K | S | T | T | D | Y | H | V | A | M | G | D | G | E | S | D | T | S | S | I |
| VH4-39 | | | | | | | | | | | | | | | | | | | | |
| 045-M002V000 | T | S | S | S | S | Y | G | I | P | I | G | Y | G | S | S | N | Q | S | A | T |
| 045-M002V002 | T | S | S | S | S | Y | G | I | P | I | G | Y | G | S | S | N | Q | S | A | T |
| 045-M002V004 | T | S | S | S | S | Y | G | I | P | I | G | Y | G | S | S | N | Q | S | A | T |
| 045-M002V005 | T | S | S | S | S | Y | G | I | P | I | G | Y | G | S | S | N | Q | S | A | T |
| 045-M002V006 | T | S | S | S | S | Y | G | I | P | I | G | Y | G | S | S | N | Q | S | A | T |
| 045-M002V008 | T | S | S | S | S | Y | G | I | P | I | G | Y | G | S | S | N | Q | S | A | T |
| 045-M002V018 | T | S | S | S | S | Y | G | I | P | I | G | Y | G | S | S | N | Q | N | A | T |
| 045-M002V020 | T | S | S | S | S | Y | G | I | P | I | G | Y | G | S | S | N | Q | S | P | T |

*LLQ

### Example 3—Combination Designs Specific to Each Germline VH Gene Family to Generally Stabilize VH Domains

Based on the data from Example 1 and Example 2, two sets of combinatorial designs were generated for germline gene families 1, 3, and 4. The specific combinatorial designs are provided in Table 6.

TABLE 6

| Germline | | |
|---|---|---|
| Gene | Optimization | Optimization |
| Family | Design 1 (Opt1) | Design 2 (Opt 2) |
| VH1 | 17C-82aC, 39R, 48I | 17C-82aC, 16D, 39R, 48I |
| VH3 | 23C-77C, 39R, 49A, 74E | 23C-77C, 39R, 49A, 84E |
| VH4 | 17C-82aC, 10T, 39R, 49A | 23C-77C, 39R, 49A |

Nine separate VH domains with unique HCDR3s were tested with the two design combinations that were specific for each germline. The nine individual germlines included three VH1 family (one VH1-8 and two VH1-69.2 with different HCDR3s), five VH3 family (one VH3-11, one VH3-15, two VH3-20 with different HCDR3s, and one VH3-48), and one VH4 family (VH4-39) germlines. The molecules were synthesized as gblocks by IDT and cloned into the expression vector with a C-terminal 8×Histidine Tag. The constructs were His-tagged at the C-terminus for purification.

The expression plasmids were transfected in duplicate into HEK293 cells and supernatants were harvested as described above. The supernatants were titered using GatorBio biointerferometry after dilution 1-to-20 in PBS buffer. A purified, his-tagged 15 kDa V-class Ig-fold protein was used to develop the standard curve. For DSF experiments, the proteins were affinity purified by incubation with a His60 Nickel resin (Takara), washing with a neutral pH buffer with 10-30 mM imidazole buffer, and eluted using 200-400 mM imidazole. Eluted proteins were directly used for DSF measurements, as described above.

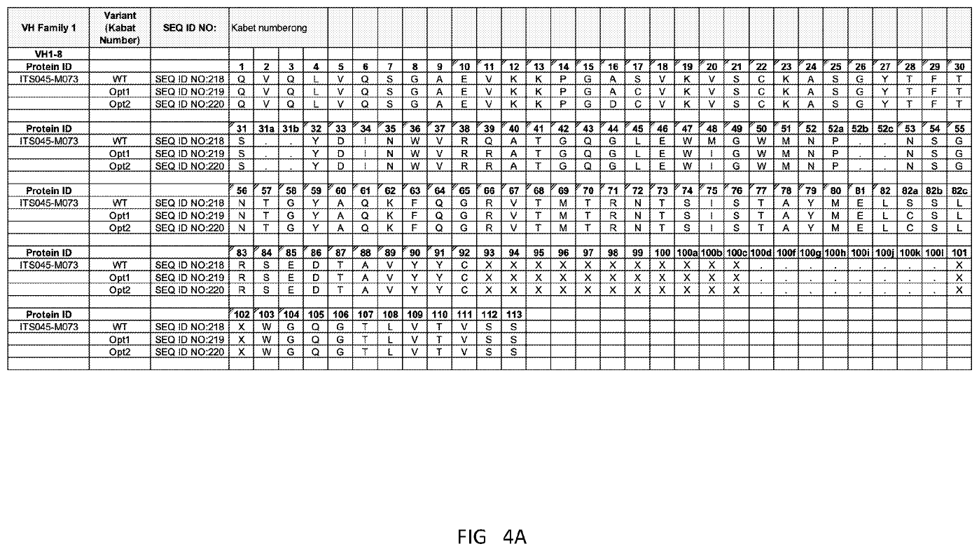

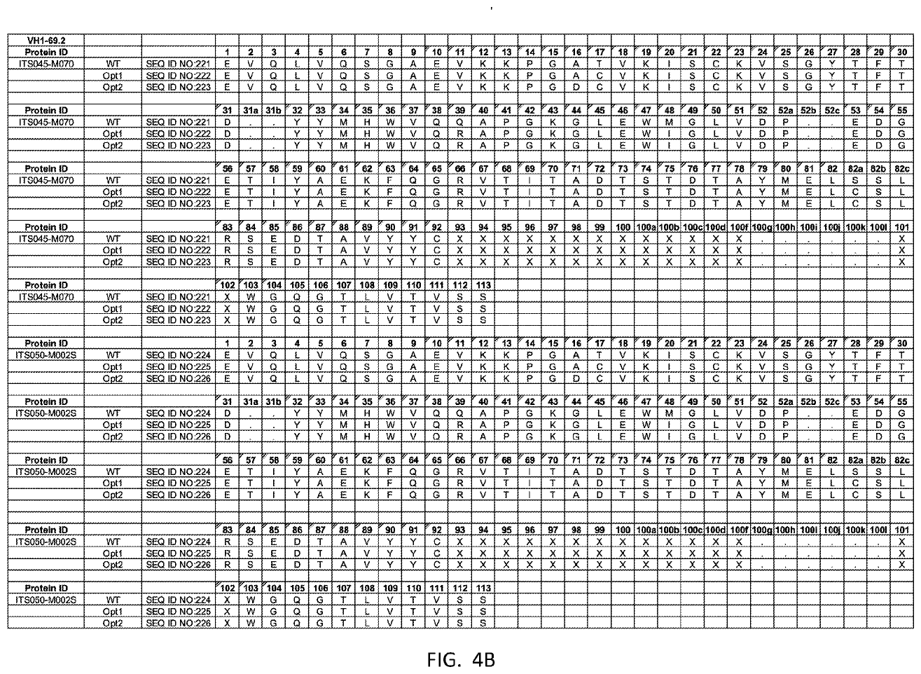

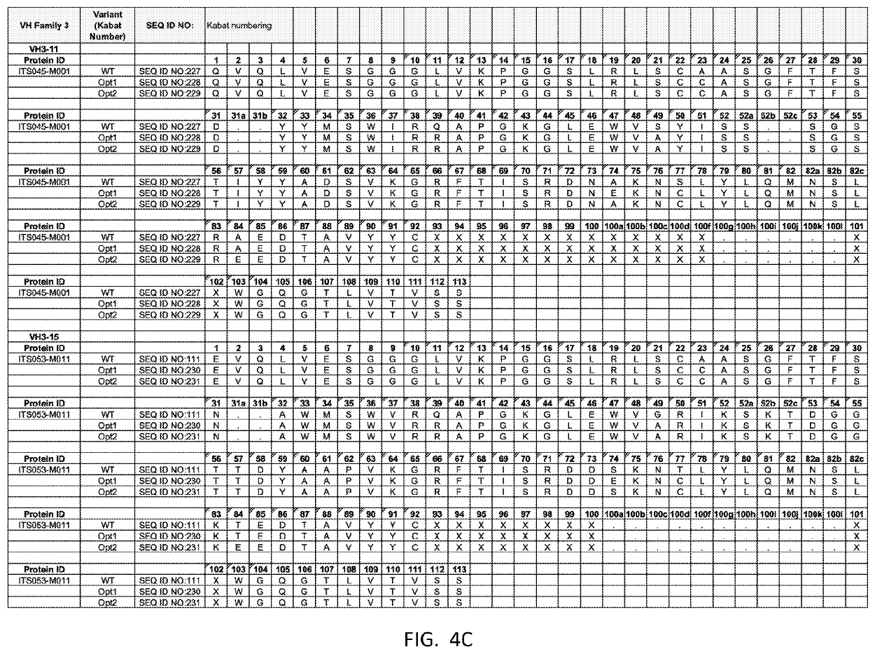

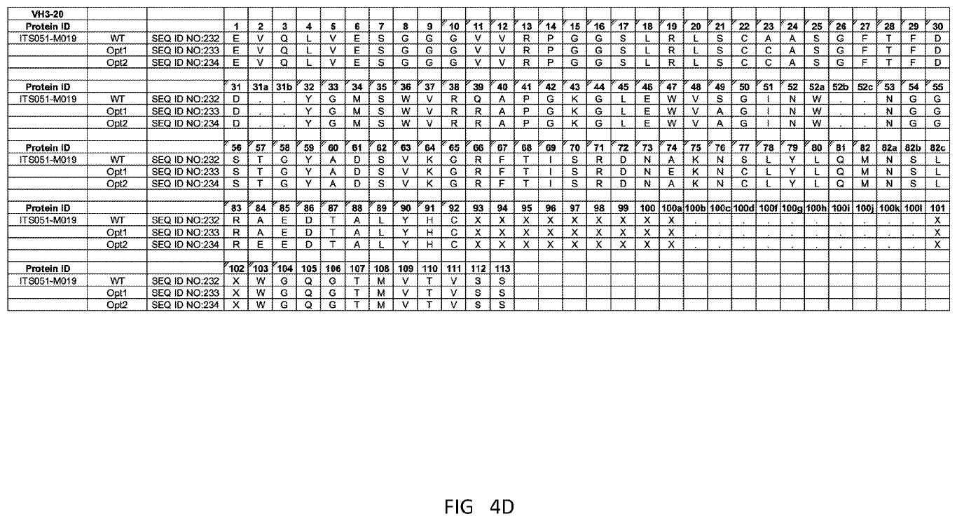

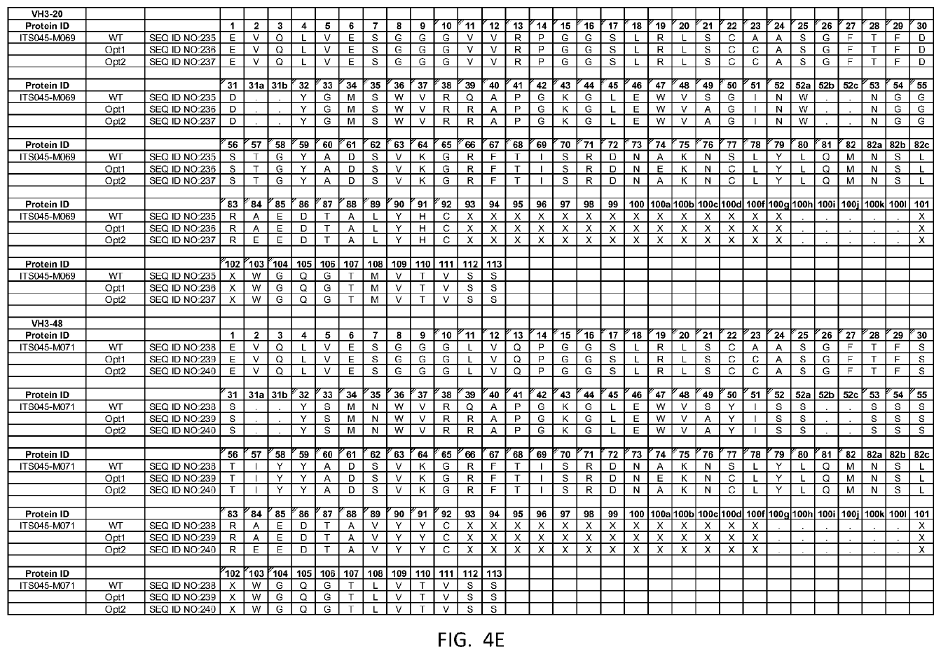

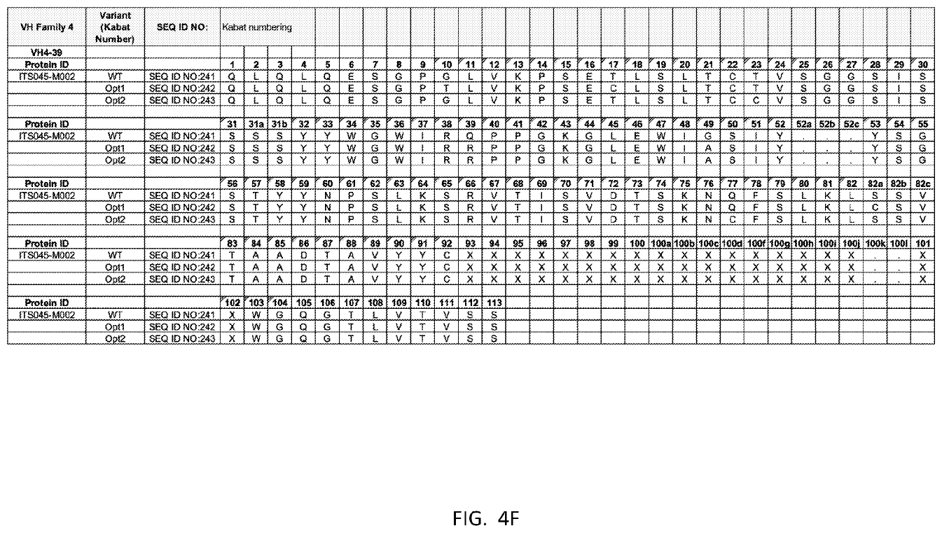

Both the VH1 Opt1 and VH1 Opt2 designs significantly improved both the expression and thermal stability of the tested VH domains (FIGS. 4A-4F).

One of the wild-type VH1-69.2 VH domains expressed very poorly and could not be detected in the expressed supernatants (lower limited of quantitation ˜1 μg/mL), whereas both VH1 Opt1 and VH1 Opt2 variants had significantly improved expression, at roughly 100 μg/mL. The other VH1 domains also showed significant increases in both expression and thermal stability (Table 7).

Both the VH3 Opt1 and VH3 Opt2 designs led to significant increases in thermal stability for all the VH3 domains and improved expression for all but one VH3 domain (Table 7).

The one VH4 germline molecule that was evaluated did not express as a WT molecule but expressed well with the optimizing VH4_Opt1 design mutations, including the 17C-82aC (Table 7).

Table 7 shows the expression titers, the change in expression titers vs. wild type, and results of stability experiments for several of the disulfide stabilized VH domains that include additional substitutions according to the disclosure.

TABLE 7

| | | | | Fold | | | | | | | | | | | | | |
|---|---|---|---|---|---|---|---|---|---|---|---|---|---|---|---|---|---|
| | | | | Expression | | | | | | | | | | | | | |
| | Variant | | | Change | DSF | | Amino Acid Substitutions | | | | | | | | | | |
| VH Family 1 | (Kabat | Titer | Error | versus | Tₘ | Found in | (Kabat Number) | | | | | | | | | | |
| Protein ID | Number) | (∞g/mL) | (n = 2) | Wild-Type | (° C.) | SEQ ID NO: | 10 | 16 | 17 | 23 | 39 | 48 | 49 | 74 | 77 | 82a | 84 |
| VH1-8 | | | | | | | | | | | | | | | | | |
| ITS045-M073 | WT | 129.9 | 13.3 | 1 | 61.5 | SEQ ID NO: 218 | E | A | S | K | Q | M | G | S | T | S | S |
| | Opt1 | 443 | 9 | 3.4 | 73 | SEQ ID NO: 219 | E | A | C | K | R | I | G | S | T | C | S |
| | Opt2 | 404 | 10 | 3.1 | 76 | SEQ ID NO: 220 | E | D | C | K | R | I | G | S | T | C | S |
| VH1-69.2 | | | | | | | | | | | | | | | | | |
| ITS045-M070 | WT | 665 | 7 | 1 | n.d. | SEQ ID NO: 221 | E | A | T | K | Q | M | G | S | T | S | S |
| | Opt1 | 1106 | 538 | 1.7 | 88.5 | SEQ ID NO: 222 | E | A | C | K | R | I | G | S | T | C | S |
| | Opt2 | 1238 | 306 | 1.9 | 85 | SEQ ID NO: 223 | E | D | C | K | R | I | G | S | T | C | S |
| ITS050-M002S | WT | 1 | 0 | 1 | n.d. | SEQ ID NO: 224 | E | A | T | K | Q | M | G | S | T | S | S |
| | Opt1 | 122.5 | 58.5 | 122.5 | 78.3 | SEQ ID NO: 225 | E | A | C | K | R | I | G | S | T | C | S |
| | Opt2 | 93.5 | 66.5 | 93.5 | n.d. | SEQ ID NO: 226 | E | D | C | K | R | I | G | S | T | C | S |
| | | | | Fold | | | | | | | | | | | | | |
| | | | | Expression | | | | | | | | | | | | | |
| | Variant | | | Change | DSF | | Amino Acid Substitutions | | | | | | | | | | |
| VH Family 3 | (Kabat | Titer | Error | versus | Tₘ | Found in | (Kabat Number) | | | | | | | | | | |
| Protein ID | Number) | (∞g/mL) | (n = 2) | Wild-Type | (° C.) | SEQ ID NO: | 10 | 16 | 17 | 23 | 39 | 48 | 49 | 74 | 77 | 82a | 84 |
| VH3-11 | | | | | | | | | | | | | | | | | |
| ITS045-M001 | WT | 241 | 9 | 1 | 64.5 | SEQ ID NO: 227 | G | G | S | A | Q | V | S | A | S | N | A |
| | Opt1 | 479 | 45 | 2.0 | 83.5 | SEQ ID NO: 228 | G | G | S | C | R | V | A | E | C | N | A |
| | Opt2 | 382 | 34 | 1.6 | 85.5 | SEQ ID NO: 229 | G | G | S | C | R | V | A | A | C | N | E |
| VH3-15 | | | | | | | | | | | | | | | | | |
| ITS053-M011 | WT | 1005 | 571 | 1 | 64.5 | SEQ ID NO: 111 | G | G | S | A | Q | V | G | S | T | N | T |
| | Opt1 | 580 | 120 | 0.6 | 79 | SEQ ID NO: 230 | G | G | S | C | R | V | A | E | C | N | T |
| | Opt2 | 413 | 9 | 0.4 | 81 | SEQ ID NO: 231 | G | G | S | C | R | V | A | S | C | N | E |
| VH3-20 | | | | | | | | | | | | | | | | | |
| ITS051-M019 | WT | 1 | 0 | 1 | n.d. | SEQ ID NO: 232 | G | G | S | A | Q | V | S | A | S | N | A |
| | Opt1 | 595 | 125 | 595 | n.d. | SEQ ID NO: 233 | G | G | S | C | R | V | A | E | C | N | A |
| | Opt2 | 489 | 65 | 489 | n.d. | SEQ ID NO: 234 | G | G | S | C | R | V | A | A | C | N | E |
| ITS045-M069 | WT | 140.5 | 13.5 | 1 | 58 | SEQ ID NO: 235 | G | G | S | A | Q | V | S | A | S | N | A |
| | Opt1 | 467 | 219 | 3.3 | 79.5 | SEQ ID NO: 236 | G | G | S | C | R | V | A | E | C | N | A |
| | Opt2 | 277 | 49 | 2.0 | 80.5 | SEQ ID NO: 237 | G | G | S | C | R | V | A | A | C | N | E |
| VH3-48 | | | | | | | | | | | | | | | | | |
| ITS045-M071 | WT | 1 | 0 | 1 | 57.5 | SEQ ID NO: 238 | G | G | S | A | Q | V | S | A | S | N | A |
| | Opt1 | 305 | 221 | 305 | 75 | SEQ ID NO: 239 | G | G | S | C | R | V | A | E | C | N | A |
| | Opt2 | 69.05 | 68.95 | 69.1 | 77 | SEQ ID NO: 240 | G | G | S | C | R | V | A | A | C | N | E |

| | | | | Fold | | | | | | | | | | | | | |
|---|---|---|---|---|---|---|---|---|---|---|---|---|---|---|---|---|---|
| | | | | Expression | | | | | | | | | | | | | |
| | Variant | | | Change | DSF | | Amino Acid Substitutions | | | | | | | | | | |
| VH Family 4 | (Kabat | Titer | Error | versus | Tₘ | Found in | (Kabat Number) | | | | | | | | | | |
| Protein ID | Number) | (~g/mL) | (n=2) | Wild-Type | (° C.) | SEQ ID NO: | 10 | 16 | 17 | 23 | 39 | 48 | 49 | 74 | 77 | 82a | 84 |
| VH4-39 | | | | | | | | | | | | | | | | | |
| ITS045-M002 | WT | 1 | 0 | 1 | n.d. | SEQ ID NO: 241 | G | E | T | T | Q | I | G | S | Q | S | A |
| | Opt1 | 215.05 | 214.95 | 215.1 | n.d. | SEQ ID NO: 242 | T | E | C | T | R | I | A | S | Q | C | A |
| | Opt2 | 1 | 0 | 1 | n.d. | SEQ ID NO: 243 | G | E | T | C | R | I | A | S | C | S | A |

*LLQ = 1 ug/mL

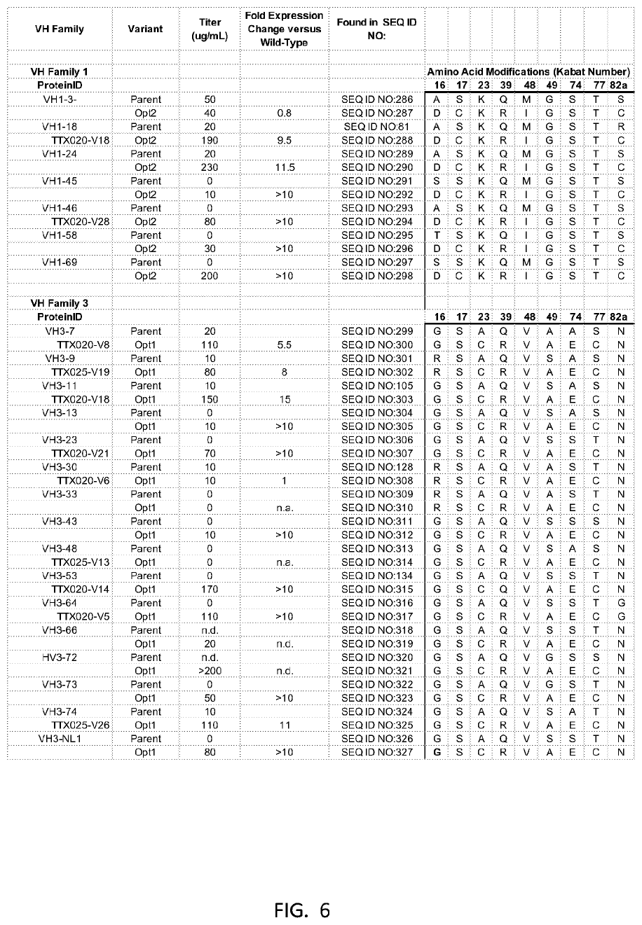

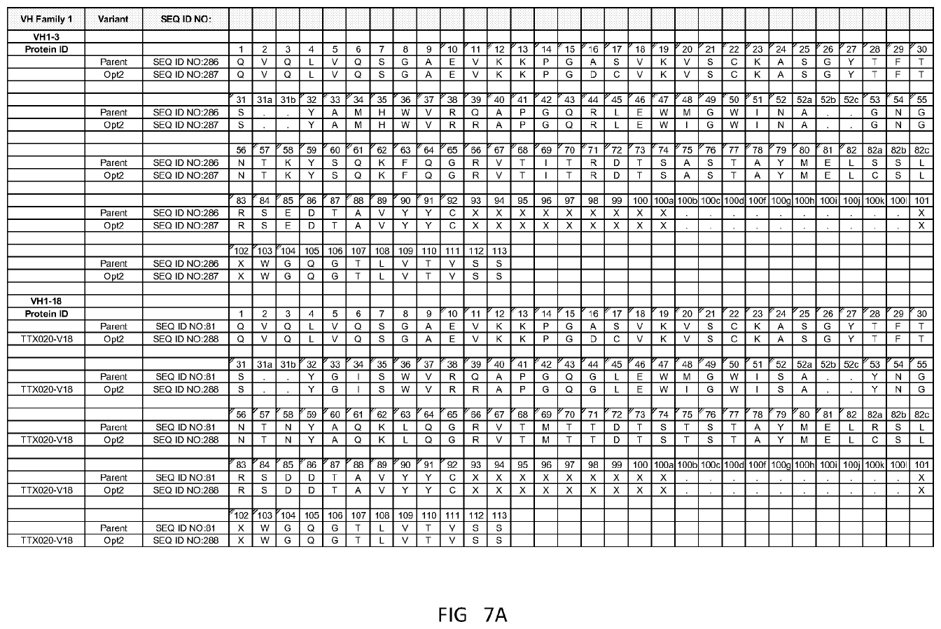

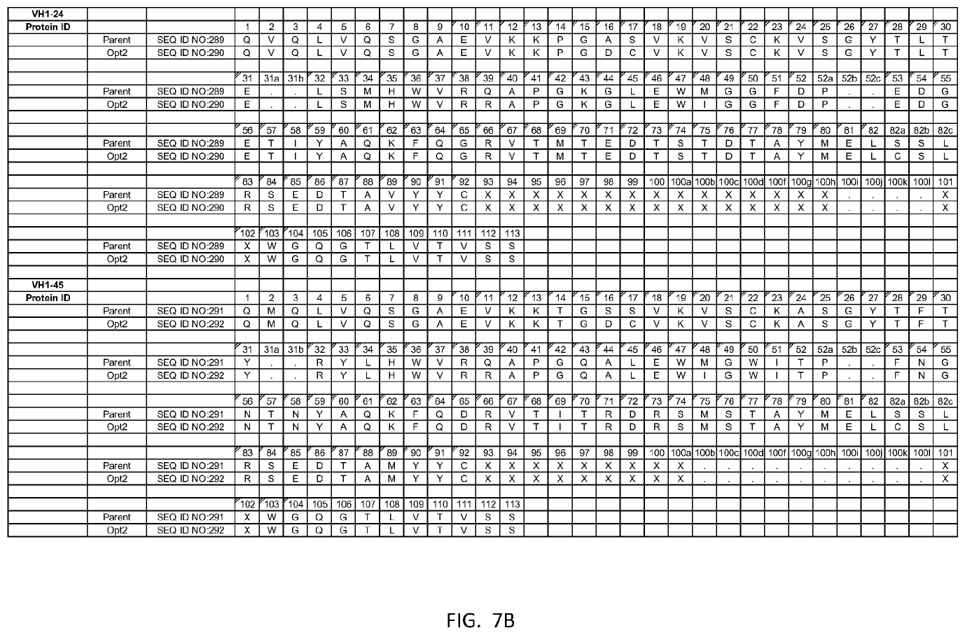

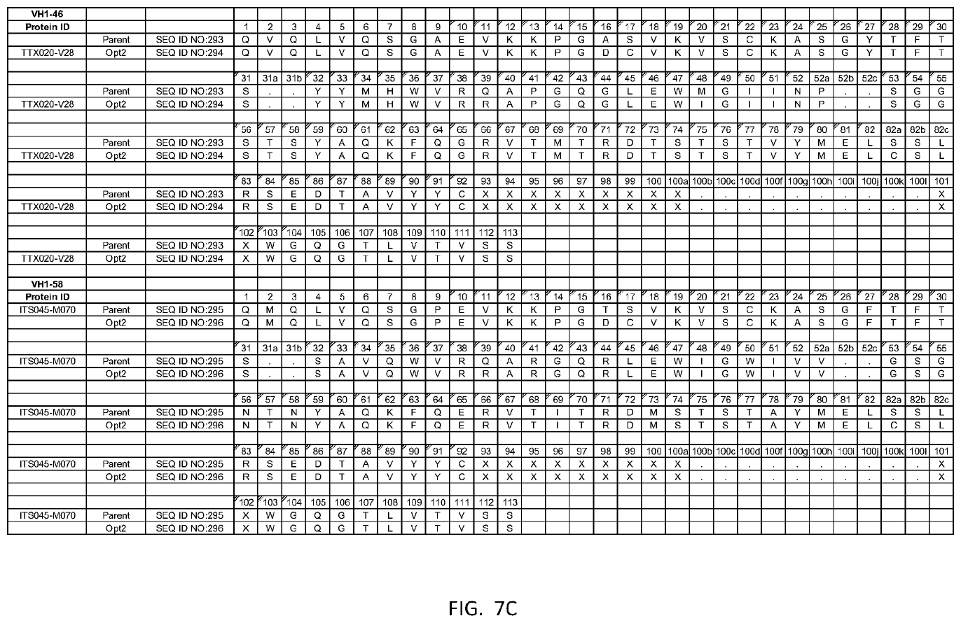

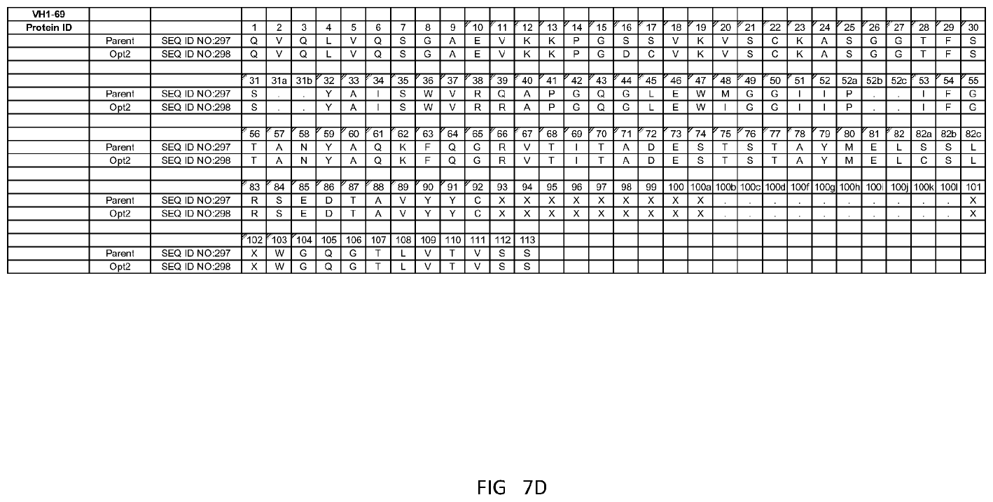

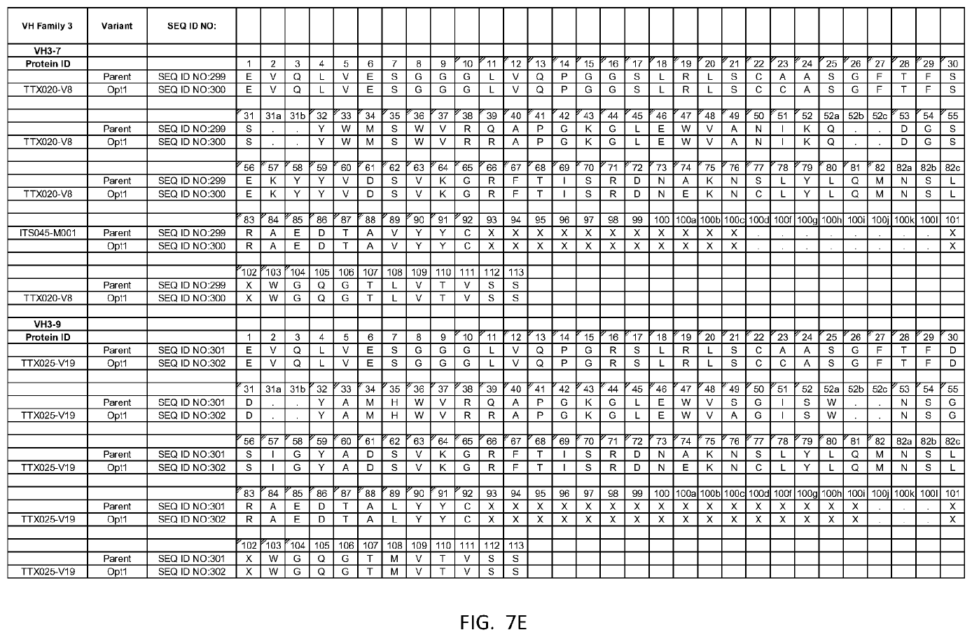

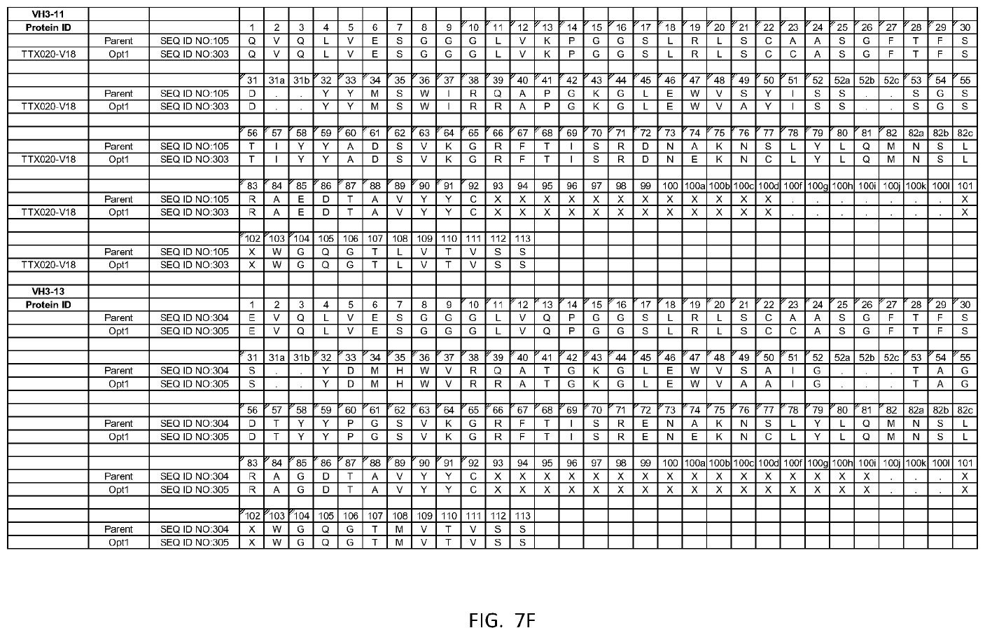

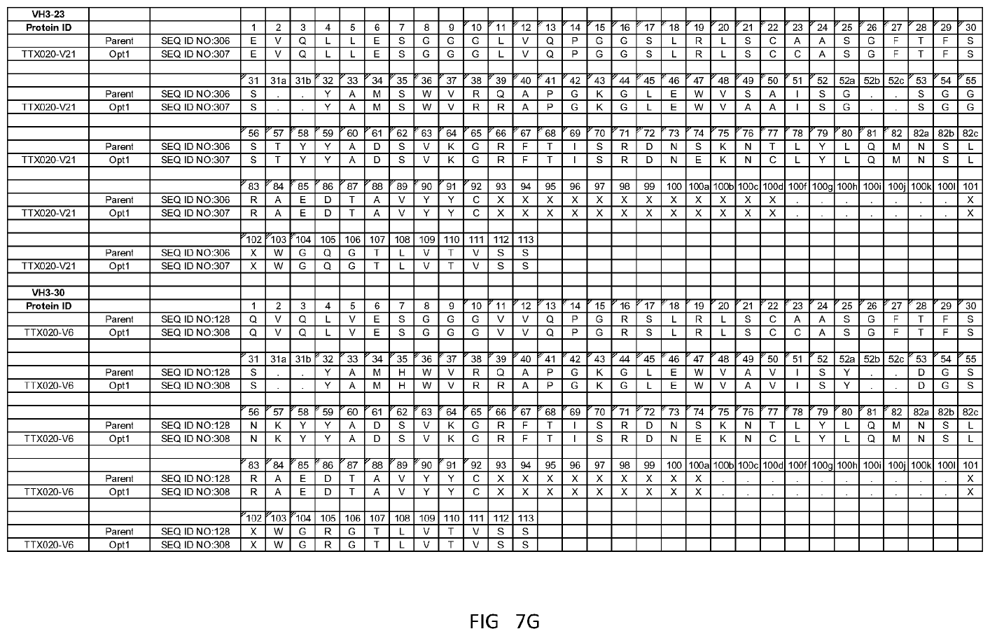

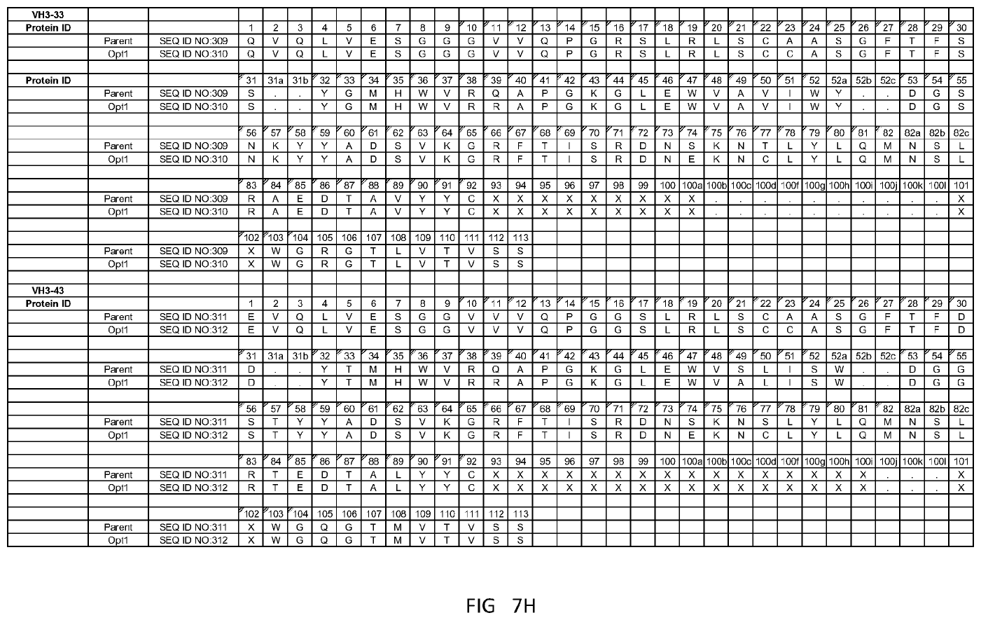

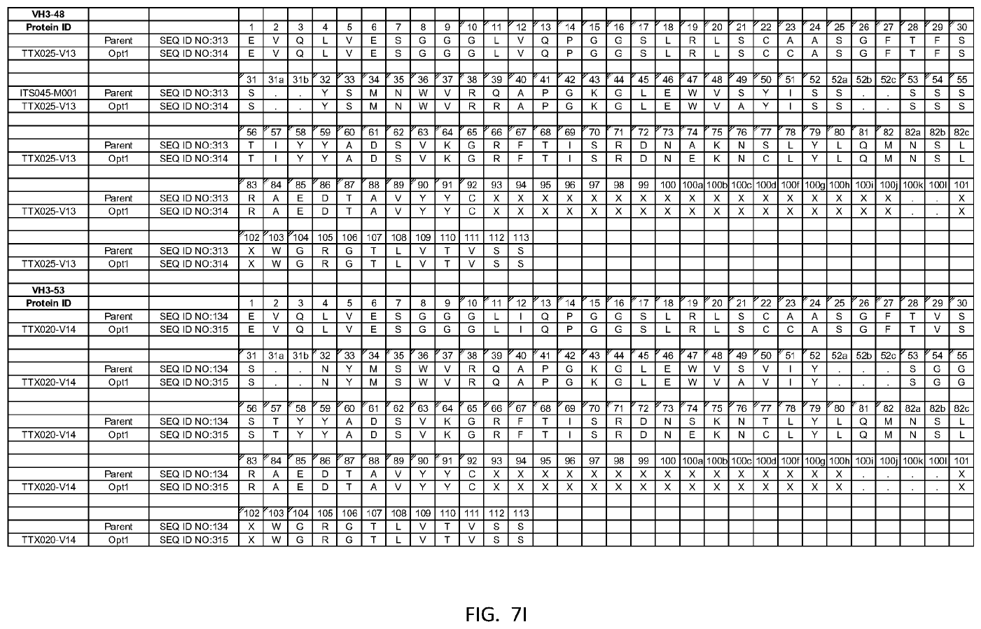

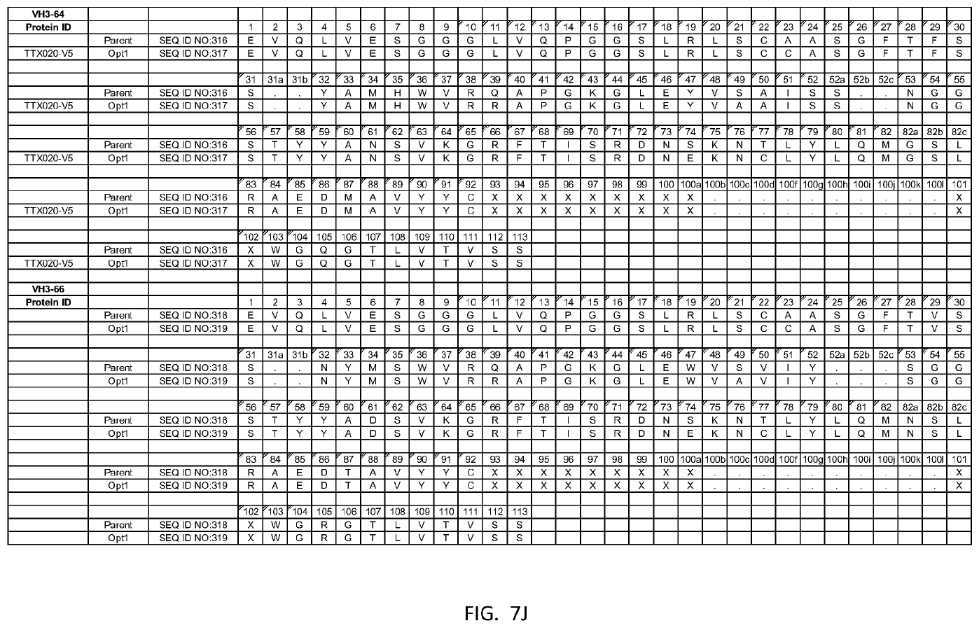

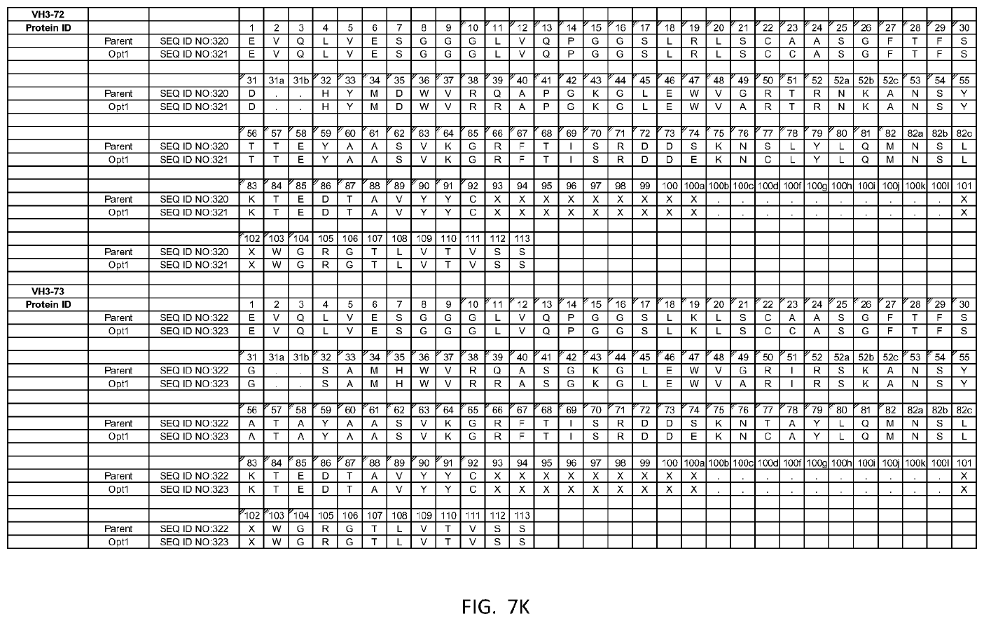

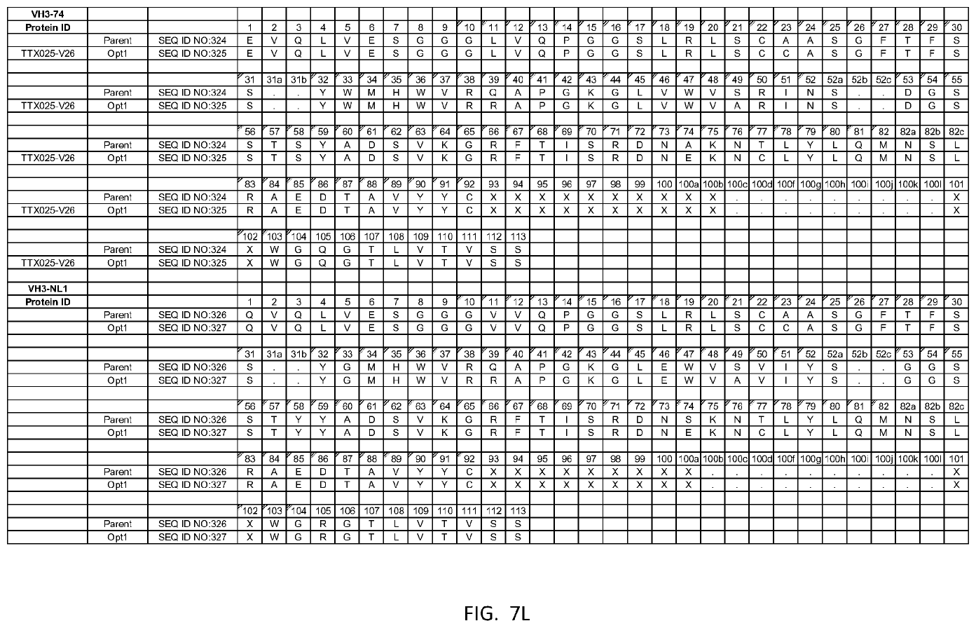

Similar methods were used to identify additional stabilized sequences for VH family 1 and VH family 3 as shown in FIGS. 6, and 7A-7L. Family 1 members were modified according to Option 2 in Table 6. Family 3 members were modified according to Option 1 in Table 6. FIG. 6 provides the stability and expression data for each variant along with a summary of the substitutions for each sequence. FIGS. 7A-7L provides sequence information for each variant.

The increases in expression and thermal stability for each of the domains was primarily at what we observe for standard antibodies. For example, the measurable Tm values for the optimized VH domains range from 73-89° C., which puts these in a thermal stability range that is the same or higher than natural antibody Fab domains. Overall, these enhancing designs represent general stability/expression solutions for VH domain that can be used scaffolds for recombinantly derived libraries used for phage, yeast, or mammalian display as well as within therapeutic antibody-like modalities.

### Example 4-VH Germline Family 4 Variants

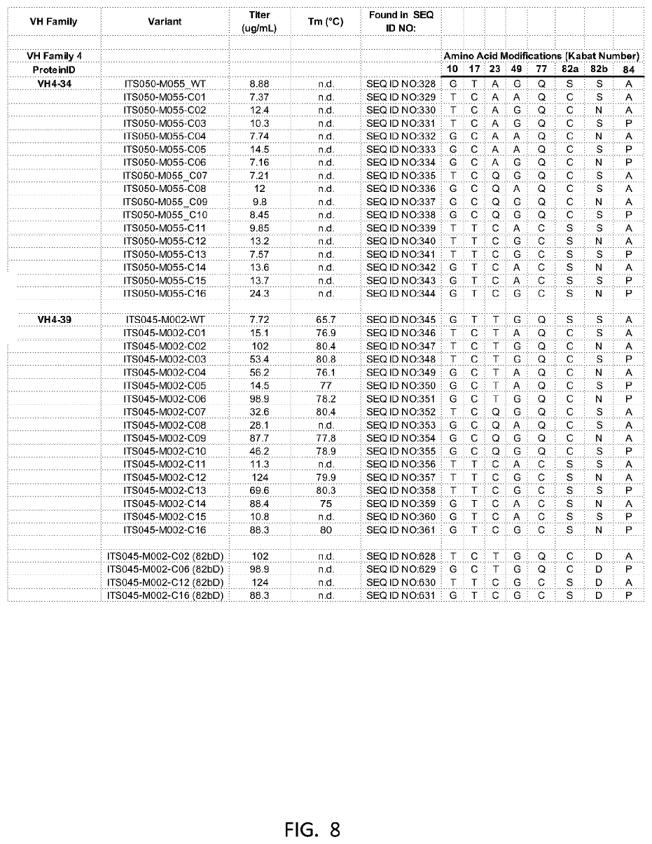

VH4 family members 4-34 and 4-39 were modified with one of the disulfide pairs 17C/82aC or 23C/77C and one or more of the following amino acid substitutions 10T, 23Q, 49A, 82bN, 82bD, and 84P. A summary of the substitutions along with their effect on VH stability and expression (determined according to Example 1) is shown in FIG. 8.

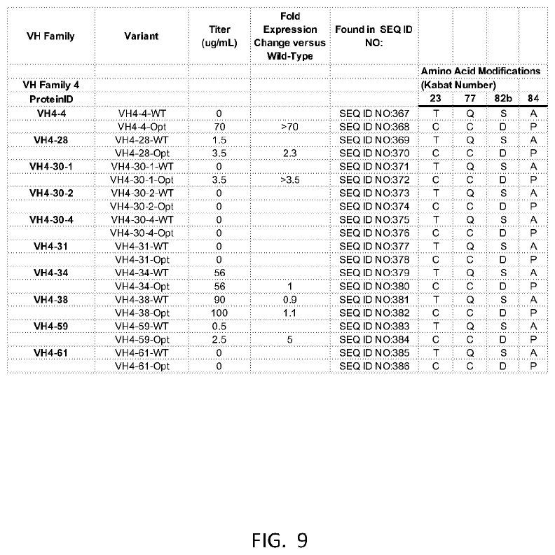

FIG. 9 shows a summary of substitutions and their effect on molecular stability and expression (determined according to Example 1) for germline family 4 VH family members 4-4, 4-28, 4-30-1, 4-30-2, 4-30-4, 4-31, 4-34, 4-38, 4-59 and 4-61. Each variant includes 23C/77C along with 82aD and 84P.

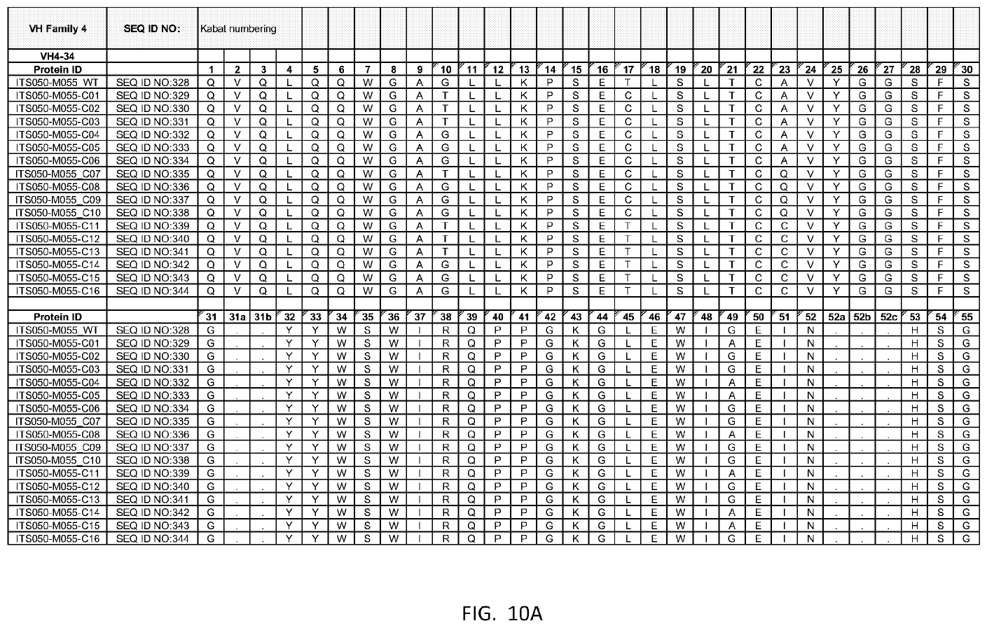

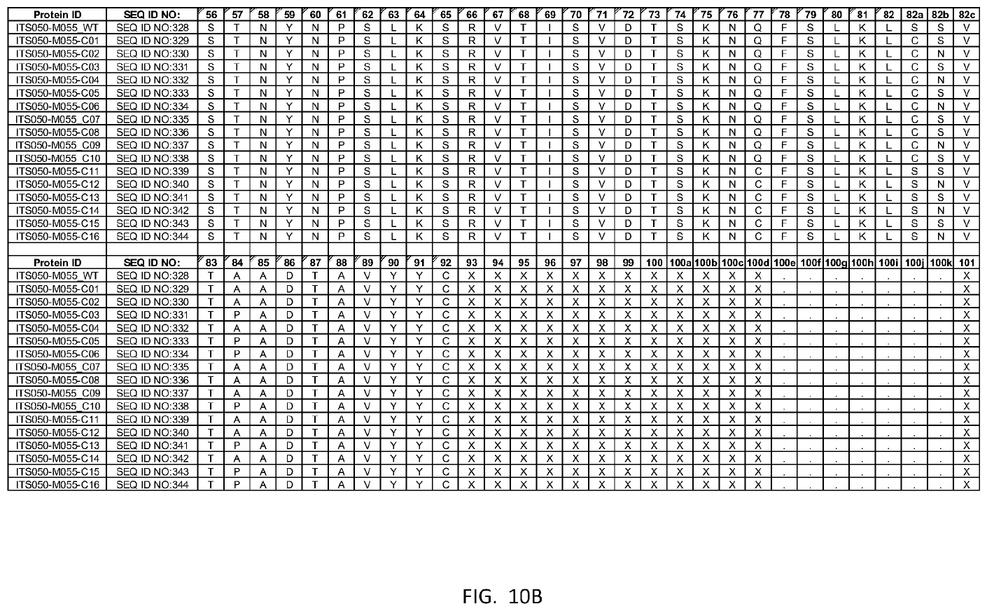

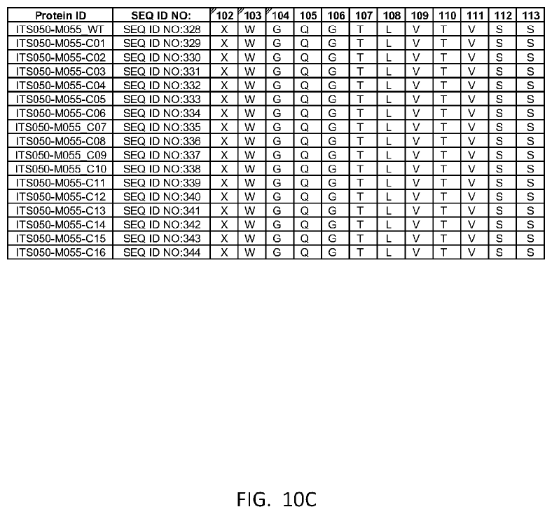

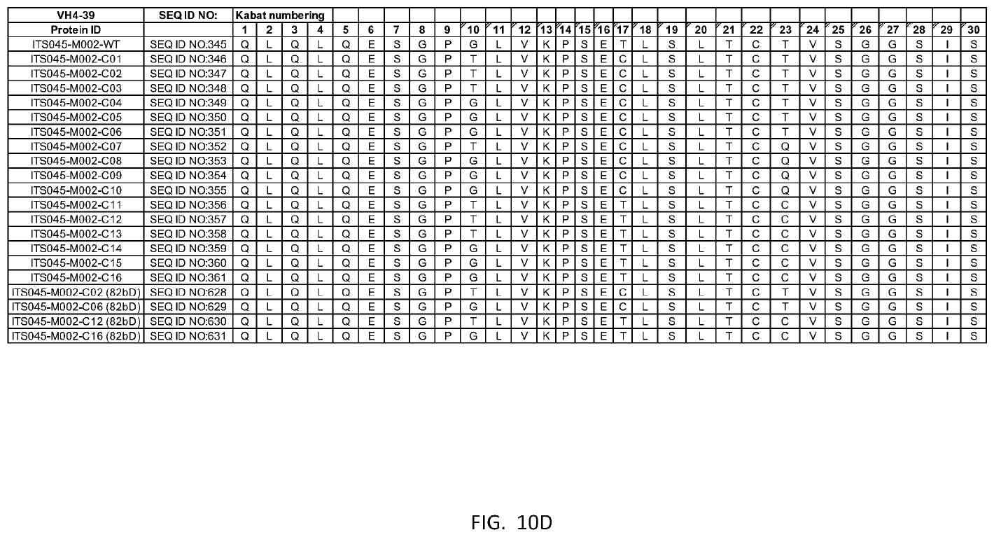

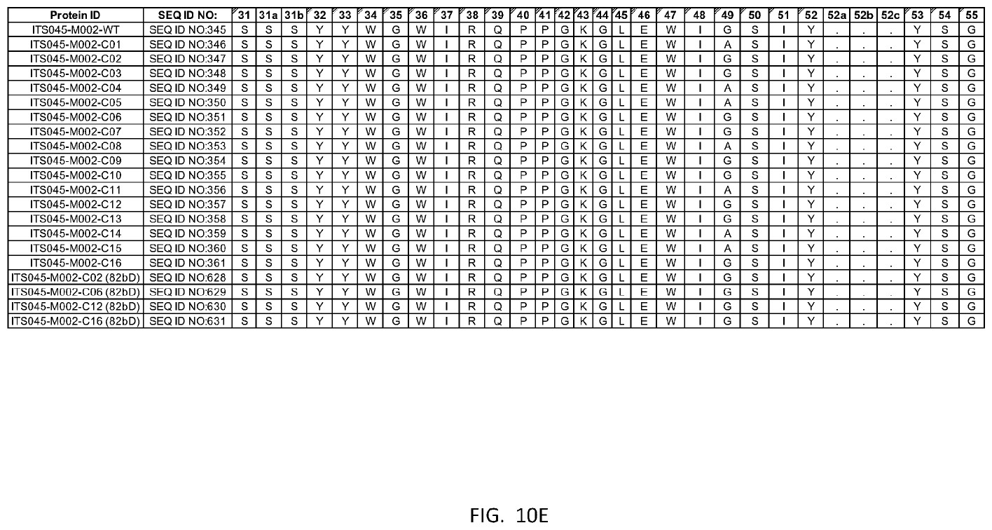

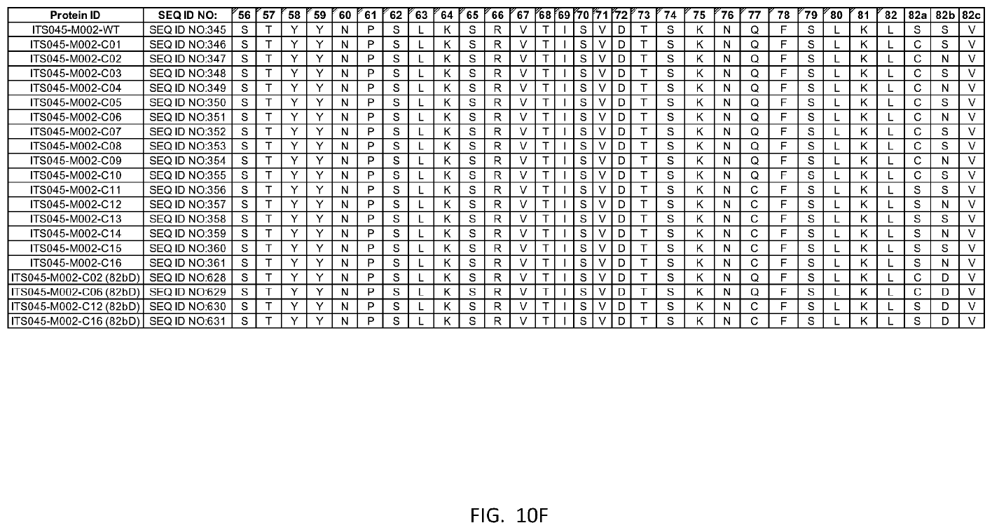

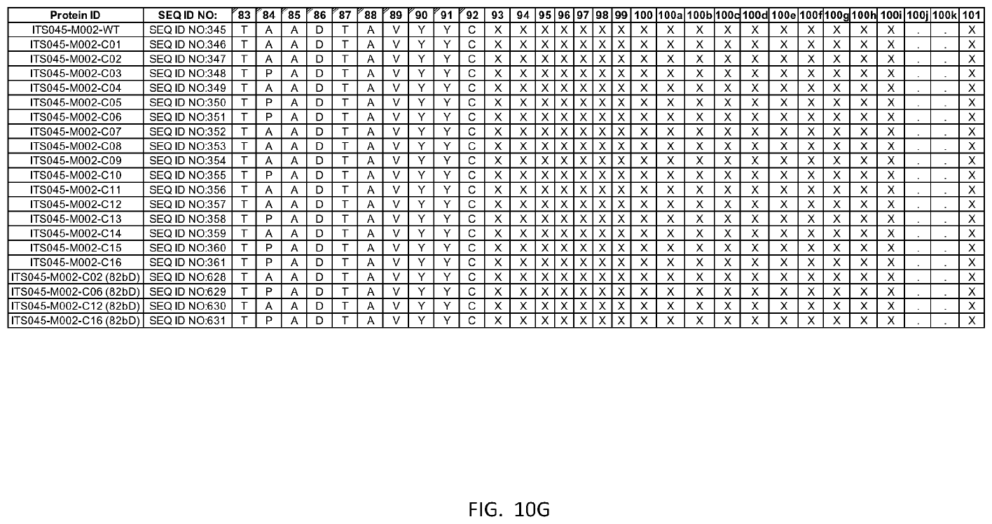

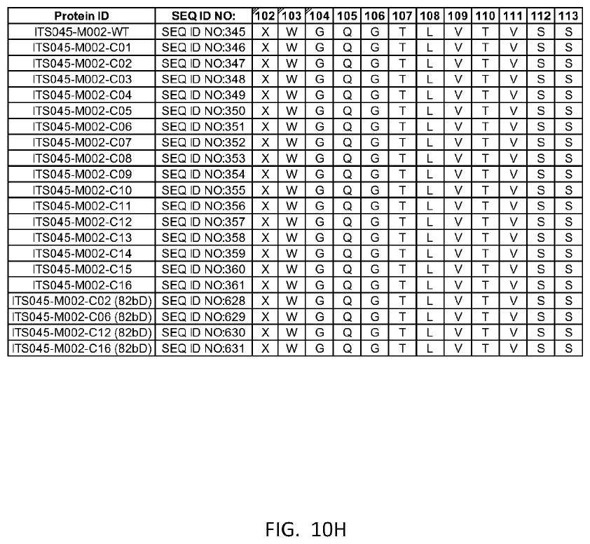

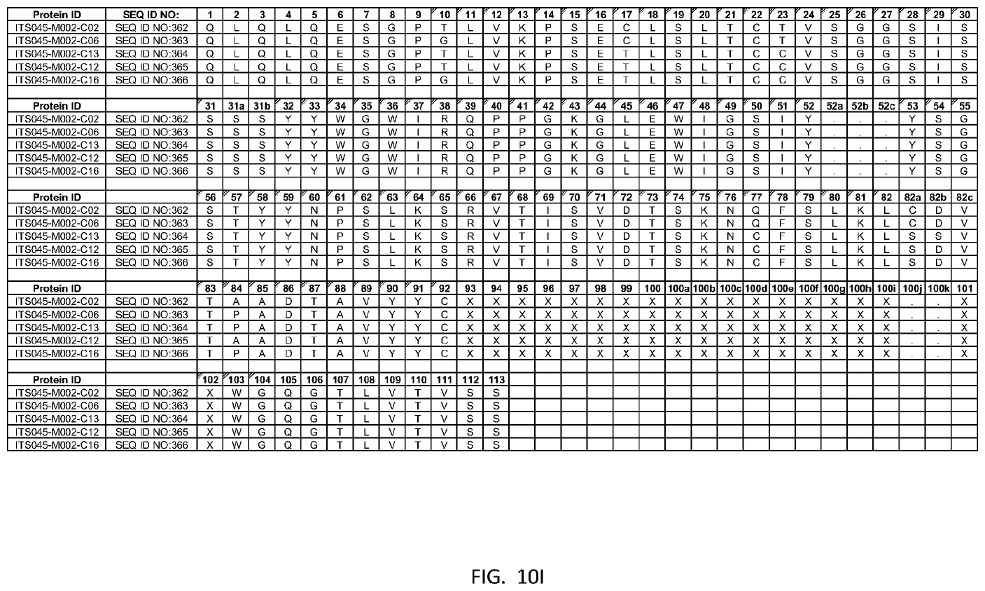

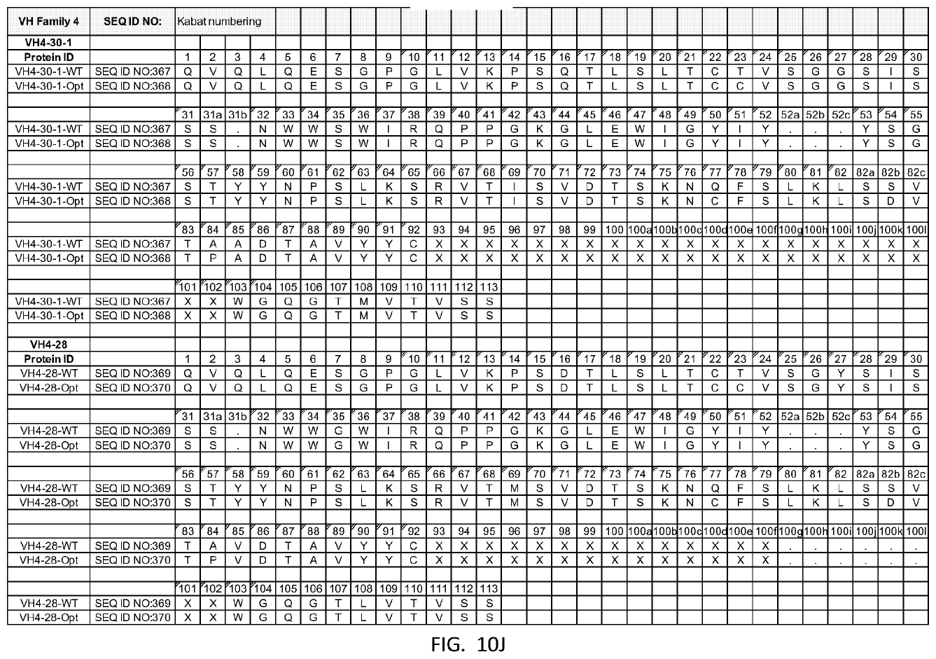

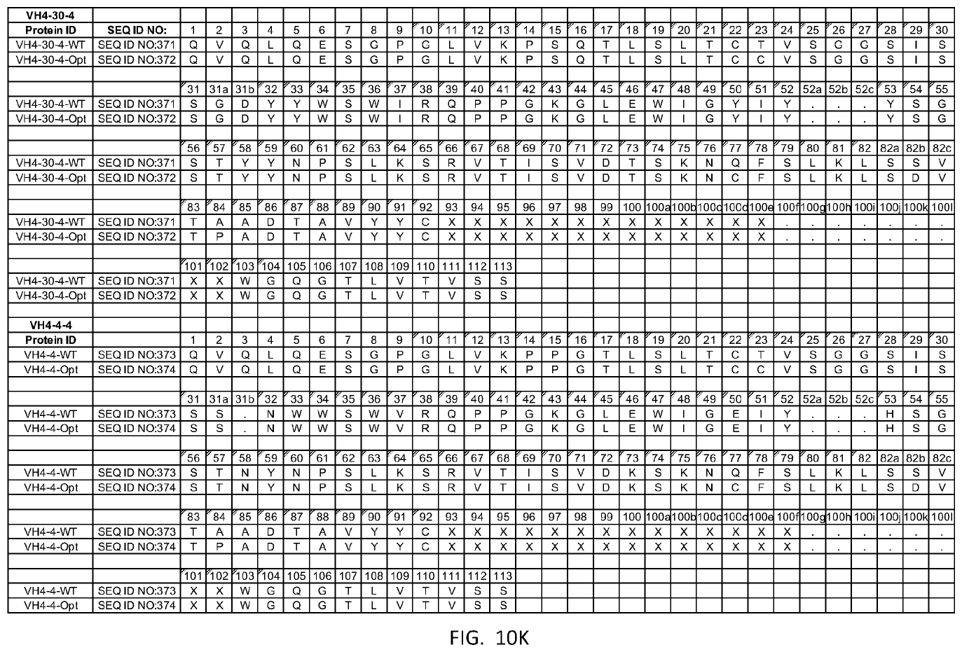

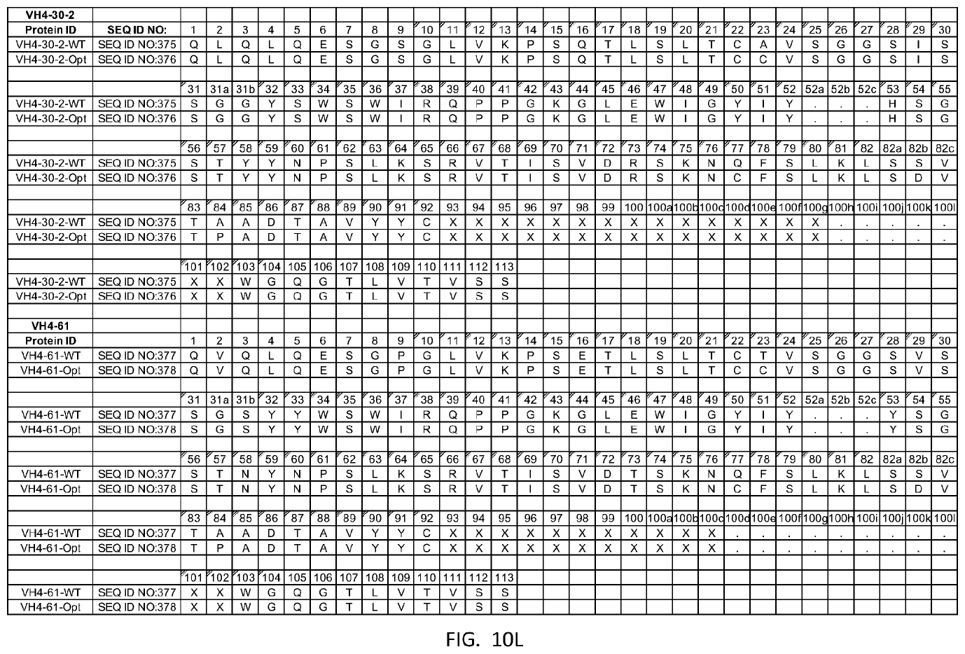

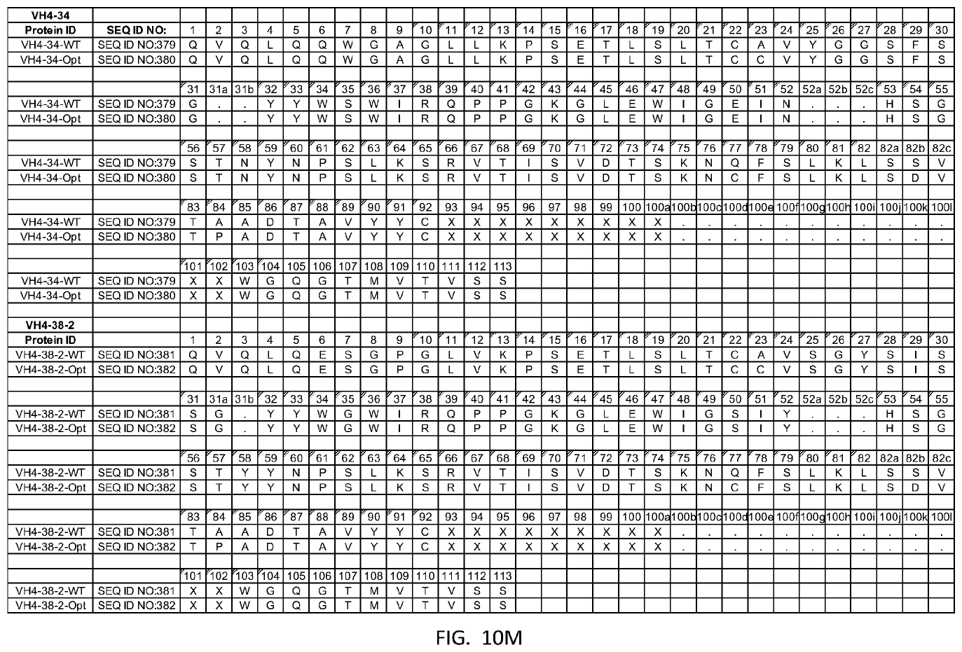

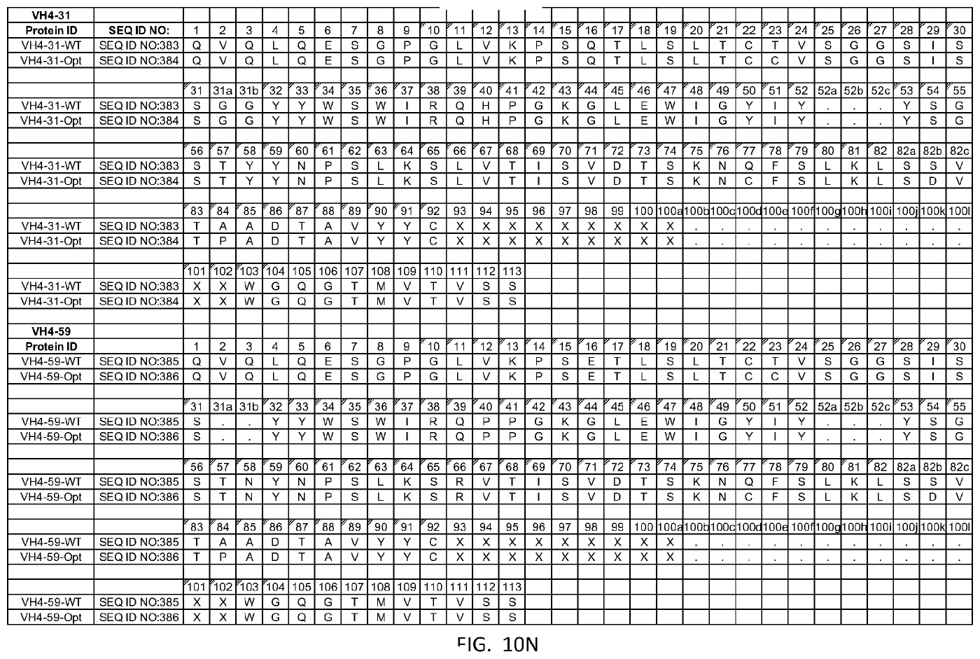

Amino acid sequences for the variants summarized in FIGS. 8 and 9 are shown in FIGS. 10A-10N.

### Example 5-VH Germline Family 2 Germline Variants

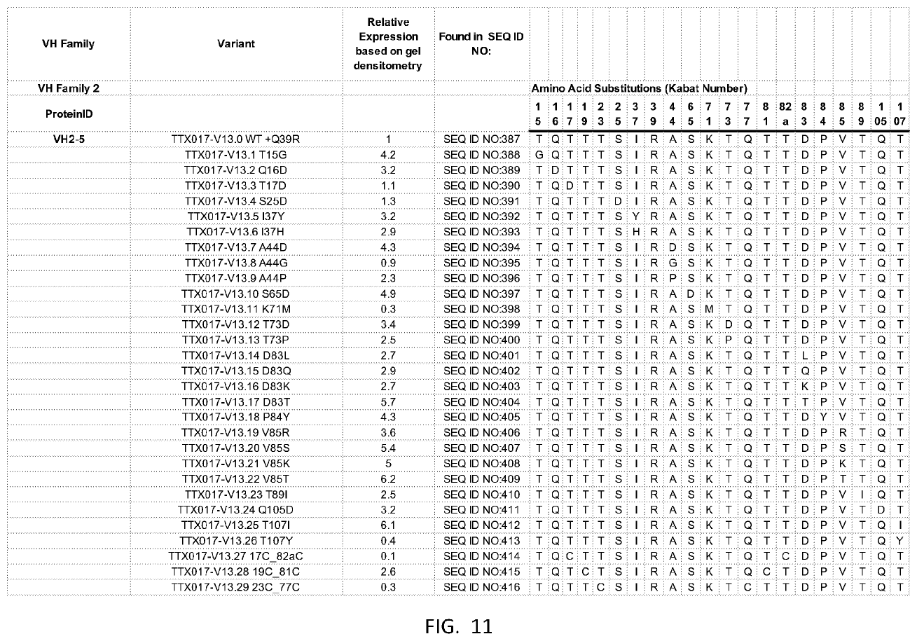

A VH Family 2 member VH2-5 having an existing 39R substitution (parent) was further modified with one of the following substitutions: 15G, 16D, 17D, 25D, 37Y, 44D, 44G, 44P, 65D, 71M, 73D, 73P, 83L, 83Q, 83T, 84Y, 85R, 85S, 85K, V85T, 89I, 105D, 107I, 107Y, or combination of substitutions 17C/82aC, 19C/81C, and 23C/77C. A summary of the substitutions is shown in FIG. 11, along with expression data.

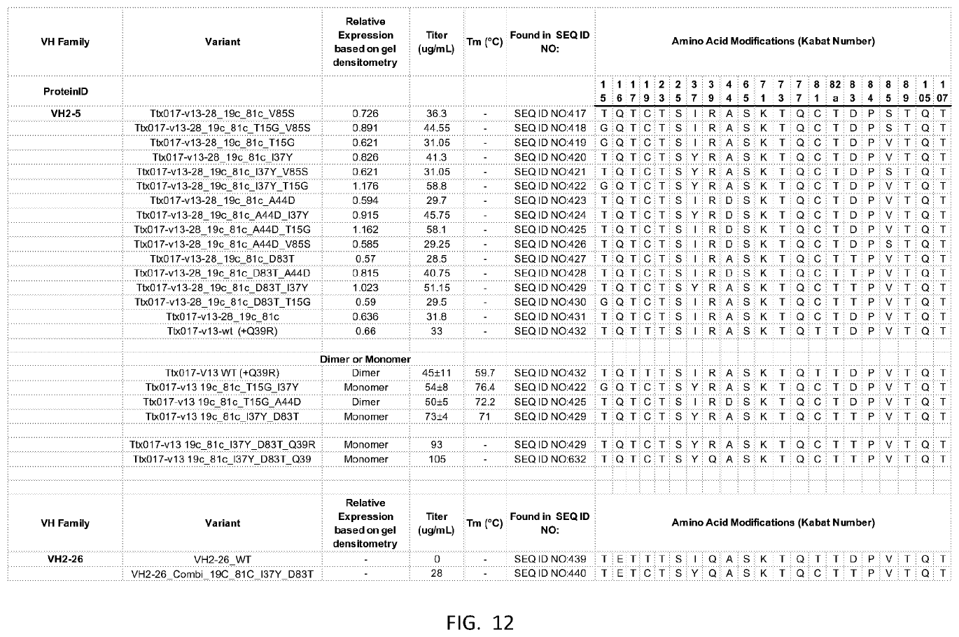

FIG. 12 shows VH Family 2 member V2-5 having an existing 39R substitution and the 19C/81C substitution alone or with a number of other substitutions or combinations thereof, including the following: 15G, 37Y, 44D, 83T, 85S, 15G/37Y, 15G/44D, 15G/83T, 15G/85S, 37Y/44D, 37Y/83T, 37Y/85S, 44D/83T, and 44D/85S. Expression data is shown for each variant.

Examples including the 37Y substitution reflect that, when present, 37Y reduced dimerization of the VH domains. In particular, variants with 19C/81C and one of the following combinations avoided dimerization: 15G/37Y and 37Y/D83T. These examples show a comparison of 37Y/83T variants with WT 39Q and substitution 39R. Both sequences avoided dimerization. These data show that when 37Y is present, 39R is not necessary to eliminate VH domain homodimerization. Variant 19C/81C/37Y/83T appeared to have the most significant improvement in expression over the WT sequence.

FIG. 12 also shows the data indicating improved expression for the VH family 2-26 variant 19C/81C/37Y/83T over the germline sequence.

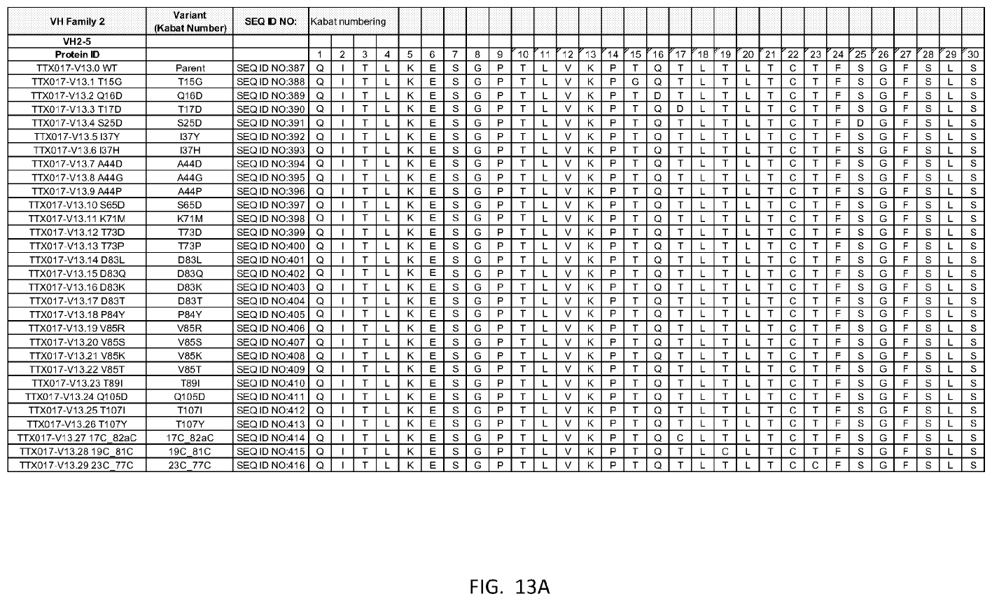

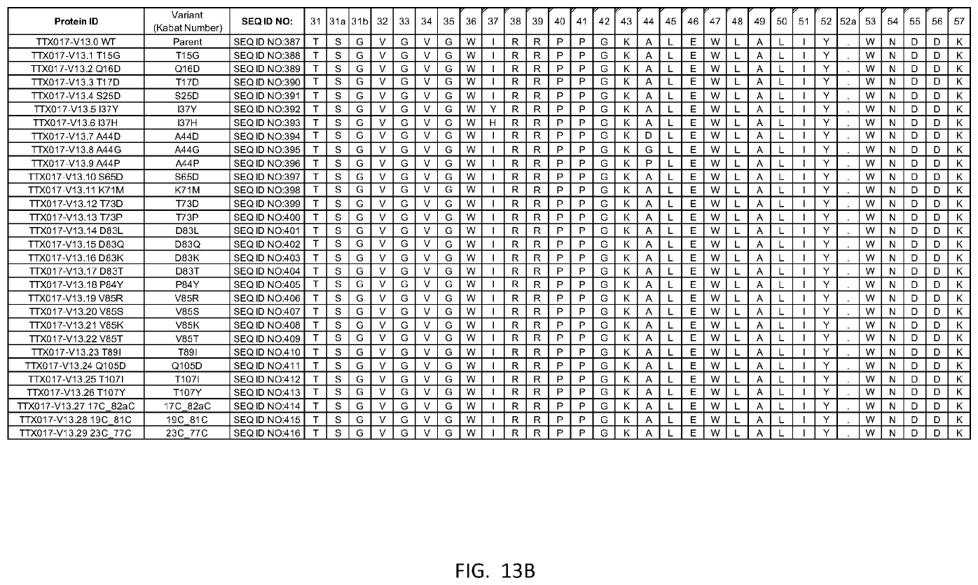

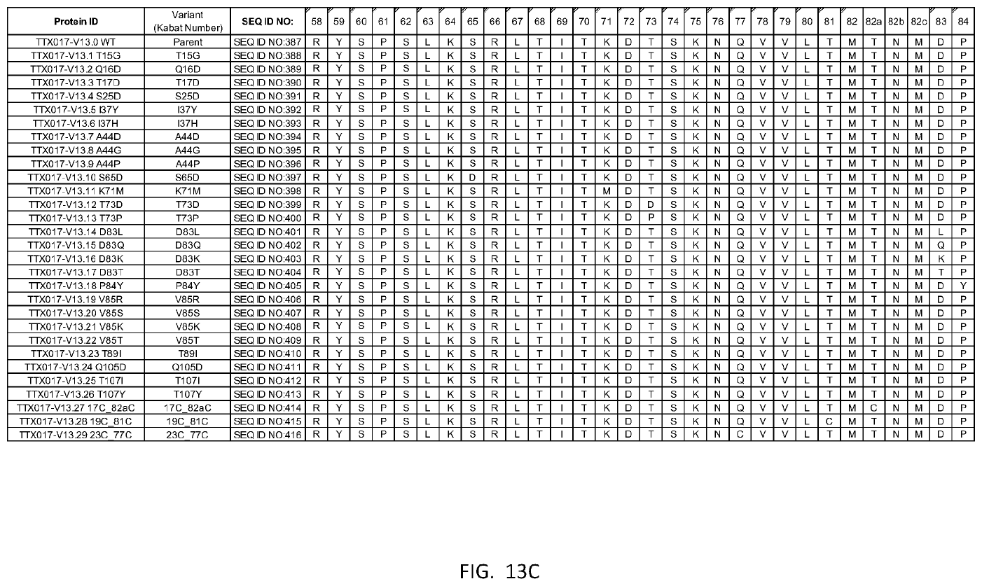

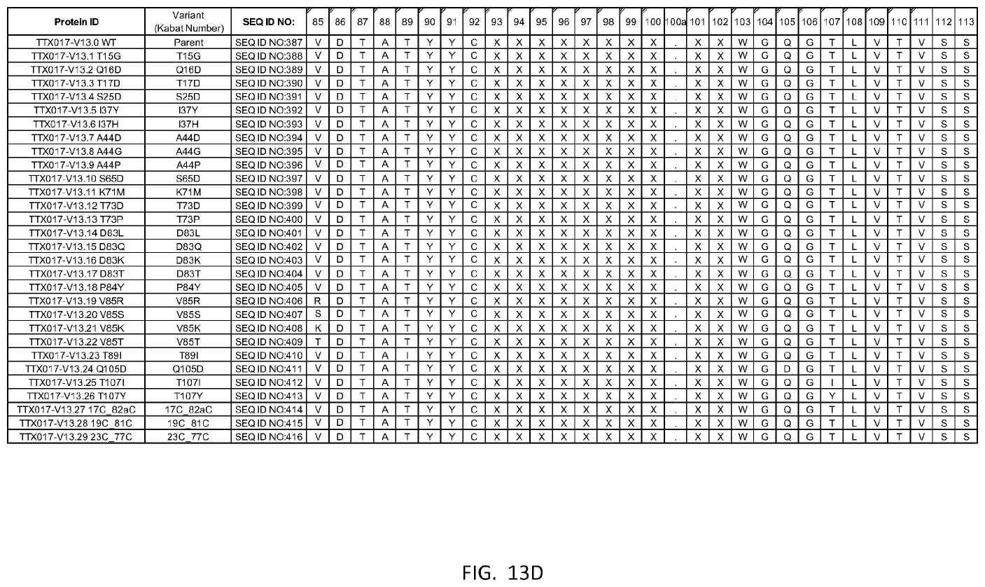

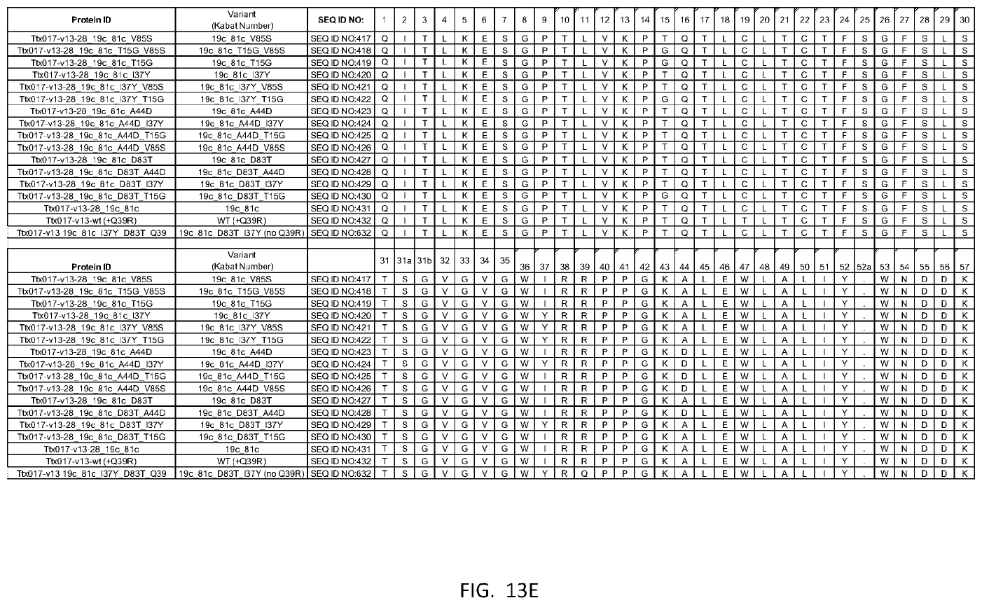

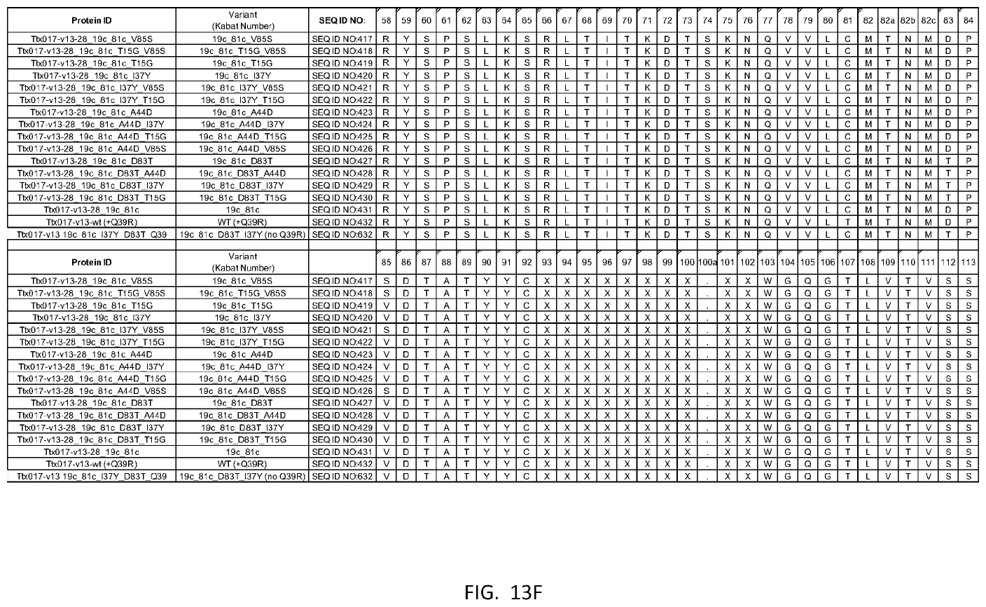

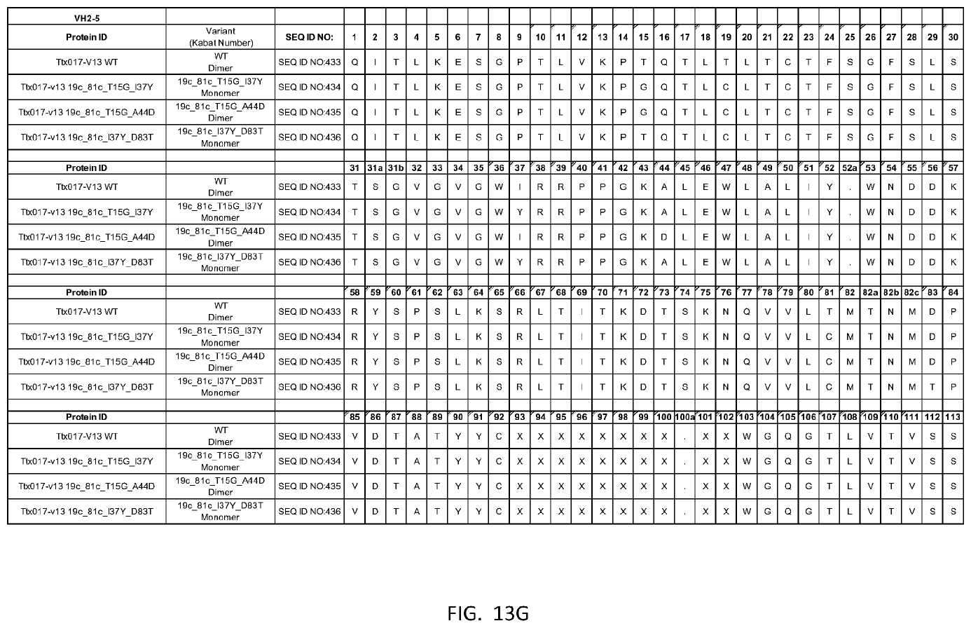

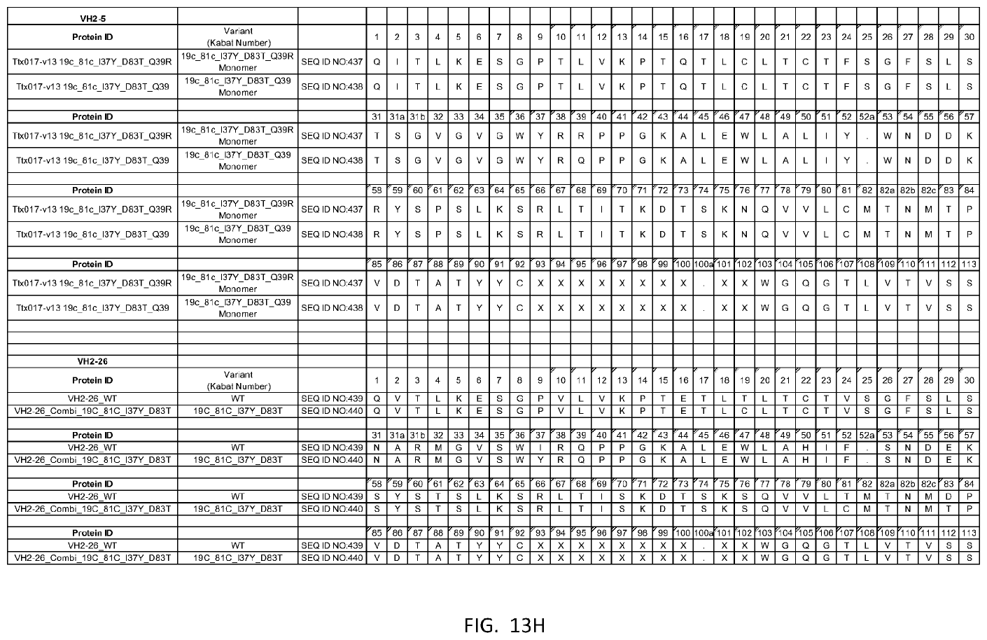

Amino acid sequences for the variants in FIGS. 11 and 12 are shown in FIG. 13A-13H.

### Example 6-VH Germline Family 5 Variants

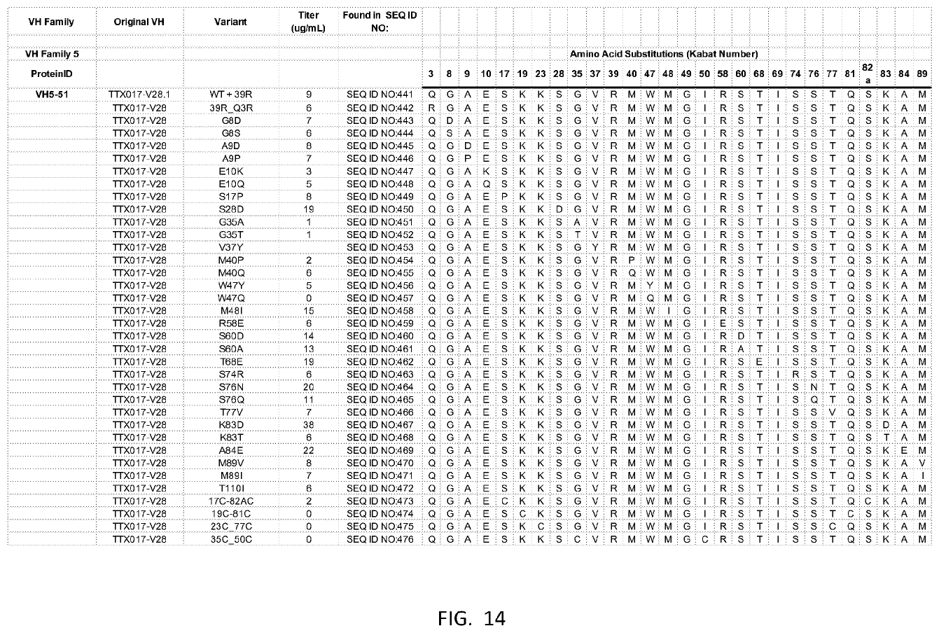

VH Family 5 members having an existing 39R substitution in VH5-51 were further modified with one of the following substitutions: 8D, 8S, 9D, 9P, 10K, 10Q, 17P, 28D, 35A, 35T, 37Y, 40P, 40Q, 47Y, 47Q, 48I, 58E, 60D, 60A, 68E, 74R, 76N, 76Q, 77V, 83D, 83T, 84E, 89V, 89I, 110I or combination of substitutions 17C/82aC, 19C/81C, 23C/77C or 35C/50C. A summary of the substitutions is shown in FIG. 14 along with expression data.

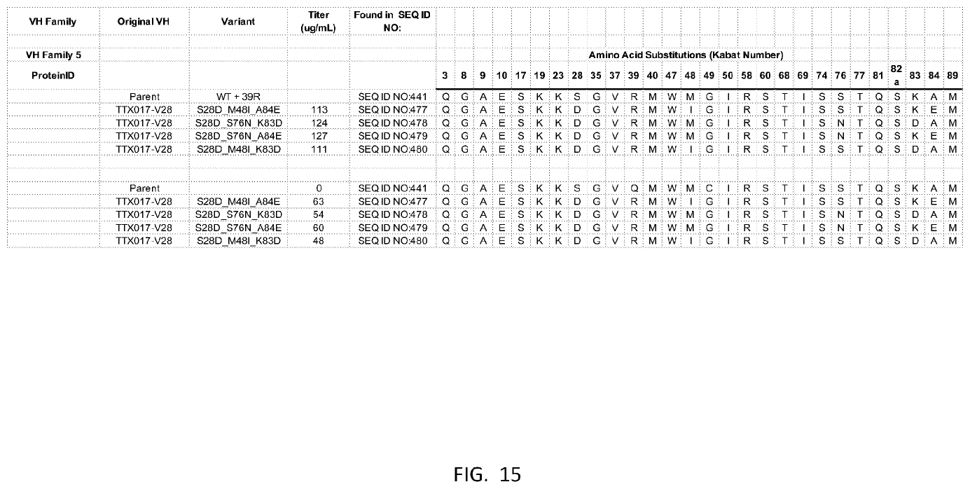

FIG. 15 shows two sets of expression data for the germline and optimized variants of VH5-51 that has been modified with 39R and including the following substitutions: 28D/48I/84 E, S28D/76N/K83D, 28D/39R/76N/84E and 28D/48I/83 D.

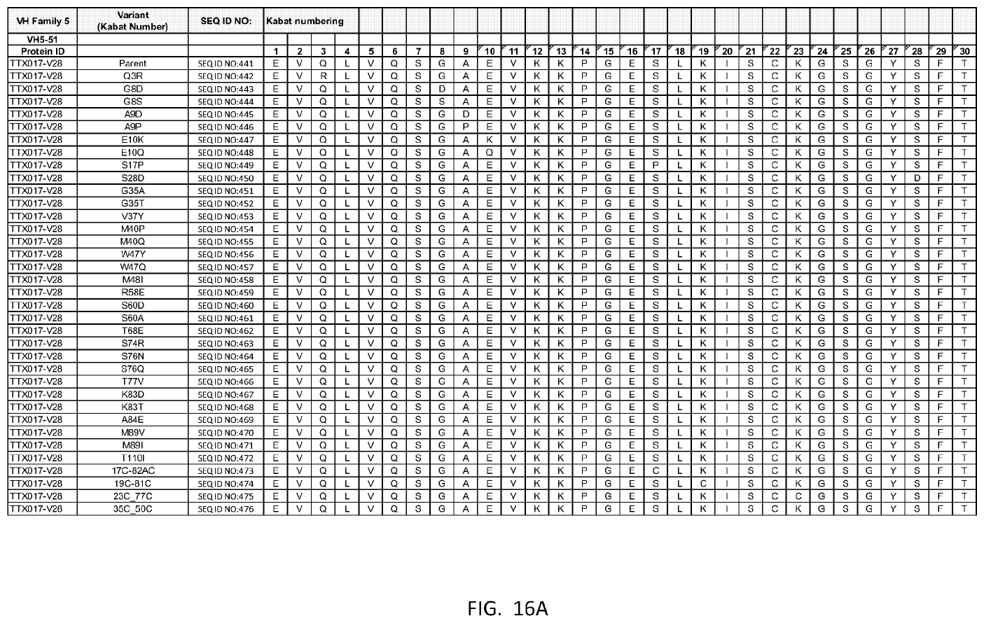

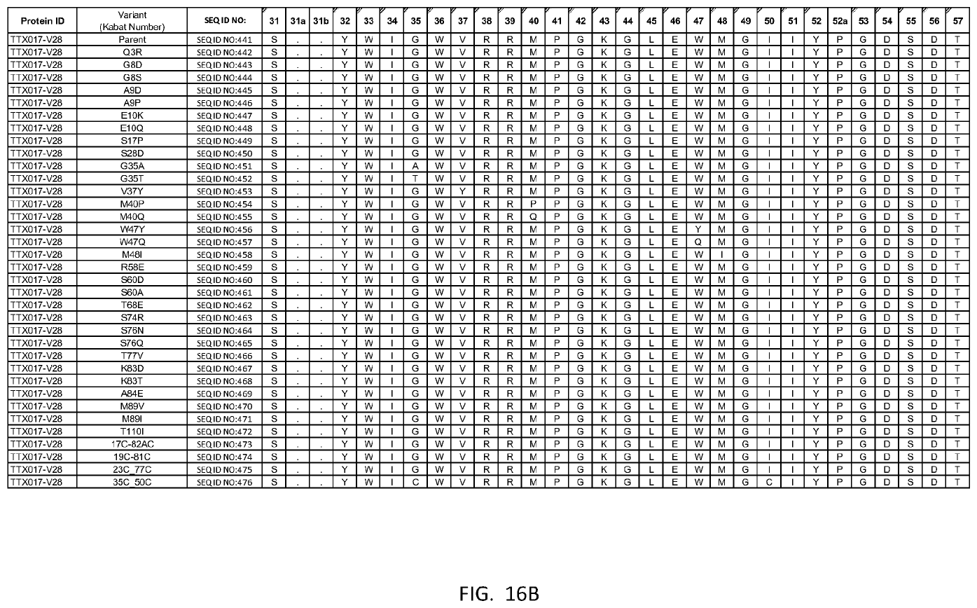

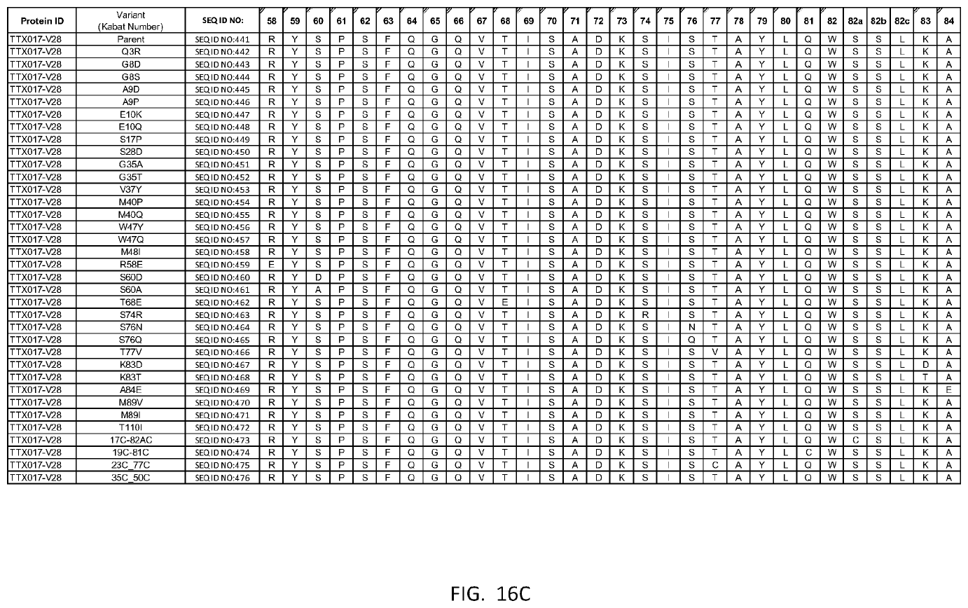

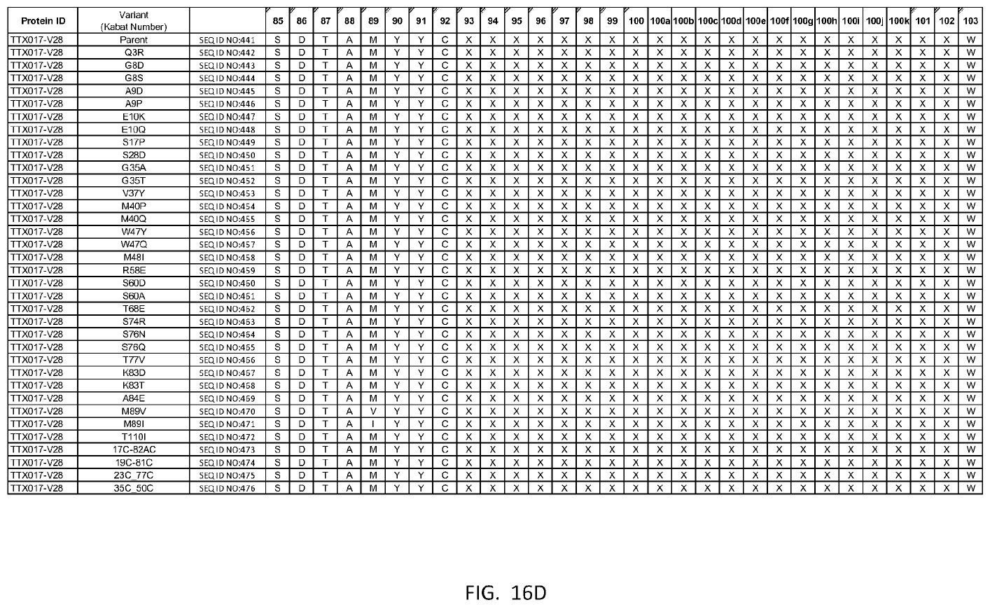

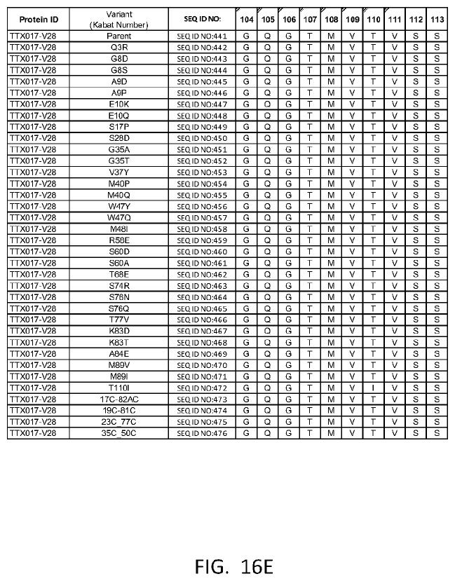

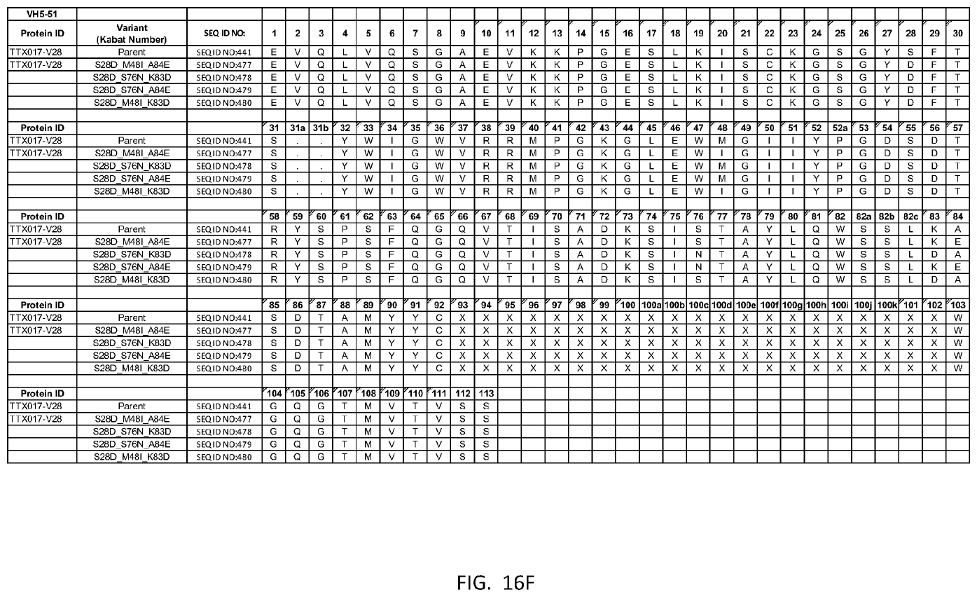

FIGS. 16A-16F provides the sequences for the VH family 5 variants from FIG. 15.

### Example 7: Reducing Constitutive Dimerization of VH Domains

In nature, the vast majority of antibody VH domains, including human VHs, heterodimerize with VL domains from antibody LCs to form a full antigen binding fragment or Fab. However, antibody VH and VL domains are highly homologous in structure and use similar residue positions to bury residues within the VH/VL interface. Given the homology, a proclivity for VH domains to homodimerize using residues at the VH/VL interface has been shown to exist for a fully human VH domain derived from a phage display library (Baral T N, Chao, S Y, Li, S, et al., 2012 Crystal structure of a human single domain antibody dimer formed through VH-VH non-covalent interactions. PLoS One 7, e30149; “Gr6 homodimer”). This VH domain forms a constitutive homodimer whose structure has been solved. Within the structure, residues that typically form interactions with antibody VL domains are buried within the VH dimerization interface and are also on the periphery of the VH dimerization interface, including positions 35, 37, 45, 49, and 91 (according to the Kabat numbering system).

The published structure of the Gr6 VH homodimer (PDB code: 3QYC) was evaluated for residue positions within the frameworks that are distal to the complementarity determining regions (CDRs) and involved in homodimer interactions. Two residue positions fit this description. The first was Kabat position 37, which is a valine or isoleucine in all human VH germlines. The second was Kabat position 45, which is canonically a leucine in every human VH germline. These two residues were chosen for Rosetta software-based computation-based screening for residues that destabilize the Gr6 VH homodimer while having a minimal impact on the stability of monomeric Gr6.

Kabat residue valine 37 in the 3QYC structure (residue 39 in the Gr6 structure) was computationally mutated to all possible amino acids and the calculated stability of the mutant was compared to the wildtype protein. This calculation was performed for the VH homodimer as well as a VH monomer (Table 8). The structure of the monomer was created by removing one of the chains in the 3QYC crystal structure. During the energy calculations, residues near the site of mutation were allowed to adopt alternative conformations to accommodate the mutation. The substitutions V37Y and V37F were of interest because they were predicted to most destabilize the homodimer (>10 kcal/mol) without destabilizing the monomer. The substitutions V37P and V37R were also predicted to destabilize the dimer but were also predicted to destabilize the monomer. A computational scan of all possible point mutations was also performed for Kabat residue leucine 45 (residue 47 in the Gr6 structure), which is also buried at the homodimer interface. For this position, there was no substitution predicted to significantly destabilize the homodimer while leaving the stability of the monomer unperturbed. However, substitutions to build up a charge-charge repulsions within the interface at position 45 were more destabilizing to the dimer compared to the monomer based on Rosetta energy calculations. Table 8 shows impact the of residue substitutions at VH Kabat positions 37 and 45 as measured using Rosetta

TABLE 8

| | | | Delta Rosetta |
|---|---|---|---|
| | Change in Rosetta | Change in Rosetta | Energy |
| | Energy Units | Energy Units | Unit Change for |
| Substitution | (Dimer) | (Monomer) | Monomer vs Dimer |
| V37G | 8.66 | 5.91 | −2.75 |
| V37P | 14.86 | 13.73 | −1.13 |
| V37F | 9.42 | −1.17 | −10.59 |
| V37Y | 11.22 | −1.46 | −12.68 |
| V37R | 14.55 | 2.33 | −12.22 |
| L45D | 6.08 | 3.45 | −2.63 |
| L45E | 5.62 | 3.32 | −2.30 |
| L45R | 3.82 | 1.85 | −1.97 |

The impact of substitutions at residues 37 and 45 on Gr6 VH homodimerization was assessed. A mammalian expression plasmid encoding for the Gr6 VH domain with a C-terminal 8×-Histidine tag was generated as described elsewhere herein. The variants were generated by DNA synthesis and cloning into the mammalian expression plasmid. The plasmids were then transfected into 25 mL Expi293 cells as previously described, which were then cultured for 5 days prior to harvest. The proteins were purified from Expi293 supernatants using a His60 Nickel resin (Takara; Cat. #635657) and a AKTA Pure instrument (Cytiva). Following elution, the proteins were analyzed by HPLC (Thermo Vanquish FLEX) using a Zenix-C SEC 150 column with a 3 μm particle size and 150 Angstrom pore size resin (Sepax Technologies). A low protein molecular weight (LMW) standard (Cell Mosaic Inc.) was used in parallel. The running buffer was 50 mM sodium phosphate, 150 mM NaCl, pH 6.8 with a flow rate of 1 mL/min at 25° C.

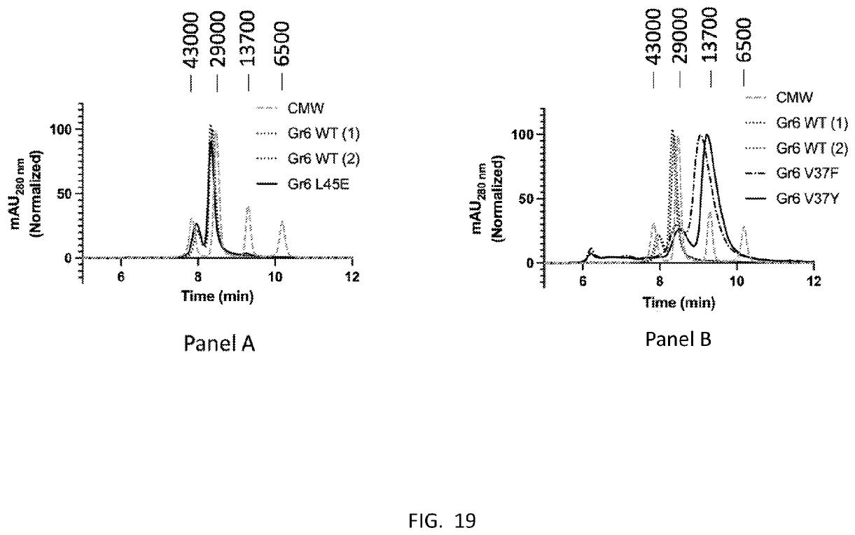

The HPLC analyses demonstrated that the 37Y mutation significantly reduced the level of dimerization with the Gr6 protein. Based on the molecular weight standard, Gr6 ran at a molecular weight slightly larger than 30 kDa, consistent with forming a homodimer (FIG. 19, Panel A). Substitution of L45E had no impact on VH dimerization as the VH protein eluted from the HPLC column at the same time as the unmodified Gr6 protein (FIG. 19, Panel A). Substitution of V37 to F or Y did result the Gr6 protein eluting at a molecular weight consistent with a monomer (FIG. 19, Panel B). The V37Y variant eluted slightly slower than the V37F protein. It is possible that the V37F protein exists in a monomer/dimer equilibrium. The V37Y protein eluted at a molecular weight more precisely in-line with that of a monomer (FIG. 19, Panel B). V37R was also assessed. It negatively impacted both expression and the biophysical properties of Gr6.

37F and 37Y were consistently indicated to be stabilizing by Rosetta across multiple VH germline monomers. 37Y proved one of the most stabilizing single substitutions for both the VH1 and VH2 family germlines where it was tested here, and it is an integral piece of the combination designs for the VH2 family. Notably, when evaluating the monomer/dimer propensity of the TTX017-v13-VH2-5 VH protein, the molecule was intrinsically a dimer. Stabilization combination designs lacking the 37Y substitution maintained this dimeric status while combination designs that included the 37Y substitution become monomeric.

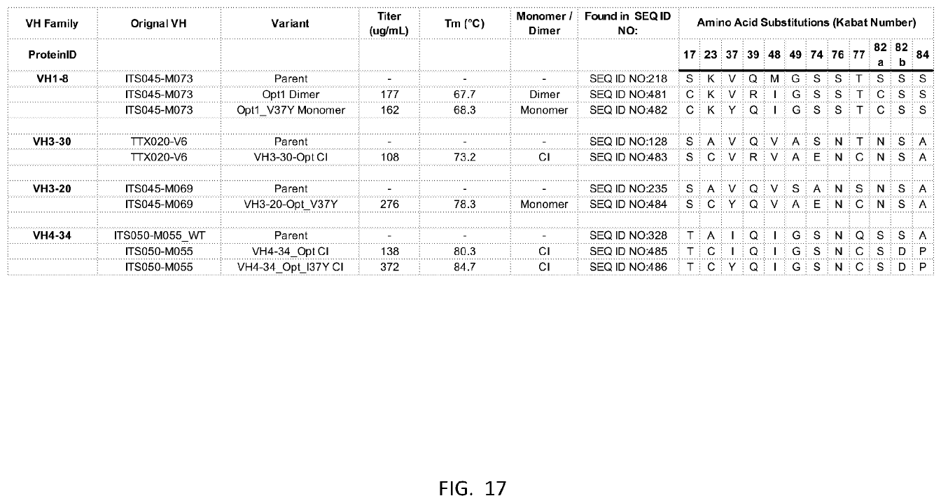

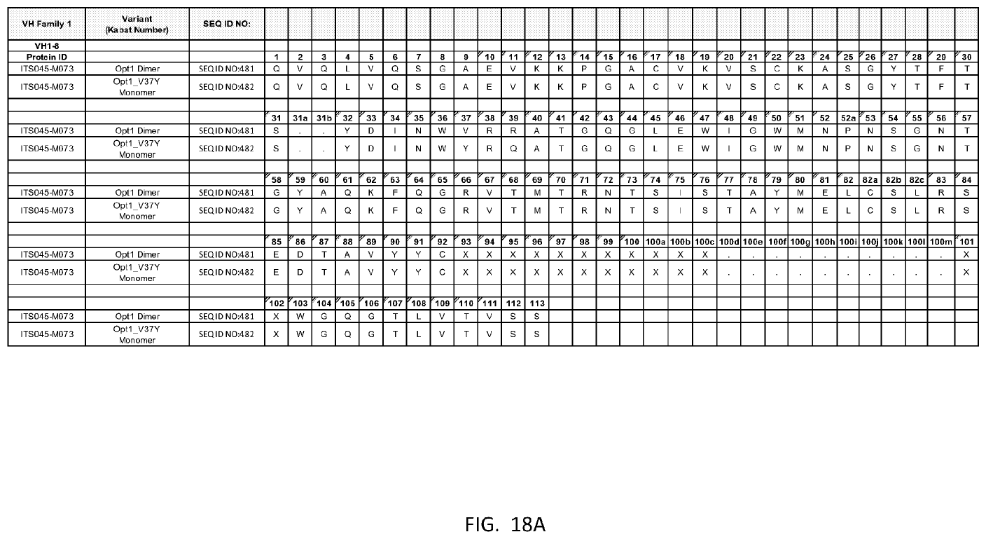

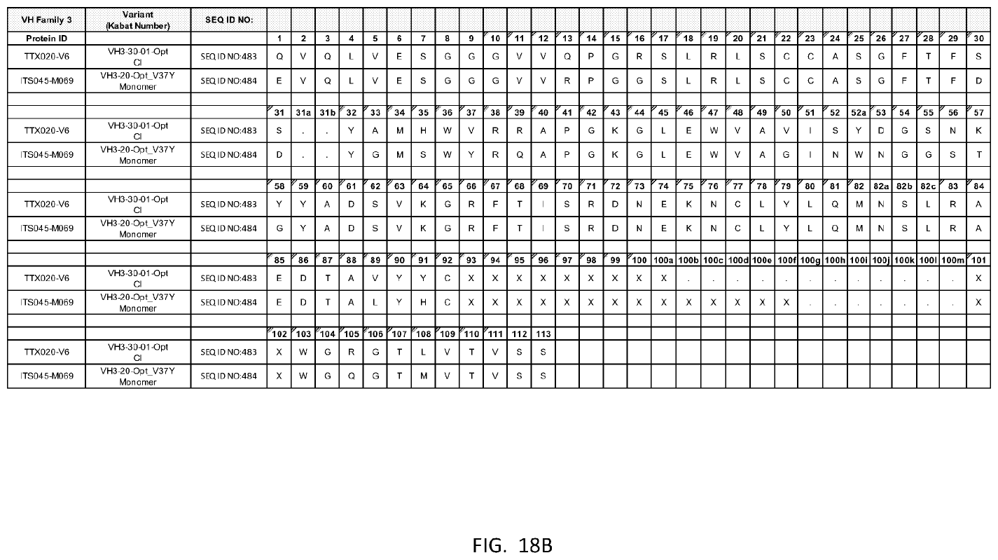

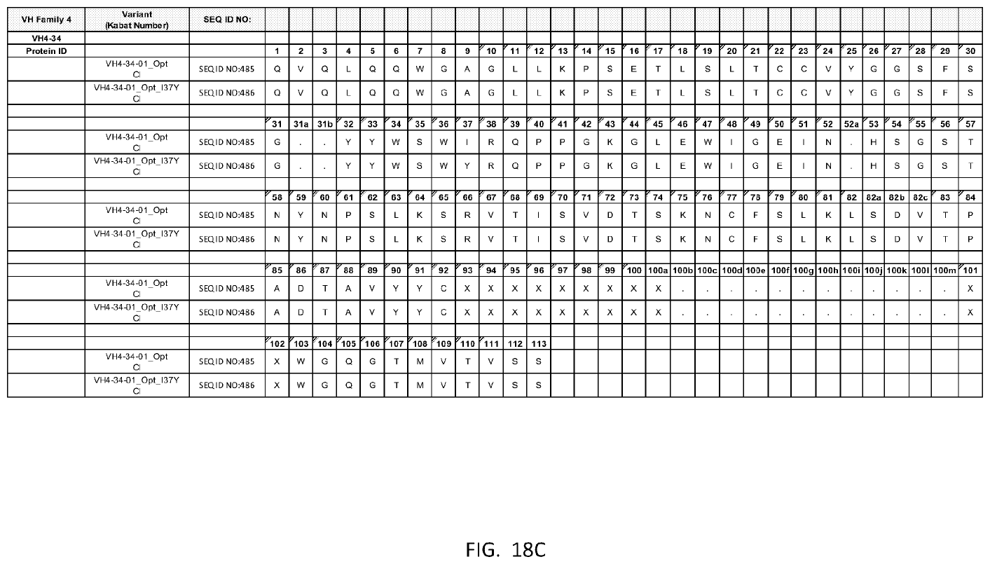

To assess whether the 37Y variant was amenable to being added to additional VH1, VH3, and VH4 family members, we measured the impact it makes on VH domains from each family. We found that for VH1-8, VH3-20, and VH4-34 variants with existing stabilization designs, adding the 37Y did not impact expression and, in some cases, improved expression. For VH1-8 and VH3-20, which could be assessed for their oligomeric state via size exclusion chromatography, the VHs containing 37Y behaved as monomers. FIG. 17 provides a summary of the amino acid substitution strategy for the germline members tested. Complete amino acid sequences are provided in FIG. 18A-18C.

### Example 8: Impact of CDR3 on Stability of Modified VH Domains

In order to confirm that the sequence of CDR3 does not affect the impact of the stabilizing substitutions on the VH domains as described herein, expression and stability of VH molecules having identical sequences, other than CDR3, were determined.

In VH family 1-69.2, VH molecules ITS050-M022 and ITS045-M070 (FIG. 4B) have identical sequences other than CDR3 (not shown) and have affinity for different targets. Each molecule was tested with two different sets of combinations of substitutions as shown below in Table 9.

TABLE 9

| | | SEQ | | | Fold | DSF |
|---|---|---|---|---|---|---|
| Molecule | | ID | | | Expression | Tₘ |
| ID/Target/ | Substitutions | NO: | Expression | Error | vs. WT | (° C.) |
| ITS045-M070/ | WT | 221 | 665 | 7 | 1 | n.d. |
| IL2Rbeta/ | 17C/82aC, | 222 | 1106 | 538 | 1.66 | 88.5 |
| | 39R, 48I | | | | | |
| | 17C/82aC, | 223 | 1238 | 306 | 1.86 | 85 |
| | 16D, 48I | | | | | |
| ITS050-M002S/ | WT | 225 | 1 | 0 | 1 | n.d. |
| 41BB | 17C/82aC, | 224 | 122.5 | 58.5 | 122.5 | 78.3 |
| | 39R, 48I | | | | | |
| | 17C/82aC, | 226 | 93.5 | 66.5 | 93.5 | n.d. |
| | 16D, 48I | | | | | |

In VH 3-20 family, VH molecules ITS051-M019 and ITS045-M069 have identical V-gene sequences other than CDR3 and FR4 (different J-chain) and have affinity for different targets. Each molecule was tested with two different sets of combinations of substitutions as shown in Table 10.

TABLE 10

| | | SEQ | | | Fold | DSF |
|---|---|---|---|---|---|---|
| Molecule | | ID | | | Expression | Tₘ |
| ID/Target | Substitutions | NO: | Expression | Error | vs. WT | (° C.) |
| ITS051-M019/ | WT | 232 | 1 | 0 | 1 | n.d. |
| IL2Rgama | 23C/77C, 39R, | 233 | 595 | 125 | 595 | n.d. |
| | 49A, 74E | | | | | |
| | 23C/77C, 39R, | 234 | 489 | 65 | 489 | n.d. |
| | 49A, 84E | | | | | |
| ITS045-M069/ | WT | 235 | 140.5 | 13.5 | 1 | 58 |
| 41bb | 23C/77C, 39R, | 236 | 467 | 219 | 3.32 | 79.5 |
| | 49A, 74E | | | | | |
| | 23C/77C, 39R, | 237 | 277 | 49 | 1.97 | 80.5 |
| | 49A, 84E | | | | | |

### Example 9

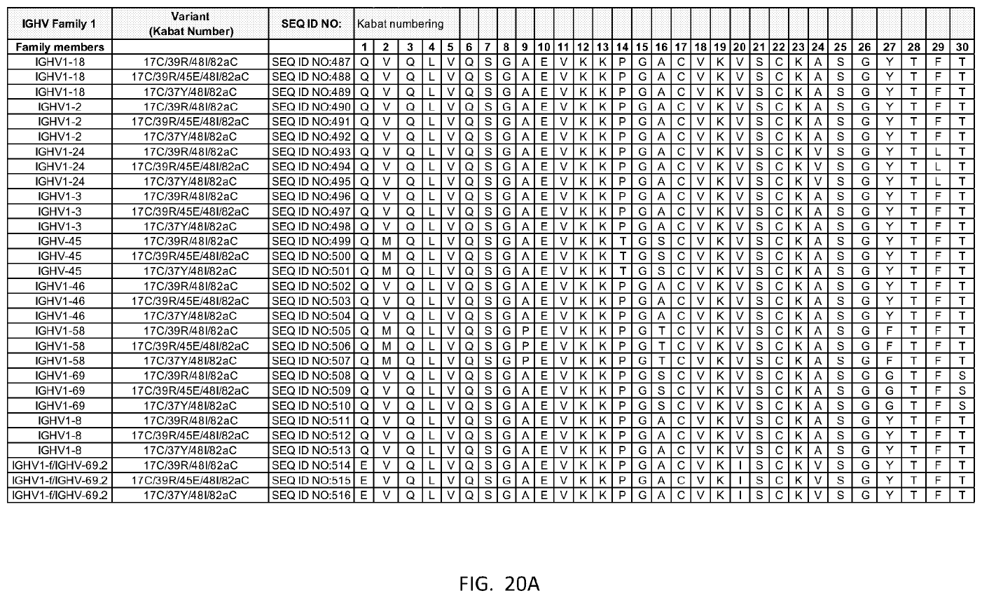

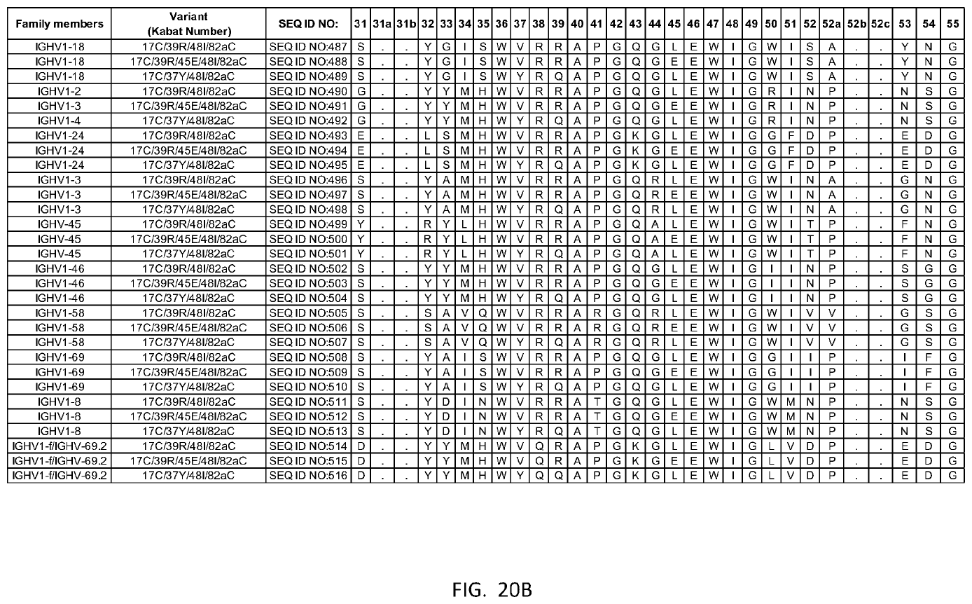

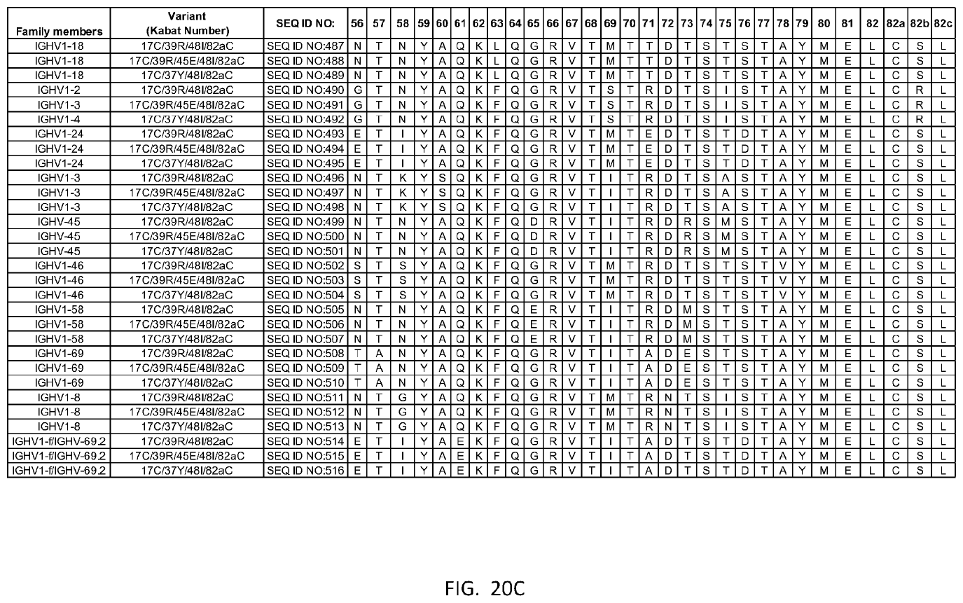

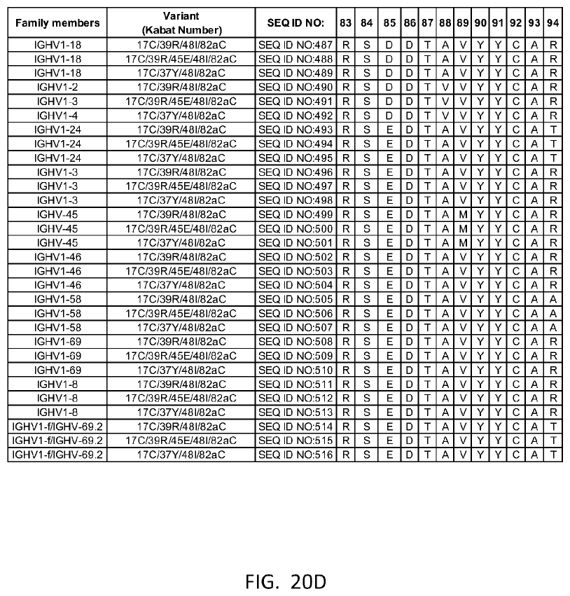

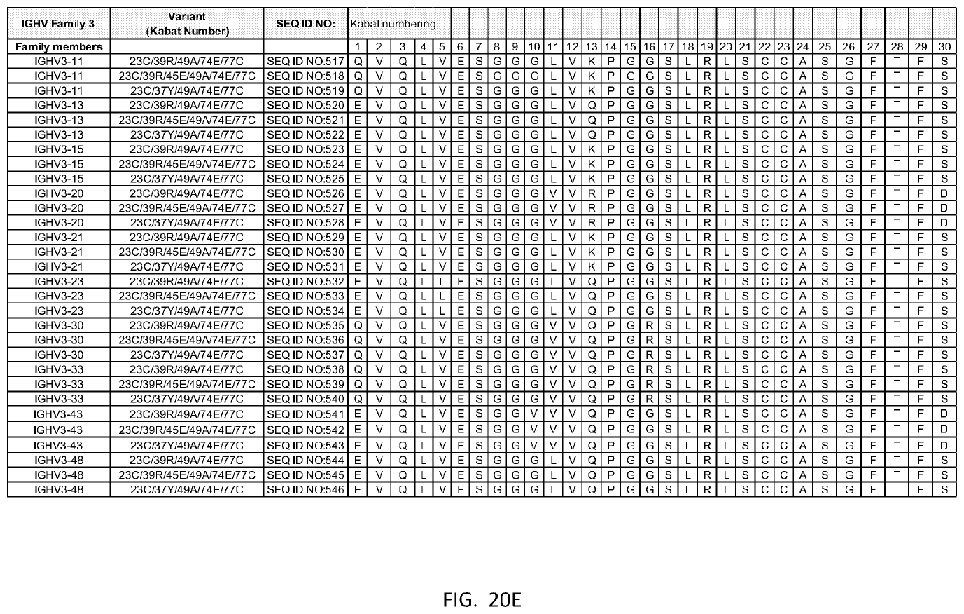

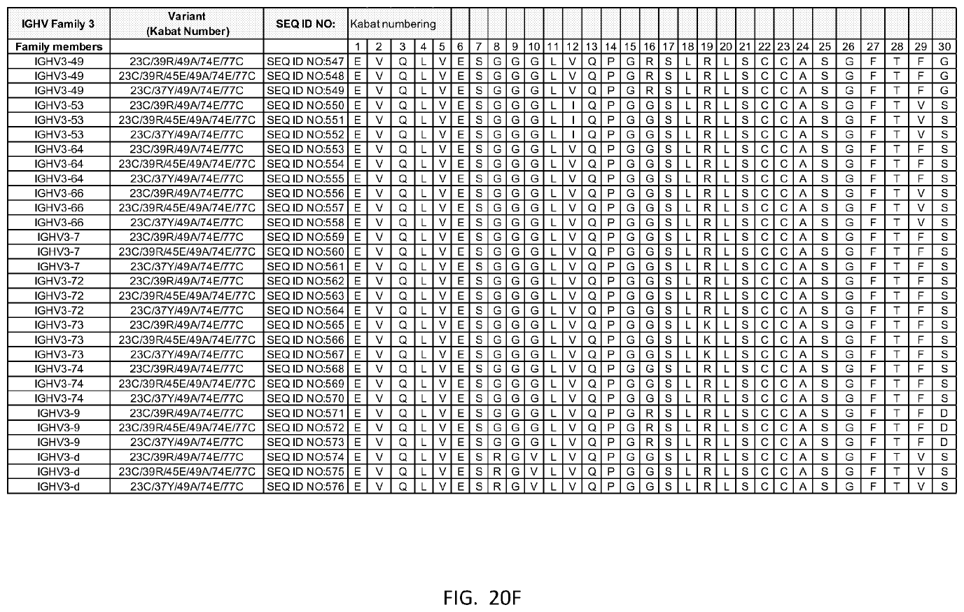

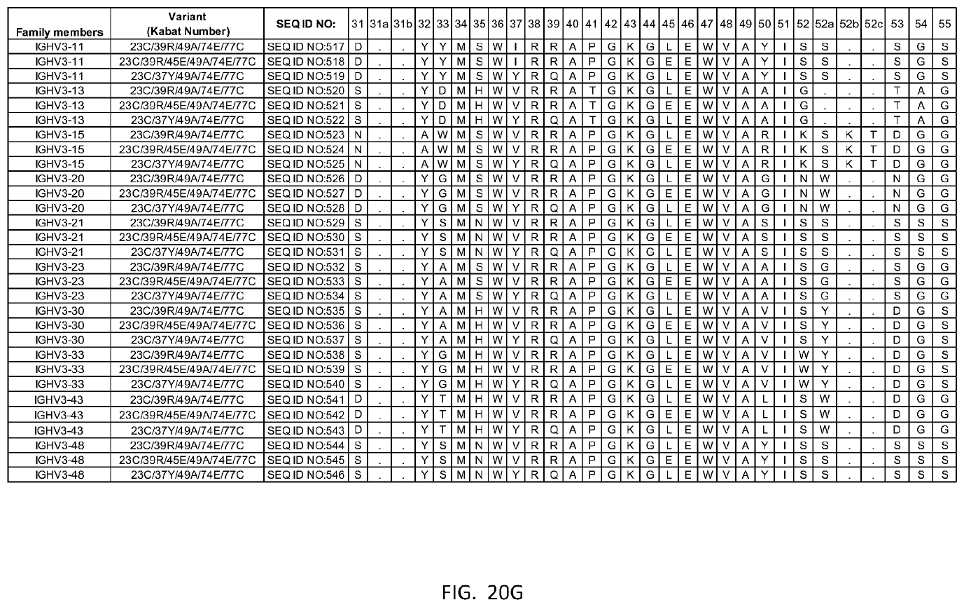

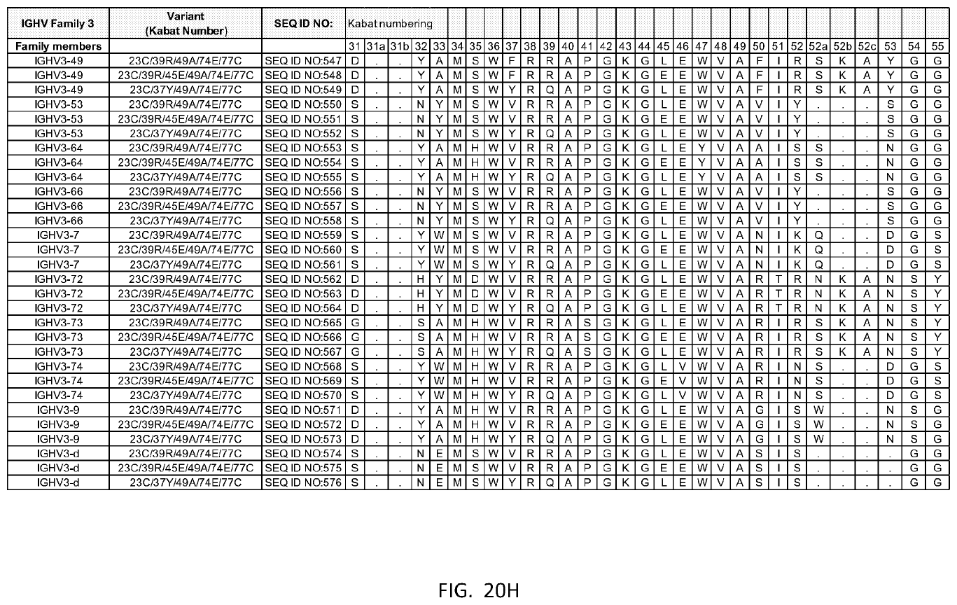

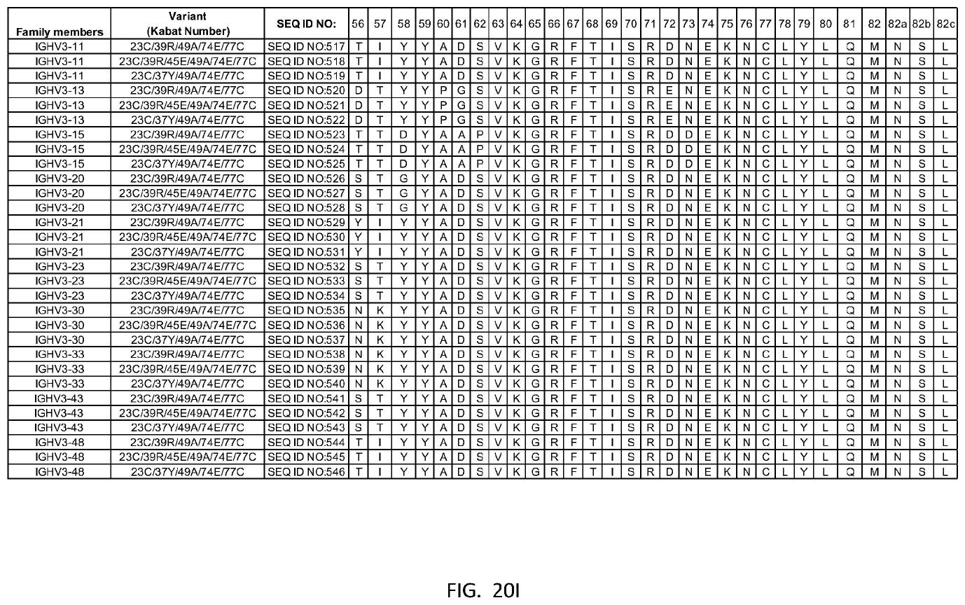

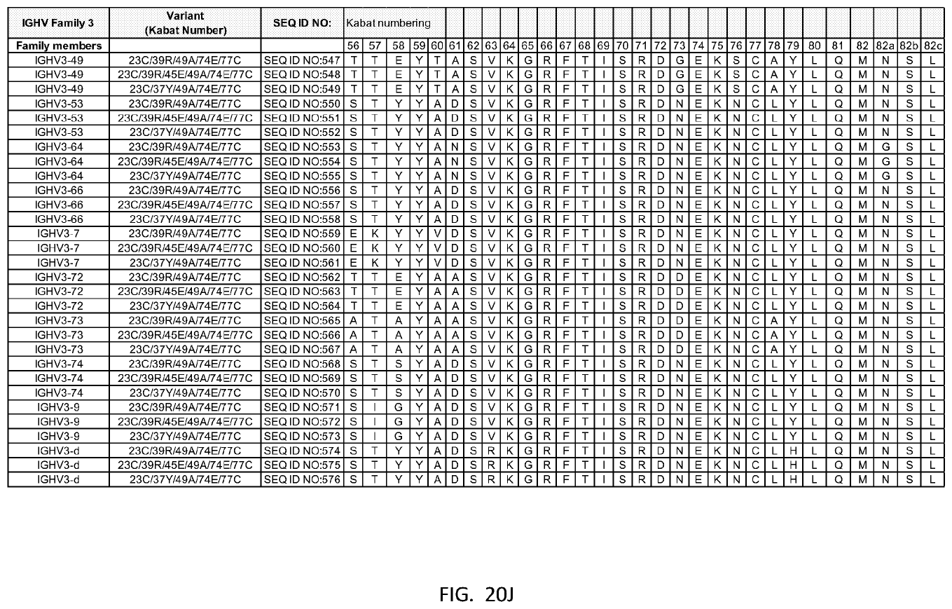

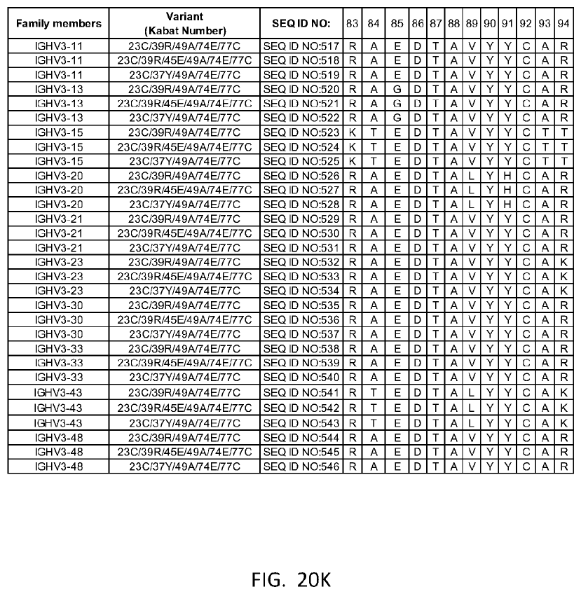

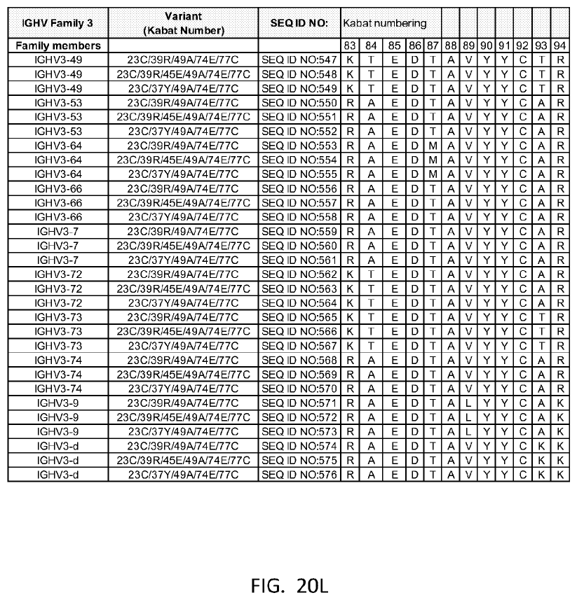

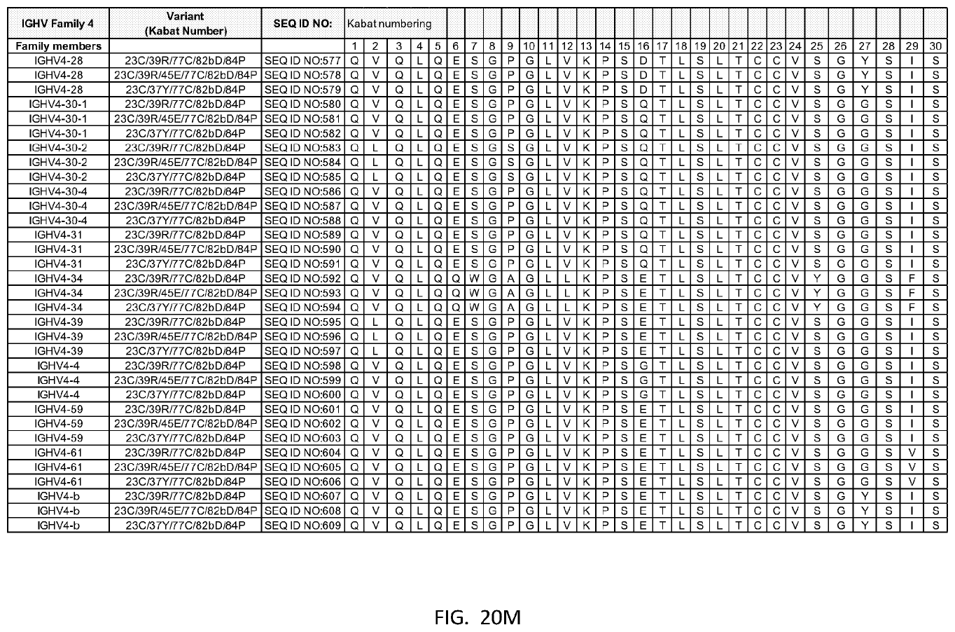

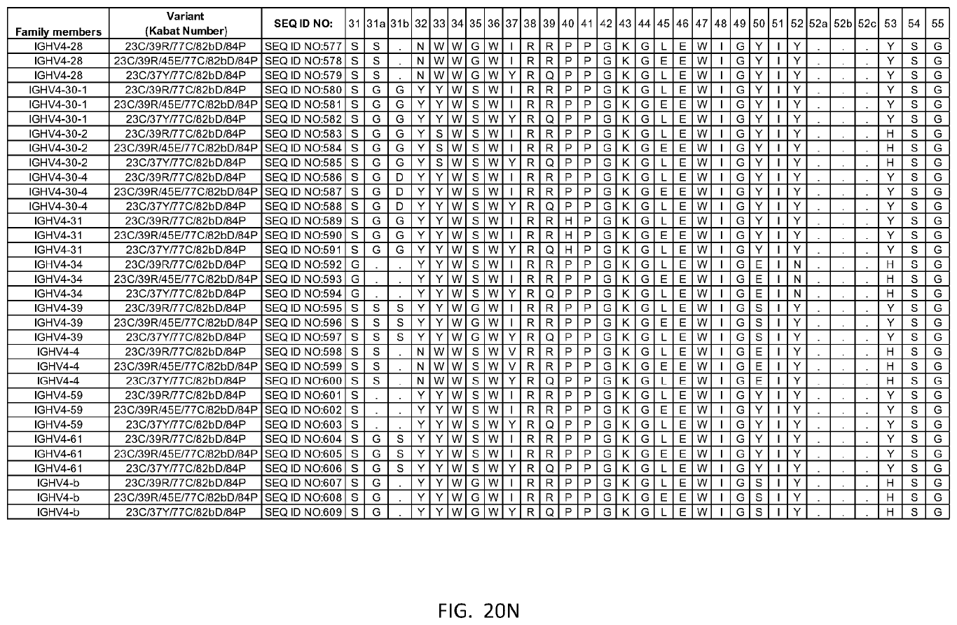

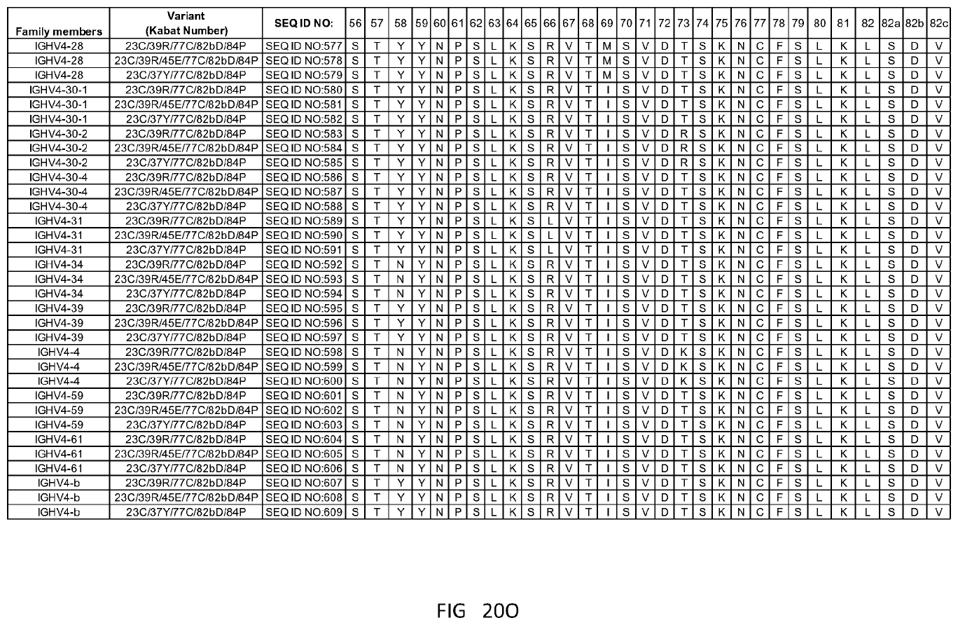

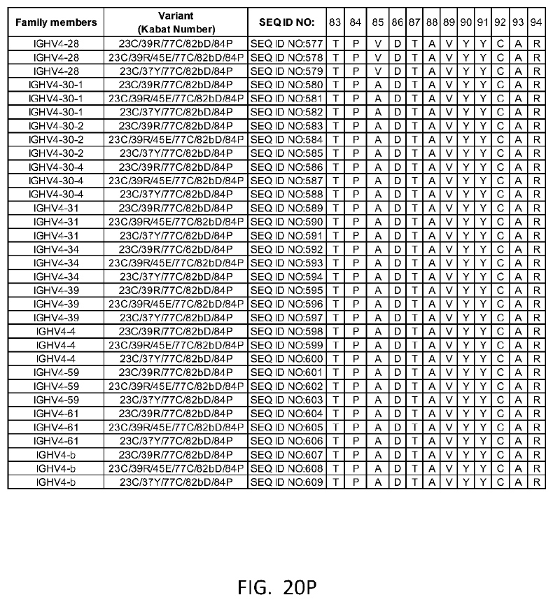

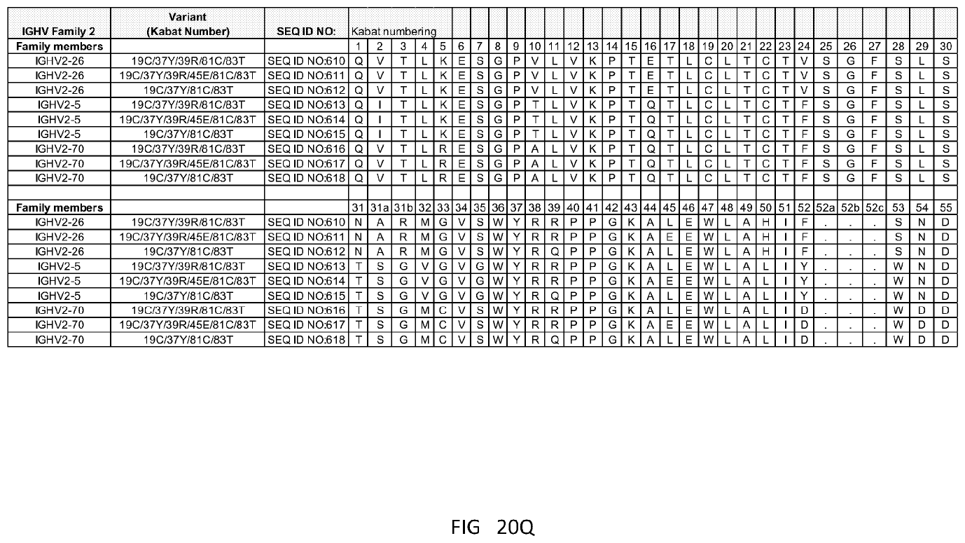

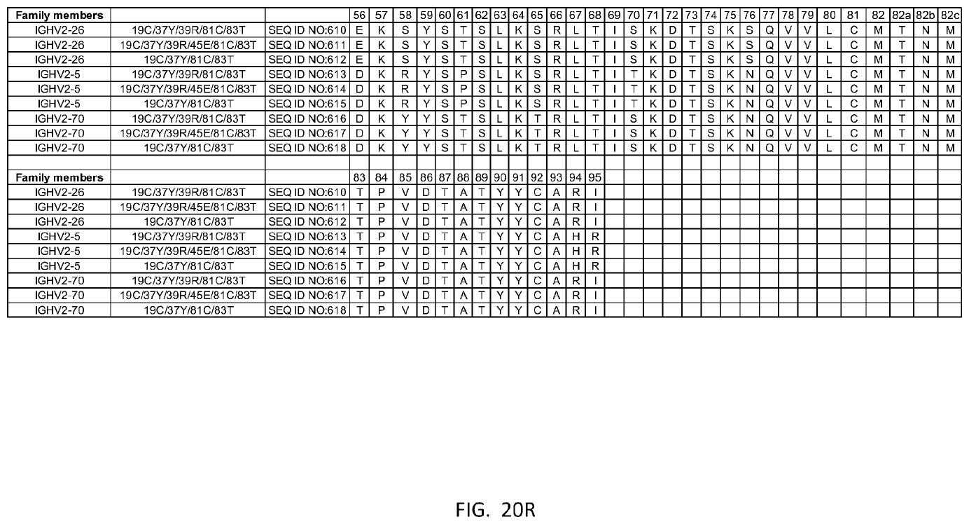

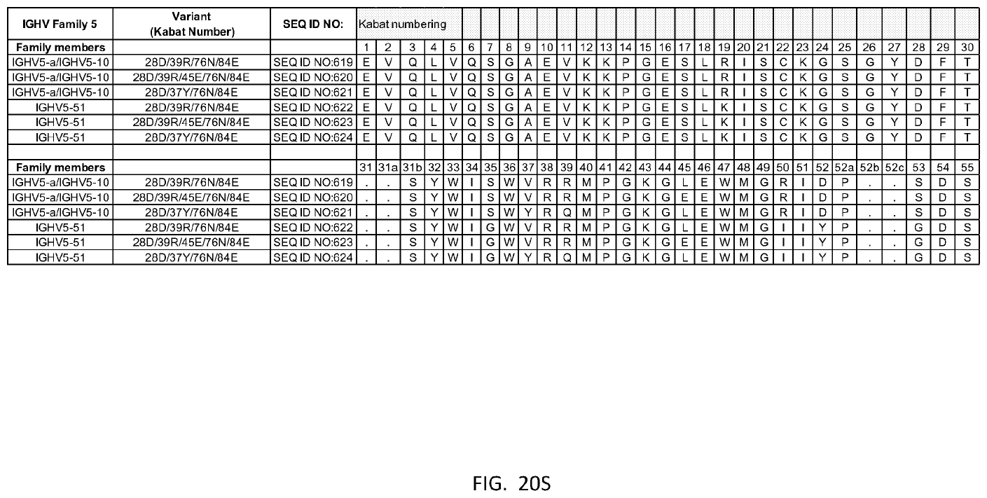

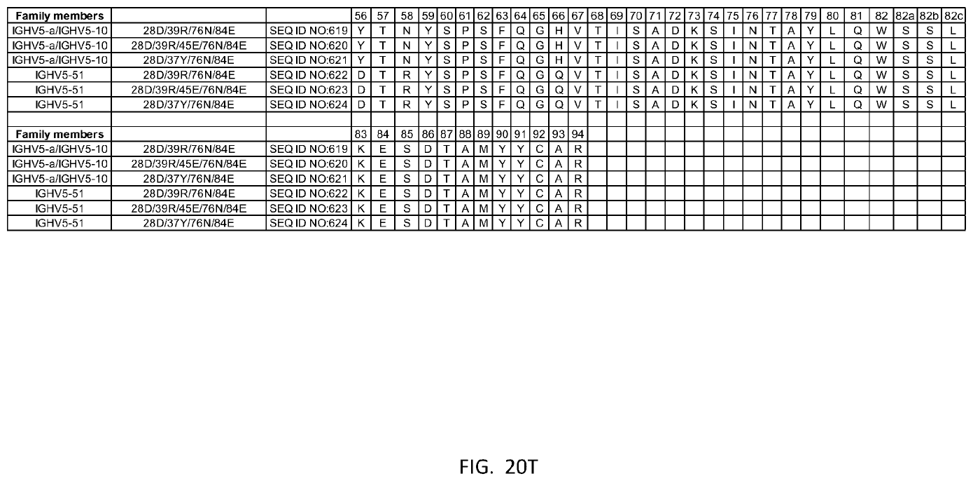

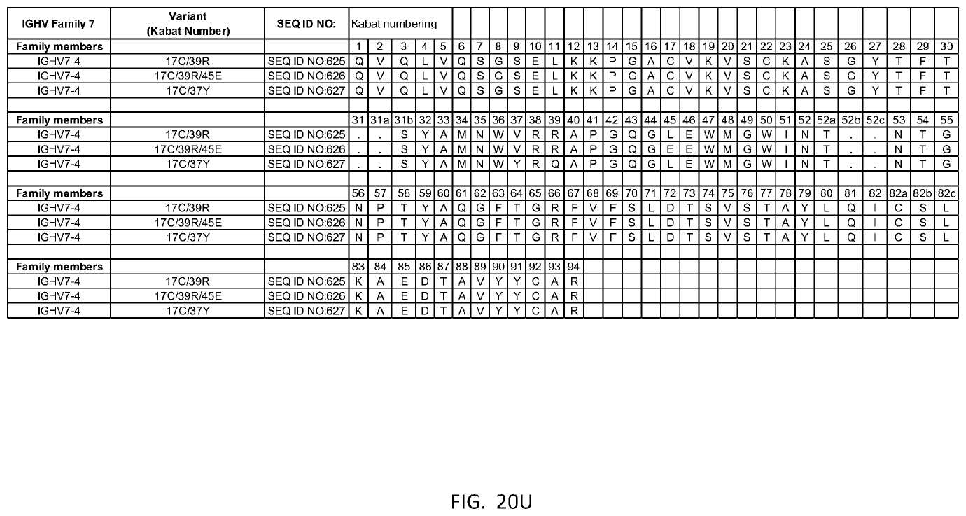

FIGS. 20A-20U show a number of examples of modified human germline IGHV sequences having combinations of substitutions according to the disclosure. These include the following:

Members of Germline Family 1 Modified with
17C/82aC, 39R, 48I
17C/82aC, 39R, 45E, 48I
17C/82aC, 39Y, 48I
   Members of Germline Family 3 Modified with
23C/77C, 39R, 49A, 74E
23C/77C, 39R, 45E, 49A, 74E
23C/77C, 37Y, 49A, 74E
   Members of Germline Family 4 Modified with
23C/77C, 39R, 82bD, 84P
23C/77C, 39R, 45E, 82bD, 84P
23C/77C, 37Y, 82bD, 84P
   Members of Germline Family 2 Modified with
19C/81C, 37Y, 39R, 83T
19C/81C, 37Y, 39R, 45E, 83T
19C/81C, 37Y, 83T
   Members of Germline Family 5 Modified with
28D, 39R, 76N, 84E
28D, 39R, 45E, 76N, 84E
28D, 37Y, 76N, 84E, and
   Members of Germline Family 7 Modified with
39R, 17C (to pair with natural C at 82a)
39R, 45E, 17C (to pair with natural C at 82a)
37Y, 17C (to pair with natural C at 82a).

### Example 10

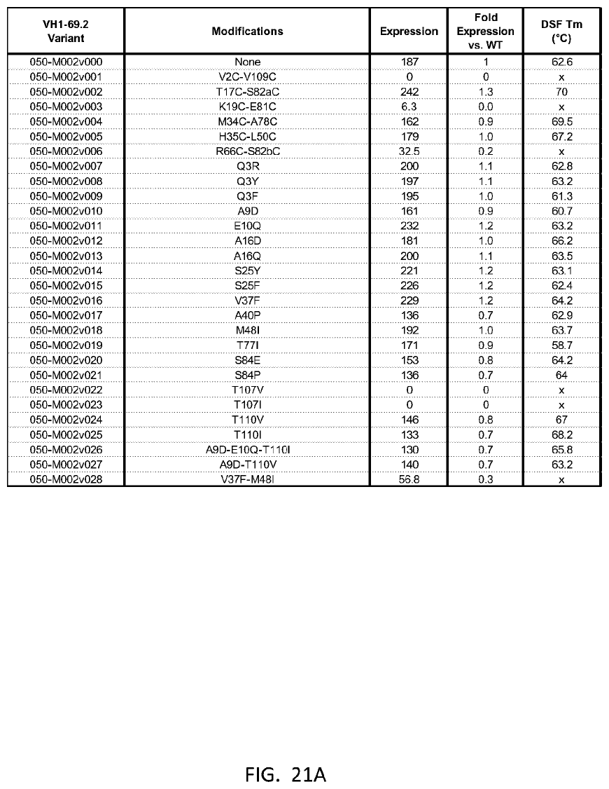

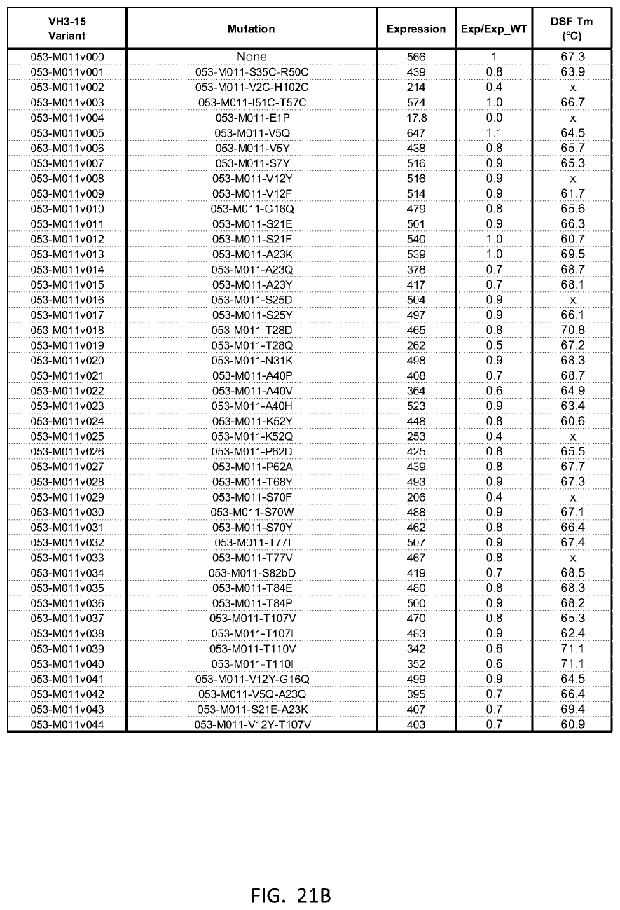

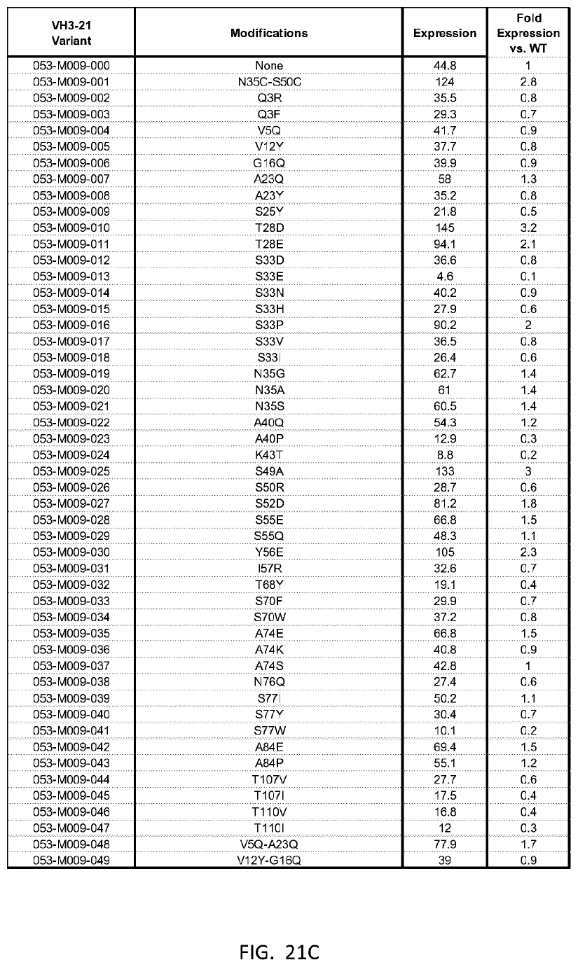

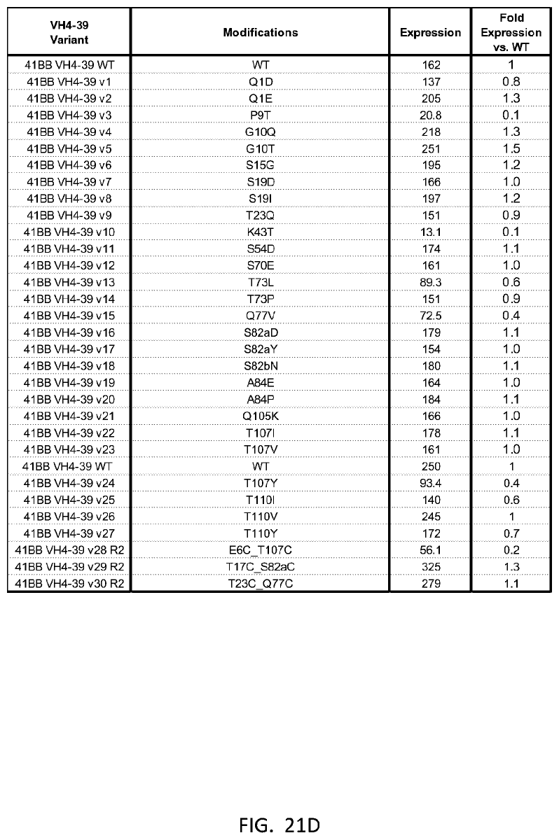

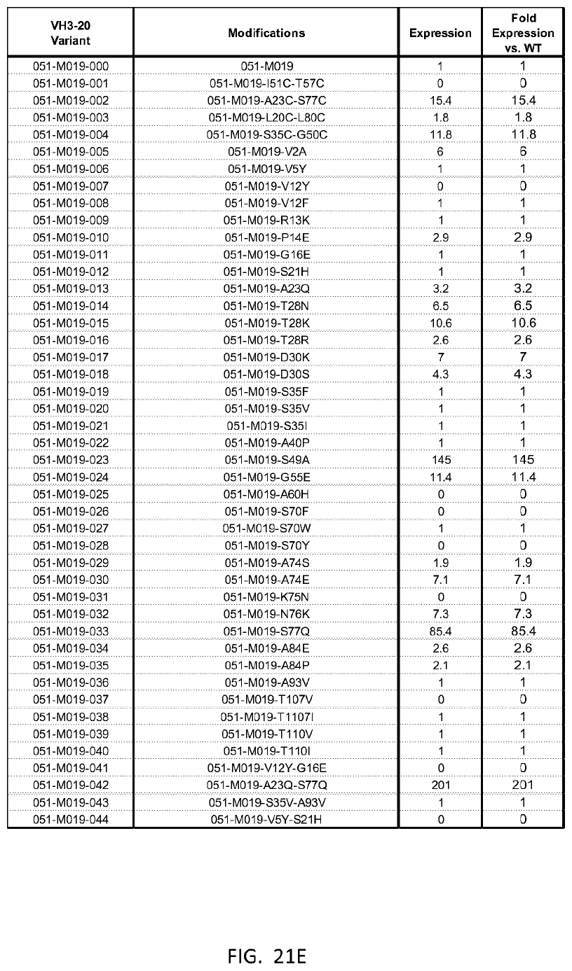

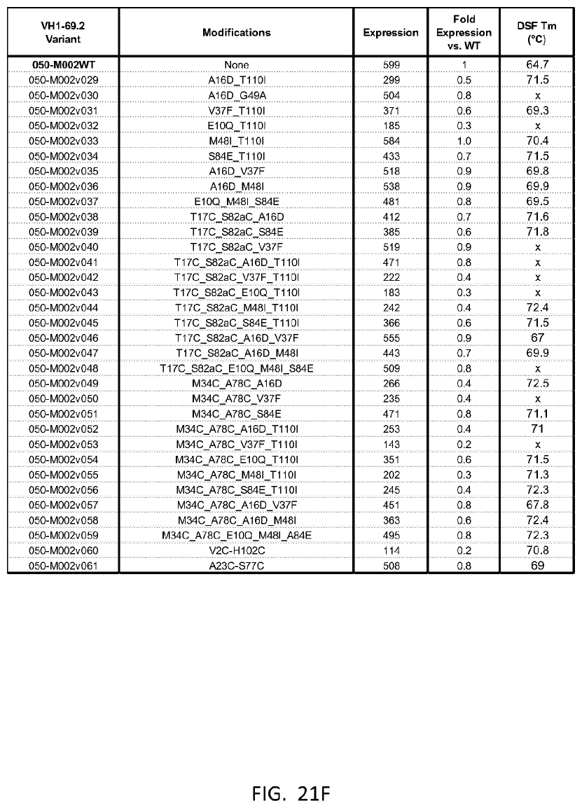

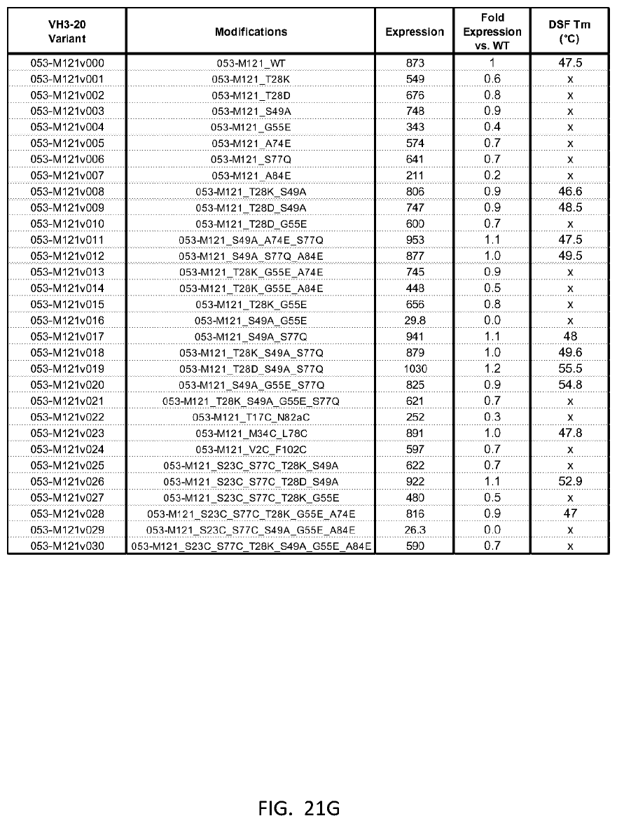

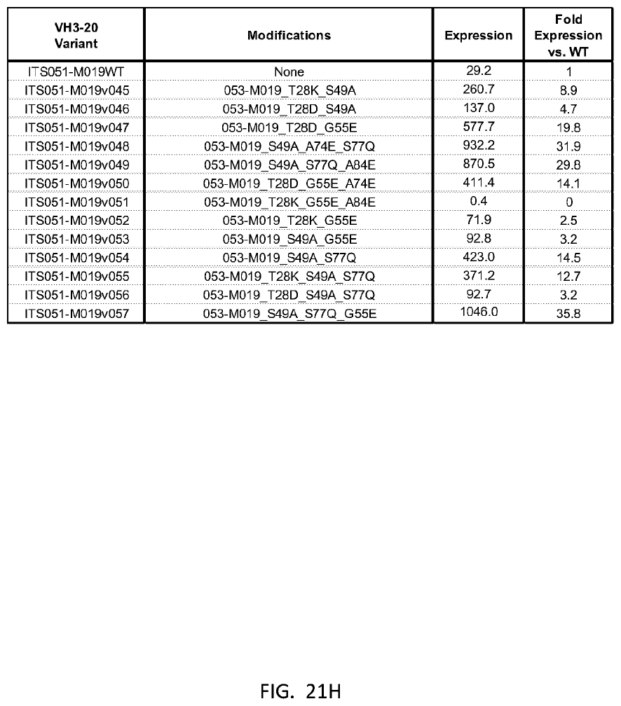

FIG. 21A-21H show summaries of additional example of modified VH domains of the disclosure in germline family members 1-69.2, 3-15, 3-21, 4-39 and 3-20. These summaries are shown along with expression and/or Tm data for each of the example molecules.

Having described the invention in detail and by reference to specific embodiments thereof, it will be apparent that substitutions and variations are possible without departing from the scope of the invention defined in the appended claims. More specifically, although some aspects of the present invention are identified herein as particularly advantageous, it is contemplated that the present invention is not necessarily limited to these particular aspects of the invention.

What is claimed is:

1. A single immunoglobulin variable domain, comprising an amino acid sequence of a human heavy chain V-gene portion (IGHV) of an antibody, wherein the IGHV amino acid sequence comprises one or more amino acid substitutions that results in one or more of increased cellular expression, increased thermal stability, decreased dimerization, and decreased light chain pairing, as compared to a wild-type IGHV sequence lacking the one or more amino acid substitutions.

2. The single immunoglobulin variable domain of claim 1, further comprising a D gene sequence.

3. The single immunoglobulin variable domain of claim 1, further comprising a J gene sequence.

4. The single immunoglobulin variable domain of any of claims 1-3, wherein the one or more substitutions comprise at least one of the following amino acids, according to the Kabat numbering system: 1E, 2A, 5Q, 10Q, 10T, 14E, 15G, 16D, 16Q, 19I, 23K, 23Q, 23Y, 25F, 25Y, 28D, 28E, 28K, 28N, 28R, 30K, 30S, 31K, 33P, 35A, 35G, 35S, 37F, 37Y, 37H, 39R, 40P, 44D, 45E, 48I, 49A, 52E, 52D, 55E, 56E, 60A, 60D, 65D, 68E, 73D, 73P, 74E, 76K, 76N, 77Q, 82bD, 82bN, 83D, 83K, 83L, 83Q, 83T, 84E, 84P, 84Y, 85K, 85R, 85S, 85T, 89I, 105D, 107I, 107Y, 110I, and 110V.

5. The single immunoglobulin variable domain of any of claims 1-4, comprising a non-natural disulfide bond comprising at least one cysteine residue at a non-naturally occurring amino acid position.

6. The single immunoglobulin variable domain of any of claims 1-5, wherein the non-natural disulfide bond is present between two cysteine residues at positions 2 and 102; 17 and 82a; 19 and 81; 23 and 77; 34 and 78; 35 and 50, according to the Kabat numbering system.

7. The single immunoglobulin variable domain of any one of claims 1-6, comprising one of the following combinations of amino acids, according to the Kabat numbering system:

| | | |
|---|---|---|
| 5Q/23Q | 28D/48I/84E | 10Q/48I/84E |
| 10T/82bD | 28D/49A | 37Y/83T |
| 10T/82bD | 28D/49A/77Q | 39R/28D |
| 10T/82bN | 28D/55E | 39R/45E |
| 10T/84P | 28D/55E/74E | 39R/48I |
| 15G/37Y | 28D/76N/83D | 39R/60A |
| 15G/44D | 28D/76N/84E | 39R/60D |
| 15G/85S | 28K/49A | 39R/68E |
| 15G/83T | 28K/49A/77Q | 39R/76N |
| 16D/37F | 28K/49A/55E/84E | 39R/83D |
| 16D/37Y | 28K/49A/55E/84E/10T/ | 39R/84E |
| 16D/39R/48I | 82bN | 39R/83T |
| 16D/48I | 28K/55E | 39R/45E/48I |
| 16D/110I | 28K/55E/74E | 39R/45E/49A/74E |
| 23Q/77Q | 37F/48I | 39R/45E/82bD/84P |
| 28D/37Y/48I/83D | 37Y (or 39R)/10T/84P | 44D/85S |
| 28D/37Y/48I/84E | 37Y (or 39R)/10T/82bD | 44D/83T |
| 28D/37Y/76N/83D | 37Y (or 39R)/82bD/84P | 45E/82bD/84P |
| 28D/37Y/76N/84E | 37Y/39R/83T | 49A/55E |
| 28D/39R/45E/76N/84E | 37Y/39R/45E/83T | 49A/55E/77Q |
| 28D/39R/48I/83D | 37Y/44D | 49A/55E/84E |
| 28D/39R/48I/84E | 37Y/48I | 49A/74E |
| 28D/39R/76N/83D | 37Y/49A/74E | 49A/74E/77Q |
| 28D/39R/76N/84E | 37Y/85S | 49A/77Q |
| 28D/48I/83D | 49A/84E | 49A/77Q/55E |
| 49A/77Q/84E | 82bD/84P | 82bN/84P |
| 45E/82bD/84P | | 83T/44D |

8. The single immunoglobulin variable domain of any one of claims 1-7, further comprising at least one of 39R, 45E, and 37Y if not already present.

9. The single immunoglobulin variable domain of any one of claims 1-8, having an origin of a human germline gene selected from germline family 1, germline family 2, germline family 3, germline family 4, germline family 5, or germline family 7.

10. The single immunoglobulin variable domain of claim 9, wherein the germline gene family 1 comprises germline gene family members 1-2 (SEQ ID NO: 1), 1-3 (SEQ ID NO: 2), 1-8 (SEQ ID NO: 3), 1-18 (SEQ ID NO: 4), 1-24 (SEQ ID NO: 5), 1-45 (SEQ ID NO: 6), 1-46 (SEQ ID NO: 7), 1-58 (SEQ ID NO: 8), 1-69 (SEQ ID NO: 9), and 1-69.2 (SEQ ID NO: 10), and alleles thereof.

11. The single immunoglobulin variable domain of claim 10, comprising one or more of the following substitutions: 10Q, 16D, 16Q, 25Y, 25F, 37F, 37Y, 39R, 45E, 48I, 84E, 84P, 110V, and 110I.

12. The single immunoglobulin variable domain of claim of claim 11, comprising one or more of the following combinations of substitutions:

| | | |
|---|---|---|
| 10Q/48I/84E | 16D/48I | 39R/45E/48I |
| 16D/37F | 16D/110I | 39R/48I |
| 16D/37Y | 37F/48I | |
| 16D/39R/48I | 37Y/48I | |

13. The single immunoglobulin variable domain of claim of claim 11, comprising one of the following combinations of substitutions:

| | | |
|---|---|---|
| 17C/82aC/10Q/48I/84E | 17C/82aC/16D/37F | 17C/82aC/16D/37Y/39R |
| 17C/82aC/16D | 17C/82aC/16D/37Y | 17C/82aC/16D/39R |
| 17C/82aC/16D/39R/48I | 17C/82aC/39R | 34C/78C/37F |
| 17C/82aC/16D/48I | 17C/82aC/39R/45E/48I | 34C/78C/84E |
| 17C/82aC/37F | 17C/82aC/39R/48I | 34C/78C/16D/37F |
| 17C/82aC/37Y | 17C/82aC/84E | 34C/78C/16D/48I |
| 17C/82aC/37Y/48I | 34C/78C/16D | 34C/78C/10Q/48I/84E |

14. The single immunoglobulin variable domain of claim of claim 12 or claim 13, wherein the combinations comprise at least one of 37Y, 39R and 45E if not already included.

15. The single immunoglobulin variable domain of claim 9, wherein the germline gene family 2 comprises germline gene family members 2-5 (SEQ ID NO: 11), 2-26 (SEQ ID NO: 12, and 2-70 (SEQ ID NO: 13), and alleles thereof.

16. The single immunoglobulin variable domain of claim 15, comprising one or more of the following substitutions: 15G, 16D, 37Y, 37H, 39R, 44D, 45E, 65D, 73D, 73P, 83L, 83Q, 83K, 83T, 84Y, 85R, 85S, 85K, 85T, 89I, 105D, and 107I.

17. The single immunoglobulin variable domain of claim of claim 16, comprising one of the following combinations of substitutions:

| | | |
|---|---|---|
| 15G/37Y | 37Y/39R/45E/83T | 37Y/83T |
| 15G/44D | 37Y/39R/83T | 39R/83T |
| 15G/85S | 37Y/44D | 44D/85S |
| 15G/83T | 37Y/85S | 44D/83 |

18. The single immunoglobulin variable domain of claim of claim 16, comprising one of the following combinations of substitutions:

| | | |
|---|---|---|
| 19C/81C/15G | 19C/81C/15G/83T | 19C/81C/37Y/44D |
| 19C/81C/15G/37Y | 19C/81C/37Y | 19C/81C/37Y/83T |
| 19C/81C/15G/44D | 19C/81C/37Y/39R/83T | 19C/81C/37Y/85S |
| 19C/81C/15G/85S | 19C/81C/37Y/39R/45E/83T | 19C/81C/39R/83T |
| 19C/81C/44D | 19C/81C/85S | 19C/81C/83T/44D |
| 19C/81C/44D/85S | 19C/81C/83T | |

19. The single immunoglobulin variable domain of claim of claim 17 or claim 18, wherein the combinations comprise at least one of 37Y, 39R and 45E if not already included.

20. The single immunoglobulin variable domain of claim 9, wherein the germline gene family 3 comprises germline gene family members 3-7 (SEQ ID NO: 14), 3-9 (SEQ ID NO: 15), 3-11 (SEQ ID NO: 16), 3-13 (SEQ ID NO: 17), 3-15 (SEQ ID NO: 18), 3-20 (SEQ ID NO: 19), 3-21 (SEQ ID NO: 20), 3-23 (SEQ ID NO: 21), 3-30 (SEQ ID NO: 22), 3-33 (SEQ ID NO: 23), 3-43 (SEQ ID NO: 24), 3-48 (SEQ ID NO: 25), 3-49 (SEQ ID NO: 26), 3-53 (SEQ ID NO: 27), 3-64 (SEQ ID NO: 28), 3-66 (SEQ ID NO: 29), 3-72 (SEQ ID NO: 30), 3-73 (SEQ ID NO: 31), 3-74 (SEQ ID NO: 32), 3-d (SEQ ID NO: 33), and 3-NL1 (SEQ ID NO: 34), and alleles thereof.

21. The single immunoglobulin variable domain of claim of claim 20, comprising one or more of the following substitutions: 2A, 5Q, 14E, 23K, 23Q, 23Y, 28D, 28E, 28N, 28K, 28R, 30K, 30S, 31K, 33P, 35G, 35A, 35S, 37Y, 39R, 40P, 45E, 49A, 52E, 52D, 55E, 56E, 74E, 76K, 77Q, 82bD, 84E, 84P, 110V, and 110I.

22. The single immunoglobulin variable domain of claim of claim 20, comprising one of the following combinations of substitutions:

| | | |
|---|---|---|
| 5Q/23Q | 28K/49A/77Q | 49A/55E/77Q |
| 23Q/77Q | 28K/55E | 49A/55E/84E |
| 28D/49A | 28K/55E/74E | 49A/74E/77Q |
| 28D/49A/77Q | 37Y/49A/74E | 49A/77Q |
| 28D/55E | 39R/45E/49A/74E | 49A/77Q/55E |
| 28D/55E/74E | 39R/49A/84E | 49A/77Q/84E |
| 28K/49A | 39R/84E | 49A/84E |
| 28K/49A/55E/84E | 49A/55E | |

23. The single immunoglobulin variable domain of claim of claim 20, comprising one of the following combinations of substitutions:

| | | |
|---|---|---|
| 23C/77C/28K/49A | 23C/77C/39R/45E/49A/74E | 34C/78C/28K |
| 23C/77C/28D/49A | 23C/77C/39R/49A/74E | 34C/78C/49A |
| 23C/77C/28K/55E | 23C/77C/39R/49A/84E | 34C/78C/55E |
| 23C/77C/28K/55E/74E | 23C/77C/39R/49A/84E | 34C/78C/74E |
| 23C/77C/28K/49A/55E/84E | 23C/77C/49A/55E/84E | 34C/78C/77Q |
| 23C/77C/37Y/49A/74E | 34C/78C/28D | 34C/78C/84E |

24. The single immunoglobulin variable domain of claim 22 or claim 23, wherein the combinations comprise at least one of 37Y, 39R and 45G if not already included.

25. The single immunoglobulin variable domain of claim 9, wherein the germline gene family 4 comprises germline gene family members 4-4 (SEQ ID NO: 35), 4-28 (SEQ ID NO: 36, 4-30-1 (SEQ ID NO: 37), 4-30-2 (SEQ ID NO: 38), 4-30-4 (SEQ ID NO: 39), 4-31 (SEQ ID NO: 40), 4-34 (SEQ ID NO: 41), 4-38-2 (SEQ ID NO: 42), 4-39 (SEQ ID NO: 43), 4-59 (SEQ ID NO: 44) and 4-61 (SEQ ID NO: 45), 4-b (SEQ ID NO: 46), and alleles thereof.

26. The single immunoglobulin variable domain of claim of claim 25, comprising one or more of the following substitutions: 1E, 10Q, 10T, 15G, 19I, 37Y, 39R, 45E, 82bD, 82bN, 84P, 107I, and 107Y.

27. The single immunoglobulin variable domain of claim of claim 25, comprising one of the following combinations of substitutions:

| | | |
|---|---|---|
| 10T/82bN | 37Y (and/or 39R)/10T/84P | 39R/45E/82bD/84P |
| 10T/84P | 37Y (and/or | 45E/82bD/84P |
| 10T/82bD | 39R)/10T/82bD | |
| 37Y (and/or 39R)/82bN/84P | 37Y (and/or 39R)/10T/82bN | |

28. The single immunoglobulin variable domain of claim of claim 25, comprising one of the following combinations of substitutions:

| | |
|---|---|
| 17C/82aC/10T | 23C/77C/45E/82bD/84P |
| 17C/82aC/10T/82bN | 23C/77C/82bD/84P |
| 17C/82aC/10T/82bD | 23C/77C/82bN/84P |
| 17C/82aC/82bN/84P | 23C/77C/37Y (and/or 39R)/10T/82bD |
| 17C/82aC/37Y (and/or 39R)/10T/82bD | 23C/77C/37Y (and/or 39R)/10T/82bN |
| 17C/82aC/37Y (and/or 39R)/10T/84P | 23C/77C/37Y (and/or 39R)/10T/84P |
| 17C/82aC/37Y (and/or 39R)/82bD/84P | 23C/77C/37Y (and/or 39R)/82bD/84P |
| 23C/77C/10T/84P | 23C/77C/37Y (and/or 39R)/82bD/84P |
| 23C/77C/39R/45E/82bD/84P | |

29. The single immunoglobulin variable domain of any of claim 27 or 28, wherein the combinations comprise at least one of 37Y, 39R, and 45E if not already included.

30. The single immunoglobulin variable domain of claim 9, wherein the germline gene family 5 comprises germline gene family members 5-51 (SEQ ID NO: 47) and 5-a (SEQ ID NO: 48, and alleles thereof.

31. The single immunoglobulin variable domain of claim of 30, comprising one or more of the following substitutions: 28D, 37Y, 39R, 45E, 48I, 60D, 60A, 68E, 76N, 83D, and 84E.

32. The single immunoglobulin variable domain of claim of claim 30, comprising one of the following combinations of substitutions:

| | | |
|---|---|---|
| 39R/28D | 39R/68E | 28D/48I/84E |
| 39R/48I | 39R/76N | 28D/76N/83D |
| 39R/60A | 39R/83D | 28D/76N/84E |
| 39R/60D | 39R/84E | 28D/48I/83D |
| 28D/39R/48I/84E | 28D/39R/48I/83D | 28D/37Y/76N/84E |
| 28D/39R/76N/83D | 28D/37Y/48I/84E | 28D/37Y/48I/83D |
| 28D/39R/76N/84E | 28D/37Y/76N/83D | 28D/39R/45E/76N/84E |

33. The single immunoglobulin variable domain of claim of 32, wherein the combinations comprise at least one of 37Y, 39R, and 45E if not already included,

34. The single immunoglobulin variable domain of claim 9, wherein the germline gene comprises germline gene family member 6-1 (SEQ ID NO: 49) and alleles thereof.

35. The single immunoglobulin variable domain of claim 9, wherein the germline gene comprises germline gene family member 7-4-1 (SEQ ID NO: 50) and alleles thereof.

36. The single immunoglobulin variable domain of claim of claim 35, comprising one of the following combinations of substitutions:
17C/82aC/39R
17C/82aC/39R/45E
17C/82aC/37Y
35C/50C/39R
35C/50C/39R/45E
35C/50C/37Y

37. The single immunoglobulin variable domain of claim of 36, wherein the combinations comprise at least one of 37Y, 39R, and 45E if not already included.

38. A polynucleotide encoding the single immunoglobulin variable domain of any one of claims 1-37.

39. A pharmaceutical acceptable composition, comprising the single immunoglobulin variable domain of any one of claims 1-37.

40. A polypeptide comprising at least one framework sequence selected from FR1, FR2, FR3, and FR4 of a single immunoglobulin variable domain of any of claim 4 through claim 36, wherein the framework sequence comprises at least one of the substitutions or combinations thereof.

41. A VH domain library comprising a plurality of the single immunoglobulin variable domains of any of claims 1 to 37.

42. A polynucleotide library comprising a plurality of polynucleotides encoding for a plurality of the single immunoglobulin variable domain of any of claims 1 to 37.

43. A method for identifying an antigen binding molecule; comprising,
(i) contacting the single immunoglobulin variable domain library of claim 42 with a target, and
(ii) identifying single immunoglobulin variable domains of the library binding to the target.

44. A single immunoglobulin variable domain, comprising an amino acid sequence of a framework region of a human heavy chain V-gene portion (IGHV) of an antibody, wherein the IGHV amino acid sequence comprises one or more amino acid substitutions or combinations thereof:
1E, 2A, 5Q, 10Q, 10T, 14E, 15G, 16D, 16Q, 19I, 23K, 23Q, 23Y, 25F, 25Y, 28D,
28E, 28K, 28N, 28R, 30K, 30S, 31K, 33P, 35A, 35G, 35S, 37F, 37Y, 37H, 39R,
40P, 44D, 45E, 48I, 49A, 52E, 52D, 55E, 56E, 60A, 60D, 65D, 68E, 73D, 73P,
74E, 76K, 76N, 77Q, 82bD, 82bN, 83D, 83K, 83L, 83Q, 83T, 84E, 84P, 84Y,
85K, 85R, 85S, 85T, 89I, 105D, 107I, 107Y, 110I, 110V.

45. The single immunoglobulin variable domain of claim 44, comprising one of the following combinations of amino acids, according to the Kabat numbering system:

| | | |
|---|---|---|
| 5Q/23Q | 10T/82bD | 10T/82bN |
| 10Q/48I/84E | 10T/82bD | 10T/84P |
| 15G/37Y | 28D/55E | 39R/48I |
| 15G/44D | 28D/55E/74E | 39R/60A |
| 15G/85S | 28D/76N/83D | 39R/60D |
| 15G/83T | 28D/76N/84E | 39R/68E |
| 16D/37F | 28K/49A | 39R/76N |
| 16D/37Y | 28K/49A/77Q | 39R/83D |
| 16D/39R/48I | 28K/49A/55E/84E | 39R/84E |
| 16D/48I | 28K/49A/55E/84E/10T/ | 39R/83T |
| 16D/110I | 82bN | 39R/45E/48I |
| 23Q/77Q | 28K/55E | 39R/45E/49A/74E |
| 28D/37Y/48I/83D | 28K/55E/74E | 39R/45E/82bD/84P |
| 28D/37Y/48I/84E | 37F/48I | 44D/85S |
| 28D/37Y/76N/83D | 37Y (or 39R)/10T/84P | 44D/83T |
| 28D/37Y/76N/84E | 37Y (or 39R)/10T/82bD | 45E/82bD/84P |
| 28D/39R/45E/76N/84E | 37Y (or 39R)/82bD/84P | 49A/55E |
| 28D/39R/48I/83D | 37Y/39R/83T | 49A/55E/77Q |
| 28D/39R/48I/84E | 37Y/39R/45E/83T | 49A/55E/84E |
| 28D/39R/76N/83D | 37Y/44D | 49A/74E |
| 28D/39R/76N/84E | 37Y/48I | 49A/74E/77Q |
| 28D/48I/83D | 37Y/49A/74E | 49A/77Q |
| 28D/48I/84E | 37Y/85S | 49A/77Q/55E |
| 28D/49A | 37Y/83T | 49A/77Q/84E |
| 28D/49A/77Q | 39R/28D | 45E/82bD/84P |
| 82bD/84P | 39R/45E | 49A/84E |
| | 82bN/84P | 83T/44D |

46. The single immunoglobulin variable domain of any one of claims 44-45, further comprising a non-natural disulfide bond comprising at least one cysteine residue at a non-naturally occurring amino acid position.

47. The single immunoglobulin variable domain of claim 46, wherein the non-natural disulfide bond is present between two cysteine residues at positions 2 and 102; 17 and 82a; 19 and 81; 23 and 77; 34 and 78; 35 and 50, according to the Kabat numbering system.

48. The single immunoglobulin variable domain of any of claims of 44-47, wherein the combinations comprise at least one of 37Y, 39R, and 45E if not already included.


SEQUENCE LISTING (633 sequences)

SEQ ID NO: 1 - protein, 98 aa, Homo sapiens
QVQLVQSGAEVKKPGASVKVSCKASGYTFTGYYMHWVRQAPGQGLEWMGRINPNSGGTNYAQKFQGRVTSTRDTSISTAYMELSRLRSDDTVVYYCAR

SEQ ID NO: 2 - protein, 98 aa, Homo sapiens
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAR

SEQ ID NO: 3 - protein, 98 aa, Homo sapiens
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCAR

SEQ ID NO: 4 - protein, 98 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAR

SEQ ID NO: 5 - protein, 98 aa, Homo sapiens
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCAT

SEQ ID NO: 6 - protein, 98 aa, Homo sapiens
QMQLVQSGAEVKKTGSSVKVSCKASGYTFTYRYLHWVRQAPGQALEWMGWITPFNGNTNYAQKFQDRVTITRDRSMSTAYMELSSLRSEDTAMYYCAR

SEQ ID NO: 7 - protein, 98 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAR

SEQ ID NO: 8 - protein, 98 aa, Homo sapiens
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAA

SEQ ID NO: 9 - protein, 98 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAR

SEQ ID NO: 10 - protein, 98 aa, Homo sapiens
EVQLVQSGAEVKKPGATVKISCKVSGYTFTDYYMHWVQQAPGKGLEWMGLVDPEDGETIYAEKFQGRVTITADTSTDTAYMELSSLRSEDTAVYYCAT

SEQ ID NO: 11 - protein, 100 aa, Homo sapiens
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAHR

SEQ ID NO: 12 - protein, 100 aa, Homo sapiens
QVTLKESGPVLVKPTETLTLTCTVSGFSLSNARMGVSWIRQPPGKALEWLAHIFSNDEKSYSTSLKSRLTISKDTSKSQVVLTMTNMDPVDTATYYCARI

SEQ ID NO: 13 - protein, 100 aa, Homo sapiens
QVTLRESGPALVKPTQTLTLTCTFSGFSLSTSGMCVSWIRQPPGKALEWLALIDWDDDKYYSTSLKTRLTISKDTSKNQVVLTMTNMDPVDTATYYCARI

SEQ ID NO: 14 - protein, 98 aa, Homo sapiens
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAR

SEQ ID NO: 15 - protein, 99 aa, Homo sapiens
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKD

SEQ ID NO: 16 - protein, 98 aa, Homo sapiens
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAR

SEQ ID NO: 17 - protein, 97 aa, Homo sapiens
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYCAR

SEQ ID NO: 18 - protein, 100 aa, Homo sapiens
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTT

SEQ ID NO: 19 - protein, 98 aa, Homo sapiens
EVQLVESGGGVVRPGGSLRLSCAASGFTFDDYGMSWVRQAPGKGLEWVSGINWNGGSTGYADSVKGRFTISRDNAKNSLYLQMNSLRAEDTALYHCAR

SEQ ID NO: 20 - protein, 98 aa, Homo sapiens
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAR

SEQ ID NO: 21 - protein, 98 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 22 - protein, 98 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCAR

SEQ ID NO: 23 - protein, 98 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAR

SEQ ID NO: 24 - protein, 99 aa, Homo sapiens
EVQLVESGGVVVQPGGSLRLSCAASGFTFDDYTMHWVRQAPGKGLEWVSLISWDGGSTYYADSVKGRFTISRDNSKNSLYLQMNSLRTEDTALYYCAKD

SEQ ID NO: 25 - protein, 98 aa, Homo sapiens
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCAR

SEQ ID NO: 26 - protein, 100 aa, Homo sapiens
EVQLVESGGGLVQPGRSLRLSCTASGFTFGDYAMSWFRQAPGKGLEWVGFIRSKAYGGTTEYTASVKGRFTISRDGSKSIAYLQMNSLKTEDTAVYYCTR

SEQ ID NO: 27 - protein, 97 aa, Homo sapiens
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 28 - protein, 98 aa, Homo sapiens
EVQLVESGGGLVQPGGSLRLSCAASGFTFSSYAMHWVRQAPGKGLEYVSAISSNGGSTYYANSVKGRFTISRDNSKNTLYLQMGSLRAEDMAVYYCAR

SEQ ID NO: 29 - protein, 97 aa, Homo sapiens
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 30 - protein, 100 aa, Homo sapiens
EVQLVESGGGLVQPGGSLRLSCAASGFTFSDHYMDWVRQAPGKGLEWVGRTRNKANSYTTEYAASVKGRFTISRDDSKNSLYLQMNSLKTEDTAVYYCAR

SEQ ID NO: 31 - protein, 100 aa, Homo sapiens
EVQLVESGGGLVQPGGSLKLSCAASGFTFSGSAMHWVRQASGKGLEWVGRIRSKANSYATAYAASVKGRFTISRDDSKNTAYLQMNSLKTEDTAVYYCTR

SEQ ID NO: 32 - protein, 98 aa, Homo sapiens
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCAR

SEQ ID NO: 33 - protein, 96 aa, Homo sapiens
EVQLVESRGVLVQPGGSLRLSCAASGFTVSSNEMSWVRQAPGKGLEWVSSISGGSTYYADSRKGRFTISRDNSKNTLHLQMNSLRAEDTAVYYCKK

SEQ ID NO: 34 - protein, 98 aa, Homo sapiens
QVQLVESGGGVVQPGGSLRLSCAASGFTFSSYGMHWVRQAPGKGLEWVSVIYSGGSSTYYADSVKGRFTISRDNSKNTLYLQMNSLRAEDTAVYYCAK

SEQ ID NO: 35 - protein, 98 aa, Homo sapiens
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCAR

SEQ ID NO: 36 - protein, 98 aa, Homo sapiens
QVQLQESGPGLVKPSDTLSLTCAVSGYSISSSNWWGWIRQPPGKGLEWIGYIYYSGSTYYNPSLKSRVTMSVDTSKNQFSLKLSSVTAVDTAVYYCAR

SEQ ID NO: 37 - protein, 99 aa, Homo sapiens
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCAR

SEQ ID NO: 38 - protein, 99 aa, Homo sapiens
QLQLQESGSGLVKPSQTLSLTCAVSGGSISSGGYSWSWIRQPPGKGLEWIGYIYHSGSTYYNPSLKSRVTISVDRSKNQFSLKLSSVTAADTAVYYCAR

SEQ ID NO: 39 - protein, 99 aa, Homo sapiens
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAR

SEQ ID NO: 40 - protein, 99 aa, Homo sapiens
QVQLQESGPGLVKPSQTLSLTCTVSGGSISSGGYYWSWIRQHPGKGLEWIGYIYYSGSTYYNPSLKSLVTISVDTSKNQFSLKLSSVTAADTAVYYCAR

SEQ ID NO: 41 - protein, 97 aa, Homo sapiens
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCAR

SEQ ID NO: 42 - protein, 99 aa, Homo sapiens
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARE

SEQ ID NO: 43 - protein, 99 aa, Homo sapiens
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAR

SEQ ID NO: 44 - protein, 97 aa, Homo sapiens
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAR

SEQ ID NO: 45 - protein, 99 aa, Homo sapiens
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAR

SEQ ID NO: 46 - protein, 98 aa, Homo sapiens
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCAR

SEQ ID NO: 47 - protein, 98 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCAR

SEQ ID NO: 48 - protein, 98 aa, Homo sapiens
EVQLVQSGAEVKKPGESLRISCKGSGYSFTSYWISWVRQMPGKGLEWMGRIDPSDSYTNYSPSFQGHVTISADKSISTAYLQWSSLKASDTAMYYCAR

SEQ ID NO: 49 - protein, 101 aa, Homo sapiens
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAR

SEQ ID NO: 50 - protein, 98 aa, Homo sapiens
QVQLVQSGSELKKPGASVKVSCKASGYTFTSYAMNWVRQAPGQGLEWMGWINTNTGNPTYAQGFTGRFVFSFDTSVSTAYLQICSLKAEDTAVYYCAR

SEQ ID NO: 51 - protein, 17 aa, synthetic construct
  REGION 1..17: note = Modified VH domain from a human immunoglobulin germline
AEYFQHWGQGTLVTVSS

SEQ ID NO: 52 - protein, 17 aa, synthetic construct
  REGION 1..17: note = Modified VH domain from a human immunoglobulin germline
YWYFDLWGRGTLVTVSS

SEQ ID NO: 53 - protein, 16 aa, synthetic construct
  REGION 1..16: note = Modified VH domain from a human immunoglobulin germline
DAFDVWGQGTMVTVSS

SEQ ID NO: 54 - protein, 15 aa, synthetic construct
  REGION 1..15: note = Modified VH domain from a human immunoglobulin germline
YFDYWGQGTLVTVSS

SEQ ID NO: 55 - protein, 16 aa, synthetic construct
  REGION 1..16: note = Modified VH domain from a human immunoglobulin germline
NWFDSWGQGTLVTVSS

SEQ ID NO: 56 - protein, 20 aa, synthetic construct
  REGION 1..20: note = Modified VH domain from a human immunoglobulin germline
YYYYYGMDVWGQGTTVTVSS

SEQ ID NO: 57-74 - skipped (18 entries)

SEQ ID NO: 75 - protein, 116 aa, synthetic construct
  REGION 1..116: note = Modified VH domain from a human immunoglobulin germline
  VARIANT 97..105: note = X is any residue
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCXXXXXXXXXWGRGTLVTVSS

SEQ ID NO: 76 - protein, 116 aa, synthetic construct
  REGION 1..116: note = Modified VH domain from a human immunoglobulin germline
  VARIANT 97..105: note = X is any residue
QVQLVQSGAEVKKPGACVKVSCKASGYTFTSYDINWVRQATGQGLEWMGWMNPNSGNTGYAQKFQGRVTMTRNTSISTAYMELCSLRSEDTAVYYCXXXXXXXXXWGRGTLVTVSS

SEQ ID NO: 77 - protein, 116 aa, synthetic construct
  REGION 1..116: note = Modified VH domain from a human immunoglobulin germline
  VARIANT 97..105: note = X is any residue
QVQLVQSGAEVKKPGASVCVSCKASGYTFTSYDINWVRQATGQGLEWMGWMNPNSGNTGYAQKFQGRVTMTRNTSISTAYMCLSSLRSEDTAVYYCXXXXXXXXXWGRGTLVTVSS

SEQ ID NO: 78 - protein, 116 aa, synthetic construct
  REGION 1..116: note = Modified VH domain from a human immunoglobulin germline
  VARIANT 97..105: note = X is any residue
QVQLVQSGAEVKKPGASVKVSCCASGYTFTSYDINWVRQATGQGLEWMGWMNPNSGNTGYAQKFQGRVTMTRNTSISCAYMELSSLRSEDTAVYYCXXXXXXXXXWGRGTLVTVSS

SEQ ID NO: 79 - protein, 116 aa, synthetic construct
  REGION 1..116: note = Modified VH domain from a human immunoglobulin germline
  VARIANT 97..105: note = X is any residue
QVQLVQSGAEVKKPGASVKVSCKASGYTFTSYDICWVRQATGQGLEWMGCMNPNSGNTGYAQKFQGRVTMTRNTSISTAYMELSSLRSEDTAVYYCXXXXXXXXXWGRGTLVTVSS

SEQ ID NO: 80 - protein, 116 aa, synthetic construct
  REGION 1..116: note = Modified VH domain from a human immunoglobulin germline
  VARIANT 97..104: note = X is any residue
QCQLVQSGAEVKKPGASVKVSCKASGYTFTSYDINWVRQATGQGLEWMGWMNPNSGNTGYAQKFQGRVTMTRNTSISTAYMELSSLRSEDTAVYYCXXXXXXXXCWGRGTLVTVSS

SEQ ID NO: 81 - protein, 118 aa, synthetic construct
  REGION 1..118: note = Modified VH domain from a human immunoglobulin germline
  VARIANT 97..107: note = X is any residue
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCXXXXXXXXXXXWGQGTLVTVSS

SEQ ID NO: 82 - protein, 118 aa, synthetic construct
  VARIANT 97..107: note = X is any residue
  REGION 1..118: note = Modified VH domain from a human immunoglobulin germline
QVQLVQSGAEVKKPGACVKVSCKASGYTFTSYGISWVRQAPGQGLEWMGWISAYNGNTNYAQKLQGRVTMTTDTSTSTAYMELCSLRSDDTAVYYCXXXXXXXXXXXWGQGTLVTVSS

SEQ ID NO: 83 - protein, 118 aa, synthetic construct
  VARIANT 97..107: note = X is any residue
  REGION 1..118: note = Modified VH domain from a human immunoglobulin germline
QVQLVQSGAEVKKPGASVCVSCKASGYTFTSYGISWVRQAPGQGLEWMGWISAYNGNTNYAQKLQGRVTMTTDTSTSTAYMCLRSLRSDDTAVYYCXXXXXXXXXXXWGQGTLVTVSS

SEQ ID NO: 84 - protein, 118 aa, synthetic construct
  VARIANT 97..107: note = X is any residue
  REGION 1..118: note = Modified VH domain from a human immunoglobulin germline
QVQLVQSGAEVKKPGASVKVSCCASGYTFTSYGISWVRQAPGQGLEWMGWISAYNGNTNYAQKLQGRVTMTTDTSTSCAYMELRSLRSDDTAVYYCXXXXXXXXXXXWGQGTLVTVSS

SEQ ID NO: 85 - protein, 118 aa, synthetic construct
  VARIANT 97..107: note = X is any residue
  REGION 1..118: note = Modified VH domain from a human immunoglobulin germline
QVQLVQSGAEVKKPGASVKVSCKASGYTFTSYGICWVRQAPGQGLEWMGCISAYNGNTNYAQKLQGRVTMTTDTSTSTAYMELRSLRSDDTAVYYCXXXXXXXXXXXWGQGTLVTVSS

SEQ ID NO: 86 - protein, 118 aa, synthetic construct
  VARIANT 97..106: note = X is any residue
  REGION 1..118: note = Modified VH domain from a human immunoglobulin germline
QCQLVQSGAEVKKPGASVKVSCKASGYTFTSYGISWVRQAPGQGLEWMGWISAYNGNTNYAQKLQGRVTMTTDTSTSTAYMELRSLRSDDTAVYYCXXXXXXXXXXCWGQGTLVTVSS

SEQ ID NO: 87 - protein, 122 aa, synthetic construct
  VARIANT 97..111: note = X is any residue
  REGION 1..122: note = Modified VH domain from a human immunoglobulin germline
EVQLVQSGAEVKKPGATVKISCKVSGYTFTDYYMHWVQQAPGKGLEWMGLVDPEDGETIYAEKFQGRVTITADTSTDTAYMELSSLRSEDTAVYYCXXXXXXXXXXXXXXXWGQGTLVTVSS

SEQ ID NO: 88 - protein, 122 aa, synthetic construct
  VARIANT 97..111: note = X is any residue
  REGION 1..122: note = Modified VH domain from a human immunoglobulin germline
EVQLVQSGAEVKKPGACVKISCKVSGYTFTDYYMHWVQQAPGKGLEWMGLVDPEDGETIYAEKFQGRVTITADTSTDTAYMELCSLRSEDTAVYYCXXXXXXXXXXXXXXXWGQGTLVTVSS

SEQ ID NO: 89 - protein, 122 aa, synthetic construct
  VARIANT 97..111: note = X is any residue
  REGION 1..122: note = Modified VH domain from a human immunoglobulin germline
EVQLVQSGAEVKKPGATVCISCKVSGYTFTDYYMHWVQQAPGKGLEWMGLVDPEDGETIYAEKFQGRVTITADTSTDTAYMCLSSLRSEDTAVYYCXXXXXXXXXXXXXXXWGQGTLVTVSS

SEQ ID NO: 90 - protein, 122 aa, synthetic construct
  VARIANT 97..111: note = X is any residue
  REGION 1..122: note = Modified VH domain from a human immunoglobulin germline
EVQLVQSGAEVKKPGATVKISCKVSGYTFTDYYCHWVQQAPGKGLEWMGLVDPEDGETIYAEKFQGRVTITADTSTDTCYMELSSLRSEDTAVYYCXXXXXXXXXXXXXXXWGQGTLVTVSS

SEQ ID NO: 91 - protein, 122 aa, synthetic construct
  VARIANT 97..111: note = X is any residue
  REGION 1..122: note = Modified VH domain from a human immunoglobulin germline
EVQLVQSGAEVKKPGATVKISCKVSGYTFTDYYMCWVQQAPGKGLEWMGCVDPEDGETIYAEKFQGRVTITADTSTDTAYMELSSLRSEDTAVYYCXXXXXXXXXXXXXXXWGQGTLVTVSS

SEQ ID NO: 92 - protein, 122 aa, synthetic construct
  VARIANT 1..122: note = Modified VH domain from a human immunoglobulin germline
  VARIANT 97..110: note = X is any residue
  REGION 1..122: note = Modified VH domain from a human immunoglobulin germline
ECQLVQSGAEVKKPGATVKISCKVSGYTFTDYYMHWVQQAPGKGLEWMGLVDPEDGETIYAEKFQGRVTITADTSTDTAYMELSSLRSEDTAVYYCXXXXXXXXXXXXXXCWGQGTLVTVSS

SEQ ID NO: 93 - protein, 123 aa, synthetic construct
  REGION 1..123: note = Modified VH domain from a human immunoglobulin germline
  VARIANT 97..112: note = X is any residue
EVQLVQSGAEVKKPGATVKISCKVSGYTFTDYYMHWVQQAPGKGLEWMGLVDPEDGETIYAEKFQGRVTITADTSTDTAYMELSSLRSEDTAVYYCXXXXXXXXXXXXXXXXWGQGTLVTVSS

SEQ ID NO: 94 - protein, 123 aa, synthetic construct
  REGION 1..123: note = Modified VH domain from a human immunoglobulin germline
  VARIANT 97..112: note = X is any residue
EVQLVQSGAEVKKPGACVKISCKVSGYTFTDYYMHWVQQAPGKGLEWMGLVDPEDGETIYAEKFQGRVTITADTSTDTAYMELCSLRSEDTAVYYCXXXXXXXXXXXXXXXXWGQGTLVTVSS

SEQ ID NO: 95 - protein, 123 aa, synthetic construct
  REGION 1..123: note = Modified VH domain from a human immunoglobulin germline
  VARIANT 97..112: note = X is any residue
EVQLVQSGAEVKKPGATVCISCKVSGYTFTDYYMHWVQQAPGKGLEWMGLVDPEDGETIYAEKFQGRVTITADTSTDTAYMCLSSLRSEDTAVYYCXXXXXXXXXXXXXXXXWGQGTLVTVSS

SEQ ID NO: 96 - protein, 123 aa, synthetic construct
  REGION 1..123: note = Modified VH domain from a human immunoglobulin germline
  VARIANT 97..112: note = X is anyresidue
EVQLVQSGAEVKKPGATVKISCCVSGYTFTDYYMHWVQQAPGKGLEWMGLVDPEDGETIYAEKFQGRVTITADTSTDCAYMELSSLRSEDTAVYYCXXXXXXXXXXXXXXXXWGQGTLVTVSS

SEQ ID NO: 97 - protein, 123 aa, synthetic construct
  REGION 1..123: note = Modified VH domain from a human immunoglobulin germline
  VARIANT 97..112: note = X is any residue
EVQLVQSGAEVKKPGATVKISCKVSGYTFTDYYMCWVQQAPGKGLEWMGCVDPEDGETIYAEKFQGRVTITADTSTDTAYMELSSLRSEDTAVYYCXXXXXXXXXXXXXXXXWGQGTLVTVSS

SEQ ID NO: 98 - protein, 123 aa, synthetic construct
  REGION 1..123: note = Modified VH domain from a human immunoglobulin germline
  VARIANT 97..111: note = X is any residue
ECQLVQSGAEVKKPGATVKISCKVSGYTFTDYYMHWVQQAPGKGLEWMGLVDPEDGETIYAEKFQGRVTITADTSTDTAYMELSSLRSEDTAVYYCXXXXXXXXXXXXXXXCWGQGTLVTVSS

SEQ ID NO: 99 - protein, 120 aa, synthetic construct
  REGION 1..120: note = Modified VH domain from a human immunoglobulin germline
  VARIANT 97..109: note = X is any residue
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCXXXXXXXXXXXXXLGQGTLVTVSS

SEQ ID NO: 100 - protein, 120 aa, synthetic construct
  REGION 1..120: note = Modified VH domain from a human immunoglobulin germline
  VARIANT 97..109: note = X is any residue
EVQLVESGGGLVQPGRCLRLSCAASGFTFDDYAMHWVRQAPGKGLEWVSGISWNSGSIGYADSVKGRFTISRDNAKNSLYLQMCSLRAEDTALYYCXXXXXXXXXXXXXLGQGTLVTVSS

SEQ ID NO: 101 - protein, 120 aa, synthetic construct
  REGION 1..120: note = Modified VH domain from a human immunoglobulin germline
  VARIANT 97..109: note = X is any residue
EVQLVESGGGLVQPGRSLCLSCAASGFTFDDYAMHWVRQAPGKGLEWVSGISWNSGSIGYADSVKGRFTISRDNAKNSLYLCMNSLRAEDTALYYCXXXXXXXXXXXXXLGQGTLVTVSS

SEQ ID NO: 102 - protein, 120 aa, synthetic construct
  REGION 1..120: note = Modified VH domain from a human immunoglobulin germline
  VARIANT 97..109: note = X is any residue
EVQLVESGGGLVQPGRSLRLSCCASGFTFDDYAMHWVRQAPGKGLEWVSGISWNSGSIGYADSVKGRFTISRDNAKNCLYLQMNSLRAEDTALYYCXXXXXXXXXXXXXLGQGTLVTVSS

SEQ ID NO: 103 - protein, 120 aa, synthetic construct
  REGION 1..120: note = Modified VH domain from a human immunoglobulin germline
  VARIANT 97..109: note = X is any residue
EVQLVESGGGLVQPGRSLRLSCAASGFTFDDYAMCWVRQAPGKGLEWVSCISWNSGSIGYADSVKGRFTISRDNAKNSLYLQMNSLRAEDTALYYCXXXXXXXXXXXXXLGQGTLVTVSS

SEQ ID NO: 104 - protein, 120 aa, synthetic construct
  REGION 1..120: note = Modified VH domain from a human immunoglobulin germline
  VARIANT 97..108: note = X is any residue
ECQLVESGGGLVQPGRSLRLSCAASGFTFDDYAMHWVRQAPGKGLEWVSGISWNSGSIGYADSVKGRFTISRDNAKNSLYLQMNSLRAEDTALYYCXXXXXXXXXXXXCLGQGTLVTVSS

SEQ ID NO: 105 - protein, 121 aa, synthetic construct
  REGION 1..121: note = Modified VH domain from a human immunoglobulin germline
  VARIANT 97..110: note = X is any residue
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCXXXXXXXXXXXXXXWGQGTLVTVSS

SEQ ID NO: 106 - protein, 121 aa, synthetic construct
  REGION 1..121: note = Modified VH domain from a human immunoglobulin germline
  VARIANT 97..110: note = X is any residue
QVQLVESGGGLVKPGGCLRLSCAASGFTFSDYYMSWIRQAPGKGLEWVSYISSSGSTIYYADSVKGRFTISRDNAKNSLYLQMCSLRAEDTAVYYCXXXXXXXXXXXXXXWGQGTLVTVSS

SEQ ID NO: 107 - protein, 121 aa, synthetic construct
  REGION 1..121: note = Modified VH domain from a human immunoglobulin germline
  VARIANT 97..110: note = X is any residue
QVQLVESGGGLVKPGGSLCLSCAASGFTFSDYYMSWIRQAPGKGLEWVSYISSSGSTIYYADSVKGRFTISRDNAKNSLYLCMNSLRAEDTAVYYCXXXXXXXXXXXXXXWGQGTLVTVSS

SEQ ID NO: 108 - protein, 121 aa, synthetic construct
  REGION 1..121: note = Modified VH domain from a human immunoglobulin germline
  VARIANT 97..110: note = X is any residue
QVQLVESGGGLVKPGGSLRLSCCASGFTFSDYYMSWIRQAPGKGLEWVSYISSSGSTIYYADSVKGRFTISRDNAKNCLYLQMNSLRAEDTAVYYCXXXXXXXXXXXXXXWGQGTLVTVSS

SEQ ID NO: 109 - protein, 121 aa, synthetic construct
  REGION 1..121: note = Modified VH domain from a human immunoglobulin germline
  VARIANT 97..110: note = X is any residue
QVQLVESGGGLVKPGGSLRLSCAASGFTFSDYYMCWIRQAPGKGLEWVSCISSSGSTIYYADSVKGRFTISRDNAKNSLYLQMNSLRAEDTAVYYCXXXXXXXXXXXXXXWGQGTLVTVSS

SEQ ID NO: 110 - protein, 121 aa, synthetic construct
  REGION 1..121: note = Modified VH domain from a human immunoglobulin germline
  VARIANT 97..109: note = X is any residue
QCQLVESGGGLVKPGGSLRLSCAASGFTFSDYYMSWIRQAPGKGLEWVSYISSSGSTIYYADSVKGRFTISRDNAKNSLYLQMNSLRAEDTAVYYCXXXXXXXXXXXXXCWGQGTLVTVSS

SEQ ID NO: 111 - protein, 119 aa, synthetic construct
  REGION 1..119: note = Modified VH domain from a human immunoglobulin germline
  VARIANT 99..108: note = X is any residue
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCXXXXXXXXXXWGQGTLVTVSS

SEQ ID NO: 112 - protein, 119 aa, synthetic construct
  REGION 1..119: note = Modified VH domain from a human immunoglobulin germline
  VARIANT 99..108: note = X is any residue
EVQLVESGGGLVKPGGCLRLSCAASGFTFSNAWMSWVRQAPGKGLEWVGRIKSKTDGGTTDYAAPVKGRFTISRDDSKNTLYLQMCSLKTEDTAVYYCXXXXXXXXXXWGQGTLVTVSS

SEQ ID NO: 113 - protein, 119 aa, synthetic construct
  REGION 1..119: note = Modified VH domain from a human immunoglobulin germline
  VARIANT 99..108: note = X is any residue
EVQLVESGGGLVKPGGSLCLSCAASGFTFSNAWMSWVRQAPGKGLEWVGRIKSKTDGGTTDYAAPVKGRFTISRDDSKNTLYLCMNSLKTEDTAVYYCXXXXXXXXXXWGQGTLVTVSS

SEQ ID NO: 114 - protein, 119 aa, synthetic construct
  REGION 1..119: note = Modified VH domain from a human immunoglobulin germline
  VARIANT 99..108: note = X is any residue
EVQLVESGGGLVKPGGSLRLSCCASGFTFSNAWMSWVRQAPGKGLEWVGRIKSKTDGGTTDYAAPVKGRFTISRDDSKNCLYLQMNSLKTEDTAVYYCXXXXXXXXXXWGQGTLVTVSS

SEQ ID NO: 115 - protein, 119 aa, synthetic construct
  REGION 1..119: note = Modified VH domain from a human immunoglobulin germline
  VARIANT 99..108: note = X is any residue
EVQLVESGGGLVKPGGSLRLSCAASGFTFSNAWMCWVRQAPGKGLEWVGCIKSKTDGGTTDYAAPVKGRFTISRDDSKNTLYLQMNSLKTEDTAVYYCXXXXXXXXXXWGQGTLVTVSS

SEQ ID NO: 116 - protein, 119 aa, synthetic construct
  REGION 1..119: note = Modified VH domain from a human immunoglobulin germline
  VARIANT 99..107: note = X is any residue
ECQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCXXXXXXXXXCWGQGTLVTVSS

SEQ ID NO: 117 - protein, 119 aa, synthetic construct
  REGION 1..119: note = Modified VH domain from a human immunoglobulin germline
  VARIANT 97..108: note = X is any residue
EVQLVESGGGVVRPGGSLRLSCAASGFTFDDYGMSWVRQAPGKGLEWVSGINWNGGSTGYADSVKGRFTISRDNAKNSLYLQMNSLRAEDTALYHCXXXXXXXXXXXXWGQGTLVTVSS

SEQ ID NO: 118 - protein, 119 aa, synthetic construct
  REGION 1..119: note = Modified VH domain from a human immunoglobulin germline
  VARIANT 97..108: note = X is any residue
EVQLVESGGGVVRPGGCLRLSCAASGFTFDDYGMSWVRQAPGKGLEWVSGINWNGGSTGYADSVKGRFTISRDNAKNSLYLQMCSLRAEDTALYHCXXXXXXXXXXXXWGQGTLVTVSS

SEQ ID NO: 119 - protein, 119 aa, synthetic construct
  REGION 1..119: note = Modified VH domain from a human immunoglobulin germline
  VARIANT 97..108: note = X is any residue
EVQLVESGGGVVRPGGSLCLSCAASGFTFDDYGMSWVRQAPGKGLEWVSGINWNGGSTGYADSVKGRFTISRDNAKNSLYLCMNSLRAEDTALYHCXXXXXXXXXXXXWGQGTLVTVSS

SEQ ID NO: 120 - protein, 119 aa, synthetic construct
  REGION 1..119: note = Modified VH domain from a human immunoglobulin germline
  VARIANT 97..108: note = X is any residue
EVQLVESGGGVVRPGGSLRLSCCASGFTFDDYGMSWVRQAPGKGLEWVSGINWNGGSTGYADSVKGRFTISRDNAKNCLYLQMNSLRAEDTALYHCXXXXXXXXXXXXWGQGTLVTVSS

SEQ ID NO: 121 - protein, 119 aa, synthetic construct
  REGION 1..119: note = Modified VH domain from a human immunoglobulin germline
  VARIANT 97..108: note = X is any residue
EVQLVESGGGVVRPGGSLRLSCAASGFTFDDYGMCWVRQAPGKGLEWVSCINWNGGSTGYADSVKGRFTISRDNAKNSLYLQMNSLRAEDTALYHCXXXXXXXXXXXXWGQGTLVTVSS

SEQ ID NO: 122 - protein, 119 aa, synthetic construct
  REGION 1..119: note = Modified VH domain from a human immunoglobulin germline
  VARIANT 97..107: note = X is any residue
ECQLVESGGGVVRPGGSLRLSCAASGFTFDDYGMSWVRQAPGKGLEWVSGINWNGGSTGYADSVKGRFTISRDNAKNSLYLQMNSLRAEDTALYHCXXXXXXXXXXXCWGQGTLVTVSS

SEQ ID NO: 123 - protein, 118 aa, synthetic construct
  REGION 1..118: note = Modified VH domain from a human immunoglobulin germline
  VARIANT 97..107: note = X is any residue
EVQLVESGGGVVRPGGSLRLSCAASGFTFDDYGMSWVRQAPGKGLEWVSGINWNGGSTGYADSVKGRFTISRDNAKNSLYLQMNSLRAEDTALYHCXXXXXXXXXXXWGQGTMVTVSS

SEQ ID NO: 124 - protein, 118 aa, synthetic construct
  REGION 1..118: note = Modified VH domain from a human immunoglobulin germline
  VARIANT 97..107: note = X is any residue
EVQLVESGGGVVRPGGSLRLSCCASGFTFDDYGMSWVRQAPGKGLEWVSGINWNGGSTGYADSVKGRFTISRDNAKNCLYLQMNSLRAEDTALYHCXXXXXXXXXXXWGQGTMVTVSS

SEQ ID NO: 125 - protein, 118 aa, synthetic construct
  REGION 1..118: note = Modified VH domain from a human immunoglobulin germline
  VARIANT 97..107: note = X is any residue
EVQLVESGGGVVRPGGSLRLSCAASGFTFDDYGMCWVRQAPGKGLEWVSCINWNGGSTGYADSVKGRFTISRDNAKNSLYLQMNSLRAEDTALYHCXXXXXXXXXXXWGQGTMVTVSS

SEQ ID NO: 126 - protein, 116 aa, synthetic construct
  REGION 1..116: note = Modified VH domain from a human immunoglobulin germline
  VARIANT 97..105: note = X is any residue
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCXXXXXXXXXWGRGTLVTVSS

SEQ ID NO: 127 - protein, 116 aa, synthetic construct
  REGION 1..116: note = Modified VH domain from a human immunoglobulin germline
  VARIANT 97..105: note = X is any residue
EVQLVESGGGLVKPGGSLRLSCAASGFTFSSYSMCWVRQAPGKGLEWVSCISSSSSYIYYADSVKGRFTISRDNAKNSLYLQMNSLRAEDTAVYYCXXXXXXXXXWGRGTLVTVSS

SEQ ID NO: 128 - protein, 118 aa, synthetic construct
  REGION 1..118: note = Modified VH domain from a human immunoglobulin germline
  VARIANT 97..107: note = X is any residue
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCXXXXXXXXXXXWGRGTLVTVSS

SEQ ID NO: 129 - protein, 118 aa, synthetic construct
  REGION 1..118: note = Modified VH domain from a human immunoglobulin germline
  VARIANT 97..107: note = X is any residue
QVQLVESGGGVVQPGRCLRLSCAASGFTFSSYAMHWVRQAPGKGLEWVAVISYDGSNKYYADSVKGRFTISRDNSKNTLYLQMCSLRAEDTAVYYCXXXXXXXXXXXWGRGTLVTVSS

SEQ ID NO: 130 - protein, 118 aa, synthetic construct
  REGION 1..118: note = Modified VH domain from a human immunoglobulin germline
  VARIANT 97..107: note = X is any residue
QVQLVESGGGVVQPGRSLCLSCAASGFTFSSYAMHWVRQAPGKGLEWVAVISYDGSNKYYADSVKGRFTISRDNSKNTLYLCMNSLRAEDTAVYYCXXXXXXXXXXXWGRGTLVTVSS

SEQ ID NO: 131 - protein, 118 aa, synthetic construct
  REGION 1..118: note = Modified VH domain from a human immunoglobulin germline
  VARIANT 97..107: note = X is any residue
QVQLVESGGGVVQPGRSLRLSCCASGFTFSSYAMHWVRQAPGKGLEWVAVISYDGSNKYYADSVKGRFTISRDNSKNCLYLQMNSLRAEDTAVYYCXXXXXXXXXXXWGRGTLVTVSS

SEQ ID NO: 132 - protein, 118 aa, synthetic construct
  REGION 1..118: note = Modified VH domain from a human immunoglobulin germline
  VARIANT 97..107: note = X is any residue
QVQLVESGGGVVQPGRSLRLSCAASGFTFSSYAMCWVRQAPGKGLEWVACISYDGSNKYYADSVKGRFTISRDNSKNTLYLQMNSLRAEDTAVYYCXXXXXXXXXXXWGRGTLVTVSS

SEQ ID NO: 133 - protein, 118 aa, synthetic construct
  REGION 1..118: note = Modified VH domain from a human immunoglobulin germline
  VARIANT 97..106: note = X is any residue
QCQLVESGGGVVQPGRSLRLSCAASGFTFSSYAMHWVRQAPGKGLEWVAVISYDGSNKYYADSVKGRFTISRDNSKNTLYLQMNSLRAEDTAVYYCXXXXXXXXXXCWGRGTLVTVSS

SEQ ID NO: 134 - protein, 123 aa, synthetic construct
  REGION 1..123: note = Modified VH domain from a human immunoglobulin germline
  VARIANT 96..112: note = X is any residue
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCXXXXXXXXXXXXXXXXXWGRGTLVTVSS

SEQ ID NO: 135 - protein, 123 aa, synthetic construct
  REGION 1..123: note = Modified VH domain from a human immunoglobulin germline
  VARIANT 96..112: note = X is any residue
EVQLVESGGGLIQPGGCLRLSCAASGFTVSSNYMSWVRQAPGKGLEWVSVIYSGGSTYYADSVKGRFTISRDNSKNTLYLQMCSLRAEDTAVYYCXXXXXXXXXXXXXXXXXWGRGTLVTVSS

SEQ ID NO: 136 - protein, 123 aa, synthetic construct
  REGION 1..123: note = Modified VH domain from a human immunoglobulin germline
  VARIANT 96..112: note = X is any residue
EVQLVESGGGLIQPGGSLCLSCAASGFTVSSNYMSWVRQAPGKGLEWVSVIYSGGSTYYADSVKGRFTISRDNSKNTLYLCMNSLRAEDTAVYYCXXXXXXXXXXXXXXXXXWGRGTLVTVSS

SEQ ID NO: 137 - protein, 123 aa, synthetic construct
  REGION 1..123: note = Modified VH domain from a human immunoglobulin germline
  VARIANT 96..112: note = X is any residue
EVQLVESGGGLIQPGGSLRLSCCASGFTVSSNYMSWVRQAPGKGLEWVSVIYSGGSTYYADSVKGRFTISRDNSKNCLYLQMNSLRAEDTAVYYCXXXXXXXXXXXXXXXXXWGRGTLVTVSS

SEQ ID NO: 138 - protein, 123 aa, synthetic construct
  REGION 1..123: note = Modified VH domain from a human immunoglobulin germline
  VARIANT 96..112: note = X is any residue
EVQLVESGGGLIQPGGSLRLSCAASGFTVSSNYMCWVRQAPGKGLEWVSCIYSGGSTYYADSVKGRFTISRDNSKNTLYLQMNSLRAEDTAVYYCXXXXXXXXXXXXXXXXXWGRGTLVTVSS

SEQ ID NO: 139 - protein, 123 aa, synthetic construct
  REGION 1..123: note = Modified VH domain from a human immunoglobulin germline
  VARIANT 96..111: note = X is any residue
ECQLVESGGGLIQPGGSLRLSCAASGFTVSSNYMSWVRQAPGKGLEWVSVIYSGGSTYYADSVKGRFTISRDNSKNTLYLQMNSLRAEDTAVYYCXXXXXXXXXXXXXXXXCWGRGTLVTVSS

SEQ ID NO: 140 - protein, 119 aa, synthetic construct
  REGION 1..119: note = Modified VH domain from a human immunoglobulin germline
  VARIANT 96..108: note = X is any residue
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCXXXXXXXXXXXXXWGQGTLVTVSS

SEQ ID NO: 141 - protein, 119 aa, synthetic construct
  REGION 1..119: note = Modified VH domain from a human immunoglobulin germline
  VARIANT 96..108: note = X is any residue
QVQLQQWGAGLLKPSECLSLTCAVYGGSFSGYYWSWIRQPPGKGLEWIGEINHSGSTNYNPSLKSRVTISVDTSKNQFSLKLCSVTAADTAVYYCXXXXXXXXXXXXXWGQGTLVTVSS

SEQ ID NO: 142 - protein, 119 aa, synthetic construct
  REGION 1..119: note = Modified VH domain from a human immunoglobulin germline
  VARIANT 96..108: note = X is any residue
QVQLQQWGAGLLKPSETLCLTCAVYGGSFSGYYWSWIRQPPGKGLEWIGEINHSGSTNYNPSLKSRVTISVDTSKNQFSLCLSSVTAADTAVYYCXXXXXXXXXXXXXWGQGTLVTVSS

SEQ ID NO: 143 - protein, 119 aa, synthetic construct
  REGION 1..119: note = Modified VH domain from a human immunoglobulin germline
  VARIANT 96..108: note = X is any residue
QVQLQQWGAGLLKPSETLSLTCCVYGGSFSGYYWSWIRQPPGKGLEWIGEINHSGSTNYNPSLKSRVTISVDTSKNCFSLKLSSVTAADTAVYYCXXXXXXXXXXXXXWGQGTLVTVSS

SEQ ID NO: 144 - protein, 119 aa, synthetic construct
  REGION 1..119: note = Modified VH domain from a human immunoglobulin germline
  VARIANT 96..108: note = X is any residue
QVQLQQWGAGLLKPSETLSLTCAVYGGSFSGYYWCWIRQPPGKGLEWIGCINHSGSTNYNPSLKSRVTISVDTSKNQFSLKLSSVTAADTAVYYCXXXXXXXXXXXXXWGQGTLVTVSS

SEQ ID NO: 145 - protein, 119 aa, synthetic construct
  REGION 1..119: note = Modified VH domain from a human immunoglobulin germline
  VARIANT 96..107: note = X is any residue
QCQLQQWGAGLLKPSETLSLTCAVYGGSFSGYYWSWIRQPPGKGLEWIGEINHSGSTNYNPSLKSRVTISVDTSKNQFSLKLSSVTAADTAVYYCXXXXXXXXXXXXCWGQGTLVTVSS

SEQ ID NO: 146 - protein, 123 aa, synthetic construct
  REGION 1..123: note = Modified VH domain from a human immunoglobulin germline
  VARIANT 98..112: note = X is any residue
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCXXXXXXXXXXXXXXXWGQGTLVTVSS

SEQ ID NO: 147 - protein, 123 aa, synthetic construct
  REGION 1..123: note = Modified VH domain from a human immunoglobulin germline
  VARIANT 98..112: note = X is any residue
QLQLQESGPGLVKPSECLSLTCTVSGGSISSSSYYWGWIRQPPGKGLEWIGSIYYSGSTYYNPSLKSRVTISVDTSKNQFSLKLCSVTAADTAVYYCXXXXXXXXXXXXXXXWGQGTLVTVSS

SEQ ID NO: 148 - protein, 123 aa, synthetic construct
  REGION 1..123: note = Modified VH domain from a human immunoglobulin germline
  VARIANT 98..112: note = X is any residue
QLQLQESGPGLVKPSETLCLTCTVSGGSISSSSYYWGWIRQPPGKGLEWIGSIYYSGSTYYNPSLKSRVTISVDTSKNQFSLCLSSVTAADTAVYYCXXXXXXXXXXXXXXXWGQGTLVTVSS

SEQ ID NO: 149 - protein, 123 aa, synthetic construct
  REGION 1..123: note = Modified VH domain from a human immunoglobulin germline
  VARIANT 98..112: note = X is any residue
QLQLQESGPGLVKPSETLSLTCCVSGGSISSSSYYWGWIRQPPGKGLEWIGSIYYSGSTYYNPSLKSRVTISVDTSKNCFSLKLSSVTAADTAVYYCXXXXXXXXXXXXXXXWGQGTLVTVSS

SEQ ID NO: 150 - protein, 123 aa, synthetic construct
  REGION 1..123: note = Modified VH domain from a human immunoglobulin germline
  VARIANT 98..112: note = X is any residue
QLQLQESGPGLVKPSETLSLTCTVSGGSISSSSYYWCWIRQPPGKGLEWIGCIYYSGSTYYNPSLKSRVTISVDTSKNQFSLKLSSVTAADTAVYYCXXXXXXXXXXXXXXXWGQGTLVTVSS

SEQ ID NO: 151 - protein, 123 aa, synthetic construct
  REGION 1..123: note = Modified VH domain from a human immunoglobulin germline
  VARIANT 98..111: note = X is any residue
QCQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCXXXXXXXXXXXXXXCWGQGTLVTVSS

SEQ ID NO: 152 - protein, 113 aa, synthetic construct
  REGION 1..113: note = Modified VH domain from a human immunoglobulin germline
  VARIANT 97..102: note = X is any residue
QVQLVQSGSELKKPGASVKVSCKASGYTFTSYAMNWVRQAPGQGLEWMGWINTNTGNPTYAQGFTGRFVFSLDTSVSTAYLQICSLKAEDTAVYYCXXXXXXWGQGTMVTVSS

SEQ ID NO: 153 - protein, 113 aa, synthetic construct
  REGION 1..113: note = Modified VH domain from a human immunoglobulin germline
  VARIANT 97..102: note = X is any residue
QVQLVQSGSELKKPGACVKVSCKASGYTFTSYAMNWVRQAPGQGLEWMGWINTNTGNPTYAQGFTGRFVFSLDTSVSTAYLQICSLKAEDTAVYYCXXXXXXWGQGTMVTVSS

SEQ ID NO: 154 - protein, 113 aa, synthetic construct
  REGION 1..113: note = Modified VH domain from a human immunoglobulin germline
  VARIANT 97..102: note = X is any residue
QVQLVQSGSELKKPGASVCVSCKASGYTFTSYAMNWVRQAPGQGLEWMGWINTNTGNPTYAQGFTGRFVFSLDTSVSTAYLCICSLKAEDTAVYYCXXXXXXWGQGTMVTVSS

SEQ ID NO: 155 - protein, 113 aa, synthetic construct
  REGION 1..113: note = Modified VH domain from a human immunoglobulin germline
  VARIANT 97..102: note = X is any residue
QVQLVQSGSELKKPGASVKVSCCASGYTFTSYAMNWVRQAPGQGLEWMGWINTNTGNPTYAQGFTGRFVFSLDTSVSCAYLQICSLKAEDTAVYYCXXXXXXWGQGTMVTVSS

SEQ ID NO: 156 - protein, 113 aa, synthetic construct
  REGION 1..113: note = Modified VH domain from a human immunoglobulin germline
  VARIANT 97..102: note = X is any residue
QVQLVQSGSELKKPGASVKVSCKASGYTFTSYAMCWVRQAPGQGLEWMGCINTNTGNPTYAQGFTGRFVFSLDTSVSTAYLQICSLKAEDTAVYYCXXXXXXWGQGTMVTVSS

SEQ ID NO: 157 - protein, 113 aa, synthetic construct
  REGION 1..113: note = Modified VH domain from a human immunoglobulin germline
  VARIANT 97..101: note = X is any residue
QCQLVQSGSELKKPGASVKVSCKASGYTFTSYAMNWVRQAPGQGLEWMGWINTNTGNPTYAQGFTGRFVFSLDTSVSTAYLQICSLKAEDTAVYYCXXXXXCWGQGTMVTVSS

SEQ ID NO: 158 - protein, 118 aa, synthetic construct
  REGION 1..118: note = Modified VH domain from a human immunoglobulin germline
  VARIANT 97..107: note = X is any residue
EAQLVESGGGVVRPGGSLRLSCAASGFTFDDYGMSWVRQAPGKGLEWVSGINWNGGSTGYADSVKGRFTISRDNAKNSLYLQMNSLRAEDTALYHCXXXXXXXXXXXWGQGTMVTVSS

SEQ ID NO: 159 - protein, 118 aa, synthetic construct
  REGION 1..118: note = Modified VH domain from a human immunoglobulin germline
  VARIANT 97..107: note = X is any residue
EVQLVESGGGVVREGGSLRLSCAASGFTFDDYGMSWVRQAPGKGLEWVSGINWNGGSTGYADSVKGRFTISRDNAKNSLYLQMNSLRAEDTALYHCXXXXXXXXXXXWGQGTMVTVSS

SEQ ID NO: 160 - protein, 118 aa, synthetic construct
  REGION 1..118: note = Modified VH domain from a human immunoglobulin germline
  VARIANT 97..107: note = X is any residue
EVQLVESGGGVVRPGGSLRLSCQASGFTFDDYGMSWVRQAPGKGLEWVSGINWNGGSTGYADSVKGRFTISRDNAKNSLYLQMNSLRAEDTALYHCXXXXXXXXXXXWGQGTMVTVSS

SEQ ID NO: 161 - protein, 118 aa, synthetic construct
  REGION 1..118: note = Modified VH domain from a human immunoglobulin germline
  VARIANT 97..107: note = X is any residue
EVQLVESGGGVVRPGGSLRLSCAASGFNFDDYGMSWVRQAPGKGLEWVSGINWNGGSTGYADSVKGRFTISRDNAKNSLYLQMNSLRAEDTALYHCXXXXXXXXXXXWGQGTMVTVSS

SEQ ID NO: 162 - protein, 118 aa, synthetic construct
  REGION 1..118: note = Modified VH domain from a human immunoglobulin germline
  VARIANT 97..107: note = X is any residue
EVQLVESGGGVVRPGGSLRLSCAASGFKFDDYGMSWVRQAPGKGLEWVSGINWNGGSTGYADSVKGRFTISRDNAKNSLYLQMNSLRAEDTALYHCXXXXXXXXXXXWGQGTMVTVSS

SEQ ID NO: 163 - protein, 118 aa, synthetic construct
  REGION 1..118: note = Modified VH domain from a human immunoglobulin germline
  VARIANT 97..107: note = X is any residue
EVQLVESGGGVVRPGGSLRLSCAASGFRFDDYGMSWVRQAPGKGLEWVSGINWNGGSTGYADSVKGRFTISRDNAKNSLYLQMNSLRAEDTALYHCXXXXXXXXXXXWGQGTMVTVSS

SEQ ID NO: 164 - protein, 118 aa, synthetic construct
  REGION 1..118: note = Modified VH domain from a human immunoglobulin germline
  VARIANT 97..107: note = X is any residue
EVQLVESGGGVVRPGGSLRLSCAASGFTFKDYGMSWVRQAPGKGLEWVSGINWNGGSTGYADSVKGRFTISRDNAKNSLYLQMNSLRAEDTALYHCXXXXXXXXXXXWGQGTMVTVSS

SEQ ID NO: 165 - protein, 118 aa, synthetic construct
  REGION 1..118: note = Modified VH domain from a human immunoglobulin germline
  VARIANT 97..107: note = X is any residue
EVQLVESGGGVVRPGGSLRLSCAASGFTFSDYGMSWVRQAPGKGLEWVSGINWNGGSTGYADSVKGRFTISRDNAKNSLYLQMNSLRAEDTALYHCXXXXXXXXXXXWGQGTMVTVSS

SEQ ID NO: 166 - protein, 118 aa, synthetic construct
  REGION 1..118: note = Modified VH domain from a human immunoglobulin germline
  VARIANT 97..107: note = X is any residue
EVQLVESGGGVVRPGGSLRLSCAASGFTFDDYGMSWVRQAPGKGLEWVAGINWNGGSTGYADSVKGRFTISRDNAKNSLYLQMNSLRAEDTALYHCXXXXXXXXXXXWGQGTMVTVSS

SEQ ID NO: 167 - protein, 118 aa, synthetic construct
  REGION 1..118: note = Modified VH domain from a human immunoglobulin germline
  VARIANT 97..107: note = X is any residue
EVQLVESGGGVVRPGGSLRLSCAASGFTFDDYGMSWVRQAPGKGLEWVSGINWNGESTGYADSVKGRFTISRDNAKNSLYLQMNSLRAEDTALYHCXXXXXXXXXXXWGQGTMVTVSS

SEQ ID NO: 168 - protein, 118 aa, synthetic construct
  REGION 1..118: note = Modified VH domain from a human immunoglobulin germline
  VARIANT 97..107: note = X is any residue
EVQLVESGGGVVRPGGSLRLSCAASGFTFDDYGMSWVRQAPGKGLEWVSGINWNGGSTGYADSVKGRFTISRDNEKNSLYLQMNSLRAEDTALYHCXXXXXXXXXXXWGQGTMVTVSS

SEQ ID NO: 169 - protein, 118 aa, synthetic construct
  REGION 1..118: note = Modified VH domain from a human immunoglobulin germline
  VARIANT 97..107: note = X is any residue
EVQLVESGGGVVRPGGSLRLSCAASGFTFDDYGMSWVRQAPGKGLEWVSGINWNGGSTGYADSVKGRFTISRDNAKKSLYLQMNSLRAEDTALYHCXXXXXXXXXXXWGQGTMVTVSS

SEQ ID NO: 170 - protein, 118 aa, synthetic construct
  REGION 1..118: note = Modified VH domain from a human immunoglobulin germline
  VARIANT 97..107: note = X is any residue
EVQLVESGGGVVRPGGSLRLSCAASGFTFDDYGMSWVRQAPGKGLEWVSGINWNGGSTGYADSVKGRFTISRDNAKNQLYLQMNSLRAEDTALYHCXXXXXXXXXXXWGQGTMVTVSS

SEQ ID NO: 171 - protein, 118 aa, synthetic construct
  REGION 1..118: note = Modified VH domain from a human immunoglobulin germline
  VARIANT 97..107: note = X is any residue
EVQLVESGGGVVRPGGSLRLSCAASGFTFDDYGMSWVRQAPGKGLEWVSGINWNGGSTGYADSVKGRFTISRDNAKNSLYLQMNSLREEDTALYHCXXXXXXXXXXXWGQGTMVTVSS

SEQ ID NO: 172 - protein, 118 aa, synthetic construct
  REGION 1..118: note = Modified VH domain from a human immunoglobulin germline
  VARIANT 97..107: note = X is any residue
EVQLVESGGGVVRPGGSLRLSCAASGFTFDDYGMSWVRQAPGKGLEWVSGINWNGGSTGYADSVKGRFTISRDNAKNSLYLQMNSLRPEDTALYHCXXXXXXXXXXXWGQGTMVTVSS

SEQ ID NO: 173 - protein, 118 aa, synthetic construct
  REGION 1..118: note = Modified VH domain from a human immunoglobulin germline
  VARIANT 97..107: note = X is any residue
EVQLVESGGGVVRPGGSLRLSCQASGFTFDDYGMSWVRQAPGKGLEWVSGINWNGGSTGYADSVKGRFTISRDNAKNQLYLQMNSLRAEDTALYHCXXXXXXXXXXXWGQGTMVTVSS

SEQ ID NO: 174 - protein, 116 aa, synthetic construct
  REGION 1..116: note = Modified VH domain from a human immunoglobulin germline
  VARIANT 97..105: note = X is any residue
EVQLVESGGGLVKPGGSLRLSCQASGFTFSSYSMNWVRQAPGKGLEWVSSISSSSSYIYYADSVKGRFTISRDNAKNSLYLQMNSLRAEDTAVYYCXXXXXXXXXWGRGTLVTVSS

SEQ ID NO: 175 - protein, 116 aa, synthetic construct
  REGION 1..116: note = Modified VH domain from a human immunoglobulin germline
  VARIANT 97..105: note = X is any residue
EVQLVESGGGLVKPGGSLRLSCAASGFDFSSYSMNWVRQAPGKGLEWVSSISSSSSYIYYADSVKGRFTISRDNAKNSLYLQMNSLRAEDTAVYYCXXXXXXXXXWGRGTLVTVSS

SEQ ID NO: 176 - protein, 116 aa, synthetic construct
  REGION 1..116: note = Modified VH domain from a human immunoglobulin germline
  VARIANT 97..105: note = X is any residue
EVQLVESGGGLVKPGGSLRLSCAASGFEFSSYSMNWVRQAPGKGLEWVSSISSSSSYIYYADSVKGRFTISRDNAKNSLYLQMNSLRAEDTAVYYCXXXXXXXXXWGRGTLVTVSS

SEQ ID NO: 177 - protein, 116 aa, synthetic construct
  REGION 1..116: note = Modified VH domain from a human immunoglobulin germline
  VARIANT 97..105: note = X is any residue
EVQLVESGGGLVKPGGSLRLSCAASGFTFSSYPMNWVRQAPGKGLEWVSSISSSSSYIYYADSVKGRFTISRDNAKNSLYLQMNSLRAEDTAVYYCXXXXXXXXXWGRGTLVTVSS

SEQ ID NO: 178 - protein, 116 aa, synthetic construct
  REGION 1..116: note = Modified VH domain from a human immunoglobulin germline
  VARIANT 97..105: note = X is any residue
EVQLVESGGGLVKPGGSLRLSCAASGFTFSSYSMGWVRQAPGKGLEWVSSISSSSSYIYYADSVKGRFTISRDNAKNSLYLQMNSLRAEDTAVYYCXXXXXXXXXWGRGTLVTVSS

SEQ ID NO: 179 - protein, 116 aa, synthetic construct
  REGION 1..116: note = Modified VH domain from a human immunoglobulin germline
  VARIANT 97..105: note = X is any residue
EVQLVESGGGLVKPGGSLRLSCAASGFTFSSYSMAWVRQAPGKGLEWVSSISSSSSYIYYADSVKGRFTISRDNAKNSLYLQMNSLRAEDTAVYYCXXXXXXXXXWGRGTLVTVSS

SEQ ID NO: 180 - protein, 116 aa, synthetic construct
  REGION 1..116: note = Modified VH domain from a human immunoglobulin germline
  VARIANT 97..105: note = X is any residue
EVQLVESGGGLVKPGGSLRLSCAASGFTFSSYSMSWVRQAPGKGLEWVSSISSSSSYIYYADSVKGRFTISRDNAKNSLYLQMNSLRAEDTAVYYCXXXXXXXXXWGRGTLVTVSS

SEQ ID NO: 181 - protein, 116 aa, synthetic construct
  REGION 1..116: note = Modified VH domain from a human immunoglobulin germline
  VARIANT 97..105: note = X is any residue
EVQLVESGGGLVKPGGSLRLSCAASGFTFSSYSMNWVRQAPGKGLEWVASISSSSSYIYYADSVKGRFTISRDNAKNSLYLQMNSLRAEDTAVYYCXXXXXXXXXWGRGTLVTVSS

SEQ ID NO: 182 - protein, 116 aa, synthetic construct
  REGION 1..116: note = Modified VH domain from a human immunoglobulin germline
  VARIANT 97..105: note = X is any residue
EVQLVESGGGLVKPGGSLRLSCAASGFTFSSYSMNWVRQAPGKGLEWVSSIDSSSSYIYYADSVKGRFTISRDNAKNSLYLQMNSLRAEDTAVYYCXXXXXXXXXWGRGTLVTVSS

SEQ ID NO: 183 - protein, 116 aa, synthetic construct
  REGION 1..116: note = Modified VH domain from a human immunoglobulin germline
  VARIANT 97..105: note = X is any residue
EVQLVESGGGLVKPGGSLRLSCAASGFTFSSYSMNWVRQAPGKGLEWVSSISSSSEYIYYADSVKGRFTISRDNAKNSLYLQMNSLRAEDTAVYYCXXXXXXXXXWGRGTLVTVSS

SEQ ID NO: 184 - protein, 116 aa, synthetic construct
  REGION 1..116: note = Modified VH domain from a human immunoglobulin germline
  VARIANT 97..105: note = X is any residue
EVQLVESGGGLVKPGGSLRLSCAASGFTFSSYSMNWVRQAPGKGLEWVSSISSSSSEIYYADSVKGRFTISRDNAKNSLYLQMNSLRAEDTAVYYCXXXXXXXXXWGRGTLVTVSS

SEQ ID NO: 185 - protein, 116 aa, synthetic construct
  REGION 1..116: note = Modified VH domain from a human immunoglobulin germline
  VARIANT 97..105: note = X is any residue
EVQLVESGGGLVKPGGSLRLSCAASGFTFSSYSMNWVRQAPGKGLEWVSSISSSSSYIYYADSVKGRFTISRDNEKNSLYLQMNSLRAEDTAVYYCXXXXXXXXXWGRGTLVTVSS

SEQ ID NO: 186 - protein, 116 aa, synthetic construct
  REGION 1..116: note = Modified VH domain from a human immunoglobulin germline
  VARIANT 97..105: note = X is any residue
EVQLVESGGGLVKPGGSLRLSCAASGFTFSSYSMNWVRQAPGKGLEWVSSISSSSSYIYYADSVKGRFTISRDNAKNSLYLQMNSLREEDTAVYYCXXXXXXXXXWGRGTLVTVSS

SEQ ID NO: 187 - protein, 116 aa, synthetic construct
  REGION 1..116: note = Modified VH domain from a human immunoglobulin germline
  VARIANT 97..105: note = X is any residue
EVQLVESGGGLVKPGGSLRLSCAASGFTFSSYSMNWVRQAPGKGLEWVSSISSSSSYIYYADSVKGRFTISRDNAKNSLYLQMNSLRPEDTAVYYCXXXXXXXXXWGRGTLVTVSS

SEQ ID NO: 188 - protein, 116 aa, synthetic construct
  REGION 1..116: note = Modified VH domain from a human immunoglobulin germline
  VARIANT 97..105: note = X is any residue
EVQLQESGGGLVKPGGSLRLSCQASGFTFSSYSMNWVRQAPGKGLEWVSSISSSSSYIYYADSVKGRFTISRDNAKNSLYLQMNSLRAEDTAVYYCXXXXXXXXXWGRGTLVTVSS

SEQ ID NO: 189 - protein, 119 aa, synthetic construct
  REGION 1..119: note = Modified VH domain from a human immunoglobulin germline
  VARIANT 99..108: note = X is any residue
EVQLVESGGGLVKPGGSLRLSCKASGFTFSNAWMSWVRQAPGKGLEWVGRIKSKTDGGTTDYAAPVKGRFTISRDDSKNTLYLQMNSLKTEDTAVYYCXXXXXXXXXXWGQGTLVTVSS

SEQ ID NO: 190 - protein, 119 aa, synthetic construct
  REGION 1..119: note = Modified VH domain from a human immunoglobulin germline
  VARIANT 99..108: note = X is any residue
EVQLVESGGGLVKPGGSLRLSCQASGFTFSNAWMSWVRQAPGKGLEWVGRIKSKTDGGTTDYAAPVKGRFTISRDDSKNTLYLQMNSLKTEDTAVYYCXXXXXXXXXXWGQGTLVTVSS

SEQ ID NO: 191 - protein, 119 aa, synthetic construct
  REGION 1..119: note = Modified VH domain from a human immunoglobulin germline
  VARIANT 99..108: note = X is any residue
EVQLVESGGGLVKPGGSLRLSCYASGFTFSNAWMSWVRQAPGKGLEWVGRIKSKTDGGTTDYAAPVKGRFTISRDDSKNTLYLQMNSLKTEDTAVYYCXXXXXXXXXXWGQGTLVTVSS

SEQ ID NO: 192 - protein, 119 aa, synthetic construct
  REGION 1..119: note = Modified VH domain from a human immunoglobulin germline
  VARIANT 99..108: note = X is any residue
EVQLVESGGGLVKPGGSLRLSCAASGFDFSNAWMSWVRQAPGKGLEWVGRIKSKTDGGTTDYAAPVKGRFTISRDDSKNTLYLQMNSLKTEDTAVYYCXXXXXXXXXXWGQGTLVTVSS

SEQ ID NO: 193 - protein, 119 aa, synthetic construct
  REGION 1..119: note = Modified VH domain from a human immunoglobulin germline
  VARIANT 99..108: note = X is any residue
EVQLVESGGGLVKPGGSLRLSCAASGFTFSKAWMSWVRQAPGKGLEWVGRIKSKTDGGTTDYAAPVKGRFTISRDDSKNTLYLQMNSLKTEDTAVYYCXXXXXXXXXXWGQGTLVTVSS

SEQ ID NO: 194 - protein, 119 aa, synthetic construct
  REGION 1..119: note = Modified VH domain from a human immunoglobulin germline
  VARIANT 99..108: note = X is any residue
EVQLVESGGGLVKPGGSLRLSCAASGFTFSNAWMSWVRQPPGKGLEWVGRIKSKTDGGTTDYAAPVKGRFTISRDDSKNTLYLQMNSLKTEDTAVYYCXXXXXXXXXXWGQGTLVTVSS

SEQ ID NO: 195 - protein, 119 aa, synthetic construct
  REGION 1..119: note = Modified VH domain from a human immunoglobulin germline
  VARIANT 99..108: note = X is any residue
EVQLVESGGGLVKPGGSLRLSCAASGFTFSNAWMSWVRQAPGKGLEWVGRIKSKTDGGTTDYAAPVKGRFTISRDDSKNTLYLQMNDLKTEDTAVYYCXXXXXXXXXXWGQGTLVTVSS

SEQ ID NO: 196 - protein, 119 aa, synthetic construct
  REGION 1..119: note = Modified VH domain from a human immunoglobulin germline
  VARIANT 99..108: note = X is any residue
EVQLVESGGGLVKPGGSLRLSCAASGFTFSNAWMSWVRQAPGKGLEWVGRIKSKTDGGTTDYAAPVKGRFTISRDDSKNTLYLQMNSLKEEDTAVYYCXXXXXXXXXXWGQGTLVTVSS

SEQ ID NO: 197 - protein, 119 aa, synthetic construct
  REGION 1..119: note = Modified VH domain from a human immunoglobulin germline
  VARIANT 99..108: note = X is any residue
EVQLVESGGGLVKPGGSLRLSCAASGFTFSNAWMSWVRQAPGKGLEWVGRIKSKTDGGTTDYAAPVKGRFTISRDDSKNTLYLQMNSLKPEDTAVYYCXXXXXXXXXXWGQGTLVTVSS

SEQ ID NO: 198 - protein, 119 aa, synthetic construct
  REGION 1..119: note = Modified VH domain from a human immunoglobulin germline
  VARIANT 99..108: note = X is any residue
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCXXXXXXXXXXWGQGTLVVVSS

SEQ ID NO: 199 - protein, 119 aa, synthetic construct
  REGION 1..119: note = Modified VH domain from a human immunoglobulin germline
  VARIANT 99..108: note = X is any residue
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCXXXXXXXXXXWGQGTLVIVSS

SEQ ID NO: 200 - protein, 122 aa, synthetic construct
  REGION 1..122: note = Modified VH domain from a human immunoglobulin germline
  VARIANT 97..111: note = X is any residue
EVQLVQSGAQVKKPGATVKISCKVSGYTFTDYYMHWVQQAPGKGLEWMGLVDPEDGETIYAEKFQGRVTITADTSTDTAYMELSSLRSEDTAVYYCXXXXXXXXXXXXXXXWGQGTLVTVSS

SEQ ID NO: 201 - protein, 122 aa, synthetic construct
  REGION 1..122: note = Modified VH domain from a human immunoglobulin germline
  VARIANT 97..111: note = X is any residue
EVQLVQSGAEVKKPGDTVKISCKVSGYTFTDYYMHWVQQAPGKGLEWMGLVDPEDGETIYAEKFQGRVTITADTSTDTAYMELSSLRSEDTAVYYCXXXXXXXXXXXXXXXWGQGTLVTVSS

SEQ ID NO: 202 - protein, 122 aa, synthetic construct
  REGION 1..122: note = Modified VH domain from a human immunoglobulin germline
  VARIANT 97..111: note = X is any residue
EVQLVQSGAEVKKPGQTVKISCKVSGYTFTDYYMHWVQQAPGKGLEWMGLVDPEDGETIYAEKFQGRVTITADTSTDTAYMELSSLRSEDTAVYYCXXXXXXXXXXXXXXXWGQGTLVTVSS

SEQ ID NO: 203 - protein, 122 aa, synthetic construct
  REGION 1..122: note = Modified VH domain from a human immunoglobulin germline
  VARIANT 97..111: note = X is any residue
EVQLVQSGAEVKKPGATVKISCKVYGYTFTDYYMHWVQQAPGKGLEWMGLVDPEDGETIYAEKFQGRVTITADTSTDTAYMELSSLRSEDTAVYYCXXXXXXXXXXXXXXXWGQGTLVTVSS

SEQ ID NO: 204 - protein, 122 aa, synthetic construct
  REGION 1..122: note = Modified VH domain from a human immunoglobulin germline
  VARIANT 97..111: note = X is any residue
EVQLVQSGAEVKKPGATVKISCKVSGYTFTDYYMHWFQQAPGKGLEWMGLVDPEDGETIYAEKFQGRVTITADTSTDTAYMELSSLRSEDTAVYYCXXXXXXXXXXXXXXXWGQGTLVTVSS

SEQ ID NO: 205 - protein, 122 aa, synthetic construct
  REGION 1..122: note = Modified VH domain from a human immunoglobulin germline
  VARIANT 97..111: note = X is any residue
EVQLVQSGAEVKKPGATVKISCKVSGYTFTDYYMHWVQQAPGKGLEWIGLVDPEDGETIYAEKFQGRVTITADTSTDTAYMELSSLRSEDTAVYYCXXXXXXXXXXXXXXXWGQGTLVTVSS

SEQ ID NO: 206 - protein, 122 aa, synthetic construct
  REGION 1..122: note = Modified VH domain from a human immunoglobulin germline
  VARIANT 97..111: note = X is any residue
EVQLVQSGAEVKKPGATVKISCKVSGYTFTDYYMHWVQQAPGKGLEWMGLVDPEDGETIYAEKFQGRVTITADTSTDTAYMELSSLREEDTAVYYCXXXXXXXXXXXXXXXWGQGTLVTVSS

SEQ ID NO: 207 - protein, 122 aa, synthetic construct
  REGION 1..122: note = Modified VH domain from a human immunoglobulin germline
  VARIANT 97..111: note = X is any residue
EVQLVQSGAEVKKPGATVKISCKVSGYTFTDYYMHWVQQAPGKGLEWMGLVDPEDGETIYAEKFQGRVTITADTSTDTAYMELSSLRPEDTAVYYCXXXXXXXXXXXXXXXWGQGTLVTVSS

SEQ ID NO: 208 - protein, 122 aa, synthetic construct
  REGION 1..122: note = Modified VH domain from a human immunoglobulin germline
  VARIANT 97..111: note = X is any residue
EVQLVQSGAEVKKPGATVKISCKVSGYTFTDYYMHWVQQAPGKGLEWMGLVDPEDGETIYAEKFQGRVTITADTSTDTAYMELSSLRSEDTAVYYCXXXXXXXXXXXXXXXWGQGTLVVVSS

SEQ ID NO: 209 - protein, 122 aa, synthetic construct
  REGION 1..122: note = Modified VH domain from a human immunoglobulin germline
  VARIANT 97..111: note = X is any residue
EVQLVQSGAEVKKPGATVKISCKVSGYTFTDYYMHWVQQAPGKGLEWMGLVDPEDGETIYAEKFQGRVTITADTSTDTAYMELSSLRSEDTAVYYCXXXXXXXXXXXXXXXWGQGTLVIVSS

SEQ ID NO: 210 - protein, 127 aa, synthetic construct
  REGION 1..127: note = Modified VH domain from a human immunoglobulin germline
  VARIANT 98..116: note = X is any residue
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCXXXXXXXXXXXXXXXXXXXWGQGTLVTVSS

SEQ ID NO: 211 - protein, 118 aa, synthetic construct
  REGION 1..118: note = Modified VH domain from a human immunoglobulin germline
  VARIANT 98..107: note = X is any residue
ELQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCXXXXXXXXXXWGQGTLVTVSS

SEQ ID NO: 212 - protein, 118 aa, synthetic construct
  REGION 1..118: note = Modified VH domain from a human immunoglobulin germline
  VARIANT 98..107: note = X is any residue
QLQLQESGPQLVKPSETLSLTCTVSGGSISSSSYYWGWIRQPPGKGLEWIGSIYYSGSTYYNPSLKSRVTISVDTSKNQFSLKLSSVTAADTAVYYCXXXXXXXXXXWGQGTLVTVSS

SEQ ID NO: 213 - protein, 118 aa, synthetic construct
  REGION 1..118: note = Modified VH domain from a human immunoglobulin germline
  VARIANT 98..107: note = X is any residue
QLQLQESGPTLVKPSETLSLTCTVSGGSISSSSYYWGWIRQPPGKGLEWIGSIYYSGSTYYNPSLKSRVTISVDTSKNQFSLKLSSVTAADTAVYYCXXXXXXXXXXWGQGTLVTVSS

SEQ ID NO: 214 - protein, 118 aa, synthetic construct
  REGION 1..118: note = Modified VH domain from a human immunoglobulin germline
  VARIANT 98..107: note = X is any residue
QLQLQESGPGLVKPGETLSLTCTVSGGSISSSSYYWGWIRQPPGKGLEWIGSIYYSGSTYYNPSLKSRVTISVDTSKNQFSLKLSSVTAADTAVYYCXXXXXXXXXXWGQGTLVTVSS

SEQ ID NO: 215 - protein, 118 aa, synthetic construct
  REGION 1..118: note = Modified VH domain from a human immunoglobulin germline
  VARIANT 98..107: note = X is any residue
QLQLQESGPGLVKPSETLILTCTVSGGSISSSSYYWGWIRQPPGKGLEWIGSIYYSGSTYYNPSLKSRVTISVDTSKNQFSLKLSSVTAADTAVYYCXXXXXXXXXXWGQGTLVTVSS

SEQ ID NO: 216 - protein, 118 aa, synthetic construct
  REGION 1..118: note = Modified VH domain from a human immunoglobulin germline
  VARIANT 98..107: note = X is any residue
QLQLQESGPGLVKPSETLSLTCTVSGGSISSSSYYWGWIRQPPGKGLEWIGSIYYSGSTYYNPSLKSRVTISVDTSKNQFSLKLSNVTAADTAVYYCXXXXXXXXXXWGQGTLVTVSS

SEQ ID NO: 217 - protein, 118 aa, synthetic construct
  REGION 1..118: note = Modified VH domain from a human immunoglobulin germline
  VARIANT 98..107: note = X is any residue
QLQLQESGPGLVKPSETLSLTCTVSGGSISSSSYYWGWIRQPPGKGLEWIGSIYYSGSTYYNPSLKSRVTISVDTSKNQFSLKLSSVTPADTAVYYCXXXXXXXXXXWGQGTLVTVSS

SEQ ID NO: 218 - protein, 120 aa, synthetic construct
  REGION 1..120: note = Modified VH domain from a human immunoglobulin germline
  VARIANT 97..109: note = X is any residue
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCXXXXXXXXXXXXXWGQGTLVTVSS

SEQ ID NO: 219 - protein, 120 aa, synthetic construct
  REGION 1..120: note = Modified VH domain from a human immunoglobulin germline
  VARIANT 97..109: note = X is any residue
QVQLVQSGAEVKKPGACVKVSCKASGYTFTSYDINWVRRATGQGLEWIGWMNPNSGNTGYAQKFQGRVTMTRNTSISTAYMELCSLRSEDTAVYYCXXXXXXXXXXXXXWGQGTLVTVSS

SEQ ID NO: 220 - protein, 120 aa, synthetic construct
  REGION 1..120: note = Modified VH domain from a human immunoglobulin germline
  VARIANT 97..109: note = X is any residue
QVQLVQSGAEVKKPGDCVKVSCKASGYTFTSYDINWVRRATGQGLEWIGWMNPNSGNTGYAQKFQGRVTMTRNTSISTAYMELCSLRSEDTAVYYCXXXXXXXXXXXXXWGQGTLVTVSS

SEQ ID NO: 221 - protein, 122 aa, synthetic construct; identical to SEQ ID NO: 87
  REGION 1..122: note = Modified VH domain from a human immunoglobulin germline
  VARIANT 97..111: note = X is any residue

SEQ ID NO: 222 - protein, 122 aa, synthetic construct
  REGION 1..122: note = Modified VH domain from a human immunoglobulin germline
  VARIANT 97..111: note = X is any residue
EVQLVQSGAEVKKPGACVKISCKVSGYTFTDYYMHWVQRAPGKGLEWIGLVDPEDGETIYAEKFQGRVTITADTSTDTAYMELCSLRSEDTAVYYCXXXXXXXXXXXXXXXWGQGTLVTVSS

SEQ ID NO: 223 - protein, 122 aa, synthetic construct
  REGION 1..122: note = Modified VH domain from a human immunoglobulin germline
  VARIANT 97..111: note = X is any residue
EVQLVQSGAEVKKPGDCVKISCKVSGYTFTDYYMHWVQRAPGKGLEWIGLVDPEDGETIYAEKFQGRVTITADTSTDTAYMELCSLRSEDTAVYYCXXXXXXXXXXXXXXXWGQGTLVTVSS

SEQ ID NO: 224 - protein, 122 aa, synthetic construct; identical to SEQ ID NO: 87
  REGION 1..122: note = Modified VH domain from a human immunoglobulin germline
  VARIANT 97..111: note = X is any residue

SEQ ID NO: 225 - protein, 122 aa, synthetic construct; identical to SEQ ID NO: 222
  REGION 1..122: note = Modified VH domain from a human immunoglobulin germline
  VARIANT 97..111: note = X is any residue

SEQ ID NO: 226 - protein, 122 aa, synthetic construct; identical to SEQ ID NO: 223
  REGION 1..122: note = Modified VH domain from a human immunoglobulin germline
  VARIANT 97..111: note = X is any residue

SEQ ID NO: 227 - protein, 122 aa, synthetic construct
  REGION 1..122: note = Modified VH domain from a human immunoglobulin germline
  VARIANT 97..111: note = X is any residue
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCXXXXXXXXXXXXXXXWGQGTLVTVSS

SEQ ID NO: 228 - protein, 122 aa, synthetic construct
  REGION 1..122: note = Modified VH domain from a human immunoglobulin germline
  VARIANT 97..111: note = X is any residue
QVQLVESGGGLVKPGGSLRLSCCASGFTFSDYYMSWIRRAPGKGLEWVAYISSSGSTIYYADSVKGRFTISRDNEKNCLYLQMNSLRAEDTAVYYCXXXXXXXXXXXXXXXWGQGTLVTVSS

SEQ ID NO: 229 - protein, 122 aa, synthetic construct
  REGION 1..122: note = Modified VH domain from a human immunoglobulin germline
  VARIANT 97..111: note = X is any residue
QVQLVESGGGLVKPGGSLRLSCCASGFTFSDYYMSWIRRAPGKGLEWVAYISSSGSTIYYADSVKGRFTISRDNAKNCLYLQMNSLREEDTAVYYCXXXXXXXXXXXXXXXWGQGTLVTVSS

SEQ ID NO: 230 - protein, 119 aa, synthetic construct
  REGION 1..119: note = Modified VH domain from a human immunoglobulin germline
  VARIANT 99..108: note = X is any residue
EVQLVESGGGLVKPGGSLRLSCCASGFTFSNAWMSWVRRAPGKGLEWVARIKSKTDGGTTDYAAPVKGRFTISRDDEKNCLYLQMNSLKTEDTAVYYCXXXXXXXXXXWGQGTLVTVSS

SEQ ID NO: 231 - protein, 119 aa, synthetic construct
  REGION 1..119: note = Modified VH domain from a human immunoglobulin germline
  VARIANT 99..108: note = X is any residue
EVQLVESGGGLVKPGGSLRLSCCASGFTFSNAWMSWVRRAPGKGLEWVARIKSKTDGGTTDYAAPVKGRFTISRDDSKNCLYLQMNSLKEEDTAVYYCXXXXXXXXXXWGQGTLVTVSS

SEQ ID NO: 232 - protein, 118 aa, synthetic construct; identical to SEQ ID NO: 123
  REGION 1..118: note = Modified VH domain from a human immunoglobulin germline
  VARIANT 97..107: note = X is any residue

SEQ ID NO: 233 - protein, 118 aa, synthetic construct
  REGION 1..118: note = Modified VH domain from a human immunoglobulin germline
  VARIANT 97..107: note = X is any residue
EVQLVESGGGVVRPGGSLRLSCCASGFTFDDYGMSWVRRAPGKGLEWVAGINWNGGSTGYADSVKGRFTISRDNEKNCLYLQMNSLRAEDTALYHCXXXXXXXXXXXWGQGTMVTVSS

SEQ ID NO: 234 - protein, 118 aa, synthetic construct
  REGION 1..118: note = Modified VH domain from a human immunoglobulin germline
  VARIANT 97..107: note = X is any residue
EVQLVESGGGVVRPGGSLRLSCCASGFTFDDYGMSWVRRAPGKGLEWVAGINWNGGSTGYADSVKGRFTISRDNAKNCLYLQMNSLREEDTALYHCXXXXXXXXXXXWGQGTMVTVSS

SEQ ID NO: 235 - protein, 123 aa, synthetic construct
  REGION 1..123: note = Modified VH domain from a human immunoglobulin germline
  VARIANT 97..112: note = X is any residue
EVQLVESGGGVVRPGGSLRLSCAASGFTFDDYGMSWVRQAPGKGLEWVSGINWNGGSTGYADSVKGRFTISRDNAKNSLYLQMNSLRAEDTALYHCXXXXXXXXXXXXXXXXWGQGTMVTVSS

SEQ ID NO: 236 - protein, 123 aa, synthetic construct
  REGION 1..123: note = Modified VH domain from a human immunoglobulin germline
  VARIANT 97..112: note = X is any residue
EVQLVESGGGVVRPGGSLRLSCCASGFTFDDYGMSWVRRAPGKGLEWVAGINWNGGSTGYADSVKGRFTISRDNEKNCLYLQMNSLRAEDTALYHCXXXXXXXXXXXXXXXXWGQGTMVTVSS

SEQ ID NO: 237 - protein, 123 aa, synthetic construct
  REGION 1..123: note = Modified VH domain from a human immunoglobulin germline
  VARIANT 97..112: note = X is any residue
EVQLVESGGGVVRPGGSLRLSCCASGFTFDDYGMSWVRRAPGKGLEWVAGINWNGGSTGYADSVKGRFTISRDNAKNCLYLQMNSLREEDTALYHCXXXXXXXXXXXXXXXXWGQGTMVTVSS

SEQ ID NO: 238 - protein, 122 aa, synthetic construct
  REGION 1..122: note = Modified VH domain from a human immunoglobulin germline
  VARIANT 97..111: note = X is any residue
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCXXXXXXXXXXXXXXXWGQGTLVTVSS

SEQ ID NO: 239 - protein, 122 aa, synthetic construct
  REGION 1..122: note = Modified VH domain from a human immunoglobulin germline
  VARIANT 97..111: note = X is any residue
EVQLVESGGGLVQPGGSLRLSCCASGFTFSSYSMNWVRRAPGKGLEWVAYISSSSSTIYYADSVKGRFTISRDNEKNCLYLQMNSLRAEDTAVYYCXXXXXXXXXXXXXXXWGQGTLVTVSS

SEQ ID NO: 240 - protein, 122 aa, synthetic construct
  REGION 1..122: note = Modified VH domain from a human immunoglobulin germline
  VARIANT 97..111: note = X is any residue
EVQLVESGGGLVQPGGSLRLSCCASGFTFSSYSMNWVRRAPGKGLEWVAYISSSSSTIYYADSVKGRFTISRDNAKNCLYLQMNSLREEDTAVYYCXXXXXXXXXXXXXXXWGQGTLVTVSS

SEQ ID NO: 241 - protein, 127 aa, synthetic construct; identical to SEQ ID NO: 210
  REGION 1..127: note = Modified VH domain from a human immunoglobulin germline
  VARIANT 98..116: note = X is any residue

SEQ ID NO: 242 - protein, 127 aa, synthetic construct
  REGION 1..127: note = Modified VH domain from a human immunoglobulin germline
  VARIANT 98..116: note = X is any residue
QLQLQESGPTLVKPSECLSLTCTVSGGSISSSSYYWGWIRRPPGKGLEWIASIYYSGSTYYNPSLKSRVTISVDTSKNQFSLKLCSVTAADTAVYYCXXXXXXXXXXXXXXXXXXXWGQGTLVTVSS

SEQ ID NO: 243 - protein, 127 aa, synthetic construct
  REGION 1..127: note = Modified VH domain from a human immunoglobulin germline
  VARIANT 98..116: note = X is any residue
QLQLQESGPGLVKPSETLSLTCCVSGGSISSSSYYWGWIRRPPGKGLEWIASIYYSGSTYYNPSLKSRVTISVDTSKNCFSLKLSSVTAADTAVYYCXXXXXXXXXXXXXXXXXXXWGQGTLVTVSS

SEQ ID NO: 244 - protein, 98 aa, synthetic construct
  REGION 1..98: note = Modified VH domain from a human immunoglobulin germline
QVQLVQSGAEVKKPGDCVKVSCKASGYTFTGYYMHWVRRAPGQGLEWIGRINPNSGGTNYAQKFQGRVTSTRDTSISTAYMELCRLRSDDTVVYYCAR

SEQ ID NO: 245 - protein, 98 aa, synthetic construct
  REGION 1..98: note = Modified VH domain from a human immunoglobulin germline
QVQLVQSGAEVKKPGDCVKVSCKASGYTFTSYAMHWVRRAPGQRLEWIGWINAGNGNTKYSQKFQGRVTITRDTSASTAYMELCSLRSEDTAVYYCAR

SEQ ID NO: 246 - protein, 98 aa, synthetic construct
  REGION 1..98: note = Modified VH domain from a human immunoglobulin germline
QVQLVQSGAEVKKPGDCVKVSCKASGYTFTSYDINWVRRATGQGLEWIGWMNPNSGNTGYAQKFQGRVTMTRNTSISTAYMELCSLRSEDTAVYYCAR

SEQ ID NO: 247 - protein, 98 aa, synthetic construct
  REGION 1..98: note = Modified VH domain from a human immunoglobulin germline
QVQLVQSGAEVKKPGDCVKVSCKASGYTFTSYGISWVRRAPGQGLEWIGWISAYNGNTNYAQKLQGRVTMTTDTSTSTAYMELCSLRSDDTAVYYCAR

SEQ ID NO: 248 - protein, 98 aa, synthetic construct
  REGION 1..98: note = Modified VH domain from a human immunoglobulin germline
QVQLVQSGAEVKKPGDCVKVSCKVSGYTLTELSMHWVRRAPGKGLEWIGGFDPEDGETIYAQKFQGRVTMTEDTSTDTAYMELCSLRSEDTAVYYCAT

SEQ ID NO: 249 - protein, 98 aa, synthetic construct
  REGION 1..98: note = Modified VH domain from a human immunoglobulin germline
QMQLVQSGAEVKKTGDCVKVSCKASGYTFTYRYLHWVRRAPGQALEWIGWITPFNGNTNYAQKFQDRVTITRDRSMSTAYMELCSLRSEDTAMYYCAR

SEQ ID NO: 250 - protein, 98 aa, synthetic construct
  REGION 1..98: note = Modified VH domain from a human immunoglobulin germline
QVQLVQSGAEVKKPGDCVKVSCKASGYTFTSYYMHWVRRAPGQGLEWIGIINPSGGSTSYAQKFQGRVTMTRDTSTSTVYMELCSLRSEDTAVYYCAR

SEQ ID NO: 251 - protein, 98 aa, synthetic construct
  REGION 1..98: note = Modified VH domain from a human immunoglobulin germline
QMQLVQSGPEVKKPGDCVKVSCKASGFTFTSSAVQWVRRARGQRLEWIGWIVVGSGNTNYAQKFQERVTITRDMSTSTAYMELCSLRSEDTAVYYCAA

SEQ ID NO: 252 - protein, 98 aa, synthetic construct
  REGION 1..98: note = Modified VH domain from a human immunoglobulin germline
QVQLVQSGAEVKKPGDCVKVSCKASGGTFSSYAISWVRRAPGQGLEWIGGIIPIFGTANYAQKFQGRVTITADESTSTAYMELCSLRSEDTAVYYCAR

SEQ ID NO: 253 - protein, 98 aa, synthetic construct
  REGION 1..98: note = Modified VH domain from a human immunoglobulin germline
EVQLVQSGAEVKKPGDCVKISCKVSGYTFTDYYMHWVQRAPGKGLEWIGLVDPEDGETIYAEKFQGRVTITADTSTDTAYMELCSLRSEDTAVYYCAT

SEQ ID NO: 254 - protein, 98 aa, synthetic construct
  REGION 1..98: note = Modified VH domain from a human immunoglobulin germline
EVQLVESGGGLVQPGGSLRLSCCASGFTFSSYWMSWVRRAPGKGLEWVANIKQDGSEKYYVDSVKGRFTISRDNAKNCLYLQMNSLREEDTAVYYCAR

SEQ ID NO: 255 - protein, 99 aa, synthetic construct
  REGION 1..99: note = Modified VH domain from a human immunoglobulin germline
EVQLVESGGGLVQPGRSLRLSCCASGFTFDDYAMHWVRRAPGKGLEWVAGISWNSGSIGYADSVKGRFTISRDNAKNCLYLQMNSLREEDTALYYCAKD

SEQ ID NO: 256 - protein, 98 aa, synthetic construct
  REGION 1..98: note = Modified VH domain from a human immunoglobulin germline
QVQLVESGGGLVKPGGSLRLSCCASGFTFSDYYMSWIRRAPGKGLEWVAYISSSGSTIYYADSVKGRFTISRDNAKNCLYLQMNSLREEDTAVYYCAR

SEQ ID NO: 257 - protein, 97 aa, synthetic construct
  REGION 1..97: note = Modified VH domain from a human immunoglobulin germline
EVQLVESGGGLVQPGGSLRLSCCASGFTFSSYDMHWVRRATGKGLEWVAAIGTAGDTYYPGSVKGRFTISRENAKNCLYLQMNSLREGDTAVYYCAR

SEQ ID NO: 258 - protein, 100 aa, synthetic construct
  REGION 1..100: note = Modified VH domain from a human immunoglobulin germline
EVQLVESGGGLVKPGGSLRLSCCASGFTFSNAWMSWVRRAPGKGLEWVARIKSKTDGGTTDYAAPVKGRFTISRDDSKNCLYLQMNSLKEEDTAVYYCTT

SEQ ID NO: 259 - protein, 98 aa, synthetic construct
  REGION 1..98: note = Modified VH domain from a human immunoglobulin germline
EVQLVESGGGVVRPGGSLRLSCCASGFTFDDYGMSWVRRAPGKGLEWVAGINWNGGSTGYADSVKGRFTISRDNAKNCLYLQMNSLREEDTALYHCAR

SEQ ID NO: 260 - protein, 98 aa, synthetic construct
  REGION 1..98: note = Modified VH domain from a human immunoglobulin germline
EVQLVESGGGLVKPGGSLRLSCCASGFTFSSYSMNWVRRAPGKGLEWVASISSSSSYIYYADSVKGRFTISRDNAKNCLYLQMNSLREEDTAVYYCAR

SEQ ID NO: 261 - protein, 98 aa, synthetic construct
  REGION 1..98: note = Modified VH domain from a human immunoglobulin germline
EVQLLESGGGLVQPGGSLRLSCCASGFTFSSYAMSWVRRAPGKGLEWVAAISGSGGSTYYADSVKGRFTISRDNSKNCLYLQMNSLREEDTAVYYCAK

SEQ ID NO: 262 - protein, 98 aa, synthetic construct
  REGION 1..98: note = Modified VH domain from a human immunoglobulin germline
QVQLVESGGGVVQPGRSLRLSCCASGFTFSSYAMHWVRRAPGKGLEWVAVISYDGSNKYYADSVKGRFTISRDNSKNCLYLQMNSLREEDTAVYYCAR

SEQ ID NO: 263 - protein, 98 aa, synthetic construct
  REGION 1..98: note = Modified VH domain from a human immunoglobulin germline
QVQLVESGGGVVQPGRSLRLSCCASGFTFSSYGMHWVRRAPGKGLEWVAVIWYDGSNKYYADSVKGRFTISRDNSKNCLYLQMNSLREEDTAVYYCAR

SEQ ID NO: 264 - protein, 99 aa, synthetic construct
  REGION 1..99: note = Modified VH domain from a human immunoglobulin germline
EVQLVESGGVVVQPGGSLRLSCCASGFTFDDYTMHWVRRAPGKGLEWVALISWDGGSTYYADSVKGRFTISRDNSKNCLYLQMNSLREEDTALYYCAKD

SEQ ID NO: 265 - protein, 98 aa, synthetic construct
  REGION 1..98: note = Modified VH domain from a human immunoglobulin germline
EVQLVESGGGLVQPGGSLRLSCCASGFTFSSYSMNWVRRAPGKGLEWVAYISSSSSTIYYADSVKGRFTISRDNAKNCLYLQMNSLREEDTAVYYCAR

SEQ ID NO: 266 - protein, 100 aa, synthetic construct
  REGION 1..100: note = Modified VH domain from a human immunoglobulin germline
EVQLVESGGGLVQPGRSLRLSCCASGFTFGDYAMSWFRRAPGKGLEWVAFIRSKAYGGTTEYTASVKGRFTISRDGSKSCAYLQMNSLKEEDTAVYYCTR

SEQ ID NO: 267 - protein, 97 aa, synthetic construct
  REGION 1..97: note = Modified VH domain from a human immunoglobulin germline
EVQLVESGGGLIQPGGSLRLSCCASGFTVSSNYMSWVRRAPGKGLEWVAVIYSGGSTYYADSVKGRFTISRDNSKNCLYLQMNSLREEDTAVYYCAR

SEQ ID NO: 268 - protein, 98 aa, synthetic construct
  REGION 1..98: note = Modified VH domain from a human immunoglobulin germline
EVQLVESGGGLVQPGGSLRLSCCASGFTFSSYAMHWVRRAPGKGLEYVAAISSNGGSTYYANSVKGRFTISRDNSKNCLYLQMGSLREEDMAVYYCAR

SEQ ID NO: 269 - protein, 97 aa, synthetic construct
  REGION 1..97: note = Modified VH domain from a human immunoglobulin germline
EVQLVESGGGLVQPGGSLRLSCCASGFTVSSNYMSWVRRAPGKGLEWVAVIYSGGSTYYADSVKGRFTISRDNSKNCLYLQMNSLREEDTAVYYCAR

SEQ ID NO: 270 - protein, 100 aa, synthetic construct
  REGION 1..100: note = Modified VH domain from a human immunoglobulin germline
EVQLVESGGGLVQPGGSLRLSCCASGFTFSDHYMDWVRRAPGKGLEWVARTRNKANSYTTEYAASVKGRFTISRDDSKNCLYLQMNSLKEEDTAVYYCAR

SEQ ID NO: 271 - protein, 100 aa, synthetic construct
  REGION 1..100: note = Modified VH domain from a human immunoglobulin germline
EVQLVESGGGLVQPGGSLKLSCCASGFTFSGSAMHWVRRASGKGLEWVARIRSKANSYATAYAASVKGRFTISRDDSKNCAYLQMNSLKEEDTAVYYCTR

SEQ ID NO: 272 - protein, 98 aa, synthetic construct
  REGION 1..98: note = Modified VH domain from a human immunoglobulin germline
EVQLVESGGGLVQPGGSLRLSCCASGFTFSSYWMHWVRRAPGKGLVWVARINSDGSSTSYADSVKGRFTISRDNAKNCLYLQMNSLREEDTAVYYCAR

SEQ ID NO: 273 - protein, 96 aa, synthetic construct
  REGION 1..96: note = Modified VH domain from a human immunoglobulin germline
EVQLVESRGVLVQPGGSLRLSCCASGFTVSSNEMSWVRRAPGKGLEWVASISGGSTYYADSRKGRFTISRDNSKNCLHLQMNSLREEDTAVYYCKK

SEQ ID NO: 274 - protein, 98 aa, synthetic construct
  REGION 1..98: note = Modified VH domain from a human immunoglobulin germline
QVQLQESGPGLVKPSGTLSLTCCVSGGSISSSNWWSWVRRPPGKGLEWIAEIYHSGSTNYNPSLKSRVTISVDKSKNCFSLKLSSVTAADTAVYYCAR

SEQ ID NO: 275 - protein, 98 aa, synthetic construct
  REGION 1..98: note = Modified VH domain from a human immunoglobulin germline
QVQLQESGPGLVKPSDTLSLTCCVSGYSISSSNWWGWIRRPPGKGLEWIAYIYYSGSTYYNPSLKSRVTMSVDTSKNCFSLKLSSVTAVDTAVYYCAR

SEQ ID NO: 276 - protein, 99 aa, synthetic construct
  REGION 1..99: note = Modified VH domain from a human immunoglobulin germline
QVQLQESGPGLVKPSQTLSLTCCVSGGSISSGGYYWSWIRRPPGKGLEWIAYIYYSGSTYYNPSLKSRVTISVDTSKNCFSLKLSSVTAADTAVYYCAR

SEQ ID NO: 277 - protein, 99 aa, synthetic construct
  REGION 1..99: note = Modified VH domain from a human immunoglobulin germline
QLQLQESGSGLVKPSQTLSLTCCVSGGSISSGGYSWSWIRRPPGKGLEWIAYIYHSGSTYYNPSLKSRVTISVDRSKNCFSLKLSSVTAADTAVYYCAR

SEQ ID NO: 278 - protein, 99 aa, synthetic construct
  REGION 1..99: note = Modified VH domain from a human immunoglobulin germline
QVQLQESGPGLVKPSQTLSLTCCVSGGSISSGDYYWSWIRRPPGKGLEWIAYIYYSGSTYYNPSLKSRVTISVDTSKNCFSLKLSSVTAADTAVYYCAR

SEQ ID NO: 279 - protein, 99 aa, synthetic construct
  REGION 1..99: note = Modified VH domain from a human immunoglobulin germline
QVQLQESGPGLVKPSQTLSLTCCVSGGSISSGGYYWSWIRRHPGKGLEWIAYIYYSGSTYYNPSLKSLVTISVDTSKNCFSLKLSSVTAADTAVYYCAR

SEQ ID NO: 280 - protein, 97 aa, synthetic construct
  REGION 1..97: note = Modified VH domain from a human immunoglobulin germline
QVQLQQWGAGLLKPSETLSLTCCVYGGSFSGYYWSWIRRPPGKGLEWIAEINHSGSTNYNPSLKSRVTISVDTSKNCFSLKLSSVTAADTAVYYCAR

SEQ ID NO: 281 - protein, 99 aa, synthetic construct
  REGION 1..99: note = Modified VH domain from a human immunoglobulin germline
QLQLQESGPGLVKPSETLSLTCCVSGGSISSSSYYWGWIRRPPGKGLEWIASIYYSGSTYYNPSLKSRVTISVDTSKNCFSLKLSSVTAADTAVYYCAR

SEQ ID NO: 282 - protein, 97 aa, synthetic construct
  REGION 1..97: note = Modified VH domain from a human immunoglobulin germline
QVQLQESGPGLVKPSETLSLTCCVSGGSISSYYWSWIRRPPGKGLEWIAYIYYSGSTNYNPSLKSRVTISVDTSKNCFSLKLSSVTAADTAVYYCAR

SEQ ID NO: 283 - protein, 99 aa, synthetic construct
  REGION 1..99: note = Modified VH domain from a human immunoglobulin germline
QVQLQESGPGLVKPSETLSLTCCVSGGSVSSGSYYWSWIRRPPGKGLEWIAYIYYSGSTNYNPSLKSRVTISVDTSKNCFSLKLSSVTAADTAVYYCAR

SEQ ID NO: 284 - protein, 98 aa, synthetic construct
  REGION 1..98: note = Modified VH domain from a human immunoglobulin germline
QVQLQESGPGLVKPSETLSLTCCVSGYSISSGYYWGWIRRPPGKGLEWIASIYHSGSTYYNPSLKSRVTISVDTSKNCFSLKLSSVTAADTAVYYCAR

SEQ ID NO: 285 - protein, 98 aa, synthetic construct
  REGION 1..98: note = Modified VH domain from a human immunoglobulin germline
QVQLVQSGSELKKPGACVKVSCKASGYTFTSYAMNWVRRAPGQGLEWMGWINTNTGNPTYAQGFTGRFVFSFDTSVSTAYLQICSLKAEDTAVYYCAR

SEQ ID NO: 286 - protein, 118 aa, synthetic construct
  REGION 1..118: note = Modified VH domain from a human immunoglobulin germline
  VARIANT 97..107: note = X is any residue
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCXXXXXXXXXXXWGQGTLVTVSS

SEQ ID NO: 287 - protein, 118 aa, synthetic construct
  REGION 1..118: note = Modified VH domain from a human immunoglobulin germline
  VARIANT 97..107: note = X is any residue
QVQLVQSGAEVKKPGDCVKVSCKASGYTFTSYAMHWVRRAPGQRLEWIGWINAGNGNTKYSQKFQGRVTITRDTSASTAYMELCSLRSEDTAVYYCXXXXXXXXXXXWGQGTLVTVSS

SEQ ID NO: 288 - protein, 118 aa, synthetic construct
  REGION 1..118: note = Modified VH domain from a human immunoglobulin germline
  VARIANT 97..107: note = X is any residue
QVQLVQSGAEVKKPGDCVKVSCKASGYTFTSYGISWVRRAPGQGLEWIGWISAYNGNTNYAQKLQGRVTMTTDTSTSTAYMELCSLRSDDTAVYYCXXXXXXXXXXXWGQGTLVTVSS

SEQ ID NO: 289 - protein, 124 aa, synthetic construct
  REGION 1..124: note = Modified VH domain from a human immunoglobulin germline
  VARIANT 97..113: note = X is any residue
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCXXXXXXXXXXXXXXXXXWGQGTLVTVSS

SEQ ID NO: 290 - protein, 124 aa, synthetic construct
  REGION 1..124: note = Modified VH domain from a human immunoglobulin germline
  VARIANT 97..113: note = X is any residue
QVQLVQSGAEVKKPGDCVKVSCKVSGYTLTELSMHWVRRAPGKGLEWIGGFDPEDGETIYAQKFQGRVTMTEDTSTDTAYMELCSLRSEDTAVYYCXXXXXXXXXXXXXXXXXWGQGTLVTVSS

SEQ ID NO: 291 - protein, 118 aa, synthetic construct
  REGION 1..118: note = Modified VH domain from a human immunoglobulin germline
  VARIANT 97..107: note = X is any residue
QMQLVQSGAEVKKTGSSVKVSCKASGYTFTYRYLHWVRQAPGQALEWMGWITPFNGNTNYAQKFQDRVTITRDRSMSTAYMELSSLRSEDTAMYYCXXXXXXXXXXXWGQGTLVTVSS

SEQ ID NO: 292 - protein, 118 aa, synthetic construct
  REGION 1..118: note = Modified VH domain from a human immunoglobulin germline
  VARIANT 97..107: note = X is any residue
QMQLVQSGAEVKKTGDCVKVSCKASGYTFTYRYLHWVRRAPGQALEWIGWITPFNGNTNYAQKFQDRVTITRDRSMSTAYMELCSLRSEDTAMYYCXXXXXXXXXXXWGQGTLVTVSS

SEQ ID NO: 293 - protein, 118 aa, synthetic construct
  REGION 1..118: note = Modified VH domain from a human immunoglobulin germline
  VARIANT 97..107: note = X is any residue
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCXXXXXXXXXXXWGQGTLVTVSS

SEQ ID NO: 294 - protein, 118 aa, synthetic construct
  REGION 1..118: note = Modified VH domain from a human immunoglobulin germline
  VARIANT 97..107: note = X is any residue
QVQLVQSGAEVKKPGDCVKVSCKASGYTFTSYYMHWVRRAPGQGLEWIGIINPSGGSTSYAQKFQGRVTMTRDTSTSTVYMELCSLRSEDTAVYYCXXXXXXXXXXXWGQGTLVTVSS

SEQ ID NO: 295 - protein, 118 aa, synthetic construct
  REGION 1..118: note = Modified VH domain from a human immunoglobulin germline
  VARIANT 97..107: note = X is any residue
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCXXXXXXXXXXXWGQGTLVTVSS

SEQ ID NO: 296 - protein, 118 aa, synthetic construct
  REGION 1..118: note = Modified VH domain from a human immunoglobulin germline
  VARIANT 97..107: note = X is any residue
QMQLVQSGPEVKKPGDCVKVSCKASGFTFTSSAVQWVRRARGQRLEWIGWIVVGSGNTNYAQKFQERVTITRDMSTSTAYMELCSLRSEDTAVYYCXXXXXXXXXXXWGQGTLVTVSS

SEQ ID NO: 297 - protein, 118 aa, synthetic construct
  REGION 1..118: note = Modified VH domain from a human immunoglobulin germline
  VARIANT 97..107: note = X is any residue
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCXXXXXXXXXXXWGQGTLVTVSS

SEQ ID NO: 298 - protein, 118 aa, synthetic construct
  REGION 1..118: note = Modified VH domain from a human immunoglobulin germline
  VARIANT 97..107: note = X is any residue
QVQLVQSGAEVKKPGDCVKVSCKASGGTFSSYAISWVRRAPGQGLEWIGGIIPIFGTANYAQKFQGRVTITADESTSTAYMELCSLRSEDTAVYYCXXXXXXXXXXXWGQGTLVTVSS

SEQ ID NO: 299 - protein, 120 aa, synthetic construct
  REGION 1..120: note = Modified VH domain from a human immunoglobulin germline
  VARIANT 97..109: note = X is any residue
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCXXXXXXXXXXXXXWGQGTLVTVSS

SEQ ID NO: 300 - protein, 120 aa, synthetic construct
  REGION 1..120: note = Modified VH domain from a human immunoglobulin germline
  VARIANT 97..109: note = X is any residue
EVQLVESGGGLVQPGGSLRLSCCASGFTFSSYWMSWVRRAPGKGLEWVANIKQDGSEKYYVDSVKGRFTISRDNEKNCLYLQMNSLRAEDTAVYYCXXXXXXXXXXXXXWGQGTLVTVSS

SEQ ID NO: 301 - protein, 125 aa, synthetic construct
  REGION 1..125: note = Modified VH domain from a human immunoglobulin germline
  VARIANT 97..114: note = X is any residue
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCXXXXXXXXXXXXXXXXXXWGQGTMVTVSS

SEQ ID NO: 302 - protein, 125 aa, synthetic construct
  REGION 1..125: note = Modified VH domain from a human immunoglobulin germline
  VARIANT 97..114: note = X is any residue
EVQLVESGGGLVQPGRSLRLSCCASGFTFDDYAMHWVRRAPGKGLEWVAGISWNSGSIGYADSVKGRFTISRDNEKNCLYLQMNSLRAEDTALYYCXXXXXXXXXXXXXXXXXXWGQGTMVTVSS

SEQ ID NO: 303 - protein, 121 aa, synthetic construct
  REGION 1..121: note = Modified VH domain from a human immunoglobulin germline
  VARIANT 97..110: note = X is any residue
QVQLVESGGGLVKPGGSLRLSCCASGFTFSDYYMSWIRRAPGKGLEWVAYISSSGSTIYYADSVKGRFTISRDNEKNCLYLQMNSLRAEDTAVYYCXXXXXXXXXXXXXXWGQGTLVTVSS

SEQ ID NO: 304 - protein, 124 aa, synthetic construct
  REGION 1..124: note = Modified VH domain from a human immunoglobulin germline
  VARIANT 96..113: note = X is any residue
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYCXXXXXXXXXXXXXXXXXXWGQGTMVTVSS

SEQ ID NO: 305 - protein, 124 aa, synthetic construct
  REGION 1..124: note = Modified VH domain from a human immunoglobulin germline
  VARIANT 96..113: note = X is any residue
EVQLVESGGGLVQPGGSLRLSCCASGFTFSSYDMHWVRRATGKGLEWVAAIGTAGDTYYPGSVKGRFTISRENEKNCLYLQMNSLRAGDTAVYYCXXXXXXXXXXXXXXXXXXWGQGTMVTVSS

SEQ ID NO: 306 - protein, 121 aa, synthetic construct
  REGION 1..121: note = Modified VH domain from a human immunoglobulin germline
  VARIANT 97..110: note = X is any residue
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCXXXXXXXXXXXXXXWGQGTLVTVSS

SEQ ID NO: 307 - protein, 121 aa, synthetic construct
  REGION 1..121: note = Modified VH domain from a human immunoglobulin germline
  VARIANT 97..110: note = X is any residue
EVQLLESGGGLVQPGGSLRLSCCASGFTFSSYAMSWVRRAPGKGLEWVAAISGSGGSTYYADSVKGRFTISRDNEKNCLYLQMNSLRAEDTAVYYCXXXXXXXXXXXXXXWGQGTLVTVSS

SEQ ID NO: 308 - protein, 118 aa, synthetic construct
  REGION 1..118: note = Modified VH domain from a human immunoglobulin germline
  VARIANT 97..107: note = X is any residue
QVQLVESGGGVVQPGRSLRLSCCASGFTFSSYAMHWVRRAPGKGLEWVAVISYDGSNKYYADSVKGRFTISRDNEKNCLYLQMNSLRAEDTAVYYCXXXXXXXXXXXWGRGTLVTVSS

SEQ ID NO: 309 - protein, 118 aa, synthetic construct
  REGION 1..118: note = Modified VH domain from a human immunoglobulin germline
  VARIANT 97..107: note = X is any residue
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCXXXXXXXXXXXWGRGTLVTVSS

SEQ ID NO: 310 - protein, 118 aa, synthetic construct
  REGION 1..118: note = Modified VH domain from a human immunoglobulin germline
  VARIANT 97..107: note = X is any residue
QVQLVESGGGVVQPGRSLRLSCCASGFTFSSYGMHWVRRAPGKGLEWVAVIWYDGSNKYYADSVKGRFTISRDNEKNCLYLQMNSLRAEDTAVYYCXXXXXXXXXXXWGRGTLVTVSS

SEQ ID NO: 311 - protein, 125 aa, synthetic construct
  REGION 1..125: note = Modified VH domain from a human immunoglobulin germline
  VARIANT 97..114: note = X is any residue
EVQLVESGGVVVQPGGSLRLSCAASGFTFDDYTMHWVRQAPGKGLEWVSLISWDGGSTYYADSVKGRFTISRDNSKNSLYLQMNSLRTEDTALYYCXXXXXXXXXXXXXXXXXXWGQGTMVTVSS

SEQ ID NO: 312 - protein, 125 aa, synthetic construct
  REGION 1..125: note = Modified VH domain from a human immunoglobulin germline
  VARIANT 97..114: note = X is any residue
EVQLVESGGVVVQPGGSLRLSCCASGFTFDDYTMHWVRRAPGKGLEWVALISWDGGSTYYADSVKGRFTISRDNEKNCLYLQMNSLRTEDTALYYCXXXXXXXXXXXXXXXXXXWGQGTMVTVSS

SEQ ID NO: 313 - protein, 126 aa, synthetic construct
  REGION 1..126: note = Modified VH domain from a human immunoglobulin germline
  VARIANT 97..115: note = X is any residue
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCXXXXXXXXXXXXXXXXXXXWGRGTLVTVSS

SEQ ID NO: 314 - protein, 126 aa, synthetic construct
  REGION 1..126: note = Modified VH domain from a human immunoglobulin germline
  VARIANT 97..115: note = X is any residue
EVQLVESGGGLVQPGGSLRLSCCASGFTFSSYSMNWVRRAPGKGLEWVAYISSSSSTIYYADSVKGRFTISRDNEKNCLYLQMNSLRAEDTAVYYCXXXXXXXXXXXXXXXXXXXWGRGTLVTVSS

SEQ ID NO: 315 - protein, 123 aa, synthetic construct
  REGION 1..123: note = Modified VH domain from a human immunoglobulin germline
  VARIANT 96..112: note = X is any residue
EVQLVESGGGLIQPGGSLRLSCCASGFTVSSNYMSWVRQAPGKGLEWVAVIYSGGSTYYADSVKGRFTISRDNEKNCLYLQMNSLRAEDTAVYYCXXXXXXXXXXXXXXXXXWGRGTLVTVSS

SEQ ID NO: 316 - protein, 118 aa, synthetic construct
  REGION 1..118: note = Modified VH domain from a human immunoglobulin germline
  VARIANT 97..107: note = X is any residue
EVQLVESGGGLVQPGGSLRLSCAASGFTFSSYAMHWVRQAPGKGLEYVSAISSNGGSTYYANSVKGRFTISRDNSKNTLYLQMGSLRAEDMAVYYCXXXXXXXXXXXWGQGTLVTVSS

SEQ ID NO: 317 - protein, 118 aa, synthetic construct
  REGION 1..118: note = Modified VH domain from a human immunoglobulin germline
  VARIANT 97..107: note = X is any residue
EVQLVESGGGLVQPGGSLRLSCCASGFTFSSYAMHWVRRAPGKGLEYVAAISSNGGSTYYANSVKGRFTISRDNEKNCLYLQMGSLRAEDMAVYYCXXXXXXXXXXXWGQGTLVTVSS

SEQ ID NO: 318 - protein, 117 aa, synthetic construct
  REGION 1..117: note = Modified VH domain from a human immunoglobulin germline
  VARIANT 96..106: note = X is any residue
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCXXXXXXXXXXXWGRGTLVTVSS

SEQ ID NO: 319 - protein, 117 aa, synthetic construct
  REGION 1..117: note = Modified VH domain from a human immunoglobulin germline
  VARIANT 96..106: note = X is any residue
EVQLVESGGGLVQPGGSLRLSCCASGFTVSSNYMSWVRRAPGKGLEWVAVIYSGGSTYYADSVKGRFTISRDNEKNCLYLQMNSLRAEDTAVYYCXXXXXXXXXXXWGRGTLVTVSS

SEQ ID NO: 320 - protein, 120 aa, synthetic construct
  REGION 1..120: note = Modified VH domain from a human immunoglobulin germline
  VARIANT 99..109: note = X is any residue
EVQLVESGGGLVQPGGSLRLSCAASGFTFSDHYMDWVRQAPGKGLEWVGRTRNKANSYTTEYAASVKGRFTISRDDSKNSLYLQMNSLKTEDTAVYYCXXXXXXXXXXXWGRGTLVTVSS

SEQ ID NO: 321 - protein, 120 aa, synthetic construct
  REGION 1..120: note = Modified VH domain from a human immunoglobulin germline
  VARIANT 99..109: note = X is any residue
EVQLVESGGGLVQPGGSLRLSCCASGFTFSDHYMDWVRRAPGKGLEWVARTRNKANSYTTEYAASVKGRFTISRDDEKNCLYLQMNSLKTEDTAVYYCXXXXXXXXXXXWGRGTLVTVSS

SEQ ID NO: 322 - protein, 120 aa, synthetic construct
  REGION 1..120: note = Modified VH domain from a human immunoglobulin germline
  VARIANT 99..109: note = X is any residue
EVQLVESGGGLVQPGGSLKLSCAASGFTFSGSAMHWVRQASGKGLEWVGRIRSKANSYATAYAASVKGRFTISRDDSKNTAYLQMNSLKTEDTAVYYCXXXXXXXXXXXWGRGTLVTVSS

SEQ ID NO: 323 - protein, 120 aa, synthetic construct
  REGION 1..120: note = Modified VH domain from a human immunoglobulin germline
  VARIANT 99..109: note = X is any residue
EVQLVESGGGLVQPGGSLKLSCCASGFTFSGSAMHWVRRASGKGLEWVARIRSKANSYATAYAASVKGRFTISRDDEKNCAYLQMNSLKTEDTAVYYCXXXXXXXXXXXWGRGTLVTVSS

SEQ ID NO: 324 - protein, 119 aa, synthetic construct
  REGION 1..119: note = Modified VH domain from a human immunoglobulin germline
  VARIANT 97..108: note = X is any residue
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCXXXXXXXXXXXXWGQGTLVTVSS

SEQ ID NO: 325 - protein, 119 aa, synthetic construct
  REGION 1..119: note = Modified VH domain from a human immunoglobulin germline
  VARIANT 97..108: note = X is any residue
EVQLVESGGGLVQPGGSLRLSCCASGFTFSSYWMHWVRRAPGKGLVWVARINSDGSSTSYADSVKGRFTISRDNEKNCLYLQMNSLRAEDTAVYYCXXXXXXXXXXXXWGQGTLVTVSS

SEQ ID NO: 326 - protein, 118 aa, synthetic construct
  REGION 1..118: note = Modified VH domain from a human immunoglobulin germline
  VARIANT 97..107: note = X is any residue
QVQLVESGGGVVQPGGSLRLSCAASGFTFSSYGMHWVRQAPGKGLEWVSVIYSGGSSTYYADSVKGRFTISRDNSKNTLYLQMNSLRAEDTAVYYCXXXXXXXXXXXWGRGTLVTVSS

SEQ ID NO: 327 - protein, 118 aa, synthetic construct
  REGION 1..118: note = Modified VH domain from a human immunoglobulin germline
  VARIANT 97..107: note = X is any residue
QVQLVESGGGVVQPGGSLRLSCCASGFTFSSYGMHWVRRAPGKGLEWVAVIYSGGSSTYYADSVKGRFTISRDNEKNCLYLQMNSLRAEDTAVYYCXXXXXXXXXXXWGRGTLVTVSS

SEQ ID NO: 328 - protein, 120 aa, synthetic construct
  REGION 1..120: note = Modified VH domain from a human immunoglobulin germline
  VARIANT 96..109: note = X is any residue
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCXXXXXXXXXXXXXXWGQGTLVTVSS

SEQ ID NO: 329 - protein, 120 aa, synthetic construct
  REGION 1..120: note = Modified VH domain from a human immunoglobulin germline
  VARIANT 96..109: note = X is any residue
QVQLQQWGATLLKPSECLSLTCAVYGGSFSGYYWSWIRQPPGKGLEWIAEINHSGSTNYNPSLKSRVTISVDTSKNQFSLKLCSVTAADTAVYYCXXXXXXXXXXXXXXWGQGTLVTVSS

SEQ ID NO: 330 - protein, 120 aa, synthetic construct
  REGION 1..120: note = Modified VH domain from a human immunoglobulin germline
  VARIANT 96..109: note = X is any residue
QVQLQQWGATLLKPSECLSLTCAVYGGSFSGYYWSWIRQPPGKGLEWIGEINHSGSTNYNPSLKSRVTISVDTSKNQFSLKLCNVTAADTAVYYCXXXXXXXXXXXXXXWGQGTLVTVSS

SEQ ID NO: 331 - protein, 120 aa, synthetic construct
  REGION 1..120: note = Modified VH domain from a human immunoglobulin germline
  VARIANT 96..109: note = X is any residue
QVQLQQWGATLLKPSECLSLTCAVYGGSFSGYYWSWIRQPPGKGLEWIGEINHSGSTNYNPSLKSRVTISVDTSKNQFSLKLCSVTPADTAVYYCXXXXXXXXXXXXXXWGQGTLVTVSS

SEQ ID NO: 332 - protein, 120 aa, synthetic construct
  REGION 1..120: note = Modified VH domain from a human immunoglobulin germline
  VARIANT 96..109: note = X is any residue
QVQLQQWGAGLLKPSECLSLTCAVYGGSFSGYYWSWIRQPPGKGLEWIAEINHSGSTNYNPSLKSRVTISVDTSKNQFSLKLCNVTAADTAVYYCXXXXXXXXXXXXXXWGQGTLVTVSS

SEQ ID NO: 333 - protein, 120 aa, synthetic construct
  REGION 1..120: note = Modified VH domain from a human immunoglobulin germline
  VARIANT 96..109: note = X is any residue
QVQLQQWGAGLLKPSECLSLTCAVYGGSFSGYYWSWIRQPPGKGLEWIAEINHSGSTNYNPSLKSRVTISVDTSKNQFSLKLCSVTPADTAVYYCXXXXXXXXXXXXXXWGQGTLVTVSS

SEQ ID NO: 334 - protein, 120 aa, synthetic construct
  REGION 1..120: note = Modified VH domain from a human immunoglobulin germline
  VARIANT 96..109: note = X is any residue
QVQLQQWGAGLLKPSECLSLTCAVYGGSFSGYYWSWIRQPPGKGLEWIGEINHSGSTNYNPSLKSRVTISVDTSKNQFSLKLCNVTPADTAVYYCXXXXXXXXXXXXXXWGQGTLVTVSS

SEQ ID NO: 335 - protein, 120 aa, synthetic construct
  REGION 1..120: note = Modified VH domain from a human immunoglobulin germline
  VARIANT 96..109: note = X is any residue
QVQLQQWGATLLKPSECLSLTCQVYGGSFSGYYWSWIRQPPGKGLEWIGEINHSGSTNYNPSLKSRVTISVDTSKNQFSLKLCSVTAADTAVYYCXXXXXXXXXXXXXXWGQGTLVTVSS

SEQ ID NO: 336 - protein, 120 aa, synthetic construct
  REGION 1..120: note = Modified VH domain from a human immunoglobulin germline
  VARIANT 96..109: note = X is any residue
QVQLQQWGAGLLKPSECLSLTCQVYGGSFSGYYWSWIRQPPGKGLEWIAEINHSGSTNYNPSLKSRVTISVDTSKNQFSLKLCSVTAADTAVYYCXXXXXXXXXXXXXXWGQGTLVTVSS

SEQ ID NO: 337 - protein, 120 aa, synthetic construct
  REGION 1..120: note = Modified VH domain from a human immunoglobulin germline
  VARIANT 96..109: note = X is any residue
QVQLQQWGAGLLKPSECLSLTCQVYGGSFSGYYWSWIRQPPGKGLEWIGEINHSGSTNYNPSLKSRVTISVDTSKNQFSLKLCNVTAADTAVYYCXXXXXXXXXXXXXXWGQGTLVTVSS

SEQ ID NO: 338 - protein, 120 aa, synthetic construct
  REGION 1..120: note = Modified VH domain from a human immunoglobulin germline
  VARIANT 96..109: note = X is any residue
QVQLQQWGAGLLKPSECLSLTCQVYGGSFSGYYWSWIRQPPGKGLEWIGEINHSGSTNYNPSLKSRVTISVDTSKNQFSLKLCSVTPADTAVYYCXXXXXXXXXXXXXXWGQGTLVTVSS

SEQ ID NO: 339 - protein, 120 aa, synthetic construct
  REGION 1..120: note = Modified VH domain from a human immunoglobulin germline
  VARIANT 96..109: note = X is any residue
QVQLQQWGATLLKPSETLSLTCCVYGGSFSGYYWSWIRQPPGKGLEWIAEINHSGSTNYNPSLKSRVTISVDTSKNCFSLKLSSVTAADTAVYYCXXXXXXXXXXXXXXWGQGTLVTVSS

SEQ ID NO: 340 - protein, 120 aa, synthetic construct
  REGION 1..120: note = Modified VH domain from a human immunoglobulin germline
  VARIANT 96..109: note = X is any residue
QVQLQQWGATLLKPSETLSLTCCVYGGSFSGYYWSWIRQPPGKGLEWIGEINHSGSTNYNPSLKSRVTISVDTSKNCFSLKLSNVTAADTAVYYCXXXXXXXXXXXXXXWGQGTLVTVSS

SEQ ID NO: 341 - protein, 120 aa, synthetic construct
  REGION 1..120: note = Modified VH domain from a human immunoglobulin germline
  VARIANT 96..109: note = X is any residue
QVQLQQWGATLLKPSETLSLTCCVYGGSFSGYYWSWIRQPPGKGLEWIGEINHSGSTNYNPSLKSRVTISVDTSKNCFSLKLSSVTPADTAVYYCXXXXXXXXXXXXXXWGQGTLVTVSS

SEQ ID NO: 342 - protein, 120 aa, synthetic construct
  REGION 1..120: note = Modified VH domain from a human immunoglobulin germline
  VARIANT 96..109: note = X is any residue
QVQLQQWGAGLLKPSETLSLTCCVYGGSFSGYYWSWIRQPPGKGLEWIAEINHSGSTNYNPSLKSRVTISVDTSKNCFSLKLSNVTAADTAVYYCXXXXXXXXXXXXXXWGQGTLVTVSS

SEQ ID NO: 343 - protein, 120 aa, synthetic construct
  REGION 1..120: note = Modified VH domain from a human immunoglobulin germline
  VARIANT 96..109: note = X is any residue
QVQLQQWGAGLLKPSETLSLTCCVYGGSFSGYYWSWIRQPPGKGLEWIAEINHSGSTNYNPSLKSRVTISVDTSKNCFSLKLSSVTPADTAVYYCXXXXXXXXXXXXXXWGQGTLVTVSS

SEQ ID NO: 344 - protein, 120 aa, synthetic construct
  REGION 1..120: note = Modified VH domain from a human immunoglobulin germline
  VARIANT 96..109: note = X is any residue
QVQLQQWGAGLLKPSETLSLTCCVYGGSFSGYYWSWIRQPPGKGLEWIGEINHSGSTNYNPSLKSRVTISVDTSKNCFSLKLSNVTPADTAVYYCXXXXXXXXXXXXXXWGQGTLVTVSS

SEQ ID NO: 345 - protein, 127 aa, synthetic construct; identical to SEQ ID NO: 210
  REGION 1..127: note = Modified VH domain from a human immunoglobulin germline
  VARIANT 98..116: note = X is any residue

SEQ ID NO: 346 - protein, 127 aa, synthetic construct
  REGION 1..127: note = Modified VH domain from a human immunoglobulin germline
  VARIANT 98..116: note = X is any residue
QLQLQESGPTLVKPSECLSLTCTVSGGSISSSSYYWGWIRQPPGKGLEWIASIYYSGSTYYNPSLKSRVTISVDTSKNQFSLKLCSVTAADTAVYYCXXXXXXXXXXXXXXXXXXXWGQGTLVTVSS

SEQ ID NO: 347 - protein, 127 aa, synthetic construct
  REGION 1..127: note = Modified VH domain from a human immunoglobulin germline
  VARIANT 98..116: note = X is any residue
QLQLQESGPTLVKPSECLSLTCTVSGGSISSSSYYWGWIRQPPGKGLEWIGSIYYSGSTYYNPSLKSRVTISVDTSKNQFSLKLCNVTAADTAVYYCXXXXXXXXXXXXXXXXXXXWGQGTLVTVSS

SEQ ID NO: 348 - protein, 127 aa, synthetic construct
  REGION 1..127: note = Modified VH domain from a human immunoglobulin germline
  VARIANT 98..116: note = X is any residue
QLQLQESGPTLVKPSECLSLTCTVSGGSISSSSYYWGWIRQPPGKGLEWIGSIYYSGSTYYNPSLKSRVTISVDTSKNQFSLKLCSVTPADTAVYYCXXXXXXXXXXXXXXXXXXXWGQGTLVTVSS

SEQ ID NO: 349 - protein, 127 aa, synthetic construct
  REGION 1..127: note = Modified VH domain from a human immunoglobulin germline
  VARIANT 98..116: note = X is any residue
QLQLQESGPGLVKPSECLSLTCTVSGGSISSSSYYWGWIRQPPGKGLEWIASIYYSGSTYYNPSLKSRVTISVDTSKNQFSLKLCNVTAADTAVYYCXXXXXXXXXXXXXXXXXXXWGQGTLVTVSS

SEQ ID NO: 350 - protein, 127 aa, synthetic construct
  REGION 1..127: note = Modified VH domain from a human immunoglobulin germline
  VARIANT 98..116: note = X is any residue
QLQLQESGPGLVKPSECLSLTCTVSGGSISSSSYYWGWIRQPPGKGLEWIASIYYSGSTYYNPSLKSRVTISVDTSKNQFSLKLCSVTPADTAVYYCXXXXXXXXXXXXXXXXXXXWGQGTLVTVSS

SEQ ID NO: 351 - protein, 127 aa, synthetic construct
  REGION 1..127: note = Modified VH domain from a human immunoglobulin germline
  VARIANT 98..116: note = X is any residue
QLQLQESGPGLVKPSECLSLTCTVSGGSISSSSYYWGWIRQPPGKGLEWIGSIYYSGSTYYNPSLKSRVTISVDTSKNQFSLKLCNVTPADTAVYYCXXXXXXXXXXXXXXXXXXXWGQGTLVTVSS

SEQ ID NO: 352 - protein, 127 aa, synthetic construct
  REGION 1..127: note = Modified VH domain from a human immunoglobulin germline
  VARIANT 98..116: note = X is any residue
QLQLQESGPTLVKPSECLSLTCQVSGGSISSSSYYWGWIRQPPGKGLEWIGSIYYSGSTYYNPSLKSRVTISVDTSKNQFSLKLCSVTAADTAVYYCXXXXXXXXXXXXXXXXXXXWGQGTLVTVSS

SEQ ID NO: 353 - protein, 127 aa, synthetic construct
  REGION 1..127: note = Modified VH domain from a human immunoglobulin germline
  VARIANT 98..116: note = X is any residue
QLQLQESGPGLVKPSECLSLTCQVSGGSISSSSYYWGWIRQPPGKGLEWIASIYYSGSTYYNPSLKSRVTISVDTSKNQFSLKLCSVTAADTAVYYCXXXXXXXXXXXXXXXXXXXWGQGTLVTVSS

SEQ ID NO: 354 - protein, 127 aa, synthetic construct
  REGION 1..127: note = Modified VH domain from a human immunoglobulin germline
  VARIANT 98..116: note = X is any residue
QLQLQESGPGLVKPSECLSLTCQVSGGSISSSSYYWGWIRQPPGKGLEWIGSIYYSGSTYYNPSLKSRVTISVDTSKNQFSLKLCNVTAADTAVYYCXXXXXXXXXXXXXXXXXXXWGQGTLVTVSS

SEQ ID NO: 355 - protein, 127 aa, synthetic construct
  REGION 1..127: note = Modified VH domain from a human immunoglobulin germline
  VARIANT 98..116: note = X is any residue
QLQLQESGPGLVKPSECLSLTCQVSGGSISSSSYYWGWIRQPPGKGLEWIGSIYYSGSTYYNPSLKSRVTISVDTSKNQFSLKLCSVTPADTAVYYCXXXXXXXXXXXXXXXXXXXWGQGTLVTVSS

SEQ ID NO: 356 - protein, 127 aa, synthetic construct
  REGION 1..127: note = Modified VH domain from a human immunoglobulin germline
  VARIANT 98..116: note = X is any residue
QLQLQESGPTLVKPSETLSLTCCVSGGSISSSSYYWGWIRQPPGKGLEWIASIYYSGSTYYNPSLKSRVTISVDTSKNCFSLKLSSVTAADTAVYYCXXXXXXXXXXXXXXXXXXXWGQGTLVTVSS

SEQ ID NO: 357 - protein, 127 aa, synthetic construct
  REGION 1..127: note = Modified VH domain from a human immunoglobulin germline
  VARIANT 98..116: note = X is any residue
QLQLQESGPTLVKPSETLSLTCCVSGGSISSSSYYWGWIRQPPGKGLEWIGSIYYSGSTYYNPSLKSRVTISVDTSKNCFSLKLSNVTAADTAVYYCXXXXXXXXXXXXXXXXXXXWGQGTLVTVSS

SEQ ID NO: 358 - protein, 127 aa, synthetic construct
  REGION 1..127: note = Modified VH domain from a human immunoglobulin germline
  VARIANT 98..116: note = X is any residue
QLQLQESGPTLVKPSETLSLTCCVSGGSISSSSYYWGWIRQPPGKGLEWIGSIYYSGSTYYNPSLKSRVTISVDTSKNCFSLKLSSVTPADTAVYYCXXXXXXXXXXXXXXXXXXXWGQGTLVTVSS

SEQ ID NO: 359 - protein, 127 aa, synthetic construct
  REGION 1..127: note = Modified VH domain from a human immunoglobulin germline
  VARIANT 98..116: note = X is any residue
QLQLQESGPGLVKPSETLSLTCCVSGGSISSSSYYWGWIRQPPGKGLEWIASIYYSGSTYYNPSLKSRVTISVDTSKNCFSLKLSNVTAADTAVYYCXXXXXXXXXXXXXXXXXXXWGQGTLVTVSS

SEQ ID NO: 360 - protein, 127 aa, synthetic construct
  REGION 1..127: note = Modified VH domain from a human immunoglobulin germline
  VARIANT 98..116: note = X is any residue
QLQLQESGPGLVKPSETLSLTCCVSGGSISSSSYYWGWIRQPPGKGLEWIASIYYSGSTYYNPSLKSRVTISVDTSKNCFSLKLSSVTPADTAVYYCXXXXXXXXXXXXXXXXXXXWGQGTLVTVSS

SEQ ID NO: 361 - protein, 127 aa, synthetic construct
  REGION 1..127: note = Modified VH domain from a human immunoglobulin germline
  VARIANT 98..116: note = X is any residue
QLQLQESGPGLVKPSETLSLTCCVSGGSISSSSYYWGWIRQPPGKGLEWIGSIYYSGSTYYNPSLKSRVTISVDTSKNCFSLKLSNVTPADTAVYYCXXXXXXXXXXXXXXXXXXXWGQGTLVTVSS

SEQ ID NO: 362 - protein, 127 aa, synthetic construct
  REGION 1..127: note = Modified VH domain from a human immunoglobulin germline
  VARIANT 98..116: note = X is any residue
QLQLQESGPTLVKPSECLSLTCTVSGGSISSSSYYWGWIRQPPGKGLEWIGSIYYSGSTYYNPSLKSRVTISVDTSKNQFSLKLCDVTAADTAVYYCXXXXXXXXXXXXXXXXXXXWGQGTLVTVSS

SEQ ID NO: 363 - protein, 127 aa, synthetic construct
  REGION 1..127: note = Modified VH domain from a human immunoglobulin germline
  VARIANT 98..116: note = X is any residue
QLQLQESGPGLVKPSECLSLTCTVSGGSISSSSYYWGWIRQPPGKGLEWIGSIYYSGSTYYNPSLKSRVTISVDTSKNQFSLKLCDVTPADTAVYYCXXXXXXXXXXXXXXXXXXXWGQGTLVTVSS

SEQ ID NO: 364 - protein, 127 aa, synthetic construct; identical to SEQ ID NO: 358
  REGION 1..127: note = Modified VH domain from a human immunoglobulin germline
  VARIANT 98..116: note = X is any residue

SEQ ID NO: 365 - protein, 127 aa, synthetic construct
  REGION 1..127: note = Modified VH domain from a human immunoglobulin germline
  VARIANT 98..116: note = X is any residue
QLQLQESGPTLVKPSETLSLTCCVSGGSISSSSYYWGWIRQPPGKGLEWIGSIYYSGSTYYNPSLKSRVTISVDTSKNCFSLKLSDVTAADTAVYYCXXXXXXXXXXXXXXXXXXXWGQGTLVTVSS

SEQ ID NO: 366 - protein, 127 aa, synthetic construct
  REGION 1..127: note = Modified VH domain from a human immunoglobulin germline
  VARIANT 98..116: note = X is any residue
QLQLQESGPGLVKPSETLSLTCCVSGGSISSSSYYWGWIRQPPGKGLEWIGSIYYSGSTYYNPSLKSRVTISVDTSKNCFSLKLSDVTPADTAVYYCXXXXXXXXXXXXXXXXXXXWGQGTLVTVSS

SEQ ID NO: 367 - protein, 130 aa, synthetic construct
  REGION 1..130: note = Modified VH domain from a human immunoglobulin germline
  VARIANT 98..119: note = X is any residue
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCXXXXXXXXXXXXXXXXXXXXXXWGQGTMVTVSS

SEQ ID NO: 368 - protein, 130 aa, synthetic construct
  REGION 1..130: note = Modified VH domain from a human immunoglobulin germline
  VARIANT 98..119: note = X is any residue
QVQLQESGPGLVKPSQTLSLTCCVSGGSISSGGYYWSWIRQPPGKGLEWIGYIYYSGSTYYNPSLKSRVTISVDTSKNCFSLKLSDVTPADTAVYYCXXXXXXXXXXXXXXXXXXXXXXWGQGTMVTVSS

SEQ ID NO: 369 - protein, 123 aa, synthetic construct
  REGION 1..123: note = Modified VH domain from a human immunoglobulin germline
  VARIANT 97..112: note = X is any residue
QVQLQESGPGLVKPSDTLSLTCAVSGYSISSSNWWGWIRQPPGKGLEWIGYIYYSGSTYYNPSLKSRVTMSVDTSKNQFSLKLSSVTAVDTAVYYCXXXXXXXXXXXXXXXXWGQGTLVTVSS

SEQ ID NO: 370 - protein, 123 aa, synthetic construct
  REGION 1..123: note = Modified VH domain from a human immunoglobulin germline
  VARIANT 97..112: note = X is any residue
QVQLQESGPGLVKPSDTLSLTCCVSGYSISSSNWWGWIRQPPGKGLEWIGYIYYSGSTYYNPSLKSRVTMSVDTSKNCFSLKLSDVTPVDTAVYYCXXXXXXXXXXXXXXXXWGQGTLVTVSS

SEQ ID NO: 371 - protein, 123 aa, synthetic construct
  REGION 1..123: note = Modified VH domain from a human immunoglobulin germline
  VARIANT 98..112: note = X is any residue
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCXXXXXXXXXXXXXXXWGQGTLVTVSS

SEQ ID NO: 372 - protein, 123 aa, synthetic construct
  REGION 1..123: note = Modified VH domain from a human immunoglobulin germline
  VARIANT 98..112: note = X is any residue
QVQLQESGPGLVKPSQTLSLTCCVSGGSISSGDYYWSWIRQPPGKGLEWIGYIYYSGSTYYNPSLKSRVTISVDTSKNCFSLKLSDVTPADTAVYYCXXXXXXXXXXXXXXXWGQGTLVTVSS

SEQ ID NO: 373 - protein, 123 aa, synthetic construct
  REGION 1..123: note = Modified VH domain from a human immunoglobulin germline
  VARIANT 97..112: note = X is any residue
QVQLQESGPGLVKPPGTLSLTCAVSGGSISSSNWWSWVRQPPGKGLEWIGEIYHSGSTNYNPSLKSRVTISVDKSKNQFSLKLSSVTAADTAVYYCXXXXXXXXXXXXXXXXWGQGTLVTVSS

SEQ ID NO: 374 - protein, 123 aa, synthetic construct
  REGION 1..123: note = Modified VH domain from a human immunoglobulin germline
  VARIANT 97..112: note = X is any residue
QVQLQESGPGLVKPPGTLSLTCCVSGGSISSSNWWSWVRQPPGKGLEWIGEIYHSGSTNYNPSLKSRVTISVDKSKNCFSLKLSDVTPADTAVYYCXXXXXXXXXXXXXXXXWGQGTLVTVSS

SEQ ID NO: 375 - protein, 125 aa, synthetic construct
  REGION 1..125: note = Modified VH domain from a human immunoglobulin germline
  VARIANT 98..114: note = X is any residue
QLQLQESGSGLVKPSQTLSLTCAVSGGSISSGGYSWSWIRQPPGKGLEWIGYIYHSGSTYYNPSLKSRVTISVDRSKNQFSLKLSSVTAADTAVYYCXXXXXXXXXXXXXXXXXWGQGTLVTVSS

SEQ ID NO: 376 - protein, 125 aa, synthetic construct
  REGION 1..125: note = Modified VH domain from a human immunoglobulin germline
  VARIANT 98..114: note = X is any residue
QLQLQESGSGLVKPSQTLSLTCCVSGGSISSGGYSWSWIRQPPGKGLEWIGYIYHSGSTYYNPSLKSRVTISVDRSKNCFSLKLSDVTPADTAVYYCXXXXXXXXXXXXXXXXXWGQGTLVTVSS

SEQ ID NO: 377 - protein, 121 aa, synthetic construct
  REGION 1..121: note = Modified VH domain from a human immunoglobulin germline
  VARIANT 98..110: note = X is any residue
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCXXXXXXXXXXXXXWGQGTLVTVSS

SEQ ID NO: 378 - protein, 121 aa, synthetic construct
  REGION 1..121: note = Modified VH domain from a human immunoglobulin germline
  VARIANT 98..110: note = X is any residue
QVQLQESGPGLVKPSETLSLTCCVSGGSVSSGSYYWSWIRQPPGKGLEWIGYIYYSGSTNYNPSLKSRVTISVDTSKNCFSLKLSDVTPADTAVYYCXXXXXXXXXXXXXWGQGTLVTVSS

SEQ ID NO: 379 - protein, 117 aa, synthetic construct
  REGION 1..117: note = Modified VH domain from a human immunoglobulin germline
  VARIANT 96..106: note = X is any residue
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCXXXXXXXXXXXWGQGTMVTVSS

SEQ ID NO: 380 - protein, 117 aa, synthetic construct
  REGION 1..117: note = Modified VH domain from a human immunoglobulin germline
  VARIANT 96..106: note = X is any residue
QVQLQQWGAGLLKPSETLSLTCCVYGGSFSGYYWSWIRQPPGKGLEWIGEINHSGSTNYNPSLKSRVTISVDTSKNCFSLKLSDVTPADTAVYYCXXXXXXXXXXXWGQGTMVTVSS

SEQ ID NO: 381 - protein, 118 aa, synthetic construct
  REGION 1..118: note = Modified VH domain from a human immunoglobulin germline
  VARIANT 97..107: note = X is any residue
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCXXXXXXXXXXXWGQGTMVTVSS

SEQ ID NO: 382 - protein, 118 aa, synthetic construct
  REGION 1..118: note = Modified VH domain from a human immunoglobulin germline
  VARIANT 97..107: note = X is any residue
QVQLQESGPGLVKPSETLSLTCCVSGYSISSGYYWGWIRQPPGKGLEWIGSIYHSGSTYYNPSLKSRVTISVDTSKNCFSLKLSDVTPADTAVYYCXXXXXXXXXXXWGQGTMVTVSS

SEQ ID NO: 383 - protein, 119 aa, synthetic construct
  REGION 1..119: note = Modified VH domain from a human immunoglobulin germline
  VARIANT 98..108: note = X is any residue
QVQLQESGPGLVKPSQTLSLTCTVSGGSISSGGYYWSWIRQHPGKGLEWIGYIYYSGSTYYNPSLKSLVTISVDTSKNQFSLKLSSVTAADTAVYYCXXXXXXXXXXXWGQGTMVTVSS

SEQ ID NO: 384 - protein, 119 aa, synthetic construct
  REGION 1..119: note = Modified VH domain from a human immunoglobulin germline
  VARIANT 98..108: note = X is any residue
QVQLQESGPGLVKPSQTLSLTCCVSGGSISSGGYYWSWIRQHPGKGLEWIGYIYYSGSTYYNPSLKSLVTISVDTSKNCFSLKLSDVTPADTAVYYCXXXXXXXXXXXWGQGTMVTVSS

SEQ ID NO: 385 - protein, 117 aa, synthetic construct
  REGION 1..117: note = Modified VH domain from a human immunoglobulin germline
  VARIANT 96..106: note = X is any residue
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCXXXXXXXXXXXWGQGTMVTVSS

SEQ ID NO: 386 - protein, 117 aa, synthetic construct
  REGION 1..117: note = Modified VH domain from a human immunoglobulin germline
  VARIANT 96..106: note = X is any residue
QVQLQESGPGLVKPSETLSLTCCVSGGSISSYYWSWIRQPPGKGLEWIGYIYYSGSTNYNPSLKSRVTISVDTSKNCFSLKLSDVTPADTAVYYCXXXXXXXXXXXWGQGTMVTVSS

SEQ ID NO: 387 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFSGFSLSTSGVGVGWIRRPPGKALEWLALIYWNDDKRYSPSLKSRLTITKDTSKNQVVLTMTNMDPVDTATYYCXXXXXXXXXXWGQGTLVTVSS

SEQ ID NO: 388 - protein, 118 aa, synthetic construct
  REGION 1..118: note = Modified VH domain from a human immunoglobulin germline
  VARIANT 98..107: note = X is any residue
QITLKESGPTLVKPGQTLTLTCTFSGFSLSTSGVGVGWIRRPPGKALEWLALIYWNDDKRYSPSLKSRLTITKDTSKNQVVLTMTNMDPVDTATYYCXXXXXXXXXXWGQGTLVTVSS

SEQ ID NO: 389 - protein, 118 aa, synthetic construct
  REGION 1..118: note = Modified VH domain from a human immunoglobulin germline
  VARIANT 98..107: note = X is any residue
QITLKESGPTLVKPTDTLTLTCTFSGFSLSTSGVGVGWIRRPPGKALEWLALIYWNDDKRYSPSLKSRLTITKDTSKNQVVLTMTNMDPVDTATYYCXXXXXXXXXXWGQGTLVTVSS

SEQ ID NO: 390 - protein, 118 aa, synthetic construct
  REGION 1..118: note = Modified VH domain from a human immunoglobulin germline
  VARIANT 98..107: note = X is any residue
QITLKESGPTLVKPTQDLTLTCTFSGFSLSTSGVGVGWIRRPPGKALEWLALIYWNDDKRYSPSLKSRLTITKDTSKNQVVLTMTNMDPVDTATYYCXXXXXXXXXXWGQGTLVTVSS

SEQ ID NO: 391 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFDGFSLSTSGVGVGWIRRPPGKALEWLALIYWNDDKRYSPSLKSRLTITKDTSKNQVVLTMTNMDPVDTATYYCXXXXXXXXXXWGQGTLVTVSS

SEQ ID NO: 392 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFSGFSLSTSGVGVGWYRRPPGKALEWLALIYWNDDKRYSPSLKSRLTITKDTSKNQVVLTMTNMDPVDTATYYCXXXXXXXXXXWGQGTLVTVSS

SEQ ID NO: 393 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFSGFSLSTSGVGVGWHRRPPGKALEWLALIYWNDDKRYSPSLKSRLTITKDTSKNQVVLTMTNMDPVDTATYYCXXXXXXXXXXWGQGTLVTVSS

SEQ ID NO: 394 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFSGFSLSTSGVGVGWIRRPPGKDLEWLALIYWNDDKRYSPSLKSRLTITKDTSKNQVVLTMTNMDPVDTATYYCXXXXXXXXXXWGQGTLVTVSS

SEQ ID NO: 395 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFSGFSLSTSGVGVGWIRRPPGKGLEWLALIYWNDDKRYSPSLKSRLTITKDTSKNQVVLTMTNMDPVDTATYYCXXXXXXXXXXWGQGTLVTVSS

SEQ ID NO: 396 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFSGFSLSTSGVGVGWIRRPPGKPLEWLALIYWNDDKRYSPSLKSRLTITKDTSKNQVVLTMTNMDPVDTATYYCXXXXXXXXXXWGQGTLVTVSS

SEQ ID NO: 397 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFSGFSLSTSGVGVGWIRRPPGKALEWLALIYWNDDKRYSPSLKDRLTITKDTSKNQVVLTMTNMDPVDTATYYCXXXXXXXXXXWGQGTLVTVSS

SEQ ID NO: 398 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFSGFSLSTSGVGVGWIRRPPGKALEWLALIYWNDDKRYSPSLKSRLTITMDTSKNQVVLTMTNMDPVDTATYYCXXXXXXXXXXWGQGTLVTVSS

SEQ ID NO: 399 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFSGFSLSTSGVGVGWIRRPPGKALEWLALIYWNDDKRYSPSLKSRLTITKDDSKNQVVLTMTNMDPVDTATYYCXXXXXXXXXXWGQGTLVTVSS

SEQ ID NO: 400 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFSGFSLSTSGVGVGWIRRPPGKALEWLALIYWNDDKRYSPSLKSRLTITKDPSKNQVVLTMTNMDPVDTATYYCXXXXXXXXXXWGQGTLVTVSS

SEQ ID NO: 401 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFSGFSLSTSGVGVGWIRRPPGKALEWLALIYWNDDKRYSPSLKSRLTITKDTSKNQVVLTMTNMLPVDTATYYCXXXXXXXXXXWGQGTLVTVSS

SEQ ID NO: 402 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFSGFSLSTSGVGVGWIRRPPGKALEWLALIYWNDDKRYSPSLKSRLTITKDTSKNQVVLTMTNMQPVDTATYYCXXXXXXXXXXWGQGTLVTVSS

SEQ ID NO: 403 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFSGFSLSTSGVGVGWIRRPPGKALEWLALIYWNDDKRYSPSLKSRLTITKDTSKNQVVLTMTNMKPVDTATYYCXXXXXXXXXXWGQGTLVTVSS

SEQ ID NO: 404 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFSGFSLSTSGVGVGWIRRPPGKALEWLALIYWNDDKRYSPSLKSRLTITKDTSKNQVVLTMTNMTPVDTATYYCXXXXXXXXXXWGQGTLVTVSS

SEQ ID NO: 405 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFSGFSLSTSGVGVGWIRRPPGKALEWLALIYWNDDKRYSPSLKSRLTITKDTSKNQVVLTMTNMDYVDTATYYCXXXXXXXXXXWGQGTLVTVSS

SEQ ID NO: 406 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFSGFSLSTSGVGVGWIRRPPGKALEWLALIYWNDDKRYSPSLKSRLTITKDTSKNQVVLTMTNMDPRDTATYYCXXXXXXXXXXWGQGTLVTVSS

SEQ ID NO: 407 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFSGFSLSTSGVGVGWIRRPPGKALEWLALIYWNDDKRYSPSLKSRLTITKDTSKNQVVLTMTNMDPSDTATYYCXXXXXXXXXXWGQGTLVTVSS

SEQ ID NO: 408 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFSGFSLSTSGVGVGWIRRPPGKALEWLALIYWNDDKRYSPSLKSRLTITKDTSKNQVVLTMTNMDPKDTATYYCXXXXXXXXXXWGQGTLVTVSS

SEQ ID NO: 409 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFSGFSLSTSGVGVGWIRRPPGKALEWLALIYWNDDKRYSPSLKSRLTITKDTSKNQVVLTMTNMDPTDTATYYCXXXXXXXXXXWGQGTLVTVSS

SEQ ID NO: 410 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFSGFSLSTSGVGVGWIRRPPGKALEWLALIYWNDDKRYSPSLKSRLTITKDTSKNQVVLTMTNMDPVDTAIYYCXXXXXXXXXXWGQGTLVTVSS

SEQ ID NO: 411 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFSGFSLSTSGVGVGWIRRPPGKALEWLALIYWNDDKRYSPSLKSRLTITKDTSKNQVVLTMTNMDPVDTATYYCXXXXXXXXXXWGDGTLVTVSS

SEQ ID NO: 412 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFSGFSLSTSGVGVGWIRRPPGKALEWLALIYWNDDKRYSPSLKSRLTITKDTSKNQVVLTMTNMDPVDTATYYCXXXXXXXXXXWGQGILVTVSS

SEQ ID NO: 413 - protein, 118 aa, synthetic construct
  REGION 1..118: note = Modified VH domain from a human immunoglobulin germline
  VARIANT 98..107: note = X is any residue
QITLKESGPTLVKPTQTLTLTCTFSGFSLSTSGVGVGWIRRPPGKALEWLALIYWNDDKRYSPSLKSRLTITKDTSKNQVVLTMTNMDPVDTATYYCXXXXXXXXXXWGQGYLVTVSS

SEQ ID NO: 414 - protein, 118 aa, synthetic construct
  REGION 1..118: note = Modified VH domain from a human immunoglobulin germline
  VARIANT 98..107: note = X is any residue
QITLKESGPTLVKPTQCLTLTCTFSGFSLSTSGVGVGWIRRPPGKALEWLALIYWNDDKRYSPSLKSRLTITKDTSKNQVVLTMCNMDPVDTATYYCXXXXXXXXXXWGQGTLVTVSS

SEQ ID NO: 415 - protein, 118 aa, synthetic construct
  REGION 1..118: note = Modified VH domain from a human immunoglobulin germline
  VARIANT 98..107: note = X is any residue
QITLKESGPTLVKPTQTLCLTCTFSGFSLSTSGVGVGWIRRPPGKALEWLALIYWNDDKRYSPSLKSRLTITKDTSKNQVVLCMTNMDPVDTATYYCXXXXXXXXXXWGQGTLVTVSS

SEQ ID NO: 416 - protein, 118 aa, synthetic construct
  REGION 1..118: note = Modified VH domain from a human immunoglobulin germline
  VARIANT 98..107: note = X is any residue
QITLKESGPTLVKPTQTLTLTCCFSGFSLSTSGVGVGWIRRPPGKALEWLALIYWNDDKRYSPSLKSRLTITKDTSKNCVVLTMTNMDPVDTATYYCXXXXXXXXXXWGQGTLVTVSS

SEQ ID NO: 417 - protein, 118 aa, synthetic construct
  REGION 1..118: note = Modified VH domain from a human immunoglobulin germline
  VARIANT 98..107: note = X is any residue
QITLKESGPTLVKPTQTLCLTCTFSGFSLSTSGVGVGWIRRPPGKALEWLALIYWNDDKRYSPSLKSRLTITKDTSKNQVVLCMTNMDPSDTATYYCXXXXXXXXXXWGQGTLVTVSS

SEQ ID NO: 418 - protein, 118 aa, synthetic construct
  REGION 1..118: note = Modified VH domain from a human immunoglobulin germline
  VARIANT 98..107: note = X is any residue
QITLKESGPTLVKPGQTLCLTCTFSGFSLSTSGVGVGWIRRPPGKALEWLALIYWNDDKRYSPSLKSRLTITKDTSKNQVVLCMTNMDPSDTATYYCXXXXXXXXXXWGQGTLVTVSS

SEQ ID NO: 419 - protein, 118 aa, synthetic construct
  REGION 1..118: note = Modified VH domain from a human immunoglobulin germline
  VARIANT 98..107: note = X is any residue
QITLKESGPTLVKPGQTLCLTCTFSGFSLSTSGVGVGWIRRPPGKALEWLALIYWNDDKRYSPSLKSRLTITKDTSKNQVVLCMTNMDPVDTATYYCXXXXXXXXXXWGQGTLVTVSS

SEQ ID NO: 420 - protein, 118 aa, synthetic construct
  REGION 1..118: note = Modified VH domain from a human immunoglobulin germline
  VARIANT 98..107: note = X is any residue
QITLKESGPTLVKPTQTLCLTCTFSGFSLSTSGVGVGWYRRPPGKALEWLALIYWNDDKRYSPSLKSRLTITKDTSKNQVVLCMTNMDPVDTATYYCXXXXXXXXXXWGQGTLVTVSS

SEQ ID NO: 421 - protein, 118 aa, synthetic construct
  REGION 1..118: note = Modified VH domain from a human immunoglobulin germline
  VARIANT 98..107: note = X is any residue
QITLKESGPTLVKPTQTLCLTCTFSGFSLSTSGVGVGWYRRPPGKALEWLALIYWNDDKRYSPSLKSRLTITKDTSKNQVVLCMTNMDPSDTATYYCXXXXXXXXXXWGQGTLVTVSS

SEQ ID NO: 422 - protein, 118 aa, synthetic construct
  REGION 1..118: note = Modified VH domain from a human immunoglobulin germline
  VARIANT 98..107: note = X is any residue
QITLKESGPTLVKPGQTLCLTCTFSGFSLSTSGVGVGWYRRPPGKALEWLALIYWNDDKRYSPSLKSRLTITKDTSKNQVVLCMTNMDPVDTATYYCXXXXXXXXXXWGQGTLVTVSS

SEQ ID NO: 423 - protein, 118 aa, synthetic construct
  REGION 1..118: note = Modified VH domain from a human immunoglobulin germline
  VARIANT 98..107: note = X is any residue
QITLKESGPTLVKPTQTLCLTCTFSGFSLSTSGVGVGWIRRPPGKDLEWLALIYWNDDKRYSPSLKSRLTITKDTSKNQVVLCMTNMDPVDTATYYCXXXXXXXXXXWGQGTLVTVSS

SEQ ID NO: 424 - protein, 118 aa, synthetic construct
  REGION 1..118: note = Modified VH domain from a human immunoglobulin germline
  VARIANT 98..107: note = X is any residue
QITLKESGPTLVKPTQTLCLTCTFSGFSLSTSGVGVGWYRRPPGKDLEWLALIYWNDDKRYSPSLKSRLTITKDTSKNQVVLCMTNMDPVDTATYYCXXXXXXXXXXWGQGTLVTVSS

SEQ ID NO: 425 - protein, 118 aa, synthetic construct; identical to SEQ ID NO: 423
  REGION 1..118: note = Modified VH domain from a human immunoglobulin germline
  VARIANT 98..107: note = X is any residue

SEQ ID NO: 426 - protein, 118 aa, synthetic construct
  REGION 1..118: note = Modified VH domain from a human immunoglobulin germline
  VARIANT 98..107: note = X is any residue
QITLKESGPTLVKPTQTLCLTCTFSGFSLSTSGVGVGWIRRPPGKDLEWLALIYWNDDKRYSPSLKSRLTITKDTSKNQVVLCMTNMDPSDTATYYCXXXXXXXXXXWGQGTLVTVSS

SEQ ID NO: 427 - protein, 118 aa, synthetic construct
  REGION 1..118: note = Modified VH domain from a human immunoglobulin germline
  VARIANT 98..107: note = X is any residue
QITLKESGPTLVKPTQTLCLTCTFSGFSLSTSGVGVGWIRRPPGKALEWLALIYWNDDKRYSPSLKSRLTITKDTSKNQVVLCMTNMTPVDTATYYCXXXXXXXXXXWGQGTLVTVSS

SEQ ID NO: 428 - protein, 118 aa, synthetic construct
  REGION 1..118: note = Modified VH domain from a human immunoglobulin germline
  VARIANT 98..107: note = X is any residue
QITLKESGPTLVKPTQTLCLTCTFSGFSLSTSGVGVGWIRRPPGKDLEWLALIYWNDDKRYSPSLKSRLTITKDTSKNQVVLCMTNMTPVDTATYYCXXXXXXXXXXWGQGTLVTVSS

SEQ ID NO: 429 - protein, 118 aa, synthetic construct
  REGION 1..118: note = Modified VH domain from a human immunoglobulin germline
  VARIANT 98..107: note = X is any residue
QITLKESGPTLVKPTQTLCLTCTFSGFSLSTSGVGVGWYRRPPGKALEWLALIYWNDDKRYSPSLKSRLTITKDTSKNQVVLCMTNMTPVDTATYYCXXXXXXXXXXWGQGTLVTVSS

SEQ ID NO: 430 - protein, 118 aa, synthetic construct
  REGION 1..118: note = Modified VH domain from a human immunoglobulin germline
  VARIANT 98..107: note = X is any residue
QITLKESGPTLVKPGQTLCLTCTFSGFSLSTSGVGVGWIRRPPGKALEWLALIYWNDDKRYSPSLKSRLTITKDTSKNQVVLCMTNMTPVDTATYYCXXXXXXXXXXWGQGTLVTVSS

SEQ ID NO: 431 - protein, 118 aa, synthetic construct; identical to SEQ ID NO: 415
  REGION 1..118: note = Modified VH domain from a human immunoglobulin germline
  VARIANT 98..107: note = X is any residue

SEQ ID NO: 432 - protein, 118 aa, synthetic construct; identical to SEQ ID NO: 387
  REGION 1..118: note = Modified VH domain from a human immunoglobulin germline
  VARIANT 98..107: note = X is any residue

SEQ ID NO: 433 - protein, 118 aa, synthetic construct; identical to SEQ ID NO: 387
  REGION 1..118: note = Modified VH domain from a human immunoglobulin germline
  VARIANT 98..107: note = X is any residue

SEQ ID NO: 434 - protein, 118 aa, synthetic construct; identical to SEQ ID NO: 422
  REGION 1..118: note = Modified VH domain from a human immunoglobulin germline
  VARIANT 98..107: note = X is any residue

SEQ ID NO: 435 - protein, 118 aa, synthetic construct
  REGION 1..118: note = Modified VH domain from a human immunoglobulin germline
  VARIANT 98..107: note = X is any residue
QITLKESGPTLVKPGQTLCLTCTFSGFSLSTSGVGVGWIRRPPGKDLEWLALIYWNDDKRYSPSLKSRLTITKDTSKNQVVLCMTNMDPVDTATYYCXXXXXXXXXXWGQGTLVTVSS

SEQ ID NO: 436 - protein, 118 aa, synthetic construct; identical to SEQ ID NO: 429
  REGION 1..118: note = Modified VH domain from a human immunoglobulin germline
  VARIANT 98..107: note = X is any residue

SEQ ID NO: 437 - protein, 118 aa, synthetic construct; identical to SEQ ID NO: 429
  REGION 1..118: note = Modified VH domain from a human immunoglobulin germline
  VARIANT 98..107: note = X is any residue

SEQ ID NO: 438 - protein, 118 aa, synthetic construct
  REGION 1..118: note = Modified VH domain from a human immunoglobulin germline
  VARIANT 98..107: note = X is any residue
QITLKESGPTLVKPTQTLCLTCTFSGFSLSTSGVGVGWYRQPPGKALEWLALIYWNDDKRYSPSLKSRLTITKDTSKNQVVLCMTNMTPVDTATYYCXXXXXXXXXXWGQGTLVTVSS

SEQ ID NO: 439 - protein, 118 aa, synthetic construct
  REGION 1..118: note = Modified VH domain from a human immunoglobulin germline
  VARIANT 98..107: note = X is any residue
QVTLKESGPVLVKPTETLTLTCTVSGFSLSNARMGVSWIRQPPGKALEWLAHIFSNDEKSYSTSLKSRLTISKDTSKSQVVLTMTNMDPVDTATYYCXXXXXXXXXXWGQGTLVTVSS

SEQ ID NO: 440 - protein, 118 aa, synthetic construct
  REGION 1..118: note = Modified VH domain from a human immunoglobulin germline
  VARIANT 98..107: note = X is any residue
QVTLKESGPVLVKPTETLCLTCTVSGFSLSNARMGVSWYRQPPGKALEWLAHIFSNDEKSYSTSLKSRLTISKDTSKSQVVLCMTNMTPVDTATYYCXXXXXXXXXXWGQGTLVTVSS

SEQ ID NO: 441 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWIGWVRRMPGKGLEWMGIIYPGDSDTRYSPSFQGQVTISADKSISTAYLQWSSLKASDTAMYYCXXXXXXXXXXXXXXXXXXXXXWGQGTMVTVSS

SEQ ID NO: 442 - protein, 128 aa, synthetic construct
  REGION 1..128: note = Modified VH domain from a human immunoglobulin germline
  VARIANT 97..117: note = X is any residue
EVRLVQSGAEVKKPGESLKISCKGSGYSFTSYWIGWVRRMPGKGLEWMGIIYPGDSDTRYSPSFQGQVTISADKSISTAYLQWSSLKASDTAMYYCXXXXXXXXXXXXXXXXXXXXXWGQGTMVTVSS

SEQ ID NO: 443 - protein, 128 aa, synthetic construct
  REGION 1..128: note = Modified VH domain from a human immunoglobulin germline
  VARIANT 97..117: note = X is any residue
EVQLVQSDAEVKKPGESLKISCKGSGYSFTSYWIGWVRRMPGKGLEWMGIIYPGDSDTRYSPSFQGQVTISADKSISTAYLQWSSLKASDTAMYYCXXXXXXXXXXXXXXXXXXXXXWGQGTMVTVSS

SEQ ID NO: 444 - protein, 128 aa, synthetic construct
  REGION 1..128: note = Modified VH domain from a human immunoglobulin germline
  VARIANT 97..117: note = X is any residue
EVQLVQSSAEVKKPGESLKISCKGSGYSFTSYWIGWVRRMPGKGLEWMGIIYPGDSDTRYSPSFQGQVTISADKSISTAYLQWSSLKASDTAMYYCXXXXXXXXXXXXXXXXXXXXXWGQGTMVTVSS

SEQ ID NO: 445 - protein, 128 aa, synthetic construct
  REGION 1..128: note = Modified VH domain from a human immunoglobulin germline
  VARIANT 97..117: note = X is any residue
EVQLVQSGDEVKKPGESLKISCKGSGYSFTSYWIGWVRRMPGKGLEWMGIIYPGDSDTRYSPSFQGQVTISADKSISTAYLQWSSLKASDTAMYYCXXXXXXXXXXXXXXXXXXXXXWGQGTMVTVSS

SEQ ID NO: 446 - protein, 128 aa, synthetic construct
  REGION 1..128: note = Modified VH domain from a human immunoglobulin germline
  VARIANT 97..117: note = X is any residue
EVQLVQSGPEVKKPGESLKISCKGSGYSFTSYWIGWVRRMPGKGLEWMGIIYPGDSDTRYSPSFQGQVTISADKSISTAYLQWSSLKASDTAMYYCXXXXXXXXXXXXXXXXXXXXXWGQGTMVTVSS

SEQ ID NO: 447 - protein, 128 aa, synthetic construct
  REGION 1..128: note = Modified VH domain from a human immunoglobulin germline
  VARIANT 97..117: note = X is any residue
EVQLVQSGAKVKKPGESLKISCKGSGYSFTSYWIGWVRRMPGKGLEWMGIIYPGDSDTRYSPSFQGQVTISADKSISTAYLQWSSLKASDTAMYYCXXXXXXXXXXXXXXXXXXXXXWGQGTMVTVSS

SEQ ID NO: 448 - protein, 128 aa, synthetic construct
  REGION 1..128: note = Modified VH domain from a human immunoglobulin germline
  VARIANT 97..117: note = X is any residue
EVQLVQSGAQVKKPGESLKISCKGSGYSFTSYWIGWVRRMPGKGLEWMGIIYPGDSDTRYSPSFQGQVTISADKSISTAYLQWSSLKASDTAMYYCXXXXXXXXXXXXXXXXXXXXXWGQGTMVTVSS

SEQ ID NO: 449 - protein, 128 aa, synthetic construct
  REGION 1..128: note = Modified VH domain from a human immunoglobulin germline
  VARIANT 97..117: note = X is any residue
EVQLVQSGAEVKKPGEPLKISCKGSGYSFTSYWIGWVRRMPGKGLEWMGIIYPGDSDTRYSPSFQGQVTISADKSISTAYLQWSSLKASDTAMYYCXXXXXXXXXXXXXXXXXXXXXWGQGTMVTVSS

SEQ ID NO: 450 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYDFTSYWIGWVRRMPGKGLEWMGIIYPGDSDTRYSPSFQGQVTISADKSISTAYLQWSSLKASDTAMYYCXXXXXXXXXXXXXXXXXXXXXWGQGTMVTVSS

SEQ ID NO: 451 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWIAWVRRMPGKGLEWMGIIYPGDSDTRYSPSFQGQVTISADKSISTAYLQWSSLKASDTAMYYCXXXXXXXXXXXXXXXXXXXXXWGQGTMVTVSS

SEQ ID NO: 452 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWITWVRRMPGKGLEWMGIIYPGDSDTRYSPSFQGQVTISADKSISTAYLQWSSLKASDTAMYYCXXXXXXXXXXXXXXXXXXXXXWGQGTMVTVSS

SEQ ID NO: 453 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWIGWYRRMPGKGLEWMGIIYPGDSDTRYSPSFQGQVTISADKSISTAYLQWSSLKASDTAMYYCXXXXXXXXXXXXXXXXXXXXXWGQGTMVTVSS

SEQ ID NO: 454 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWIGWVRRPPGKGLEWMGIIYPGDSDTRYSPSFQGQVTISADKSISTAYLQWSSLKASDTAMYYCXXXXXXXXXXXXXXXXXXXXXWGQGTMVTVSS

SEQ ID NO: 455 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWIGWVRRQPGKGLEWMGIIYPGDSDTRYSPSFQGQVTISADKSISTAYLQWSSLKASDTAMYYCXXXXXXXXXXXXXXXXXXXXXWGQGTMVTVSS

SEQ ID NO: 456 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWIGWVRRMPGKGLEYMGIIYPGDSDTRYSPSFQGQVTISADKSISTAYLQWSSLKASDTAMYYCXXXXXXXXXXXXXXXXXXXXXWGQGTMVTVSS

SEQ ID NO: 457 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWIGWVRRMPGKGLEQMGIIYPGDSDTRYSPSFQGQVTISADKSISTAYLQWSSLKASDTAMYYCXXXXXXXXXXXXXXXXXXXXXWGQGTMVTVSS

SEQ ID NO: 458 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWIGWVRRMPGKGLEWIGIIYPGDSDTRYSPSFQGQVTISADKSISTAYLQWSSLKASDTAMYYCXXXXXXXXXXXXXXXXXXXXXWGQGTMVTVSS

SEQ ID NO: 459 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWIGWVRRMPGKGLEWMGIIYPGDSDTEYSPSFQGQVTISADKSISTAYLQWSSLKASDTAMYYCXXXXXXXXXXXXXXXXXXXXXWGQGTMVTVSS

SEQ ID NO: 460 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWIGWVRRMPGKGLEWMGIIYPGDSDTRYDPSFQGQVTISADKSISTAYLQWSSLKASDTAMYYCXXXXXXXXXXXXXXXXXXXXXWGQGTMVTVSS

SEQ ID NO: 461 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWIGWVRRMPGKGLEWMGIIYPGDSDTRYAPSFQGQVTISADKSISTAYLQWSSLKASDTAMYYCXXXXXXXXXXXXXXXXXXXXXWGQGTMVTVSS

SEQ ID NO: 462 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWIGWVRRMPGKGLEWMGIIYPGDSDTRYSPSFQGQVEISADKSISTAYLQWSSLKASDTAMYYCXXXXXXXXXXXXXXXXXXXXXWGQGTMVTVSS

SEQ ID NO: 463 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWIGWVRRMPGKGLEWMGIIYPGDSDTRYSPSFQGQVTISADKRISTAYLQWSSLKASDTAMYYCXXXXXXXXXXXXXXXXXXXXXWGQGTMVTVSS

SEQ ID NO: 464 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWIGWVRRMPGKGLEWMGIIYPGDSDTRYSPSFQGQVTISADKSINTAYLQWSSLKASDTAMYYCXXXXXXXXXXXXXXXXXXXXXWGQGTMVTVSS

SEQ ID NO: 465 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWIGWVRRMPGKGLEWMGIIYPGDSDTRYSPSFQGQVTISADKSIQTAYLQWSSLKASDTAMYYCXXXXXXXXXXXXXXXXXXXXXWGQGTMVTVSS

SEQ ID NO: 466 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWIGWVRRMPGKGLEWMGIIYPGDSDTRYSPSFQGQVTISADKSISVAYLQWSSLKASDTAMYYCXXXXXXXXXXXXXXXXXXXXXWGQGTMVTVSS

SEQ ID NO: 467 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWIGWVRRMPGKGLEWMGIIYPGDSDTRYSPSFQGQVTISADKSISTAYLQWSSLDASDTAMYYCXXXXXXXXXXXXXXXXXXXXXWGQGTMVTVSS

SEQ ID NO: 468 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWIGWVRRMPGKGLEWMGIIYPGDSDTRYSPSFQGQVTISADKSISTAYLQWSSLTASDTAMYYCXXXXXXXXXXXXXXXXXXXXXWGQGTMVTVSS

SEQ ID NO: 469 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWIGWVRRMPGKGLEWMGIIYPGDSDTRYSPSFQGQVTISADKSISTAYLQWSSLKESDTAMYYCXXXXXXXXXXXXXXXXXXXXXWGQGTMVTVSS

SEQ ID NO: 470 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWIGWVRRMPGKGLEWMGIIYPGDSDTRYSPSFQGQVTISADKSISTAYLQWSSLKASDTAVYYCXXXXXXXXXXXXXXXXXXXXXWGQGTMVTVSS

SEQ ID NO: 471 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWIGWVRRMPGKGLEWMGIIYPGDSDTRYSPSFQGQVTISADKSISTAYLQWSSLKASDTAIYYCXXXXXXXXXXXXXXXXXXXXXWGQGTMVTVSS

SEQ ID NO: 472 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWIGWVRRMPGKGLEWMGIIYPGDSDTRYSPSFQGQVTISADKSISTAYLQWSSLKASDTAMYYCXXXXXXXXXXXXXXXXXXXXXWGQGTMVIVSS

SEQ ID NO: 473 - protein, 128 aa, synthetic construct
  REGION 1..128: note = Modified VH domain from a human immunoglobulin germline
  VARIANT 97..117: note = X is any residue
EVQLVQSGAEVKKPGECLKISCKGSGYSFTSYWIGWVRRMPGKGLEWMGIIYPGDSDTRYSPSFQGQVTISADKSISTAYLQWCSLKASDTAMYYCXXXXXXXXXXXXXXXXXXXXXWGQGTMVTVSS

SEQ ID NO: 474 - protein, 128 aa, synthetic construct
  REGION 1..128: note = Modified VH domain from a human immunoglobulin germline
  VARIANT 97..117: note = X is any residue
EVQLVQSGAEVKKPGESLCISCKGSGYSFTSYWIGWVRRMPGKGLEWMGIIYPGDSDTRYSPSFQGQVTISADKSISTAYLCWSSLKASDTAMYYCXXXXXXXXXXXXXXXXXXXXXWGQGTMVTVSS

SEQ ID NO: 475 - protein, 128 aa, synthetic construct
  REGION 1..128: note = Modified VH domain from a human immunoglobulin germline
  VARIANT 97..117: note = X is any residue
EVQLVQSGAEVKKPGESLKISCCGSGYSFTSYWIGWVRRMPGKGLEWMGIIYPGDSDTRYSPSFQGQVTISADKSISCAYLQWSSLKASDTAMYYCXXXXXXXXXXXXXXXXXXXXXWGQGTMVTVSS

SEQ ID NO: 476 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYSFTSYWICWVRRMPGKGLEWMGCIYPGDSDTRYSPSFQGQVTISADKSISTAYLQWSSLKASDTAMYYCXXXXXXXXXXXXXXXXXXXXXWGQGTMVTVSS

SEQ ID NO: 477 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYDFTSYWIGWVRRMPGKGLEWIGIIYPGDSDTRYSPSFQGQVTISADKSISTAYLQWSSLKESDTAMYYCXXXXXXXXXXXXXXXXXXXXXWGQGTMVTVSS

SEQ ID NO: 478 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYDFTSYWIGWVRRMPGKGLEWMGIIYPGDSDTRYSPSFQGQVTISADKSINTAYLQWSSLDASDTAMYYCXXXXXXXXXXXXXXXXXXXXXWGQGTMVTVSS

SEQ ID NO: 479 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYDFTSYWIGWVRRMPGKGLEWMGIIYPGDSDTRYSPSFQGQVTISADKSINTAYLQWSSLKESDTAMYYCXXXXXXXXXXXXXXXXXXXXXWGQGTMVTVSS

SEQ ID NO: 480 - protein, 128 aa, synthetic construct
  REGION 1..128: note = Modified VH domain from a human immunoglobulin germline
  VARIANT 97..117: note = X is any residue
EVQLVQSGAEVKKPGESLKISCKGSGYDFTSYWIGWVRRMPGKGLEWIGIIYPGDSDTRYSPSFQGQVTISADKSISTAYLQWSSLDASDTAMYYCXXXXXXXXXXXXXXXXXXXXXWGQGTMVTVSS

SEQ ID NO: 481 - protein, 120 aa, synthetic construct; identical to SEQ ID NO: 219
  REGION 1..120: note = Modified VH domain from a human immunoglobulin germline
  VARIANT 97..109: note = X is any residue

SEQ ID NO: 482 - protein, 120 aa, synthetic construct
  REGION 1..120: note = Modified VH domain from a human immunoglobulin germline
  VARIANT 97..109: note = X is any residue
QVQLVQSGAEVKKPGACVKVSCKASGYTFTSYDINWYRQATGQGLEWIGWMNPNSGNTGYAQKFQGRVTMTRNTSISTAYMELCSLRSEDTAVYYCXXXXXXXXXXXXXWGQGTLVTVSS

SEQ ID NO: 483 - protein, 118 aa, synthetic construct; identical to SEQ ID NO: 308
  REGION 1..118: note = Modified VH domain from a human immunoglobulin germline
  VARIANT 97..107: note = X is any residue

SEQ ID NO: 484 - protein, 123 aa, synthetic construct
  REGION 1..123: note = Modified VH domain from a human immunoglobulin germline
  VARIANT 97..112: note = X is any residue
EVQLVESGGGVVRPGGSLRLSCCASGFTFDDYGMSWYRQAPGKGLEWVAGINWNGGSTGYADSVKGRFTISRDNEKNCLYLQMNSLRAEDTALYHCXXXXXXXXXXXXXXXXWGQGTMVTVSS

SEQ ID NO: 485 - protein, 117 aa, synthetic construct; identical to SEQ ID NO: 380
  REGION 1..117: note = Modified VH domain from a human immunoglobulin germline
  VARIANT 96..106: note = X is any residue

SEQ ID NO: 486 - protein, 117 aa, synthetic construct
  REGION 1..117: note = Modified VH domain from a human immunoglobulin germline
  VARIANT 96..106: note = X is any residue
QVQLQQWGAGLLKPSETLSLTCCVYGGSFSGYYWSWYRQPPGKGLEWIGEINHSGSTNYNPSLKSRVTISVDTSKNCFSLKLSDVTPADTAVYYCXXXXXXXXXXXWGQGTMVTVSS

SEQ ID NO: 487 - protein, 98 aa, synthetic construct
  REGION 1..98: note = Modified VH domain from a human immunoglobulin germline
QVQLVQSGAEVKKPGACVKVSCKASGYTFTSYGISWVRRAPGQGLEWIGWISAYNGNTNYAQKLQGRVTMTTDTSTSTAYMELCSLRSDDTAVYYCAR

SEQ ID NO: 488 - protein, 98 aa, synthetic construct
  REGION 1..98: note = Modified VH domain from a human immunoglobulin germline
QVQLVQSGAEVKKPGACVKVSCKASGYTFTSYGISWVRRAPGQGEEWIGWISAYNGNTNYAQKLQGRVTMTTDTSTSTAYMELCSLRSDDTAVYYCAR

SEQ ID NO: 489 - protein, 98 aa, synthetic construct
  REGION 1..98: note = Modified VH domain from a human immunoglobulin germline
QVQLVQSGAEVKKPGACVKVSCKASGYTFTSYGISWYRQAPGQGLEWIGWISAYNGNTNYAQKLQGRVTMTTDTSTSTAYMELCSLRSDDTAVYYCAR

SEQ ID NO: 490 - protein, 98 aa, synthetic construct
  REGION 1..98: note = Modified VH domain from a human immunoglobulin germline
QVQLVQSGAEVKKPGACVKVSCKASGYTFTGYYMHWVRRAPGQGLEWIGRINPNSGGTNYAQKFQGRVTSTRDTSISTAYMELCRLRSDDTVVYYCAR

SEQ ID NO: 491 - protein, 98 aa, synthetic construct
  REGION 1..98: note = Modified VH domain from a human immunoglobulin germline
QVQLVQSGAEVKKPGACVKVSCKASGYTFTGYYMHWVRRAPGQGEEWIGRINPNSGGTNYAQKFQGRVTSTRDTSISTAYMELCRLRSDDTVVYYCAR

SEQ ID NO: 492 - protein, 98 aa, synthetic construct
  REGION 1..98: note = Modified VH domain from a human immunoglobulin germline
QVQLVQSGAEVKKPGACVKVSCKASGYTFTGYYMHWYRQAPGQGLEWIGRINPNSGGTNYAQKFQGRVTSTRDTSISTAYMELCRLRSDDTVVYYCAR

SEQ ID NO: 493 - protein, 98 aa, synthetic construct
  REGION 1..98: note = Modified VH domain from a human immunoglobulin germline
QVQLVQSGAEVKKPGACVKVSCKVSGYTLTELSMHWVRRAPGKGLEWIGGFDPEDGETIYAQKFQGRVTMTEDTSTDTAYMELCSLRSEDTAVYYCAT

SEQ ID NO: 494 - protein, 98 aa, synthetic construct
  REGION 1..98: note = Modified VH domain from a human immunoglobulin germline
QVQLVQSGAEVKKPGACVKVSCKVSGYTLTELSMHWVRRAPGKGEEWIGGFDPEDGETIYAQKFQGRVTMTEDTSTDTAYMELCSLRSEDTAVYYCAT

SEQ ID NO: 495 - protein, 98 aa, synthetic construct
  REGION 1..98: note = Modified VH domain from a human immunoglobulin germline
QVQLVQSGAEVKKPGACVKVSCKVSGYTLTELSMHWYRQAPGKGLEWIGGFDPEDGETIYAQKFQGRVTMTEDTSTDTAYMELCSLRSEDTAVYYCAT

SEQ ID NO: 496 - protein, 98 aa, synthetic construct
  REGION 1..98: note = Modified VH domain from a human immunoglobulin germline
QVQLVQSGAEVKKPGACVKVSCKASGYTFTSYAMHWVRRAPGQRLEWIGWINAGNGNTKYSQKFQGRVTITRDTSASTAYMELCSLRSEDTAVYYCAR

SEQ ID NO: 497 - protein, 98 aa, synthetic construct
  REGION 1..98: note = Modified VH domain from a human immunoglobulin germline
QVQLVQSGAEVKKPGACVKVSCKASGYTFTSYAMHWVRRAPGQREEWIGWINAGNGNTKYSQKFQGRVTITRDTSASTAYMELCSLRSEDTAVYYCAR

SEQ ID NO: 498 - protein, 98 aa, synthetic construct
  REGION 1..98: note = Modified VH domain from a human immunoglobulin germline
QVQLVQSGAEVKKPGACVKVSCKASGYTFTSYAMHWYRQAPGQRLEWIGWINAGNGNTKYSQKFQGRVTITRDTSASTAYMELCSLRSEDTAVYYCAR

SEQ ID NO: 499 - protein, 98 aa, synthetic construct
  REGION 1..98: note = Modified VH domain from a human immunoglobulin germline
QMQLVQSGAEVKKTGSCVKVSCKASGYTFTYRYLHWVRRAPGQALEWIGWITPFNGNTNYAQKFQDRVTITRDRSMSTAYMELCSLRSEDTAMYYCAR

SEQ ID NO: 500 - protein, 98 aa, synthetic construct
  REGION 1..98: note = Modified VH domain from a human immunoglobulin germline
QMQLVQSGAEVKKTGSCVKVSCKASGYTFTYRYLHWVRRAPGQAEEWIGWITPFNGNTNYAQKFQDRVTITRDRSMSTAYMELCSLRSEDTAMYYCAR

SEQ ID NO: 501 - protein, 98 aa, synthetic construct
  REGION 1..98: note = Modified VH domain from a human immunoglobulin germline
QMQLVQSGAEVKKTGSCVKVSCKASGYTFTYRYLHWYRQAPGQALEWIGWITPFNGNTNYAQKFQDRVTITRDRSMSTAYMELCSLRSEDTAMYYCAR

SEQ ID NO: 502 - protein, 98 aa, synthetic construct
  REGION 1..98: note = Modified VH domain from a human immunoglobulin germline
QVQLVQSGAEVKKPGACVKVSCKASGYTFTSYYMHWVRRAPGQGLEWIGIINPSGGSTSYAQKFQGRVTMTRDTSTSTVYMELCSLRSEDTAVYYCAR

SEQ ID NO: 503 - protein, 98 aa, synthetic construct
  REGION 1..98: note = Modified VH domain from a human immunoglobulin germline
QVQLVQSGAEVKKPGACVKVSCKASGYTFTSYYMHWVRRAPGQGEEWIGIINPSGGSTSYAQKFQGRVTMTRDTSTSTVYMELCSLRSEDTAVYYCAR

SEQ ID NO: 504 - protein, 98 aa, synthetic construct
  REGION 1..98: note = Modified VH domain from a human immunoglobulin germline
QVQLVQSGAEVKKPGACVKVSCKASGYTFTSYYMHWYRQAPGQGLEWIGIINPSGGSTSYAQKFQGRVTMTRDTSTSTVYMELCSLRSEDTAVYYCAR

SEQ ID NO: 505 - protein, 98 aa, synthetic construct
  REGION 1..98: note = Modified VH domain from a human immunoglobulin germline
QMQLVQSGPEVKKPGTCVKVSCKASGFTFTSSAVQWVRRARGQRLEWIGWIVVGSGNTNYAQKFQERVTITRDMSTSTAYMELCSLRSEDTAVYYCAA

SEQ ID NO: 506 - protein, 98 aa, synthetic construct
  REGION 1..98: note = Modified VH domain from a human immunoglobulin germline
QMQLVQSGPEVKKPGTCVKVSCKASGFTFTSSAVQWVRRARGQREEWIGWIVVGSGNTNYAQKFQERVTITRDMSTSTAYMELCSLRSEDTAVYYCAA

SEQ ID NO: 507 - protein, 98 aa, synthetic construct
  REGION 1..98: note = Modified VH domain from a human immunoglobulin germline
QMQLVQSGPEVKKPGTCVKVSCKASGFTFTSSAVQWYRQARGQRLEWIGWIVVGSGNTNYAQKFQERVTITRDMSTSTAYMELCSLRSEDTAVYYCAA

SEQ ID NO: 508 - protein, 98 aa, synthetic construct
  REGION 1..98: note = Modified VH domain from a human immunoglobulin germline
QVQLVQSGAEVKKPGSCVKVSCKASGGTFSSYAISWVRRAPGQGLEWIGGIIPIFGTANYAQKFQGRVTITADESTSTAYMELCSLRSEDTAVYYCAR

SEQ ID NO: 509 - protein, 98 aa, synthetic construct
  REGION 1..98: note = Modified VH domain from a human immunoglobulin germline
QVQLVQSGAEVKKPGSCVKVSCKASGGTFSSYAISWVRRAPGQGEEWIGGIIPIFGTANYAQKFQGRVTITADESTSTAYMELCSLRSEDTAVYYCAR

SEQ ID NO: 510 - protein, 98 aa, synthetic construct
  REGION 1..98: note = Modified VH domain from a human immunoglobulin germline
QVQLVQSGAEVKKPGSCVKVSCKASGGTFSSYAISWYRQAPGQGLEWIGGIIPIFGTANYAQKFQGRVTITADESTSTAYMELCSLRSEDTAVYYCAR

SEQ ID NO: 511 - protein, 98 aa, synthetic construct
  REGION 1..98: note = Modified VH domain from a human immunoglobulin germline
QVQLVQSGAEVKKPGACVKVSCKASGYTFTSYDINWVRRATGQGLEWIGWMNPNSGNTGYAQKFQGRVTMTRNTSISTAYMELCSLRSEDTAVYYCAR

SEQ ID NO: 512 - protein, 98 aa, synthetic construct
  REGION 1..98: note = Modified VH domain from a human immunoglobulin germline
QVQLVQSGAEVKKPGACVKVSCKASGYTFTSYDINWVRRATGQGEEWIGWMNPNSGNTGYAQKFQGRVTMTRNTSISTAYMELCSLRSEDTAVYYCAR

SEQ ID NO: 513 - protein, 98 aa, synthetic construct
  REGION 1..98: note = Modified VH domain from a human immunoglobulin germline
QVQLVQSGAEVKKPGACVKVSCKASGYTFTSYDINWYRQATGQGLEWIGWMNPNSGNTGYAQKFQGRVTMTRNTSISTAYMELCSLRSEDTAVYYCAR

SEQ ID NO: 514 - protein, 98 aa, synthetic construct
  REGION 1..98: note = Modified VH domain from a human immunoglobulin germline
EVQLVQSGAEVKKPGACVKISCKVSGYTFTDYYMHWVQRAPGKGLEWIGLVDPEDGETIYAEKFQGRVTITADTSTDTAYMELCSLRSEDTAVYYCAT

SEQ ID NO: 515 - protein, 98 aa, synthetic construct
  REGION 1..98: note = Modified VH domain from a human immunoglobulin germline
EVQLVQSGAEVKKPGACVKISCKVSGYTFTDYYMHWVQRAPGKGEEWIGLVDPEDGETIYAEKFQGRVTITADTSTDTAYMELCSLRSEDTAVYYCAT

SEQ ID NO: 516 - protein, 98 aa, synthetic construct
  REGION 1..98: note = Modified VH domain from a human immunoglobulin germline
EVQLVQSGAEVKKPGACVKISCKVSGYTFTDYYMHWYQQAPGKGLEWIGLVDPEDGETIYAEKFQGRVTITADTSTDTAYMELCSLRSEDTAVYYCAT

SEQ ID NO: 517 - protein, 98 aa, synthetic construct
  REGION 1..98: note = Modified VH domain from a human immunoglobulin germline
QVQLVESGGGLVKPGGSLRLSCCASGFTFSDYYMSWIRRAPGKGLEWVAYISSSGSTIYYADSVKGRFTISRDNEKNCLYLQMNSLRAEDTAVYYCAR

SEQ ID NO: 518 - protein, 98 aa, synthetic construct
  REGION 1..98: note = Modified VH domain from a human immunoglobulin germline
QVQLVESGGGLVKPGGSLRLSCCASGFTFSDYYMSWIRRAPGKGEEWVAYISSSGSTIYYADSVKGRFTISRDNEKNCLYLQMNSLRAEDTAVYYCAR

SEQ ID NO: 519 - protein, 98 aa, synthetic construct
  REGION 1..98: note = Modified VH domain from a human immunoglobulin germline
QVQLVESGGGLVKPGGSLRLSCCASGFTFSDYYMSWYRQAPGKGLEWVAYISSSGSTIYYADSVKGRFTISRDNEKNCLYLQMNSLRAEDTAVYYCAR

SEQ ID NO: 520 - protein, 97 aa, synthetic construct
  REGION 1..97: note = Modified VH domain from a human immunoglobulin germline
EVQLVESGGGLVQPGGSLRLSCCASGFTFSSYDMHWVRRATGKGLEWVAAIGTAGDTYYPGSVKGRFTISRENEKNCLYLQMNSLRAGDTAVYYCAR

SEQ ID NO: 521 - protein, 97 aa, synthetic construct
  REGION 1..97: note = Modified VH domain from a human immunoglobulin germline
EVQLVESGGGLVQPGGSLRLSCCASGFTFSSYDMHWVRRATGKGEEWVAAIGTAGDTYYPGSVKGRFTISRENEKNCLYLQMNSLRAGDTAVYYCAR

SEQ ID NO: 522 - protein, 97 aa, synthetic construct
  REGION 1..97: note = Modified VH domain from a human immunoglobulin germline
EVQLVESGGGLVQPGGSLRLSCCASGFTFSSYDMHWYRQATGKGLEWVAAIGTAGDTYYPGSVKGRFTISRENEKNCLYLQMNSLRAGDTAVYYCAR

SEQ ID NO: 523 - protein, 100 aa, synthetic construct
  REGION 1..100: note = Modified VH domain from a human immunoglobulin germline
EVQLVESGGGLVKPGGSLRLSCCASGFTFSNAWMSWVRRAPGKGLEWVARIKSKTDGGTTDYAAPVKGRFTISRDDEKNCLYLQMNSLKTEDTAVYYCTT

SEQ ID NO: 524 - protein, 100 aa, synthetic construct
  REGION 1..100: note = Modified VH domain from a human immunoglobulin germline
EVQLVESGGGLVKPGGSLRLSCCASGFTFSNAWMSWVRRAPGKGEEWVARIKSKTDGGTTDYAAPVKGRFTISRDDEKNCLYLQMNSLKTEDTAVYYCTT

SEQ ID NO: 525 - protein, 100 aa, synthetic construct
  REGION 1..100: note = Modified VH domain from a human immunoglobulin germline
EVQLVESGGGLVKPGGSLRLSCCASGFTFSNAWMSWYRQAPGKGLEWVARIKSKTDGGTTDYAAPVKGRFTISRDDEKNCLYLQMNSLKTEDTAVYYCTT

SEQ ID NO: 526 - protein, 98 aa, synthetic construct
  REGION 1..98: note = Modified VH domain from a human immunoglobulin germline
EVQLVESGGGVVRPGGSLRLSCCASGFTFDDYGMSWVRRAPGKGLEWVAGINWNGGSTGYADSVKGRFTISRDNEKNCLYLQMNSLRAEDTALYHCAR

SEQ ID NO: 527 - protein, 98 aa, synthetic construct
  REGION 1..98: note = Modified VH domain from a human immunoglobulin germline
EVQLVESGGGVVRPGGSLRLSCCASGFTFDDYGMSWVRRAPGKGEEWVAGINWNGGSTGYADSVKGRFTISRDNEKNCLYLQMNSLRAEDTALYHCAR

SEQ ID NO: 528 - protein, 98 aa, synthetic construct
  REGION 1..98: note = Modified VH domain from a human immunoglobulin germline
EVQLVESGGGVVRPGGSLRLSCCASGFTFDDYGMSWYRQAPGKGLEWVAGINWNGGSTGYADSVKGRFTISRDNEKNCLYLQMNSLRAEDTALYHCAR

SEQ ID NO: 529 - protein, 98 aa, synthetic construct
  REGION 1..98: note = Modified VH domain from a human immunoglobulin germline
EVQLVESGGGLVKPGGSLRLSCCASGFTFSSYSMNWVRRAPGKGLEWVASISSSSSYIYYADSVKGRFTISRDNEKNCLYLQMNSLRAEDTAVYYCAR

SEQ ID NO: 530 - protein, 98 aa, synthetic construct
  REGION 1..98: note = Modified VH domain from a human immunoglobulin germline
EVQLVESGGGLVKPGGSLRLSCCASGFTFSSYSMNWVRRAPGKGEEWVASISSSSSYIYYADSVKGRFTISRDNEKNCLYLQMNSLRAEDTAVYYCAR

SEQ ID NO: 531 - protein, 98 aa, synthetic construct
  REGION 1..98: note = Modified VH domain from a human immunoglobulin germline
EVQLVESGGGLVKPGGSLRLSCCASGFTFSSYSMNWYRQAPGKGLEWVASISSSSSYIYYADSVKGRFTISRDNEKNCLYLQMNSLRAEDTAVYYCAR

SEQ ID NO: 532 - protein, 98 aa, synthetic construct
  REGION 1..98: note = Modified VH domain from a human immunoglobulin germline
EVQLLESGGGLVQPGGSLRLSCCASGFTFSSYAMSWVRRAPGKGLEWVAAISGSGGSTYYADSVKGRFTISRDNEKNCLYLQMNSLRAEDTAVYYCAK

SEQ ID NO: 533 - protein, 98 aa, synthetic construct
  REGION 1..98: note = Modified VH domain from a human immunoglobulin germline
EVQLLESGGGLVQPGGSLRLSCCASGFTFSSYAMSWVRRAPGKGEEWVAAISGSGGSTYYADSVKGRFTISRDNEKNCLYLQMNSLRAEDTAVYYCAK

SEQ ID NO: 534 - protein, 98 aa, synthetic construct
  REGION 1..98: note = Modified VH domain from a human immunoglobulin germline
EVQLLESGGGLVQPGGSLRLSCCASGFTFSSYAMSWYRQAPGKGLEWVAAISGSGGSTYYADSVKGRFTISRDNEKNCLYLQMNSLRAEDTAVYYCAK

SEQ ID NO: 535 - protein, 98 aa, synthetic construct
  REGION 1..98: note = Modified VH domain from a human immunoglobulin germline
QVQLVESGGGVVQPGRSLRLSCCASGFTFSSYAMHWVRRAPGKGLEWVAVISYDGSNKYYADSVKGRFTISRDNEKNCLYLQMNSLRAEDTAVYYCAR

SEQ ID NO: 536 - protein, 98 aa, synthetic construct
  REGION 1..98: note = Modified VH domain from a human immunoglobulin germline
QVQLVESGGGVVQPGRSLRLSCCASGFTFSSYAMHWVRRAPGKGEEWVAVISYDGSNKYYADSVKGRFTISRDNEKNCLYLQMNSLRAEDTAVYYCAR

SEQ ID NO: 537 - protein, 98 aa, synthetic construct
  REGION 1..98: note = Modified VH domain from a human immunoglobulin germline
QVQLVESGGGVVQPGRSLRLSCCASGFTFSSYAMHWYRQAPGKGLEWVAVISYDGSNKYYADSVKGRFTISRDNEKNCLYLQMNSLRAEDTAVYYCAR

SEQ ID NO: 538 - protein, 98 aa, synthetic construct
  REGION 1..98: note = Modified VH domain from a human immunoglobulin germline
QVQLVESGGGVVQPGRSLRLSCCASGFTFSSYGMHWVRRAPGKGLEWVAVIWYDGSNKYYADSVKGRFTISRDNEKNCLYLQMNSLRAEDTAVYYCAR

SEQ ID NO: 539 - protein, 98 aa, synthetic construct
  REGION 1..98: note = Modified VH domain from a human immunoglobulin germline
QVQLVESGGGVVQPGRSLRLSCCASGFTFSSYGMHWVRRAPGKGEEWVAVIWYDGSNKYYADSVKGRFTISRDNEKNCLYLQMNSLRAEDTAVYYCAR

SEQ ID NO: 540 - protein, 98 aa, synthetic construct
  REGION 1..98: note = Modified VH domain from a human immunoglobulin germline
QVQLVESGGGVVQPGRSLRLSCCASGFTFSSYGMHWYRQAPGKGLEWVAVIWYDGSNKYYADSVKGRFTISRDNEKNCLYLQMNSLRAEDTAVYYCAR

SEQ ID NO: 541 - protein, 98 aa, synthetic construct
  REGION 1..98: note = Modified VH domain from a human immunoglobulin germline
EVQLVESGGVVVQPGGSLRLSCCASGFTFDDYTMHWVRRAPGKGLEWVALISWDGGSTYYADSVKGRFTISRDNEKNCLYLQMNSLRTEDTALYYCAK

SEQ ID NO: 542 - protein, 98 aa, synthetic construct
  REGION 1..98: note = Modified VH domain from a human immunoglobulin germline
EVQLVESGGVVVQPGGSLRLSCCASGFTFDDYTMHWVRRAPGKGEEWVALISWDGGSTYYADSVKGRFTISRDNEKNCLYLQMNSLRTEDTALYYCAK

SEQ ID NO: 543 - protein, 98 aa, synthetic construct
  REGION 1..98: note = Modified VH domain from a human immunoglobulin germline
EVQLVESGGVVVQPGGSLRLSCCASGFTFDDYTMHWYRQAPGKGLEWVALISWDGGSTYYADSVKGRFTISRDNEKNCLYLQMNSLRTEDTALYYCAK

SEQ ID NO: 544 - protein, 98 aa, synthetic construct
  REGION 1..98: note = Modified VH domain from a human immunoglobulin germline
EVQLVESGGGLVQPGGSLRLSCCASGFTFSSYSMNWVRRAPGKGLEWVAYISSSSSTIYYADSVKGRFTISRDNEKNCLYLQMNSLRAEDTAVYYCAR

SEQ ID NO: 545 - protein, 98 aa, synthetic construct
  REGION 1..98: note = Modified VH domain from a human immunoglobulin germline
EVQLVESGGGLVQPGGSLRLSCCASGFTFSSYSMNWVRRAPGKGEEWVAYISSSSSTIYYADSVKGRFTISRDNEKNCLYLQMNSLRAEDTAVYYCAR

SEQ ID NO: 546 - protein, 98 aa, synthetic construct
  REGION 1..98: note = Modified VH domain from a human immunoglobulin germline
EVQLVESGGGLVQPGGSLRLSCCASGFTFSSYSMNWYRQAPGKGLEWVAYISSSSSTIYYADSVKGRFTISRDNEKNCLYLQMNSLRAEDTAVYYCAR

SEQ ID NO: 547 - protein, 100 aa, synthetic construct
  REGION 1..100: note = Modified VH domain from a human immunoglobulin germline
EVQLVESGGGLVQPGRSLRLSCCASGFTFGDYAMSWFRRAPGKGLEWVAFIRSKAYGGTTEYTASVKGRFTISRDGEKSCAYLQMNSLKTEDTAVYYCTR

SEQ ID NO: 548 - protein, 100 aa, synthetic construct
  REGION 1..100: note = Modified VH domain from a human immunoglobulin germline
EVQLVESGGGLVQPGRSLRLSCCASGFTFGDYAMSWFRRAPGKGEEWVAFIRSKAYGGTTEYTASVKGRFTISRDGEKSCAYLQMNSLKTEDTAVYYCTR

SEQ ID NO: 549 - protein, 100 aa, synthetic construct
  REGION 1..100: note = Modified VH domain from a human immunoglobulin germline
EVQLVESGGGLVQPGRSLRLSCCASGFTFGDYAMSWYRQAPGKGLEWVAFIRSKAYGGTTEYTASVKGRFTISRDGEKSCAYLQMNSLKTEDTAVYYCTR

SEQ ID NO: 550 - protein, 97 aa, synthetic construct
  REGION 1..97: note = Modified VH domain from a human immunoglobulin germline
EVQLVESGGGLIQPGGSLRLSCCASGFTVSSNYMSWVRRAPGKGLEWVAVIYSGGSTYYADSVKGRFTISRDNEKNCLYLQMNSLRAEDTAVYYCAR

SEQ ID NO: 551 - protein, 97 aa, synthetic construct
  REGION 1..97: note = Modified VH domain from a human immunoglobulin germline
EVQLVESGGGLIQPGGSLRLSCCASGFTVSSNYMSWVRRAPGKGEEWVAVIYSGGSTYYADSVKGRFTISRDNEKNCLYLQMNSLRAEDTAVYYCAR

SEQ ID NO: 552 - protein, 97 aa, synthetic construct
  REGION 1..97: note = Modified VH domain from a human immunoglobulin germline
EVQLVESGGGLIQPGGSLRLSCCASGFTVSSNYMSWYRQAPGKGLEWVAVIYSGGSTYYADSVKGRFTISRDNEKNCLYLQMNSLRAEDTAVYYCAR

SEQ ID NO: 553 - protein, 98 aa, synthetic construct
  REGION 1..98: note = Modified VH domain from a human immunoglobulin germline
EVQLVESGGGLVQPGGSLRLSCCASGFTFSSYAMHWVRRAPGKGLEYVAAISSNGGSTYYANSVKGRFTISRDNEKNCLYLQMGSLRAEDMAVYYCAR

SEQ ID NO: 554 - protein, 98 aa, synthetic construct
  REGION 1..98: note = Modified VH domain from a human immunoglobulin germline
EVQLVESGGGLVQPGGSLRLSCCASGFTFSSYAMHWVRRAPGKGEEYVAAISSNGGSTYYANSVKGRFTISRDNEKNCLYLQMGSLRAEDMAVYYCAR

SEQ ID NO: 555 - protein, 98 aa, synthetic construct
  REGION 1..98: note = Modified VH domain from a human immunoglobulin germline
EVQLVESGGGLVQPGGSLRLSCCASGFTFSSYAMHWYRQAPGKGLEYVAAISSNGGSTYYANSVKGRFTISRDNEKNCLYLQMGSLRAEDMAVYYCAR

SEQ ID NO: 556 - protein, 97 aa, synthetic construct
  REGION 1..97: note = Modified VH domain from a human immunoglobulin germline
EVQLVESGGGLVQPGGSLRLSCCASGFTVSSNYMSWVRRAPGKGLEWVAVIYSGGSTYYADSVKGRFTISRDNEKNCLYLQMNSLRAEDTAVYYCAR

SEQ ID NO: 557 - protein, 97 aa, synthetic construct
  REGION 1..97: note = Modified VH domain from a human immunoglobulin germline
EVQLVESGGGLVQPGGSLRLSCCASGFTVSSNYMSWVRRAPGKGEEWVAVIYSGGSTYYADSVKGRFTISRDNEKNCLYLQMNSLRAEDTAVYYCAR

SEQ ID NO: 558 - protein, 97 aa, synthetic construct
  REGION 1..97: note = Modified VH domain from a human immunoglobulin germline
EVQLVESGGGLVQPGGSLRLSCCASGFTVSSNYMSWYRQAPGKGLEWVAVIYSGGSTYYADSVKGRFTISRDNEKNCLYLQMNSLRAEDTAVYYCAR

SEQ ID NO: 559 - protein, 98 aa, synthetic construct
  REGION 1..98: note = Modified VH domain from a human immunoglobulin germline
EVQLVESGGGLVQPGGSLRLSCCASGFTFSSYWMSWVRRAPGKGLEWVANIKQDGSEKYYVDSVKGRFTISRDNEKNCLYLQMNSLRAEDTAVYYCAR

SEQ ID NO: 560 - protein, 98 aa, synthetic construct
  REGION 1..98: note = Modified VH domain from a human immunoglobulin germline
EVQLVESGGGLVQPGGSLRLSCCASGFTFSSYWMSWVRRAPGKGEEWVANIKQDGSEKYYVDSVKGRFTISRDNEKNCLYLQMNSLRAEDTAVYYCAR

SEQ ID NO: 561 - protein, 98 aa, synthetic construct
  REGION 1..98: note = Modified VH domain from a human immunoglobulin germline
EVQLVESGGGLVQPGGSLRLSCCASGFTFSSYWMSWYRQAPGKGLEWVANIKQDGSEKYYVDSVKGRFTISRDNEKNCLYLQMNSLRAEDTAVYYCAR

SEQ ID NO: 562 - protein, 100 aa, synthetic construct
  REGION 1..100: note = Modified VH domain from a human immunoglobulin germline
EVQLVESGGGLVQPGGSLRLSCCASGFTFSDHYMDWVRRAPGKGLEWVARTRNKANSYTTEYAASVKGRFTISRDDEKNCLYLQMNSLKTEDTAVYYCAR

SEQ ID NO: 563 - protein, 100 aa, synthetic construct
  REGION 1..100: note = Modified VH domain from a human immunoglobulin germline
EVQLVESGGGLVQPGGSLRLSCCASGFTFSDHYMDWVRRAPGKGEEWVARTRNKANSYTTEYAASVKGRFTISRDDEKNCLYLQMNSLKTEDTAVYYCAR

SEQ ID NO: 564 - protein, 100 aa, synthetic construct
  REGION 1..100: note = Modified VH domain from a human immunoglobulin germline
EVQLVESGGGLVQPGGSLRLSCCASGFTFSDHYMDWYRQAPGKGLEWVARTRNKANSYTTEYAASVKGRFTISRDDEKNCLYLQMNSLKTEDTAVYYCAR

SEQ ID NO: 565 - protein, 100 aa, synthetic construct
  REGION 1..100: note = Modified VH domain from a human immunoglobulin germline
EVQLVESGGGLVQPGGSLKLSCCASGFTFSGSAMHWVRRASGKGLEWVARIRSKANSYATAYAASVKGRFTISRDDEKNCAYLQMNSLKTEDTAVYYCTR

SEQ ID NO: 566 - protein, 100 aa, synthetic construct
  REGION 1..100: note = Modified VH domain from a human immunoglobulin germline
EVQLVESGGGLVQPGGSLKLSCCASGFTFSGSAMHWVRRASGKGEEWVARIRSKANSYATAYAASVKGRFTISRDDEKNCAYLQMNSLKTEDTAVYYCTR

SEQ ID NO: 567 - protein, 100 aa, synthetic construct
  REGION 1..100: note = Modified VH domain from a human immunoglobulin germline
EVQLVESGGGLVQPGGSLKLSCCASGFTFSGSAMHWYRQASGKGLEWVARIRSKANSYATAYAASVKGRFTISRDDEKNCAYLQMNSLKTEDTAVYYCTR

SEQ ID NO: 568 - protein, 98 aa, synthetic construct
  REGION 1..98: note = Modified VH domain from a human immunoglobulin germline
EVQLVESGGGLVQPGGSLRLSCCASGFTFSSYWMHWVRRAPGKGLVWVARINSDGSSTSYADSVKGRFTISRDNEKNCLYLQMNSLRAEDTAVYYCAR

SEQ ID NO: 569 - protein, 98 aa, synthetic construct
  REGION 1..98: note = Modified VH domain from a human immunoglobulin germline
EVQLVESGGGLVQPGGSLRLSCCASGFTFSSYWMHWVRRAPGKGEVWVARINSDGSSTSYADSVKGRFTISRDNEKNCLYLQMNSLRAEDTAVYYCAR

SEQ ID NO: 570 - protein, 98 aa, synthetic construct
  REGION 1..98: note = Modified VH domain from a human immunoglobulin germline
EVQLVESGGGLVQPGGSLRLSCCASGFTFSSYWMHWYRQAPGKGLVWVARINSDGSSTSYADSVKGRFTISRDNEKNCLYLQMNSLRAEDTAVYYCAR

SEQ ID NO: 571 - protein, 98 aa, synthetic construct
  REGION 1..98: note = Modified VH domain from a human immunoglobulin germline
EVQLVESGGGLVQPGRSLRLSCCASGFTFDDYAMHWVRRAPGKGLEWVAGISWNSGSIGYADSVKGRFTISRDNEKNCLYLQMNSLRAEDTALYYCAK

SEQ ID NO: 572 - protein, 98 aa, synthetic construct
  REGION 1..98: note = Modified VH domain from a human immunoglobulin germline
EVQLVESGGGLVQPGRSLRLSCCASGFTFDDYAMHWVRRAPGKGEEWVAGISWNSGSIGYADSVKGRFTISRDNEKNCLYLQMNSLRAEDTALYYCAK

SEQ ID NO: 573 - protein, 98 aa, synthetic construct
  REGION 1..98: note = Modified VH domain from a human immunoglobulin germline
EVQLVESGGGLVQPGRSLRLSCCASGFTFDDYAMHWYRQAPGKGLEWVAGISWNSGSIGYADSVKGRFTISRDNEKNCLYLQMNSLRAEDTALYYCAK

SEQ ID NO: 574 - protein, 96 aa, synthetic construct
  REGION 1..96: note = Modified VH domain from a human immunoglobulin germline
EVQLVESRGVLVQPGGSLRLSCCASGFTVSSNEMSWVRRAPGKGLEWVASISGGSTYYADSRKGRFTISRDNEKNCLHLQMNSLRAEDTAVYYCKK

SEQ ID NO: 575 - protein, 96 aa, synthetic construct
  REGION 1..96: note = Modified VH domain from a human immunoglobulin germline
EVQLVESRGVLVQPGGSLRLSCCASGFTVSSNEMSWVRRAPGKGEEWVASISGGSTYYADSRKGRFTISRDNEKNCLHLQMNSLRAEDTAVYYCKK

SEQ ID NO: 576 - protein, 96 aa, synthetic construct
  REGION 1..96: note = Modified VH domain from a human immunoglobulin germline
EVQLVESRGVLVQPGGSLRLSCCASGFTVSSNEMSWYRQAPGKGLEWVASISGGSTYYADSRKGRFTISRDNEKNCLHLQMNSLRAEDTAVYYCKK

SEQ ID NO: 577 - protein, 98 aa, synthetic construct
  REGION 1..98: note = Modified VH domain from a human immunoglobulin germline
QVQLQESGPGLVKPSDTLSLTCCVSGYSISSSNWWGWIRRPPGKGLEWIGYIYYSGSTYYNPSLKSRVTMSVDTSKNCFSLKLSDVTPVDTAVYYCAR

SEQ ID NO: 578 - protein, 98 aa, synthetic construct
  REGION 1..98: note = Modified VH domain from a human immunoglobulin germline
QVQLQESGPGLVKPSDTLSLTCCVSGYSISSSNWWGWIRRPPGKGEEWIGYIYYSGSTYYNPSLKSRVTMSVDTSKNCFSLKLSDVTPVDTAVYYCAR

SEQ ID NO: 579 - protein, 98 aa, synthetic construct
  REGION 1..98: note = Modified VH domain from a human immunoglobulin germline
QVQLQESGPGLVKPSDTLSLTCCVSGYSISSSNWWGWYRQPPGKGLEWIGYIYYSGSTYYNPSLKSRVTMSVDTSKNCFSLKLSDVTPVDTAVYYCAR

SEQ ID NO: 580 - protein, 99 aa, synthetic construct
  REGION 1..99: note = Modified VH domain from a human immunoglobulin germline
QVQLQESGPGLVKPSQTLSLTCCVSGGSISSGGYYWSWIRRPPGKGLEWIGYIYYSGSTYYNPSLKSRVTISVDTSKNCFSLKLSDVTPADTAVYYCAR

SEQ ID NO: 581 - protein, 99 aa, synthetic construct
  REGION 1..99: note = Modified VH domain from a human immunoglobulin germline
QVQLQESGPGLVKPSQTLSLTCCVSGGSISSGGYYWSWIRRPPGKGEEWIGYIYYSGSTYYNPSLKSRVTISVDTSKNCFSLKLSDVTPADTAVYYCAR

SEQ ID NO: 582 - protein, 99 aa, synthetic construct
  REGION 1..99: note = Modified VH domain from a human immunoglobulin germline
QVQLQESGPGLVKPSQTLSLTCCVSGGSISSGGYYWSWYRQPPGKGLEWIGYIYYSGSTYYNPSLKSRVTISVDTSKNCFSLKLSDVTPADTAVYYCAR

SEQ ID NO: 583 - protein, 99 aa, synthetic construct
  REGION 1..99: note = Modified VH domain from a human immunoglobulin germline
QLQLQESGSGLVKPSQTLSLTCCVSGGSISSGGYSWSWIRRPPGKGLEWIGYIYHSGSTYYNPSLKSRVTISVDRSKNCFSLKLSDVTPADTAVYYCAR

SEQ ID NO: 584 - protein, 99 aa, synthetic construct
  REGION 1..99: note = Modified VH domain from a human immunoglobulin germline
QLQLQESGSGLVKPSQTLSLTCCVSGGSISSGGYSWSWIRRPPGKGEEWIGYIYHSGSTYYNPSLKSRVTISVDRSKNCFSLKLSDVTPADTAVYYCAR

SEQ ID NO: 585 - protein, 99 aa, synthetic construct
  REGION 1..99: note = Modified VH domain from a human immunoglobulin germline
QLQLQESGSGLVKPSQTLSLTCCVSGGSISSGGYSWSWYRQPPGKGLEWIGYIYHSGSTYYNPSLKSRVTISVDRSKNCFSLKLSDVTPADTAVYYCAR

SEQ ID NO: 586 - protein, 99 aa, synthetic construct
  REGION 1..99: note = Modified VH domain from a human immunoglobulin germline
QVQLQESGPGLVKPSQTLSLTCCVSGGSISSGDYYWSWIRRPPGKGLEWIGYIYYSGSTYYNPSLKSRVTISVDTSKNCFSLKLSDVTPADTAVYYCAR

SEQ ID NO: 587 - protein, 99 aa, synthetic construct
  REGION 1..99: note = Modified VH domain from a human immunoglobulin germline
QVQLQESGPGLVKPSQTLSLTCCVSGGSISSGDYYWSWIRRPPGKGEEWIGYIYYSGSTYYNPSLKSRVTISVDTSKNCFSLKLSDVTPADTAVYYCAR

SEQ ID NO: 588 - protein, 99 aa, synthetic construct
  REGION 1..99: note = Modified VH domain from a human immunoglobulin germline
QVQLQESGPGLVKPSQTLSLTCCVSGGSISSGDYYWSWYRQPPGKGLEWIGYIYYSGSTYYNPSLKSRVTISVDTSKNCFSLKLSDVTPADTAVYYCAR

SEQ ID NO: 589 - protein, 99 aa, synthetic construct
  REGION 1..99: note = Modified VH domain from a human immunoglobulin germline
QVQLQESGPGLVKPSQTLSLTCCVSGGSISSGGYYWSWIRRHPGKGLEWIGYIYYSGSTYYNPSLKSLVTISVDTSKNCFSLKLSDVTPADTAVYYCAR

SEQ ID NO: 590 - protein, 99 aa, synthetic construct
  REGION 1..99: note = Modified VH domain from a human immunoglobulin germline
QVQLQESGPGLVKPSQTLSLTCCVSGGSISSGGYYWSWIRRHPGKGEEWIGYIYYSGSTYYNPSLKSLVTISVDTSKNCFSLKLSDVTPADTAVYYCAR

SEQ ID NO: 591 - protein, 99 aa, synthetic construct
  REGION 1..99: note = Modified VH domain from a human immunoglobulin germline
QVQLQESGPGLVKPSQTLSLTCCVSGGSISSGGYYWSWYRQHPGKGLEWIGYIYYSGSTYYNPSLKSLVTISVDTSKNCFSLKLSDVTPADTAVYYCAR

SEQ ID NO: 592 - protein, 97 aa, synthetic construct
  REGION 1..97: note = Modified VH domain from a human immunoglobulin germline
QVQLQQWGAGLLKPSETLSLTCCVYGGSFSGYYWSWIRRPPGKGLEWIGEINHSGSTNYNPSLKSRVTISVDTSKNCFSLKLSDVTPADTAVYYCAR

SEQ ID NO: 593 - protein, 97 aa, synthetic construct
  REGION 1..97: note = Modified VH domain from a human immunoglobulin germline
QVQLQQWGAGLLKPSETLSLTCCVYGGSFSGYYWSWIRRPPGKGEEWIGEINHSGSTNYNPSLKSRVTISVDTSKNCFSLKLSDVTPADTAVYYCAR

SEQ ID NO: 594 - protein, 97 aa, synthetic construct
  REGION 1..97: note = Modified VH domain from a human immunoglobulin germline
QVQLQQWGAGLLKPSETLSLTCCVYGGSFSGYYWSWYRQPPGKGLEWIGEINHSGSTNYNPSLKSRVTISVDTSKNCFSLKLSDVTPADTAVYYCAR

SEQ ID NO: 595 - protein, 99 aa, synthetic construct
  REGION 1..99: note = Modified VH domain from a human immunoglobulin germline
QLQLQESGPGLVKPSETLSLTCCVSGGSISSSSYYWGWIRRPPGKGLEWIGSIYYSGSTYYNPSLKSRVTISVDTSKNCFSLKLSDVTPADTAVYYCAR

SEQ ID NO: 596 - protein, 99 aa, synthetic construct
  REGION 1..99: note = Modified VH domain from a human immunoglobulin germline
QLQLQESGPGLVKPSETLSLTCCVSGGSISSSSYYWGWIRRPPGKGEEWIGSIYYSGSTYYNPSLKSRVTISVDTSKNCFSLKLSDVTPADTAVYYCAR

SEQ ID NO: 597 - protein, 99 aa, synthetic construct
  REGION 1..99: note = Modified VH domain from a human immunoglobulin germline
QLQLQESGPGLVKPSETLSLTCCVSGGSISSSSYYWGWYRQPPGKGLEWIGSIYYSGSTYYNPSLKSRVTISVDTSKNCFSLKLSDVTPADTAVYYCAR

SEQ ID NO: 598 - protein, 98 aa, synthetic construct
  REGION 1..98: note = Modified VH domain from a human immunoglobulin germline
QVQLQESGPGLVKPSGTLSLTCCVSGGSISSSNWWSWVRRPPGKGLEWIGEIYHSGSTNYNPSLKSRVTISVDKSKNCFSLKLSDVTPADTAVYYCAR

SEQ ID NO: 599 - protein, 98 aa, synthetic construct
  REGION 1..98: note = Modified VH domain from a human immunoglobulin germline
QVQLQESGPGLVKPSGTLSLTCCVSGGSISSSNWWSWVRRPPGKGEEWIGEIYHSGSTNYNPSLKSRVTISVDKSKNCFSLKLSDVTPADTAVYYCAR

SEQ ID NO: 600 - protein, 98 aa, synthetic construct
  REGION 1..98: note = Modified VH domain from a human immunoglobulin germline
QVQLQESGPGLVKPSGTLSLTCCVSGGSISSSNWWSWYRQPPGKGLEWIGEIYHSGSTNYNPSLKSRVTISVDKSKNCFSLKLSDVTPADTAVYYCAR

SEQ ID NO: 601 - protein, 97 aa, synthetic construct
  REGION 1..97: note = Modified VH domain from a human immunoglobulin germline
QVQLQESGPGLVKPSETLSLTCCVSGGSISSYYWSWIRRPPGKGLEWIGYIYYSGSTNYNPSLKSRVTISVDTSKNCFSLKLSDVTPADTAVYYCAR

SEQ ID NO: 602 - protein, 97 aa, synthetic construct
  REGION 1..97: note = Modified VH domain from a human immunoglobulin germline
QVQLQESGPGLVKPSETLSLTCCVSGGSISSYYWSWIRRPPGKGEEWIGYIYYSGSTNYNPSLKSRVTISVDTSKNCFSLKLSDVTPADTAVYYCAR

SEQ ID NO: 603 - protein, 97 aa, synthetic construct
  REGION 1..97: note = Modified VH domain from a human immunoglobulin germline
QVQLQESGPGLVKPSETLSLTCCVSGGSISSYYWSWYRQPPGKGLEWIGYIYYSGSTNYNPSLKSRVTISVDTSKNCFSLKLSDVTPADTAVYYCAR

SEQ ID NO: 604 - protein, 99 aa, synthetic construct
  REGION 1..99: note = Modified VH domain from a human immunoglobulin germline
QVQLQESGPGLVKPSETLSLTCCVSGGSVSSGSYYWSWIRRPPGKGLEWIGYIYYSGSTNYNPSLKSRVTISVDTSKNCFSLKLSDVTPADTAVYYCAR

SEQ ID NO: 605 - protein, 99 aa, synthetic construct
  REGION 1..99: note = Modified VH domain from a human immunoglobulin germline
QVQLQESGPGLVKPSETLSLTCCVSGGSVSSGSYYWSWIRRPPGKGEEWIGYIYYSGSTNYNPSLKSRVTISVDTSKNCFSLKLSDVTPADTAVYYCAR

SEQ ID NO: 606 - protein, 99 aa, synthetic construct
  REGION 1..99: note = Modified VH domain from a human immunoglobulin germline
QVQLQESGPGLVKPSETLSLTCCVSGGSVSSGSYYWSWYRQPPGKGLEWIGYIYYSGSTNYNPSLKSRVTISVDTSKNCFSLKLSDVTPADTAVYYCAR

SEQ ID NO: 607 - protein, 98 aa, synthetic construct
  REGION 1..98: note = Modified VH domain from a human immunoglobulin germline
QVQLQESGPGLVKPSETLSLTCCVSGYSISSGYYWGWIRRPPGKGLEWIGSIYHSGSTYYNPSLKSRVTISVDTSKNCFSLKLSDVTPADTAVYYCAR

SEQ ID NO: 608 - protein, 98 aa, synthetic construct
  REGION 1..98: note = Modified VH domain from a human immunoglobulin germline
QVQLQESGPGLVKPSETLSLTCCVSGYSISSGYYWGWIRRPPGKGEEWIGSIYHSGSTYYNPSLKSRVTISVDTSKNCFSLKLSDVTPADTAVYYCAR

SEQ ID NO: 609 - protein, 98 aa, synthetic construct
  REGION 1..98: note = Modified VH domain from a human immunoglobulin germline
QVQLQESGPGLVKPSETLSLTCCVSGYSISSGYYWGWYRQPPGKGLEWIGSIYHSGSTYYNPSLKSRVTISVDTSKNCFSLKLSDVTPADTAVYYCAR

SEQ ID NO: 610 - protein, 100 aa, synthetic construct
  REGION 1..100: note = Modified VH domain from a human immunoglobulin germline
QVTLKESGPVLVKPTETLCLTCTVSGFSLSNARMGVSWYRRPPGKALEWLAHIFSNDEKSYSTSLKSRLTISKDTSKSQVVLCMTNMTPVDTATYYCARI

SEQ ID NO: 611 - protein, 100 aa, synthetic construct
  REGION 1..100: note = Modified VH domain from a human immunoglobulin germline
QVTLKESGPVLVKPTETLCLTCTVSGFSLSNARMGVSWYRRPPGKAEEWLAHIFSNDEKSYSTSLKSRLTISKDTSKSQVVLCMTNMTPVDTATYYCARI

SEQ ID NO: 612 - protein, 100 aa, synthetic construct
  REGION 1..100: note = Modified VH domain from a human immunoglobulin germline
QVTLKESGPVLVKPTETLCLTCTVSGFSLSNARMGVSWYRQPPGKALEWLAHIFSNDEKSYSTSLKSRLTISKDTSKSQVVLCMTNMTPVDTATYYCARI

SEQ ID NO: 613 - protein, 100 aa, synthetic construct
  REGION 1..100: note = Modified VH domain from a human immunoglobulin germline
QITLKESGPTLVKPTQTLCLTCTFSGFSLSTSGVGVGWYRRPPGKALEWLALIYWNDDKRYSPSLKSRLTITKDTSKNQVVLCMTNMTPVDTATYYCAHR

SEQ ID NO: 614 - protein, 100 aa, synthetic construct
  REGION 1..100: note = Modified VH domain from a human immunoglobulin germline
QITLKESGPTLVKPTQTLCLTCTFSGFSLSTSGVGVGWYRRPPGKAEEWLALIYWNDDKRYSPSLKSRLTITKDTSKNQVVLCMTNMTPVDTATYYCAHR

SEQ ID NO: 615 - protein, 100 aa, synthetic construct
  REGION 1..100: note = Modified VH domain from a human immunoglobulin germline
QITLKESGPTLVKPTQTLCLTCTFSGFSLSTSGVGVGWYRQPPGKALEWLALIYWNDDKRYSPSLKSRLTITKDTSKNQVVLCMTNMTPVDTATYYCAHR

SEQ ID NO: 616 - protein, 100 aa, synthetic construct
  REGION 1..100: note = Modified VH domain from a human immunoglobulin germline
QVTLRESGPALVKPTQTLCLTCTFSGFSLSTSGMCVSWYRRPPGKALEWLALIDWDDDKYYSTSLKTRLTISKDTSKNQVVLCMTNMTPVDTATYYCARI

SEQ ID NO: 617 - protein, 100 aa, synthetic construct
  REGION 1..100: note = Modified VH domain from a human immunoglobulin germline
QVTLRESGPALVKPTQTLCLTCTFSGFSLSTSGMCVSWYRRPPGKAEEWLALIDWDDDKYYSTSLKTRLTISKDTSKNQVVLCMTNMTPVDTATYYCARI

SEQ ID NO: 618 - protein, 100 aa, synthetic construct
  REGION 1..100: note = Modified VH domain from a human immunoglobulin germline
QVTLRESGPALVKPTQTLCLTCTFSGFSLSTSGMCVSWYRQPPGKALEWLALIDWDDDKYYSTSLKTRLTISKDTSKNQVVLCMTNMTPVDTATYYCARI

SEQ ID NO: 619 - protein, 98 aa, synthetic construct
  REGION 1..98: note = Modified VH domain from a human immunoglobulin germline
EVQLVQSGAEVKKPGESLRISCKGSGYDFTSYWISWVRRMPGKGLEWMGRIDPSDSYTNYSPSFQGHVTISADKSINTAYLQWSSLKESDTAMYYCAR

SEQ ID NO: 620 - protein, 98 aa, synthetic construct
  REGION 1..98: note = Modified VH domain from a human immunoglobulin germline
EVQLVQSGAEVKKPGESLRISCKGSGYDFTSYWISWVRRMPGKGEEWMGRIDPSDSYTNYSPSFQGHVTISADKSINTAYLQWSSLKESDTAMYYCAR

SEQ ID NO: 621 - protein, 98 aa, synthetic construct
  REGION 1..98: note = Modified VH domain from a human immunoglobulin germline
EVQLVQSGAEVKKPGESLRISCKGSGYDFTSYWISWYRQMPGKGLEWMGRIDPSDSYTNYSPSFQGHVTISADKSINTAYLQWSSLKESDTAMYYCAR

SEQ ID NO: 622 - protein, 98 aa, synthetic construct
  REGION 1..98: note = Modified VH domain from a human immunoglobulin germline
EVQLVQSGAEVKKPGESLKISCKGSGYDFTSYWIGWVRRMPGKGLEWMGIIYPGDSDTRYSPSFQGQVTISADKSINTAYLQWSSLKESDTAMYYCAR

SEQ ID NO: 623 - protein, 98 aa, synthetic construct
  REGION 1..98: note = Modified VH domain from a human immunoglobulin germline
EVQLVQSGAEVKKPGESLKISCKGSGYDFTSYWIGWVRRMPGKGEEWMGIIYPGDSDTRYSPSFQGQVTISADKSINTAYLQWSSLKESDTAMYYCAR

SEQ ID NO: 624 - protein, 98 aa, synthetic construct
  REGION 1..98: note = Modified VH domain from a human immunoglobulin germline
EVQLVQSGAEVKKPGESLKISCKGSGYDFTSYWIGWYRQMPGKGLEWMGIIYPGDSDTRYSPSFQGQVTISADKSINTAYLQWSSLKESDTAMYYCAR

SEQ ID NO: 625 - protein, 98 aa, synthetic construct
  REGION 1..98: note = Modified VH domain from a human immunoglobulin germline
QVQLVQSGSELKKPGACVKVSCKASGYTFTSYAMNWVRRAPGQGLEWMGWINTNTGNPTYAQGFTGRFVFSLDTSVSTAYLQICSLKAEDTAVYYCAR

SEQ ID NO: 626 - protein, 98 aa, synthetic construct
  REGION 1..98: note = Modified VH domain from a human immunoglobulin germline
QVQLVQSGSELKKPGACVKVSCKASGYTFTSYAMNWVRRAPGQGEEWMGWINTNTGNPTYAQGFTGRFVFSLDTSVSTAYLQICSLKAEDTAVYYCAR

SEQ ID NO: 627 - protein, 98 aa, synthetic construct
  REGION 1..98: note = Modified VH domain from a human immunoglobulin germline
QVQLVQSGSELKKPGACVKVSCKASGYTFTSYAMNWYRQAPGQGLEWMGWINTNTGNPTYAQGFTGRFVFSLDTSVSTAYLQICSLKAEDTAVYYCAR

SEQ ID NO: 628 - protein, 127 aa, synthetic construct; identical to SEQ ID NO: 362
  REGION 1..127: note = Modified VH domain from a human immunoglobulin germline
  VARIANT 98..116: note = X is any residue

SEQ ID NO: 629 - protein, 127 aa, synthetic construct; identical to SEQ ID NO: 363
  REGION 1..127: note = Modified VH domain from a human immunoglobulin germline
  VARIANT 98..116: note = X is any residue

SEQ ID NO: 630 - protein, 127 aa, synthetic construct; identical to SEQ ID NO: 365
  REGION 1..127: note = Modified VH domain from a human immunoglobulin germline
  VARIANT 98..116: note = X is any residue

SEQ ID NO: 631 - protein, 127 aa, synthetic construct; identical to SEQ ID NO: 366
  REGION 1..127: note = Modified VH domain from a human immunoglobulin germline
  VARIANT 98..116: note = X is any residue

SEQ ID NO: 632 - protein, 118 aa, synthetic construct; identical to SEQ ID NO: 438
  REGION 1..118: note = Modified VH domain from a human immunoglobulin germline
  VARIANT 98..107: note = X is any residue

SEQ ID NO: 633 - protein, 128 aa, synthetic construct; identical to SEQ ID NO: 441
  REGION 1..128: note = Modified VH domain from a human immunoglobulin germline
  VARIANT 97..117: note = X is any residue